(19) United States
(12) Patent Application Publication
(10) Pub. No.: US 2005/0169994 A1
(43) Pub. Date: Aug. 4, 2005

(54) Carvedilol free base, salts, anhydrous forms or solvates thereof, corresponding pharmaceutical compositions, controlled release formulations, and treatment or delivery methods

(76) Inventors: Matthew D. Burke, Research Triangle Park, NC (US); Mark Davis Coffin, Research Triangle Park, NC (US); Kimberly A. Lamey, King of Prussia, PA (US); Luigi G. Martini, Essex (GB); Choon K. Oh, Collegeville, PA (US); Heather Peterson, Research Triangle Park, NC (US); Jeffrey Scott Staton, Research Triangle Park, NC (US); Lihua Zhang, Research Triangle Park, NC (US)

Correspondence Address: GLAXOSMITHKLINE;Corporate Intellectual Property - UW2220, P.O. Box 1539, King of Prussia, PA, 19406-0939, US

(21) Appl. No.: 10/996,904

(22) Filed: Nov. 24, 2004

Related U.S. Application Data
(60) Provisional application No. 60/524,991, filed on Nov. 25, 2003.

(51) Int. Cl. A61K 9/22; A61K 9/26; A61K 9/24

(52) U.S. Cl. 424/469; 424/473

(57) ABSTRACT

The present invention also relates to carvedilol free base, salts, anhydrous forms, or solvates thereof, corresponding pharmaceutical compositions or controlled release formulations, and methods delivery of carvedilol forms to the lower gastrointestingal tract or methods to treat cardiovascular diseases, which may include, but are not limited to hypertension, congestive heart failure, and angina. The present invention relates to control release formulations, which comprise various cavedilol forms, which may include, but are not limited to carvedilol free base and corresponding carvedilol salts, anhydrous forms or solvates thereof.

### FIELD OF THE INVENTION

The present invention relates to carvedilol free base or carvedilol salts, anhydrous forms, or solvates thereof, corresponding pharmaceutical compositions or controlled release formulations, and delivery methods of carvedilol forms to the gastrointestinal tract or methods to treat cardiovascular diseases, which may include, but are not limited to hypertension, congestive heart failure, atherosclerosis, and angina.

The present invention relates to controlled release formulations, which comprise various cavedilol forms, which may include, but are not limited to carvedilol free base and corresponding carvedilol salts, anhydrous forms or solvates thereof.

### BACKGROUND OF THE INVENTION

Carvedilol

The compound, 1-(carbazol-4-yloxy-3-[[2-(o-methoxyphenoxy) ethyl]-amino]-2-propanol is known as Carvedilol. Carvedilol is depicted by the following chemical structure:

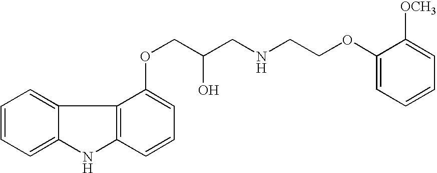

Carvedilol is disclosed in U.S. Pat. No. 4,503,067 to Wiedemann et al. (i.e., assigned to Boehringer Mannheim, GmbH, Mannheim-Waldhof, Fed. Rep. of Germany), which was issued on Mar. 5, 1985.

Currently, carvedilol is synthesized as free base for incorporation in medication that is available commercially. The aforementioned free base form of carvedilol is a racemic mixture of R(+) and S(−) enantiomers, where non-selective β-adrenoreceptor blocking activity is exhibited by the S(−) enantiomer and α-adrenergic blocking activity is exhibited by both R(+) and S(−) enantiomers. Those unique features or characteristics associated with such a racemic carvedilol mixture contributes to two complementary pharmacologic actions: i.e., mixed venous, arterial vasodilation and non-cardioselective, beta-adrenergic blockade.

Carvedilol is used for treatment of hypertension, congestive heart failure, and angina.

The currently commercially available carvedilol product is a conventional, tablet prescribed as a twice-a-day (BID) medication in the United States. The commercially available carvedilol formulation is in an immediate release or rapidly releasing carvedilol in its free base form, where the nature or the chemical and physical formulation properties are such that by the time the carvedilol leaves the stomach, it is either in solution or it is in the form of a suspension of fine particles, i.e. a form from which carvedilol can be readily absorbed.

Carvedilol contains an α-hydroxyl secondary amine functional group, which has a pKa of 7.8. Carvedilol exhibits predictable solubility behaviour in neutral or alkaline media, i.e. above a pH of 9.0, the solubility of carvedilol is relatively low (<1 μg/mL). The solubility of carvedilol increases with decreasing pH and reaches a plateau near pH=5, i.e. where saturation solubility is about 23 μg/mL at pH=7 and about 100 μg/mL at pH=5 at room temperature. At lower pH values (i.e., at a pH of 1 to 4 in various buffer systems), solubility of carvedilol is limited by the solubility of its protonated form or its corresponding salt formed in-situ. For example, a hydrochloride salt of carvedilol generated in situ in an acidic medium, which simulates gastric fluid, is less soluble in such medium.

In addition, the presence of the α-hydroxyl secondary amine group in the carvedilol chemical structure confers a propensity upon the compound to chemically react with excipients normally included in a dosage form to aid manufacture, maintain quality, or enhances dissolution rate. For example, the α-hydroxyl secondary amine group of carvedilol can react with aldehydes or ester functional groups through nucleophilic reactions. Common chemical functional group residues associated with conventionally used excipients, include ester, aldehyde or other chemical residue functional groups. This often results in marginal or unacceptable chemical stability upon storage.

Pharmaceutical Compositions/Formulations and Controlled-Release Technology

In the medical treatment of mammals, a desire to maintain a constant concentration of a pharmaceutical composition within the blood stream of human or animal patient, is dependent upon regular administration of such a composition, such as in an oral tablet form. Regularity of oral administration of various drug dosage forms is important as a typical pharmaceutical composition form is released immediately upon dissolution in a recipients stomach. Thus, any interruption in a patient's tablet supply regimen causes a consequent drug or pharmaceutical concentration reduction in the patient's blood.

Therefore, for ease of patient use, it is often desirable to maintain a controlled concentration of an pharmaceutical active drug agent or composition at a predetermined site for an extended period of time.

The use of controlled release technology allows for release of a pharmaceutical composition at a constant rate at a desired concentration into a patient's system over many hours. For example, if a controlled release tablet contains a sufficient drug or composition amount to maintain a desired concentration for twelve or more hours, there would be no need for a patient to take tablets frequently and would reduce interrupting a patient's drug regime.

As conventionally known in the art, many different examples have been developed to accomplish such results.

For example, U.S. Pat. No. 3,845,770 to Theeuwes et al. teaches a device that provides a controlled release via a core tablet including an active agent coated with a semipermeable membrane permeable only to a fluid present in the environment of use (i.e., water), where the active agent or another component of the core tablet exhibits osmotic activity and the rate of release is dependent upon the permeability of the semipermeable membrane.

U.S. Pat. No.4,624,847 to Ayer et al. describes an osmotic dispensing device, where a drug mixed with an osmopolymer or osmagent is in a compartment surrounded by a semipermeable wall with an osmotic passageway to the compartment. Other patents describing various osmotic dispensing devices include: U.S. Pat. No.4,519,801 to Edgren; U.S. Pat. No. 4,111,203 to Theeuwes; U.S. Pat. No. 4,777,049 to Magruder et al.; U.S. Pat. No. 4,612,008 to Wong et al.; U.S. Pat. No. 4,610,686 to Ayer et al.; U.S. Pat. No. 4,036,227 to Zaffaroni et al.; U.S. Pat. No. 4,553,973 to Edgren; U.S. Pat. No. 4,077,407 to Theeuwes et al.; and U.S. Pat. No. 4,609,374 to Ayer.

U.S. Pat. No.4,218,433 to Kooichi et al. describes a tablet with a water insoluble coating and a water soluble component that releases an active component at a constant rate due to an indentation formed on its surface.

U.S. Pat. No.4,687,660 to Baker et al. describes an osmotic dispensing device without a preformed single passageway to release water-soluble drugs, where based upon an osmotic gradient formed from water insoluble film coated core containing a drug is combined with excipient and an osmotic enhancing agent.

U.S. Pat. No.4,816,262 to McMullen relates to a controlled release disc-like configured tablet with a centrally extending cylindrical hole that allows for zero order or constant release of the active agent.

Devices with an impermeable coating covering various portions of the device include: U.S. Pat. No.4,814,183 to Zentner relates to a controlled release device with a charged resin core encased in a water insoluble semi-permeable material that is impermeable to core components, but permeable to the passage of an external fluid in the environment of use. U.S. Pat. No. 4,814,182 to Graham et al. describes a controlled release device which comprises an active ingredient/hydrogel mixture with at least one surface of the device having a coating impermeable to aqueous media. U.S. Pat. No. 4,792,448 to Ranade relates to a cylindrical tablet or bolus with a impermeable coated core having an active ingredient blended with inert excipients and formed into a cylindrical tablet preferably having a flat cylindrical side and a convex top and bottom.

Moreover, numerous prior art references also describe producing alternate sustained-release systems, with the aim of providing medicinal forms, which may be taken once a day, to prolong the action of a medicinal product. (see, for example, Formes Pharmaceutiques Nouvelles, Buri, Puisieux, Doelker et Benoit, Lavoisier 1985, pages 175-227).

These include monolithic systems, where dose to be administered is in the form of a solid object, such as a tablet. DE Pat. Appn. No. 39 43 242 (FR No. 2 670 112) discloses “matrix” type granules, which comprise active particles (“AP”) and inert excipent(s), useful for making tablets. Such granules, which are distinct from microcapsules, consist of a multitude of particles included in a roughly spherical matrix comprising a cellulosic polymer, a vinylic or acrylic polymer, a plasticizer and a lubricating agent.

U.S. Pat. No. 4,461,759 to Dunn describes a oral solid dosage coated tablet, which includes active particles (“AP” or “AP's”) protected from harmful effects of stomach acidity that are released at a constant rate in the gastrointestinal tract.

U.S. Pat. No. 5,028,434 to Barclay et al. and Inter.I' Appln. No. WO 91/16885 describes a monolithic tablet form using a microporous film coating that allows controlled release of active particles via osmotic pressure.

Other literature examples of microparticulate pharmaceutical systems giving a sustained release of active particles (“AP” or “APs or AP's”) include: U.S. Pat. No. 5,286,497 to Hendrickson et al., which relates to a once a day controlled release diltiazem formulation, which contains a blend of rapid release bead and delayed release coated diltiazem beads with different dissolution rates.

Consequently, the short residence time in the small intestine poses a considerable problem to those skilled in the art interested in developing sustained-absorption medicinal products intended for oral administration. The medicinal product administered orally is, in effect, subject to the natural transit of the gastrointestinal tract, thereby limiting its residence time. Now, the small intestine is the preferred location for systemic absorption and it represents the ideal site for making APs available. Thus, it is easy to appreciate the value of a pharmaceutical form having an increased residence time in the small intestine, in view of the sustained in vivo absorption of an AP, beyond normal transit time in the small intestine.

Many studies have been performed regarding the time for gastrointestinal transit. These studies show that the duration of gastric transit is very variable, in particular as a function of feeding, and that it is between a few minutes and a few hours. On the other hand, the duration of transit in the small intestine is particularly constant and, more precisely, is 3 hours plus or minus one hour (see for example S. S. Davis: Assessment of gastrointestinal transit and drug absorption, in Noval drug delivery and its therapeutic application, Ed L. F. Prescott-W. S. Nimmo, 1989, J. Wiley & Son, page 89-101).

In light of the foregoing, novel carvedilol salt, solvate, or anhydrous forms thereof, corresponding pharmaceutical compositions or controlled release formulations containing carvedilol free base or novel carvedilol salt, solvate, or anhydrous forms thereof, with greater aqueous solubility, chemical stability, prolonged residence time, absorption in the gastrointestingal tract, especially such as in the small intestine, etc. would offer many potential benefits for provision of medicinal products containing the drug carvedilol.

Examples of such benefits would include products with the ability to achieve desired or prolonged drug levels in a systemic system by sustaining absorption along the gastrointestinal tract of mammals (i.e., such as humans), particularly in regions of neutral pH, where a drug, such as carvedilol, has minimal solubility.

Surprisingly, it has now been shown that novel forms of carvedilol salts, anhydrous forms or solvates thereof, which may be isolated as, but not limited to crystalline or other solid forms, exhibit much higher aqueous solubility than the corresponding free base or other prepared carvedilol salts, which may include, but are not limited to crystalline forms or other solid forms.

Such carvedilol salts, anhydrous forms or solvates thereof, which may include, but are not limited to novel crystalline or other solid forms, also have potential to improve the stability of carvedilol in pharmaceutical compositions or controlled-release formulations due to the fact that the secondary amine functional group attached to the carvedilol core structure, a moiety pivotal to degradation processes, is protonated as a salt.

Such carvedilol salts, anhydrous forms or solvates thereof, which may include, but are not limited to novel crystalline or other solid forms alone, in pharmaceutical compositions or controlled-release formulations also have potential to lead to prolonged residence, absorption time, and/or good tolerance levels in the gastrointestinal tract, such as the small intestine, colon, etc.

In light of the above, a need exists to develop carvedilol free base or different carvedilol salts, anhydrous forms or solvates forms thereof, different corresponding compositions or controlled release formulations, respectively, which have greater aqueous solubility, chemical stability, good tolerance levels, sustained or prolonged drug or absorption properties or transit levels (i.e., such as in neutral gastrointestinal tract pH regions, etc.).

There also exists a need to develop methods of delivery of carvedilol (such as carvedilol free base or as a carvedilol salt, solvate or anhydrous form thereof) to the gastrointestinal tract or methods of treatment for cardiovascular diseases or associated disorders, which may include, but are not limited to hypertension, congestive heart failure, atherosclerosis, or angina, etc., which comprises administration of carvedilol free base or a carvedilol salt, anhydrous or solvate forms thereof, corresponding pharmaceutical compositions, or controlled release dosage formulations.

The present invention is directed to overcoming these and other problems encountered in the art.

### SUMMARY OF THE INVENTION

The present invention relates to carvedilol free base or carvedilol salts, anhydrous forms, or solvates thereof, corresponding pharmaceutical compositions or controlled release formulations, and delivery methods of carvedilol forms to the gastrointestingal tract or methods to treat cardiovascular diseases, which may include, but are not limited to hypertension, congestive heart failure, atherosclerosis, and angina.

The present invention relates to control release formulations, which comprise various cavedilol forms, which may include, but are not limited to carvedilol free base or corresponding carvedilol salts, anhydrous forms or solvates thereof.

In particular the present invention relates to a controlled release formulation or delivery device, which comprises:

a core containing a carvedilol free base or a carvedilol salt, solvate or anhydrous form thereof; a release modifying agent; and an outer coating covering the core;
where thickness of the outer coating is adapted: for substantial impermeability to entry of fluid present in an environment of use and for substantial impermeability toward release of the carvedilol free base or the carvedilol salt, solvate or anhydrous form thereof during a predetermined dosing interval; and for a controlled release dispensing exit of the carvedilol free base or the carvedilol salt, solvate or anhydrous form thereof after the predetermined dosing interval;
where the outer coating includes at least one orifice in at least one face area of the controlled delivery device extending substantially through the outer coating but not penetrating the core that communicates from the environment of use to the core allowing for release of the carvedilol free base or the carvedilol salt, solvate or anhydrous form thereof into the environment of use;
   where the at least one orifice in the at least one face area of the controlled release delivery device has a substantially dependent rate limiting release factor dependent upon exit of the carvedilol free base or the carvedilol salt, solvate or anhydrous form thereof from the at least one orifice via dissolution, diffusion or erosion; and
   where the release modifying agent enhances or hinders release of the carvedilol free base or the carvedilol salt, solvate or anhydrous form thereof depending upon solubility or effective solubility of the carvedilol free base or the carvedilol salt, solvate or anhydrous form thereof in the environment of use.

The present invention also relates to a controlled release formulation, which comprises at least one of these components:

[a] carvedilol free base, and [b] a solubility enhanced carvedilol salt, solvate or anhydrous forms thereof; or

[a] carvedilol free base, or [b] a solubility enhanced carvedilol salt, solvate or anhydrous forms thereof;

where the aforementioned controlled release formulation following oral dosage exhibits a substantially biphasic plasma profile with a first plasma concentration peak level and a first Tₘₐₓ pulse occurring within 1-4 hours of ingestion and a second a pflasma concentration peak level and second Tₘₐₓ pulse occurring within 5-8 hours after ingestion.

### BRIEF DESCRIPTION OF THE FIGURES

Carvedilol Phosphate Salts

FIG. 1 is an x-ray powder diffractogram for carvedilol dihydrogen phosphate hemihydrate (Form I).

FIG. 2 shows the thermal analysis results for carvedilol dihydrogen phosphate hemihydrate (Form I).

FIG. 3 is an FT-Raman spectrum for carvedilol dihydrogen phosphate hemihydrate (Form I).

FIG. 4 is an FT-Raman spectrum for carvedilol dihydrogen phosphate hemihydrate in the 4000-2000 cm⁻¹ region of the spectrum (Form I).

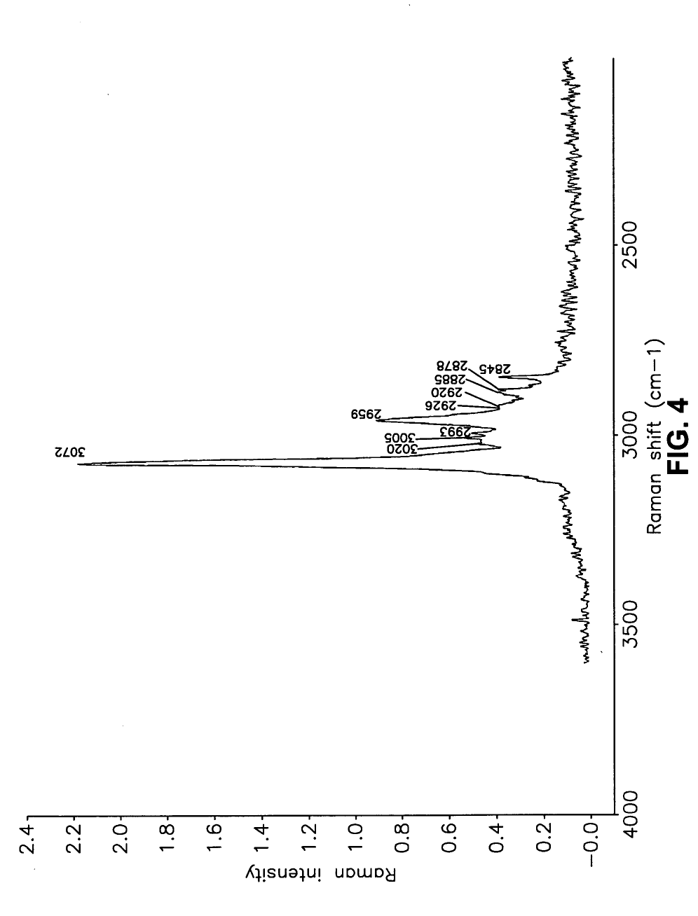

FIG. 5 is an FT-Raman spectrum for carvedilol dihydrogen phosphate hemihydrate in the 2000-400 cm⁻¹ region of the spectrum (Form I).

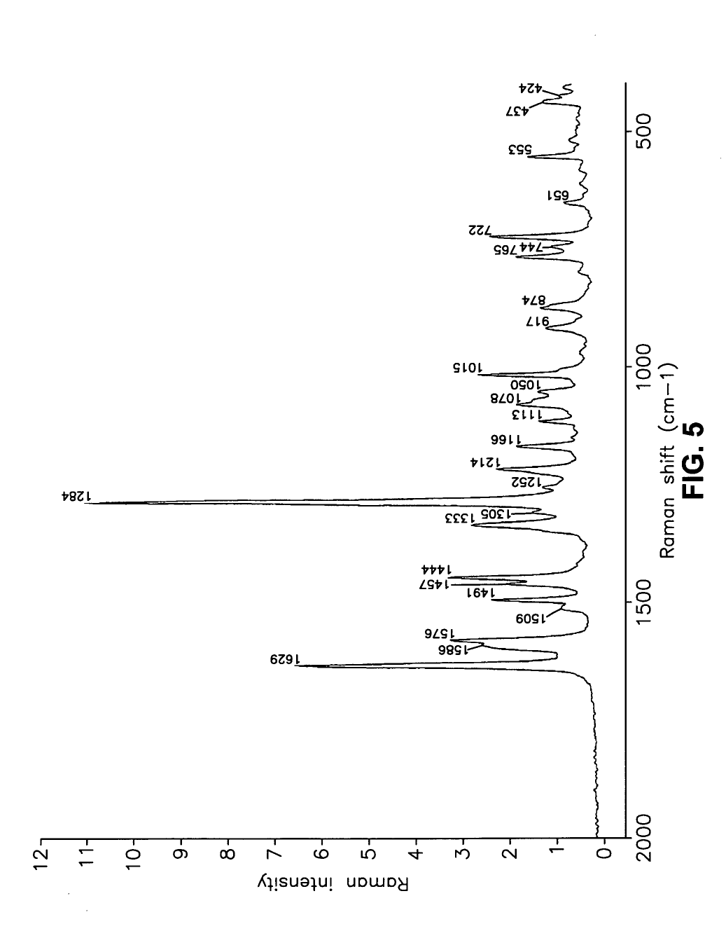

FIG. 6 is an FT-IR spectrum for carvedilol dihydrogen phosphate hemihydrate (Form I).

FIG. 7 is an FT-IR spectrum for carvedilol dihydrogen phosphate hemihydrate in the 4000-2000 cm⁻¹ region of the spectrum (Form I).

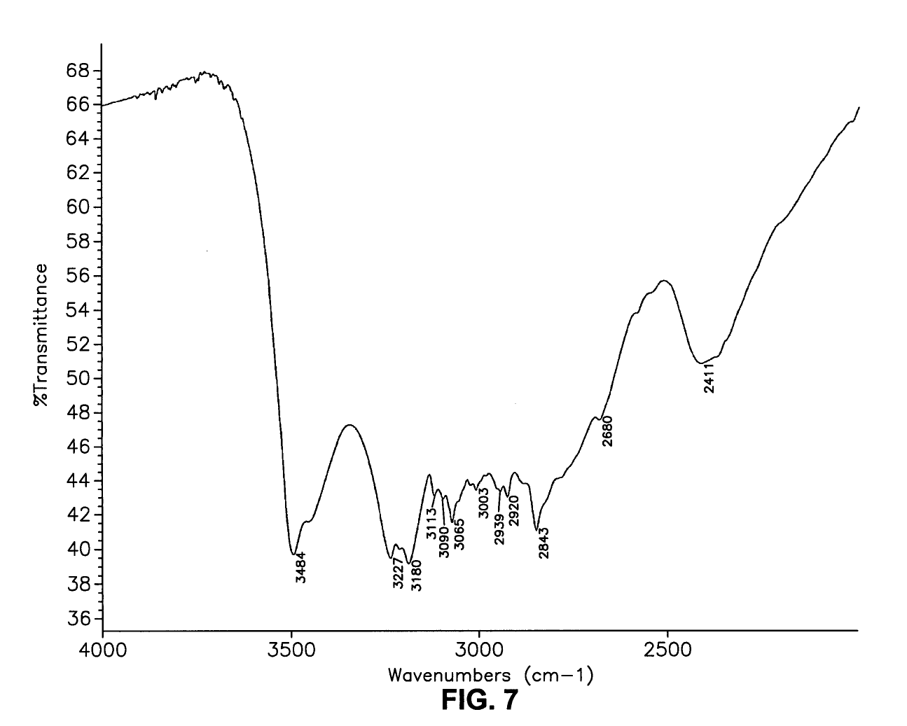

FIG. 8 is an FT-IR spectrum for carvedilol dihydrogen phosphate hemihydrate in the 2000-500 cm⁻¹ region of the spectrum (Form I).

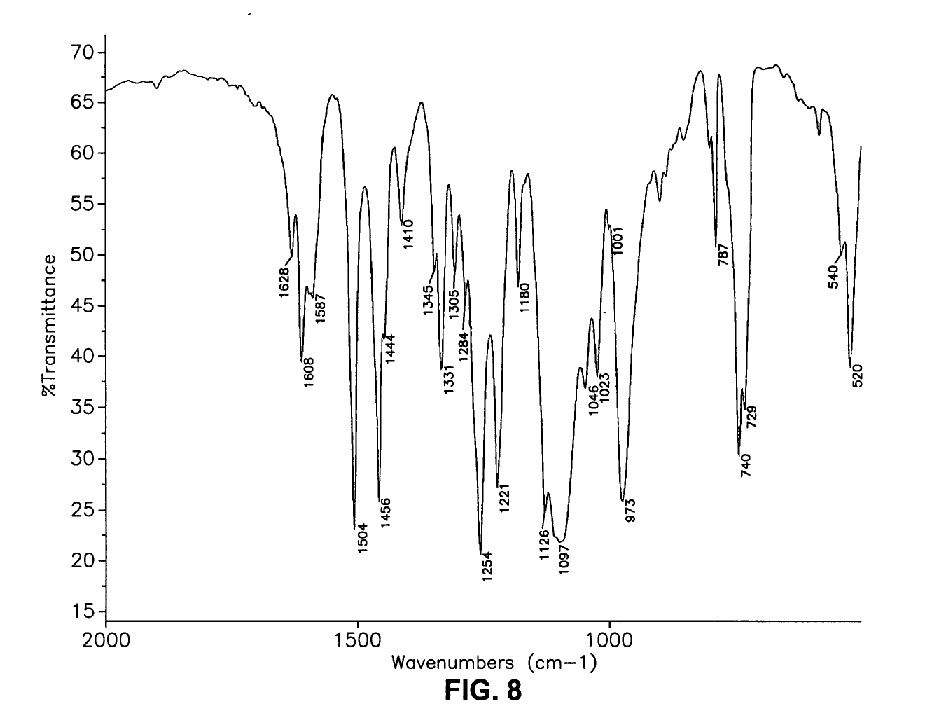

FIG. 9 is an x-ray powder diffractogram for carvedilol dihydrogen phosphate dihydrate (Form II).

FIG. 10 shows the thermal analysis results for carvedilol dihydrogen phosphate dihydrate (Form II).

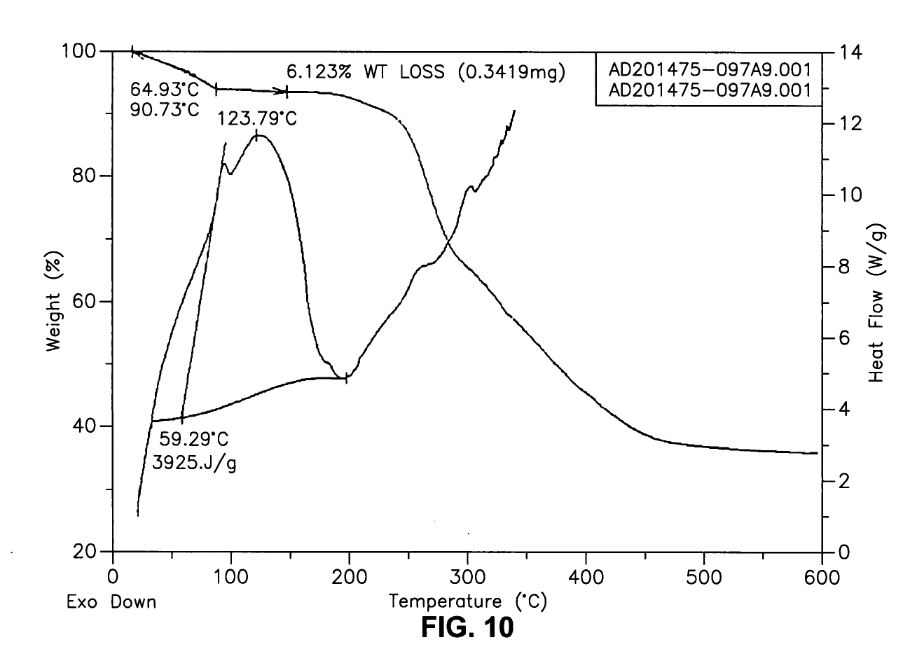

FIG. 11 is an FT-Raman spectrum for carvedilol dihydrogen phosphate dihydrate (Form II).

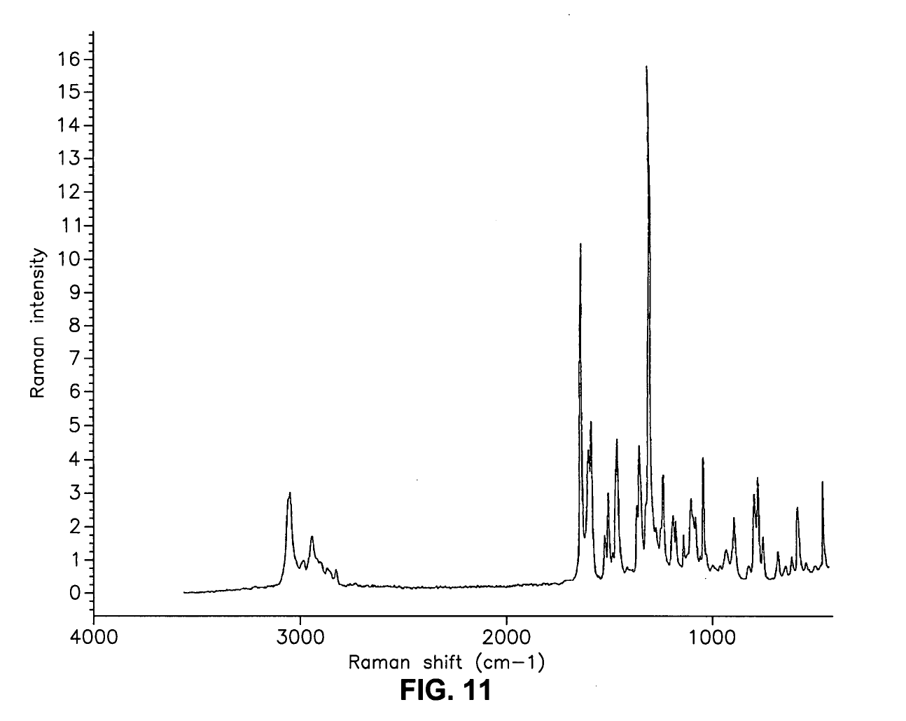

FIG. 12 is an FT-Raman spectrum for carvedilol dihydrogen phosphate dihydrate in the 4000-2000 cm⁻¹ region of the spectrum (Form II).

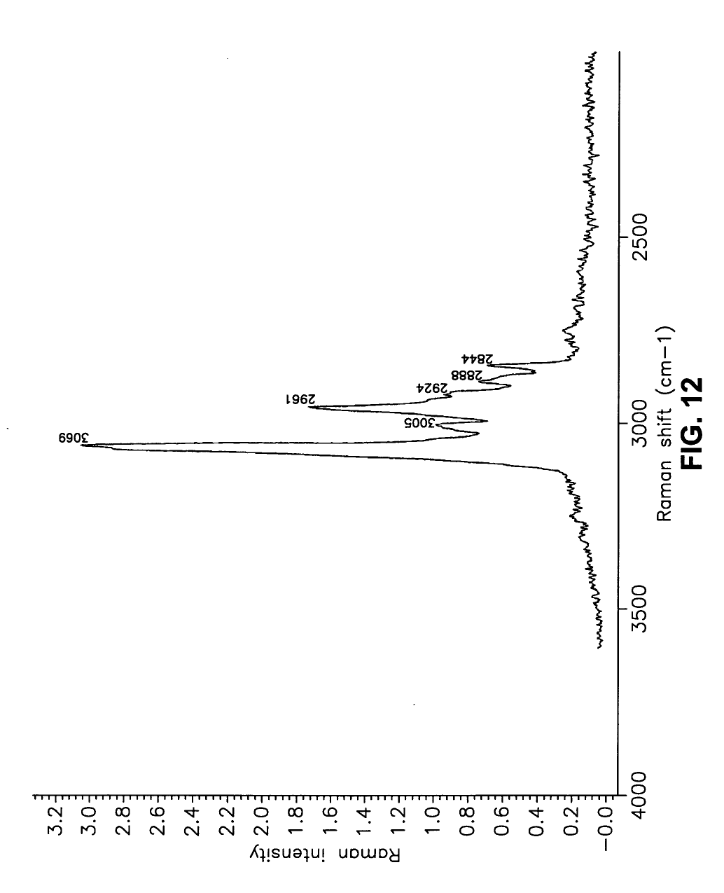

FIG. 13 is an FT-Raman spectrum for carvedilol dihydrogen phosphate dihydrate in the 2000-400 cm⁻¹ region of the spectrum (Form II).

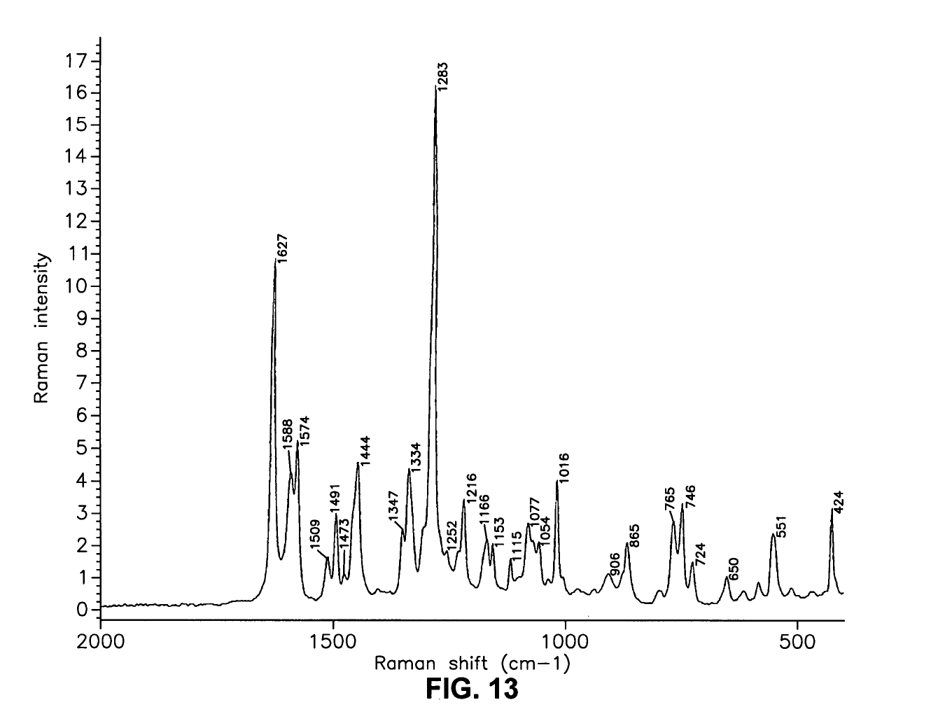

FIG. 14 is an FT-IR spectrum for carvedilol dihydrogen phosphate dihydrate (Form II).

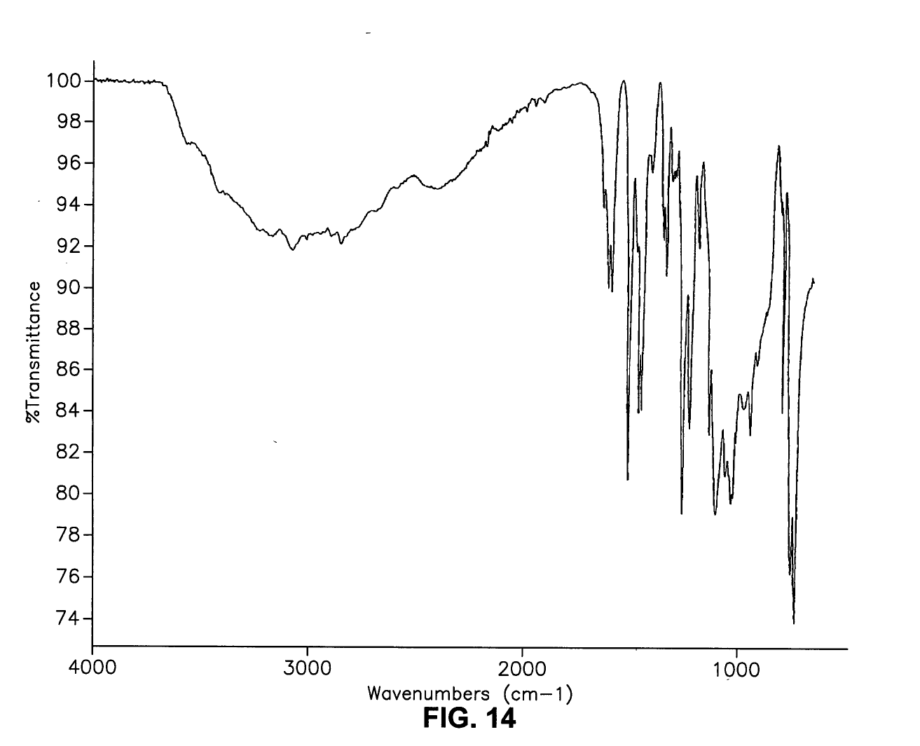

FIG. 15 is an FT-IR spectrum for carvedilol dihydrogen phosphate dihydrate in the 4000-2000 cm⁻¹ region of the spectrum (Form II).

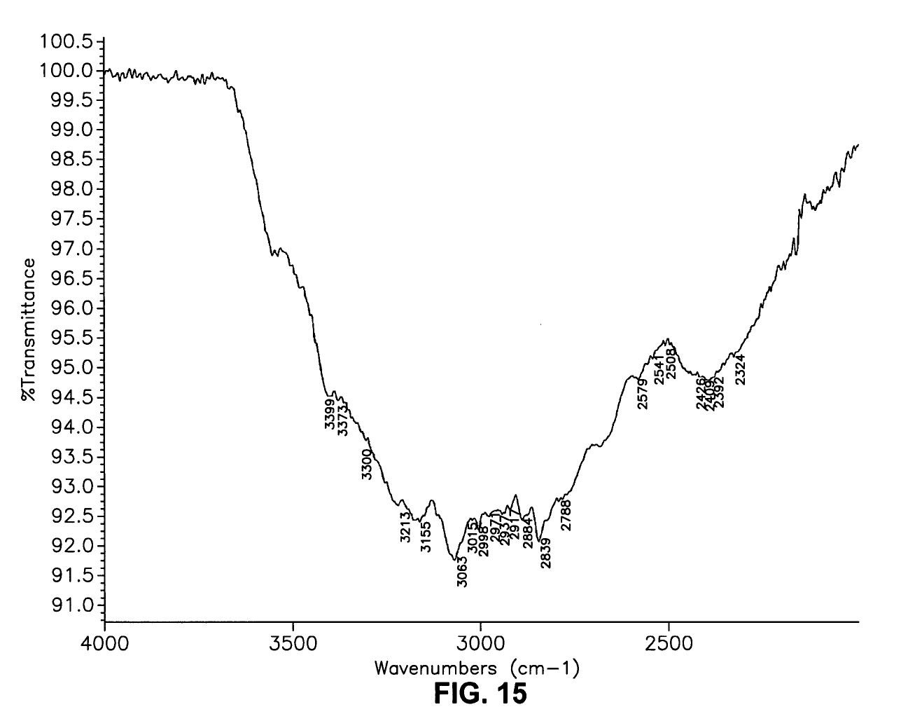

FIG. 16 is an FT-IR spectrum for carvedilol dihydrogen phosphate dihydrate in the 2000-500 cm⁻¹ region of the spectrum (Form II).

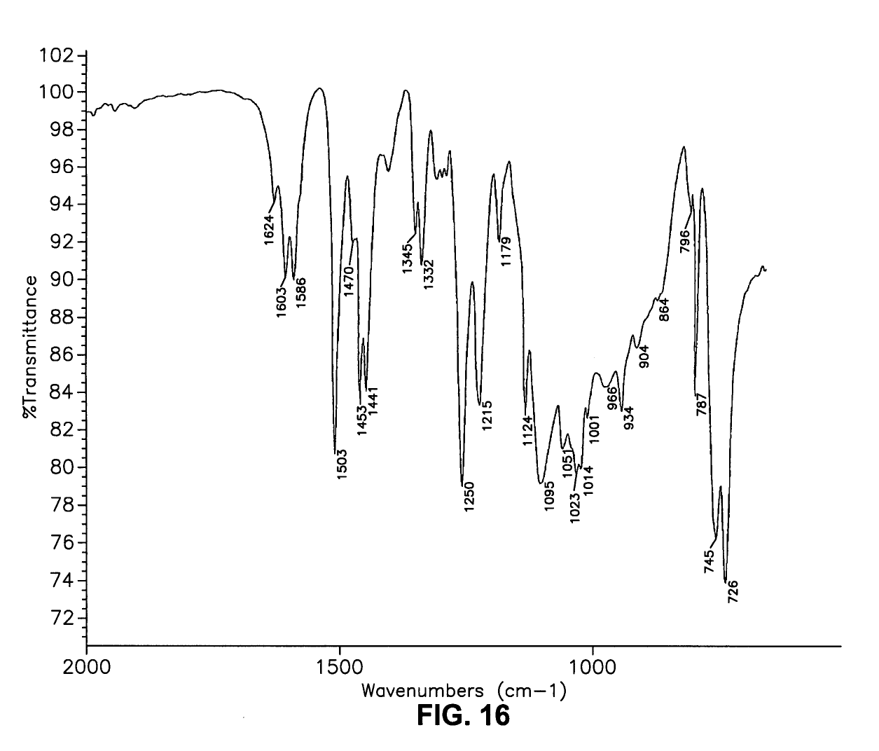

FIG. 17 shows the thermal analysis results for carvedilol dihydrogen phosphate methanol solvate (Form III).

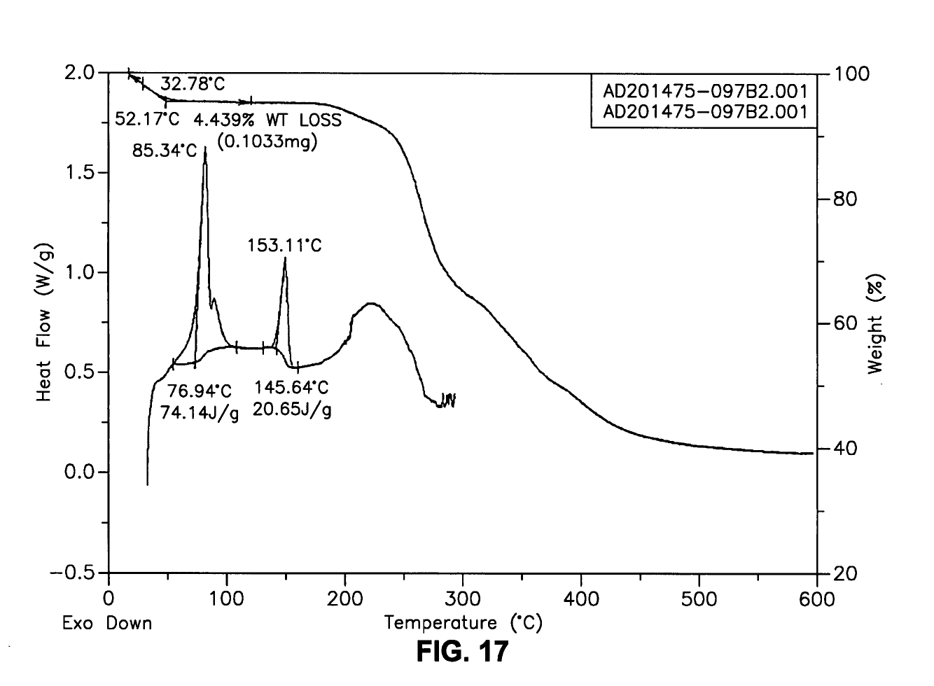

FIG. 18 is an FT-Raman spectrum for carvedilol dihydrogen phosphate methanol solvate (Form III).

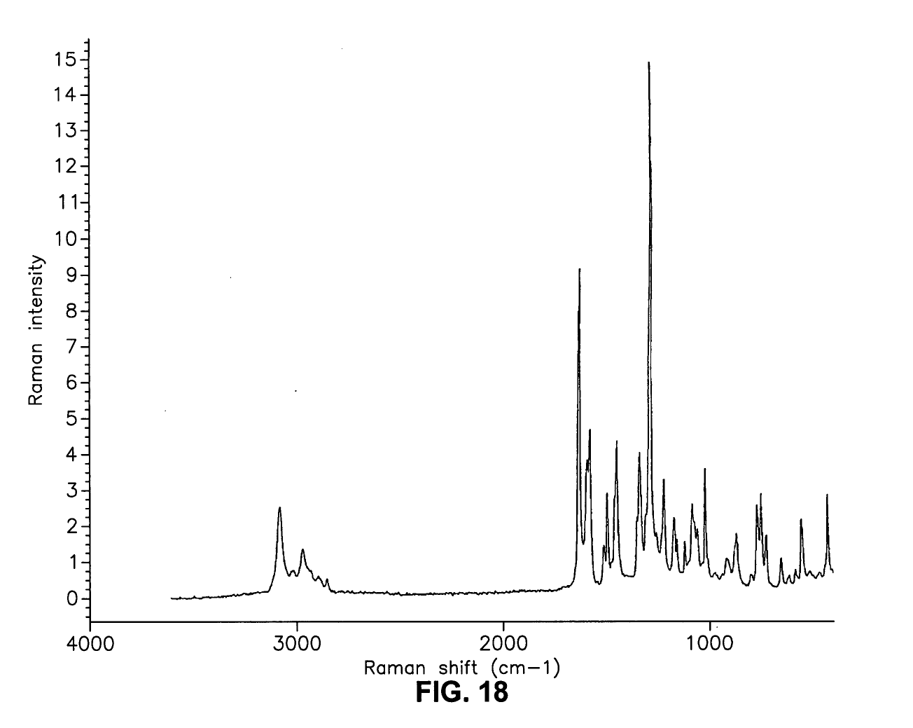

FIG. 19 is an FT-Raman spectrum for carvedilol dihydrogen phosphate methanol solvate in the 4000-2000 cm⁻¹ region of the spectrum (Form III).

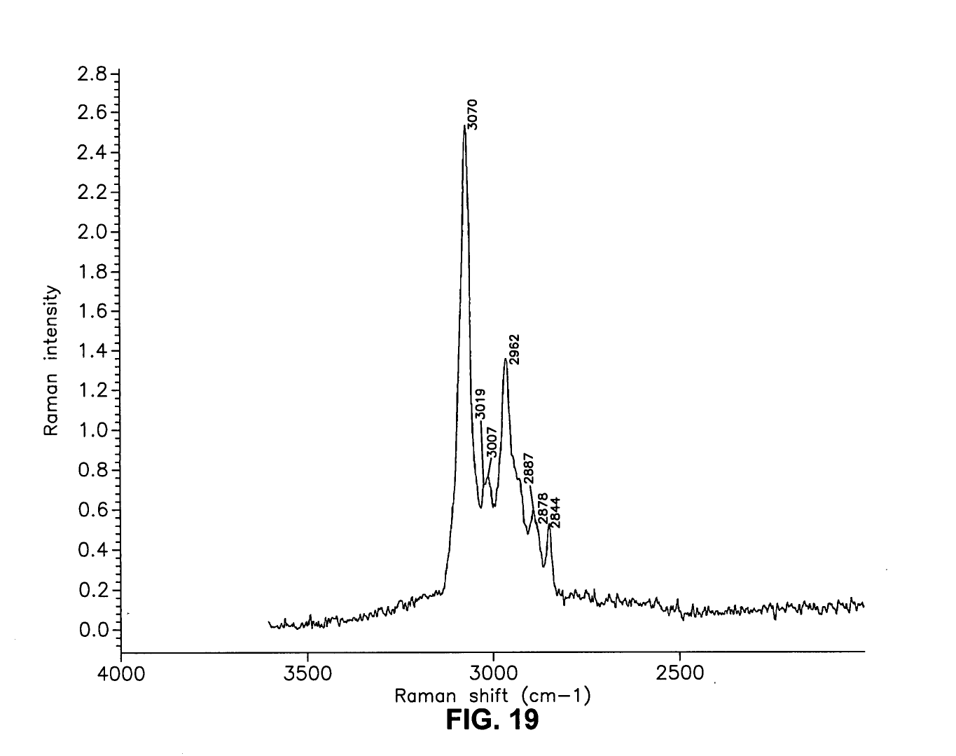

FIG. 20 is an FT-Raman spectrum for carvedilol dihydrogen phosphate methanol solvate in the 2000-400 cm⁻¹ region of the spectrum (Form III).

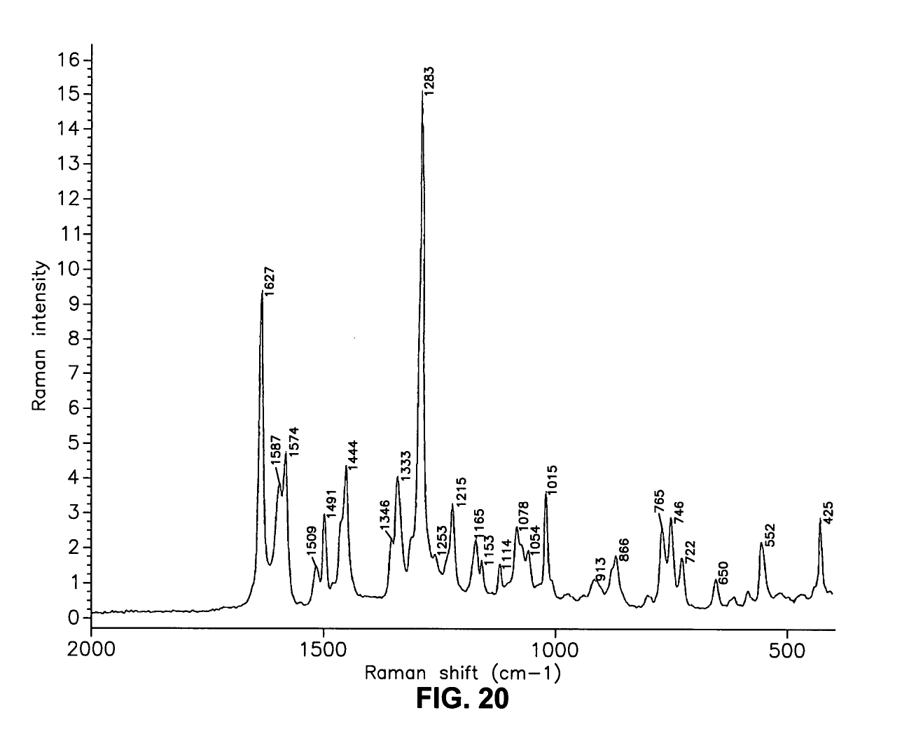

FIG. 21 is an FT-IR spectrum for carvedilol dihydrogen phosphate methanol solvate (Form III).

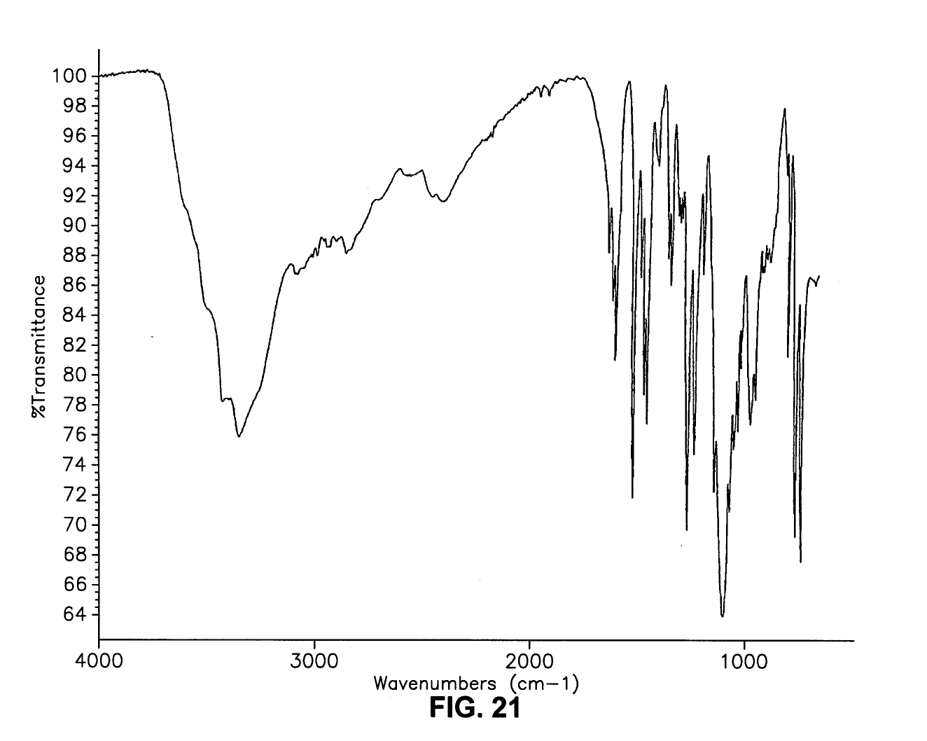

FIG. 22 is an FT-IR spectrum for carvedilol dihydrogen phosphate methanol solvate in the 4000-2000 cm⁻¹ region of the spectrum (Form III).

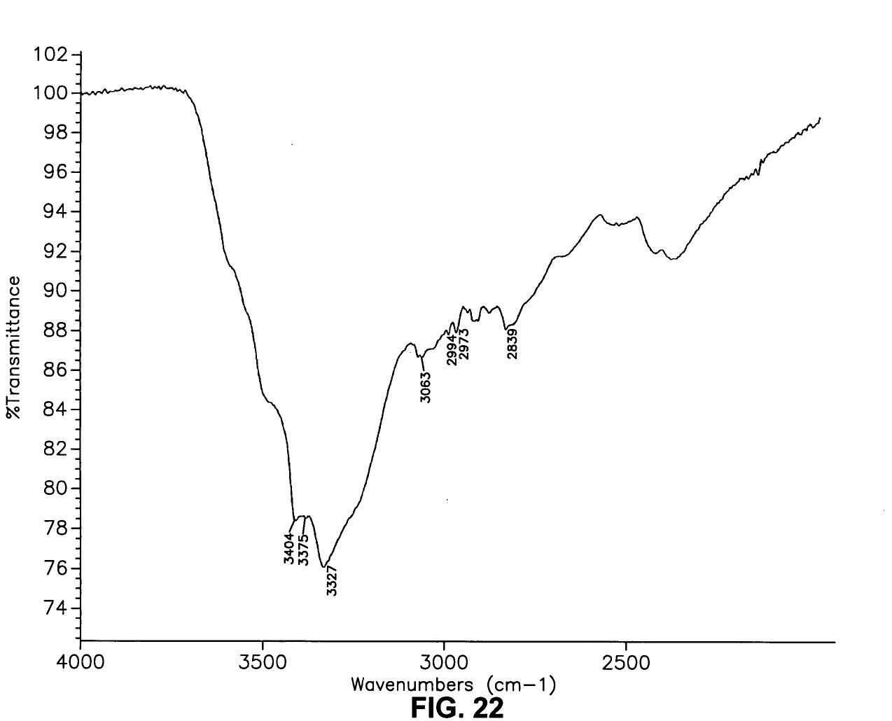

FIG. 23 is an FT-IR spectrum for carvedilol dihydrogen phosphate methanol solvate in the 2000-500 cm⁻¹ region of the spectrum (Form III).

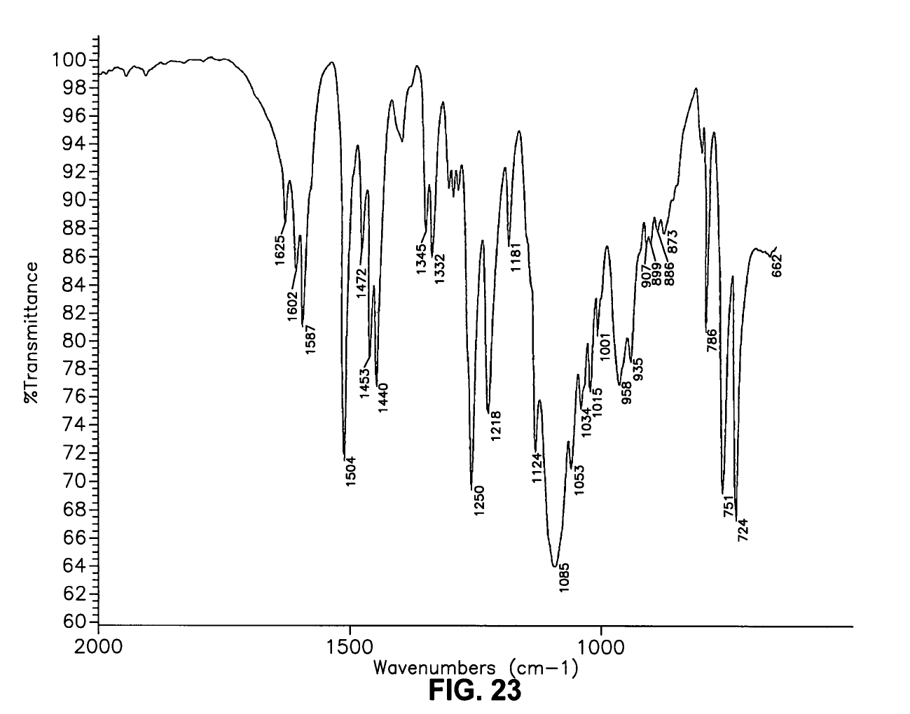

FIG. 24 is an x-ray powder diffractogram for carvedilol dihydrogen phosphate methanol solvate (Form III).

FIG. 25 is an x-ray powder diffractogram for carvedilol dihydrogen phosphate dihydrate (Form IV).

FIG. 26 is a solid state ¹³C NMR for carvedilol dihydrogen phosphate dihydrate (Form I).

FIG. 27 is a solid state ³¹P NMR for carvedilol dihydrogen phosphate dihydrate (Form I).

FIG. 28 is an x-ray powder diffractogram for carvedilol dihydrogen phosphate (Form V).

FIG. 29 is an x-ray powder diffractogram for carvedilol hydrogen phosphate (Form VI).

Carvedilol HBr Salts

FIG. 30 is an x-ray powder diffractogram for carvedilol hydrobromide monohydrate.

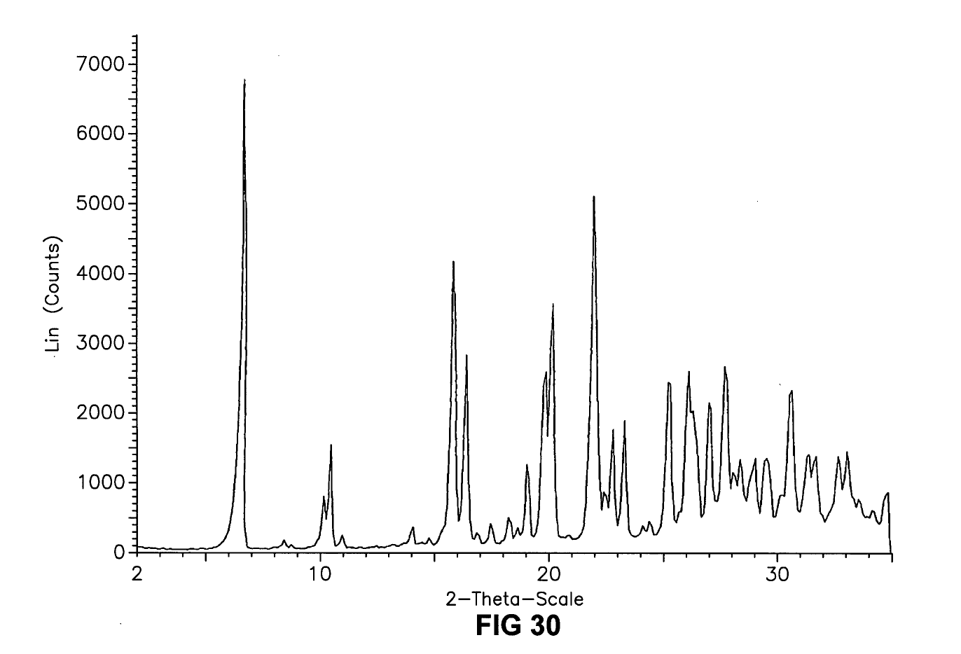

FIG. 31 is a differential scanning calorimetry thermogram for carvedilol hydrobromide monohydrate.

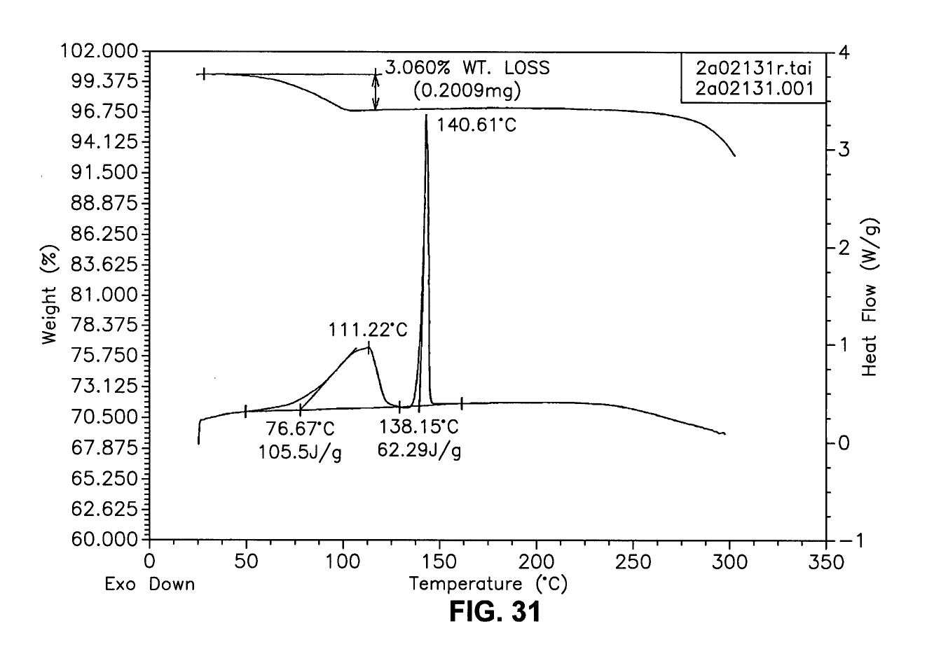

FIG. 32 is an FT-Raman spectrum for carvedilol hydrobromide monohydrate.

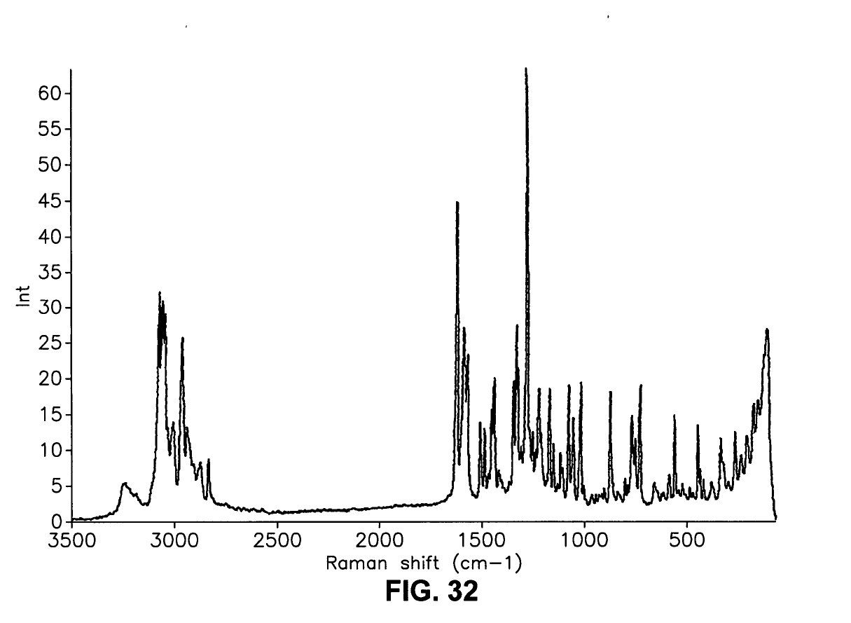

FIG. 33 is an FT-Raman spectrum for carvedilol hydrobromide monohydrate in the 4000-2000 cm⁻¹ region of the spectrum.

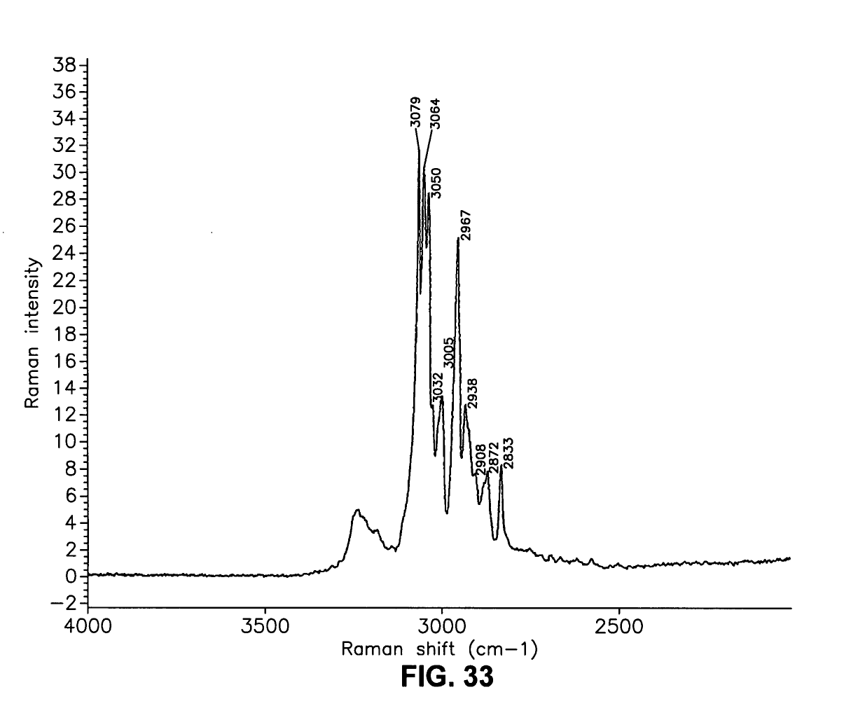

FIG. 34 is an FT-Raman spectrum for carvedilol hydrobromide monohydrate in the 2000-400 cm⁻¹ region of the spectrum.

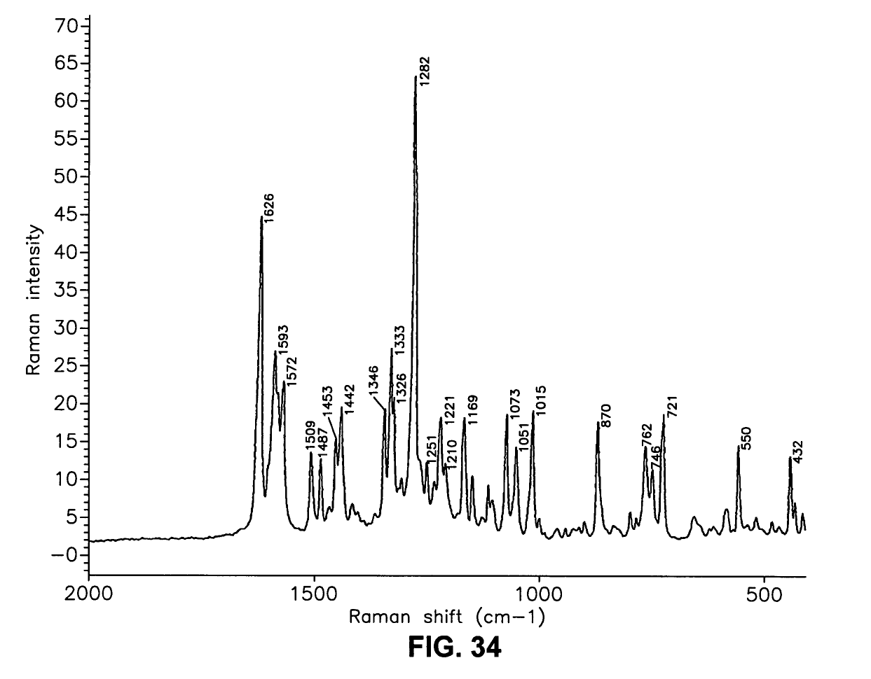

FIG. 35 is an FT-IR spectrum for carvedilol hydrobromide monohydrate.

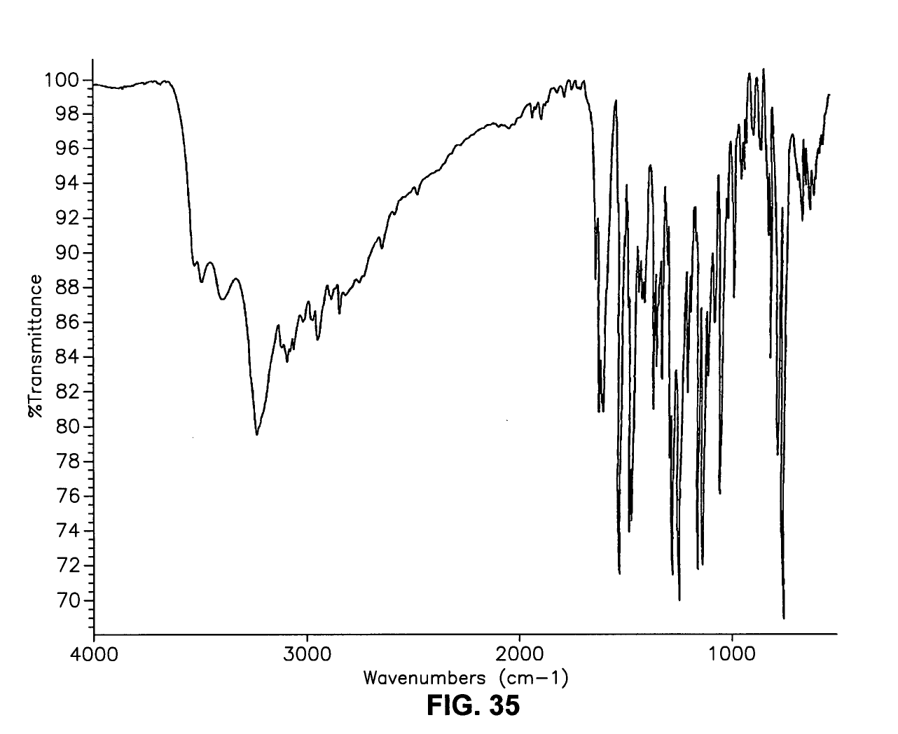

FIG. 36 is an FT-IR spectrum for carvedilol hydrobromide monohydrate in the 4000-2000 cm⁻¹ region of the spectrum.

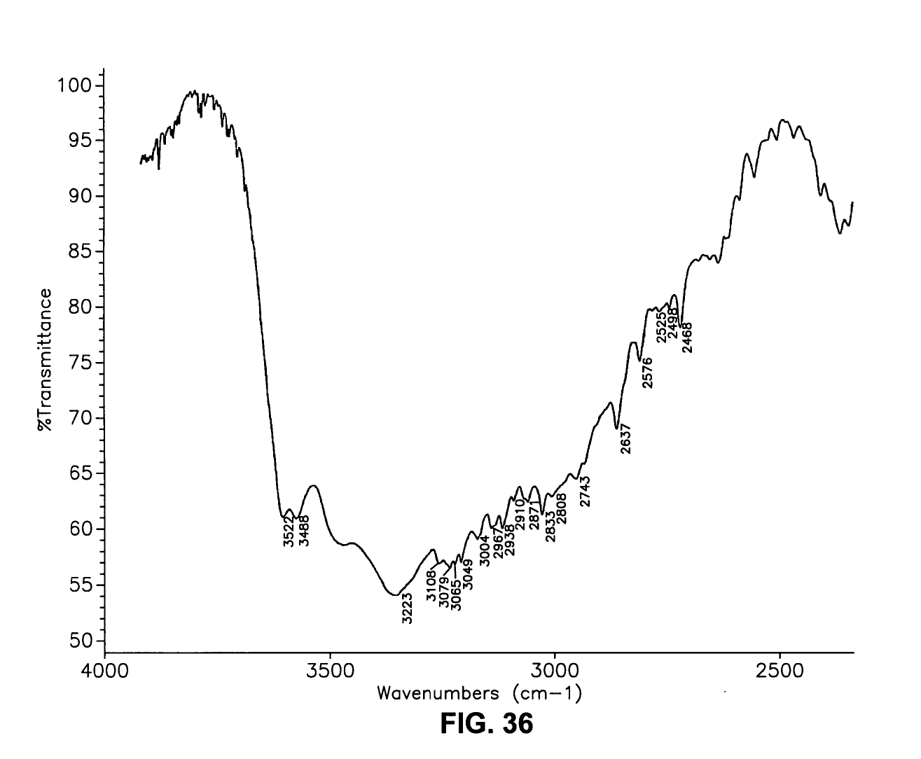

FIG. 37 is an FT-IR spectrum for carvedilol hydrobromide monohydrate in the 2000-500 cm⁻¹ region of the spectrum.

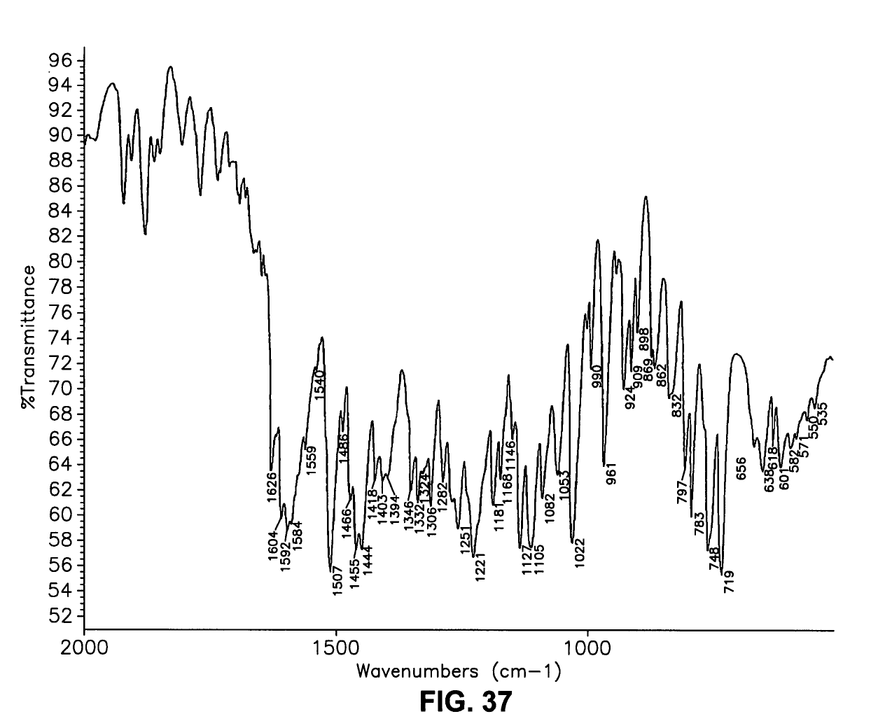

FIG. 38 is a view of a single molecule of carvedilol hydrobromide monohydrate. The hydroxyl group and the water molecule are disordered.

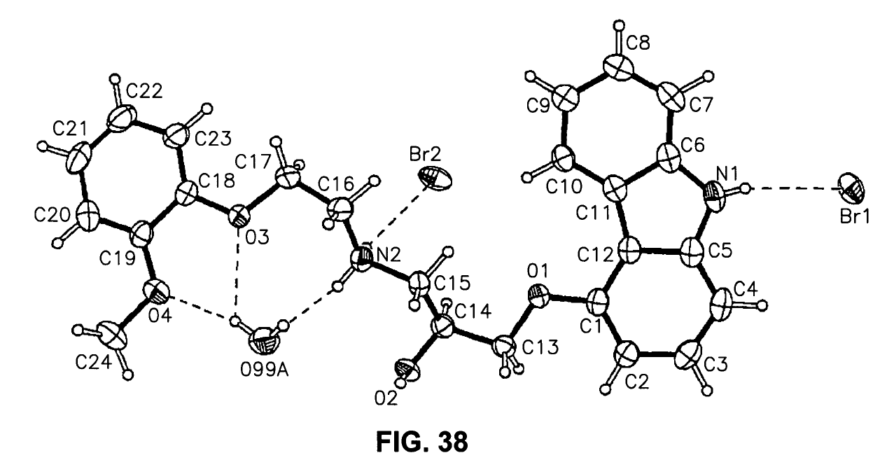

FIG. 39 are views of molecules of carvedilol hydrobromide monohydrate showing the N-H . . . Br . . . H-N interactions. The top view focuses on Br1 and the bottom view focuses on Br2. The interaction between the carvedilol cation and the bromine anion is unusual. Each carvedilol molecule makes two chemically different contacts to the bromine anions. Each bromine anion sits on a crystallographic special position (that is, on a crystallographic two-fold axis) which means that there are two half bromine anions interacting with each carvedilol cation.

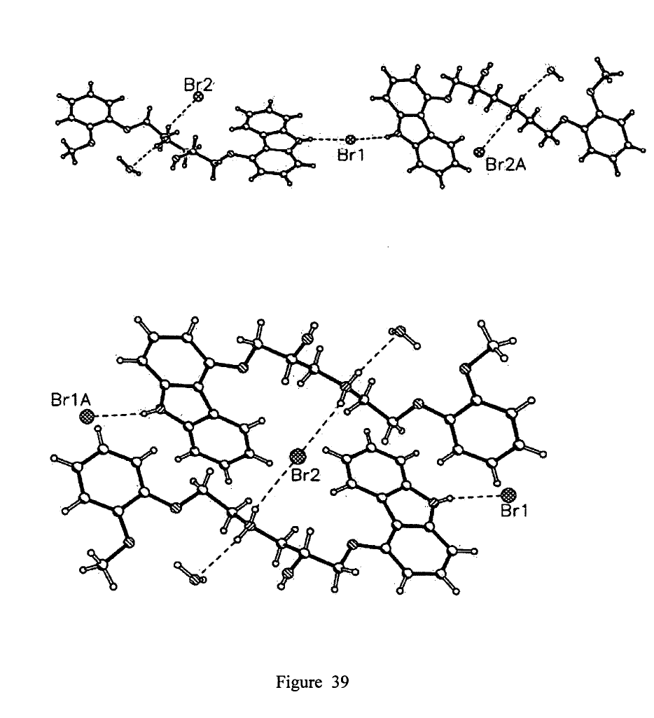

FIG. 40 is a differential scanning calorimetry thermogram for carvedilol hydrobromide dioxane solvate.

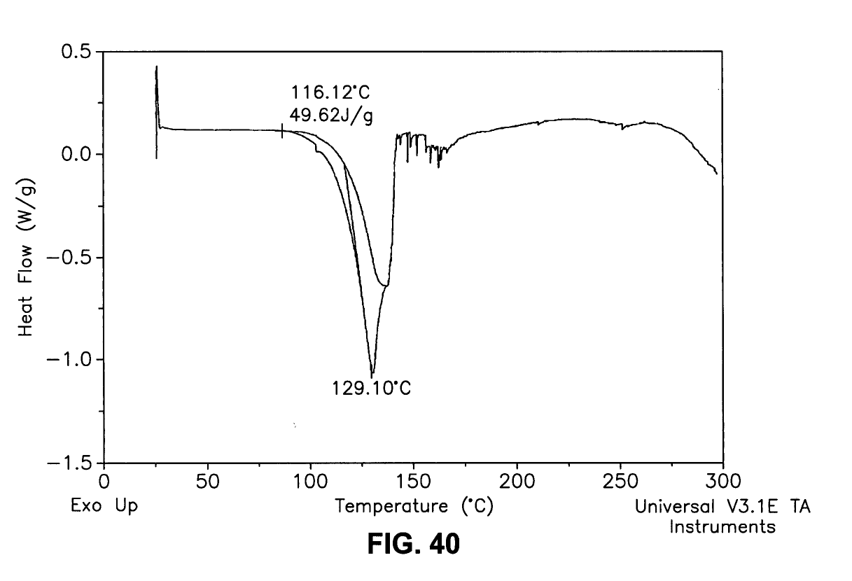

FIG. 41 is an FT-Raman spectrum for carvedilol hydrobromide dioxane solvate.

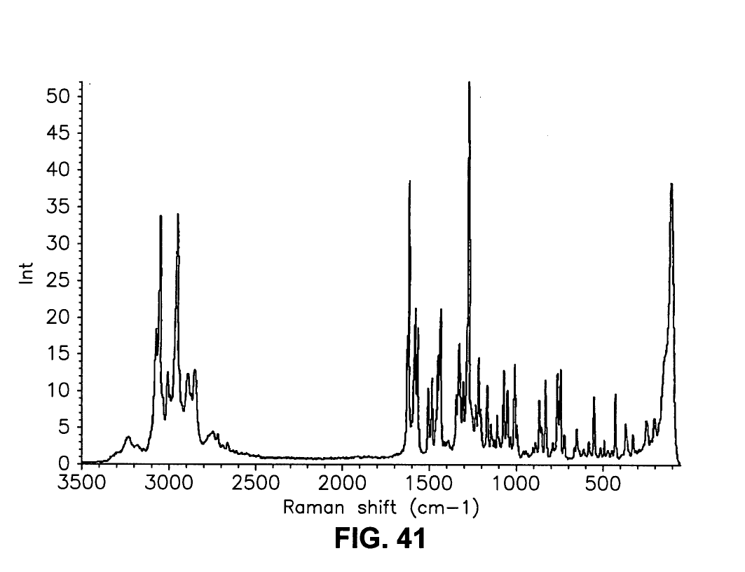

FIG. 42 is an FT-Raman spectrum for carvedilol hydrobromide dioxane solvate in the 4000-2000 cm⁻¹ region of the spectrum.

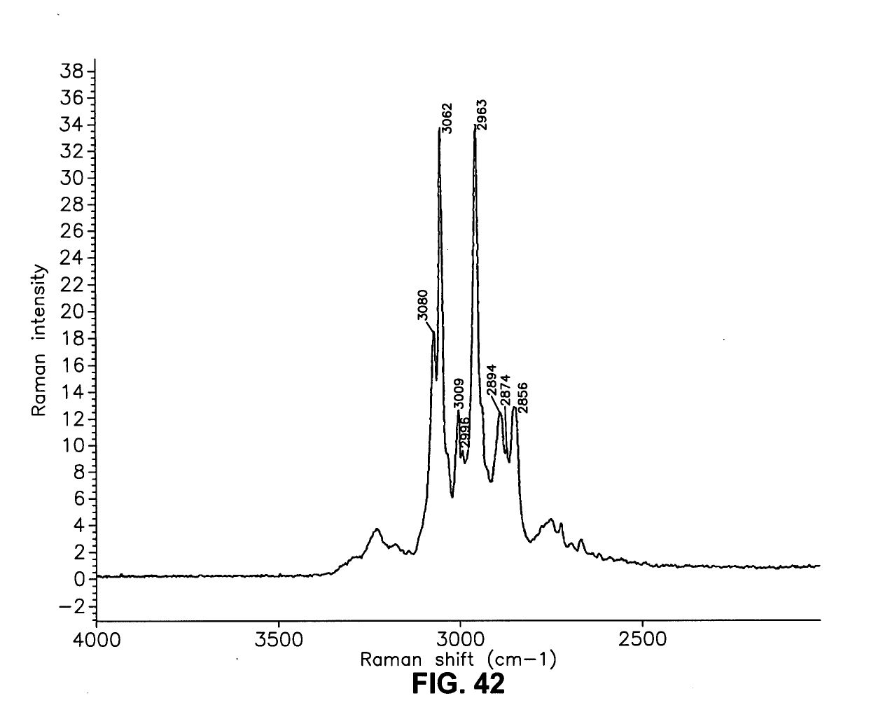

FIG. 43 is an FT-Raman spectrum for carvedilol hydrobromide dioxane solvate in the 2000-400 cm⁻¹ region of the spectrum.

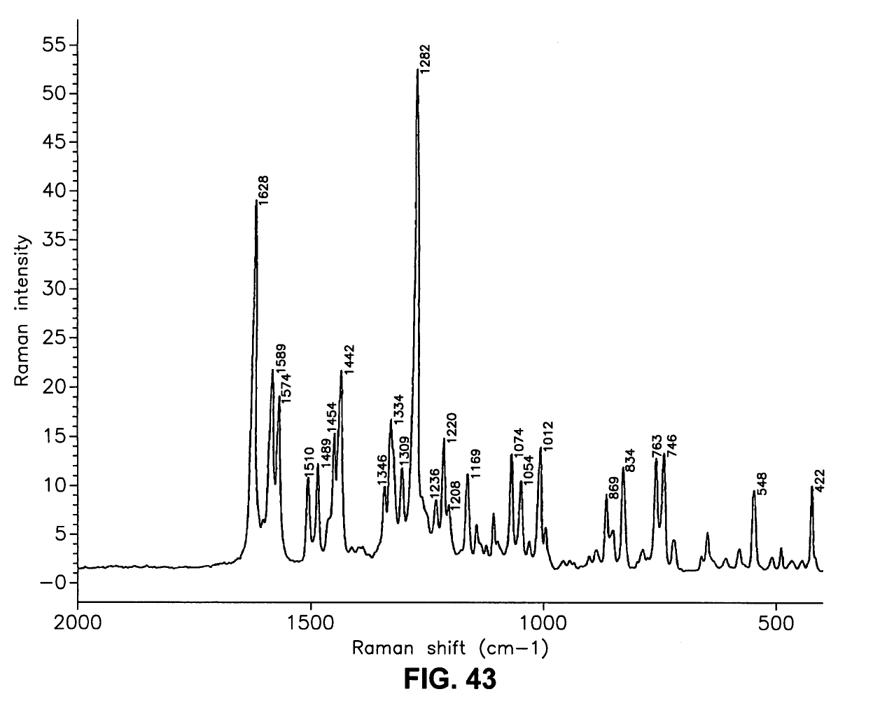

FIG. 44 is an FT-IR spectrum for carvedilol hydrobromide dioxane solvate.

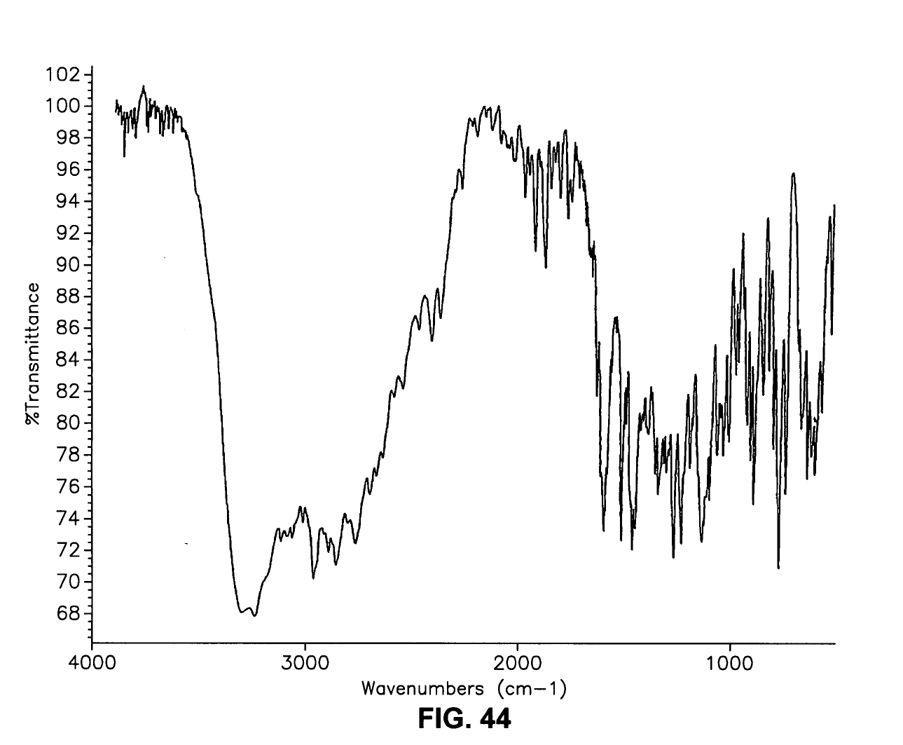

FIG. 45 is an FT-IR spectrum for carvedilol hydrobromide dioxane solvate in the 4000-2000 cm⁻¹ region of the spectrum.

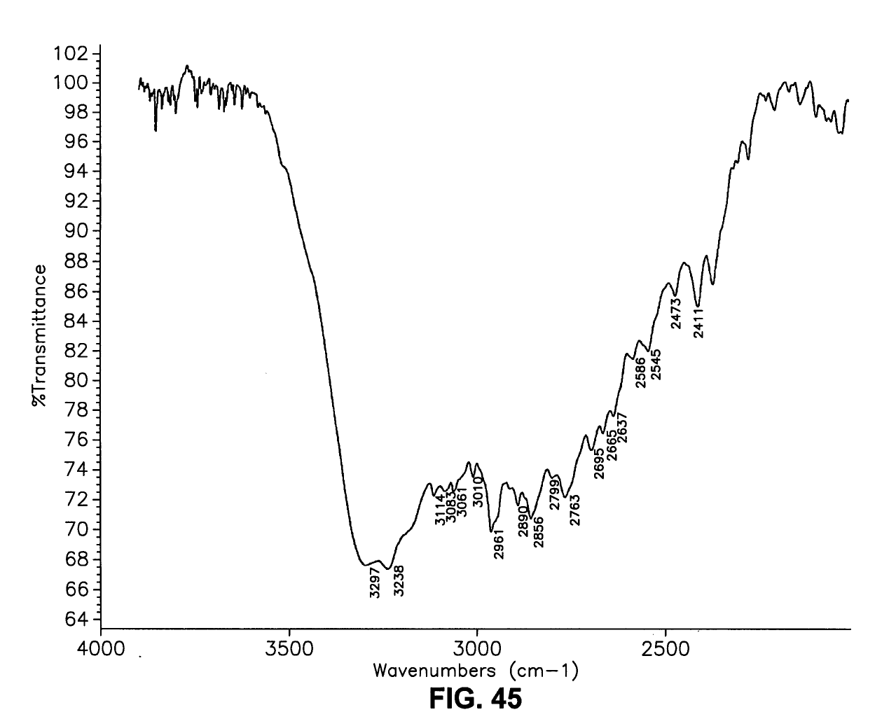

FIG. 46 is an FT-IR spectrum for carvedilol hydrobromide dioxane solvate in the 2000-500 cm⁻¹ region of the spectrum.

FIG. 47 is a differential scanning calorimetry thermogram for carvedilol hydrobromide 1-pentanol solvate.

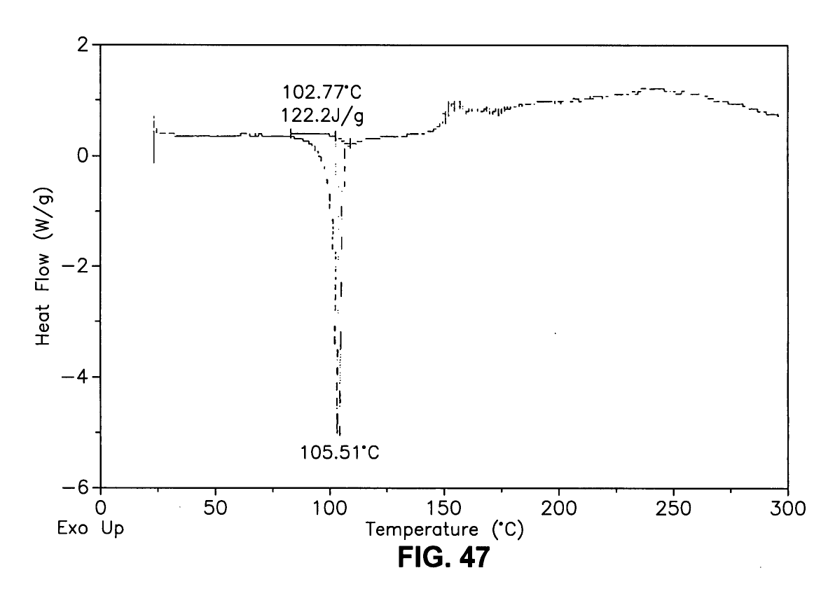

FIG. 48 is an FT-Raman spectrum for carvedilol hydrobromide 1-pentanol solvate.

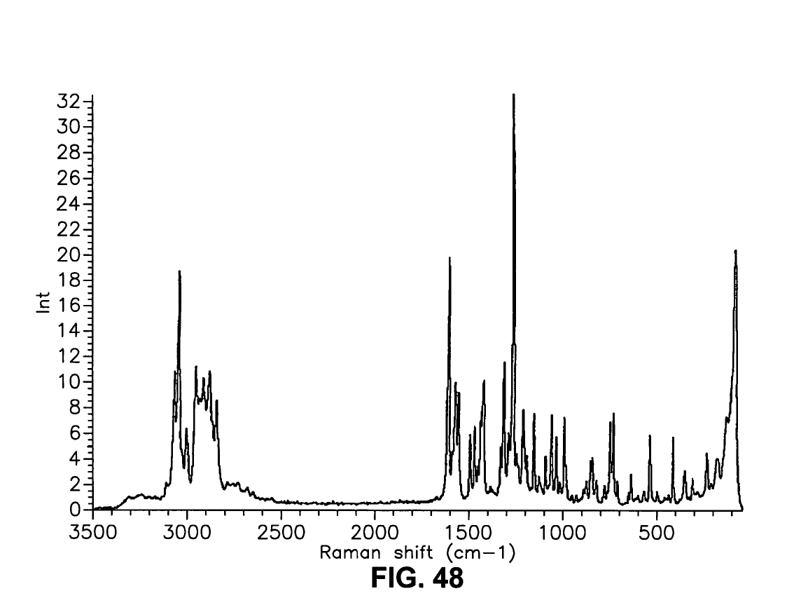

FIG. 49 is an FT-Raman spectrum for carvedilol hydrobromide 1-pentanol solvate in the 4000-2000 cm⁻¹ region of the spectrum.

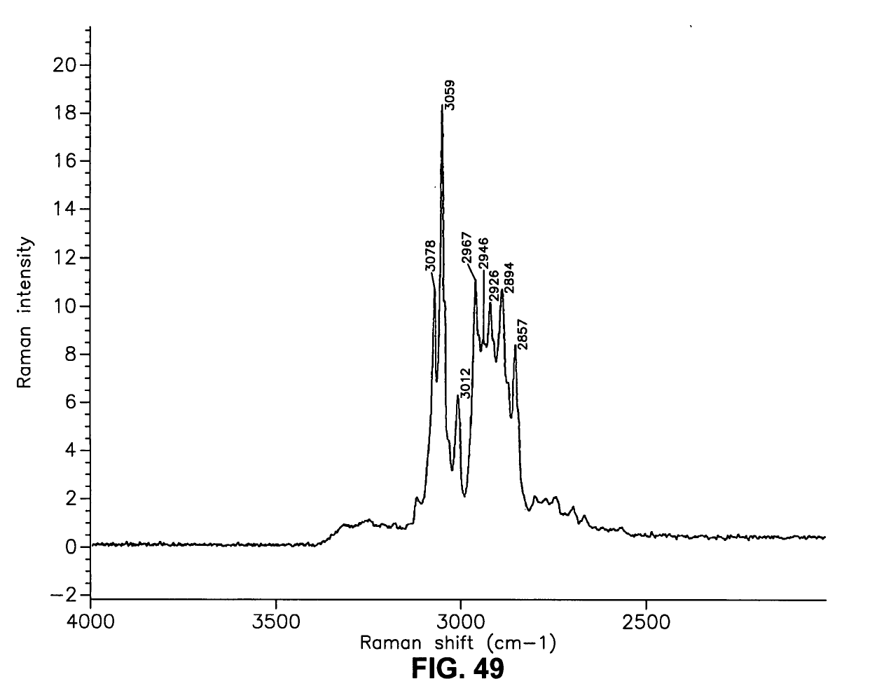

FIG. 50 is an FT-Raman spectrum for carvedilol hydrobromide 1-pentanol solvate in the 2000-400 cm⁻¹ region of the spectrum.

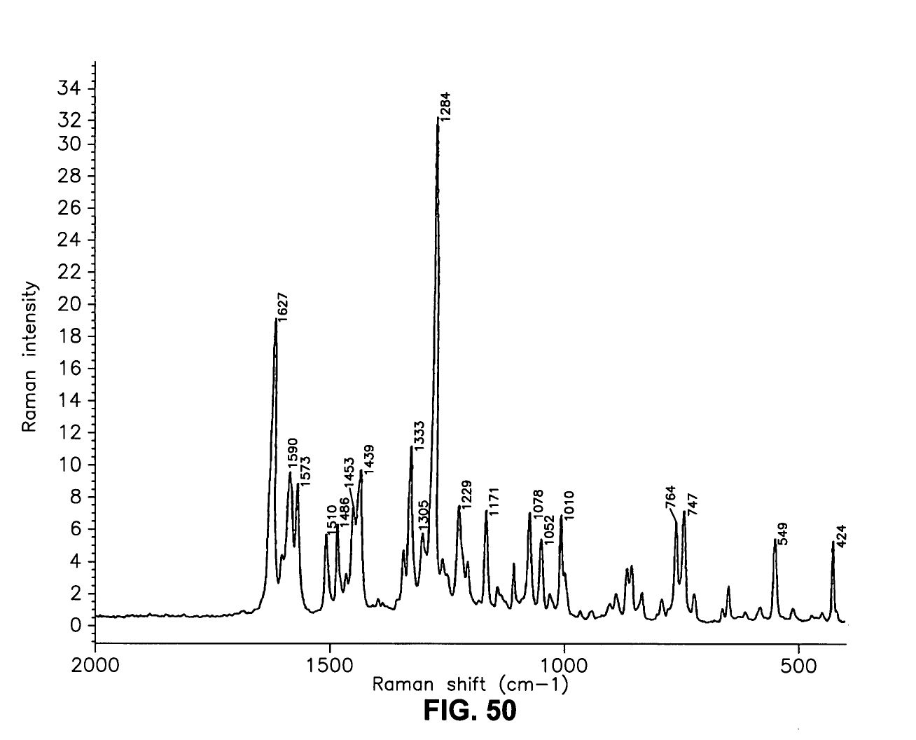

FIG. 51 is an FT-IR spectrum for carvedilol hydrobromide 1-pentanol solvate.

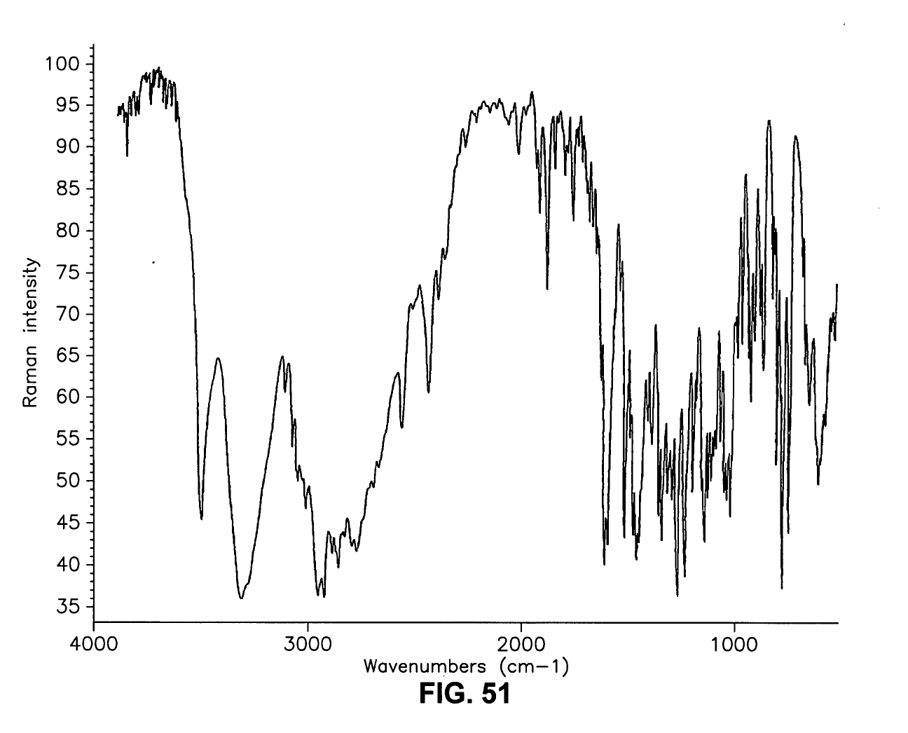

FIG. 52 is an FT-IR spectrum for carvedilol hydrobromide 1-pentanol solvate in the 4000-2000 cm⁻¹ region of the spectrum.

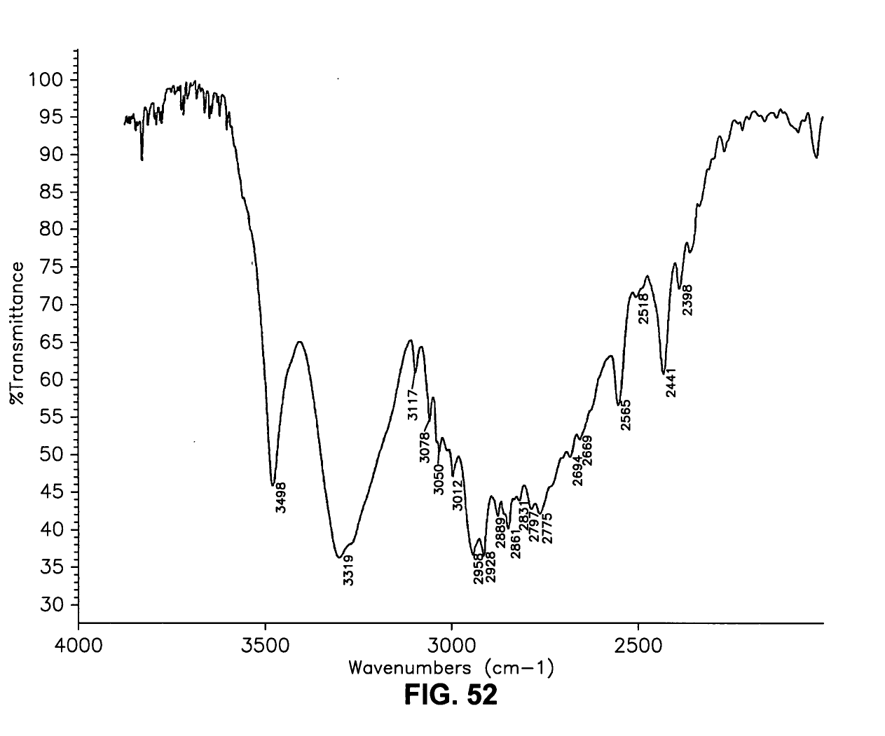

FIG. 53 is an FT-IR spectrum for carvedilol hydrobromide 1-pentanol solvate in the 2000-500 cm⁻¹ region of the spectrum.

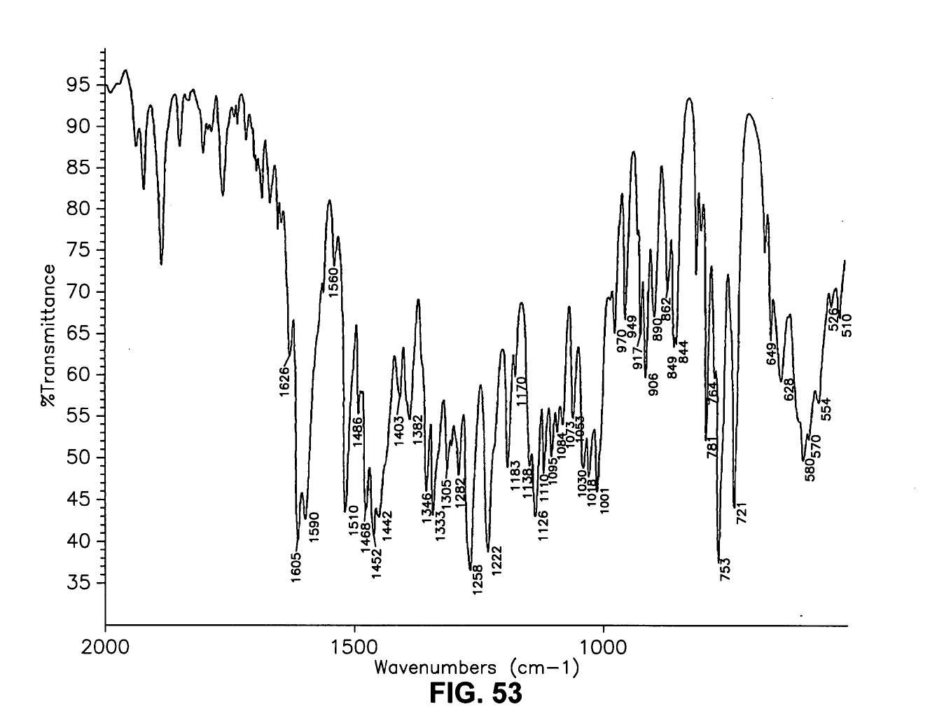

FIG. 54 is a differential scanning calorimetry thermogram for carvedilol hydrobromide 2-methyl-1-propanol solvate.

FIG. 55 is an FT-Raman spectrum for carvedilol hydrobromide 2-methyl-1-propanol solvate.

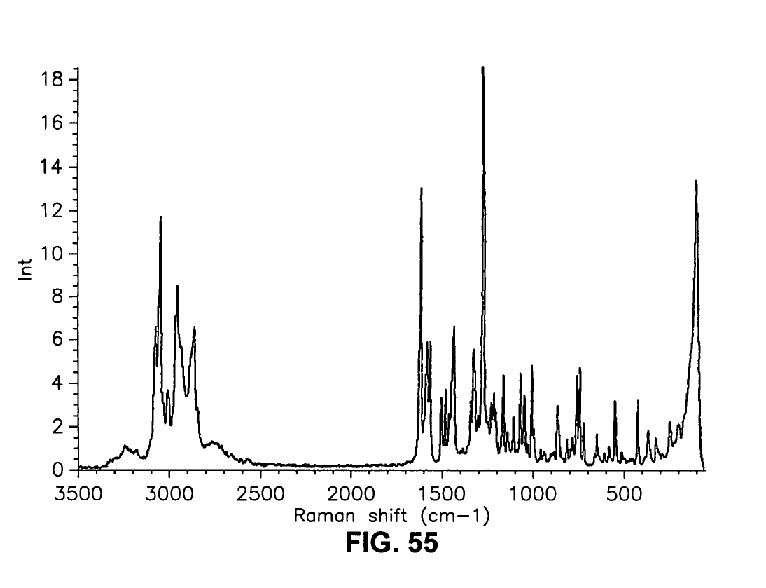

FIG. 56 is an FT-Raman spectrum for carvedilol hydrobromide 2-methyl-1-propanol solvate in the 4000-2000 cm⁻¹ region of the spectrum.

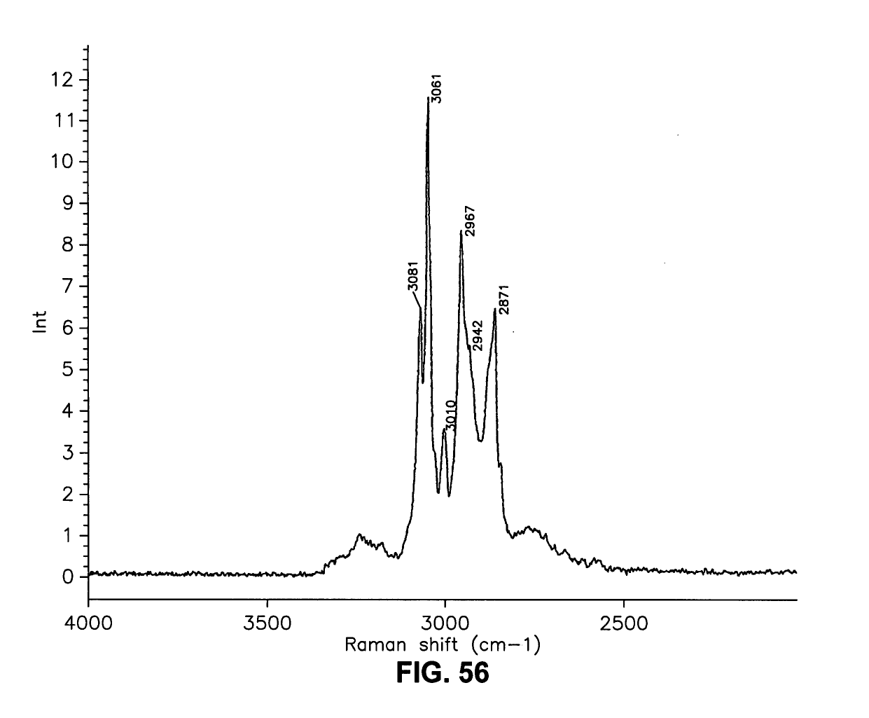

FIG. 57 is an FT-Raman spectrum for carvedilol hydrobromide 2-methyl-1-propanol solvate in the 2000-400 cm⁻¹ region of the spectrum.

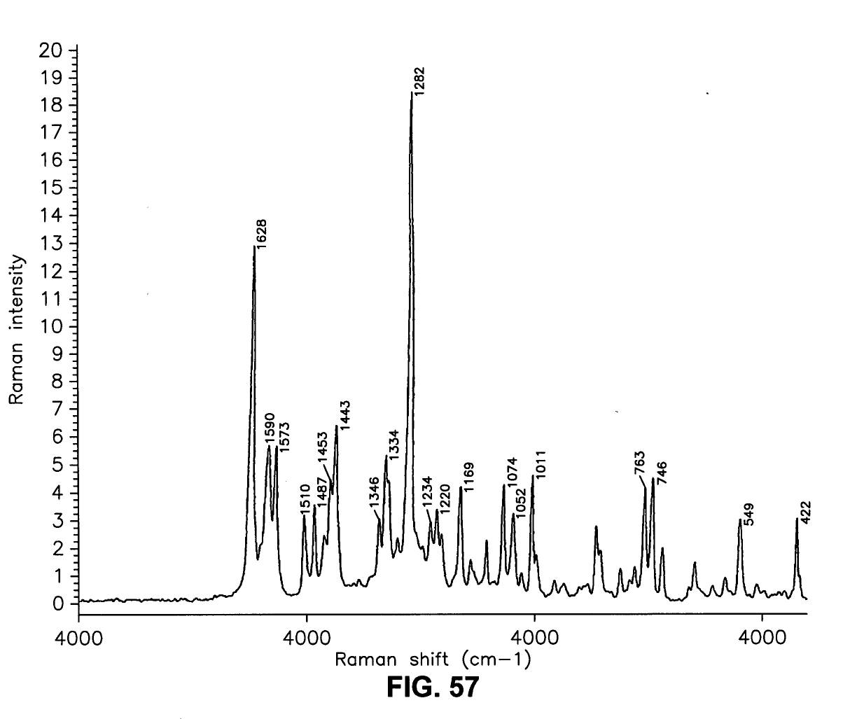

FIG. 58 is an FT-IR spectrum for carvedilol hydrobromide 2-methyl-1-propanol solvate.

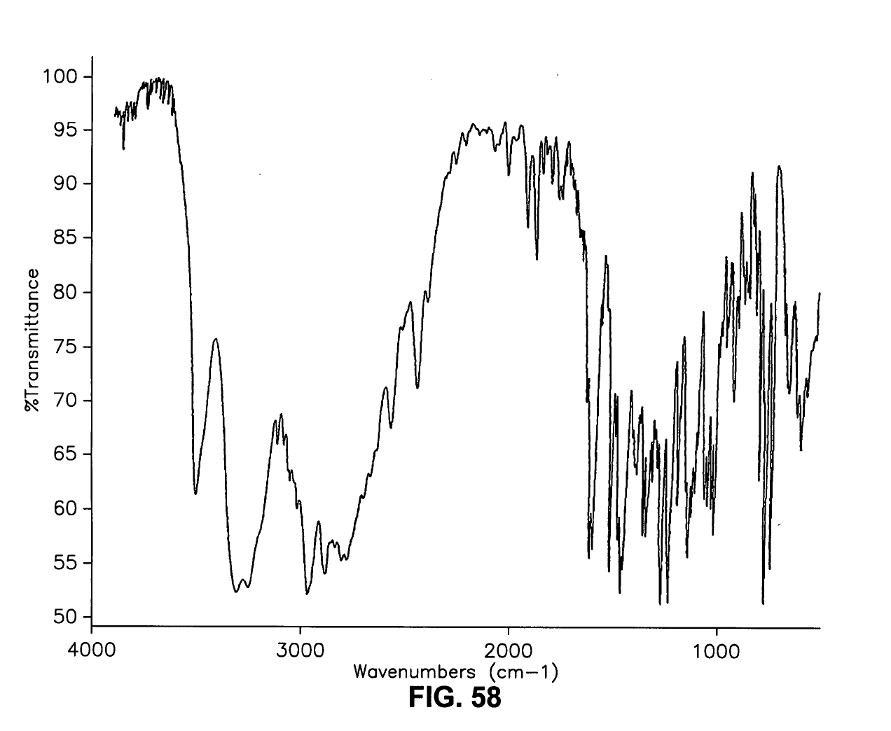

FIG. 59 is an FT-IR spectrum for carvedilol hydrobromide 2-methyl-1-propanol solvate in the 4000-2000 cm⁻¹ region of the spectrum.

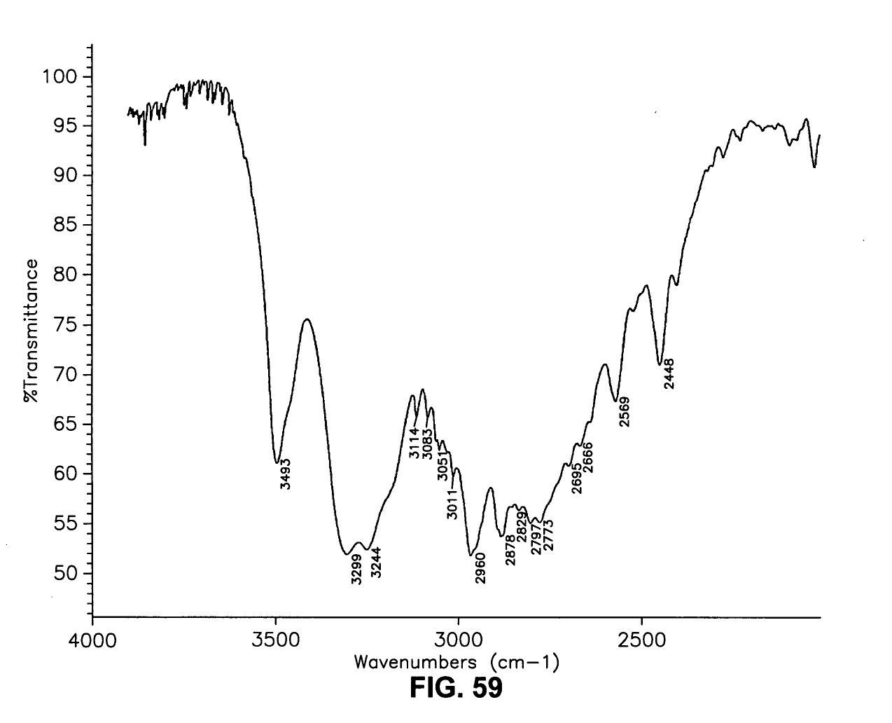

FIG. 60 is an FT-IR spectrum for carvedilol hydrobromide 2-methyl-1-propanol solvate in the 2000-500 cm⁻¹ region of the spectrum.

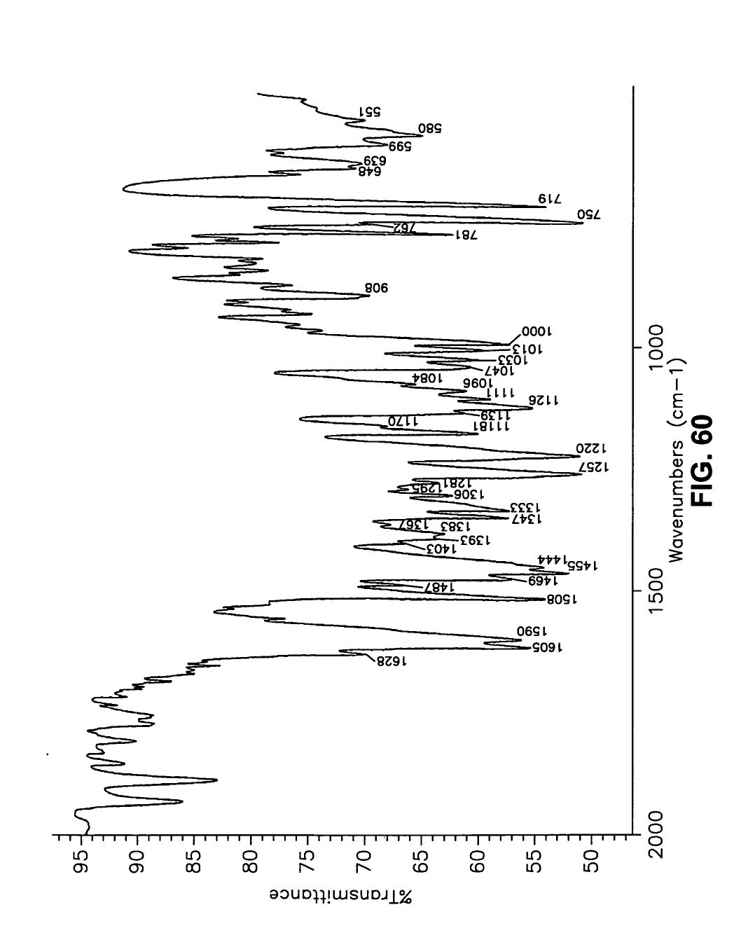

FIG. 61 is a differential scanning calorimetry thermogram for carvedilol hydrobromide trifluoroethanol solvate.

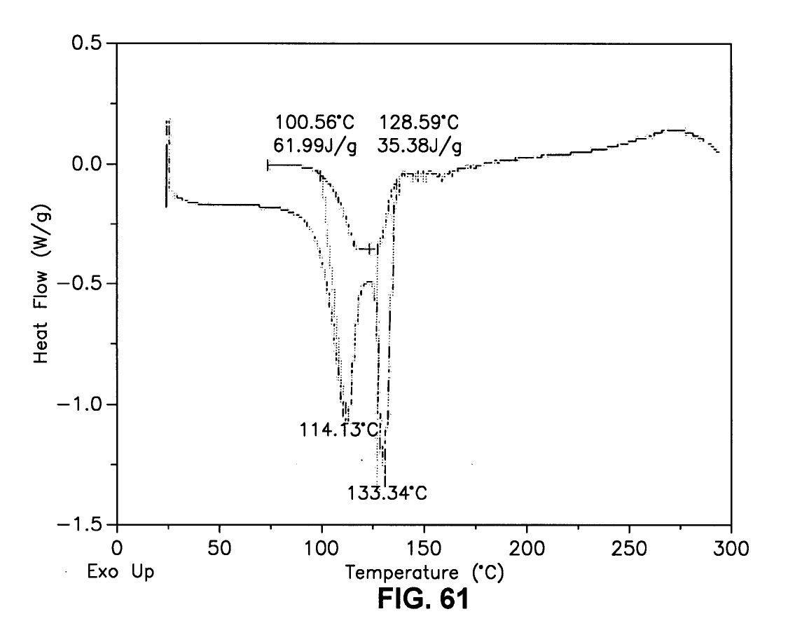

FIG. 62 is an FT-Raman spectrum for carvedilol hydrobromide trifluoroethanol solvate.

FIG. 63 is an FT-Raman spectrum for carvedilol hydrobromide trifluoroethanol solvate in the 4000-2000 cm⁻¹ region of the spectrum.

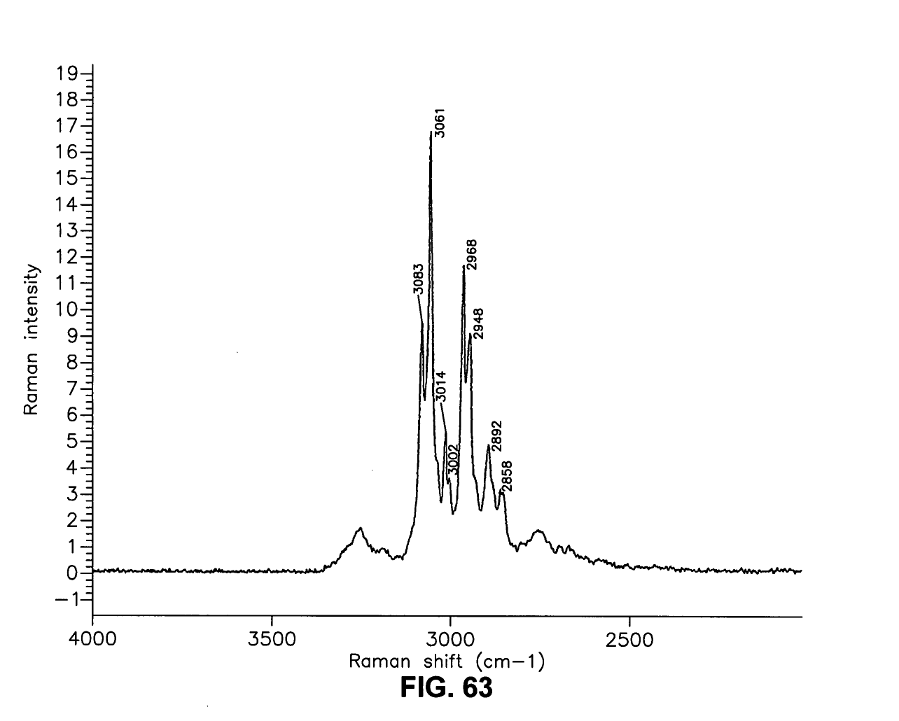

FIG. 64 is an FT-Raman spectrum for carvedilol hydrobromide trifluoroethanol solvate in the 2000-400 cm⁻¹ region of the spectrum.

FIG. 65 is an FT-IR spectrum for carvedilol hydrobromide trifluoroethanol solvate.

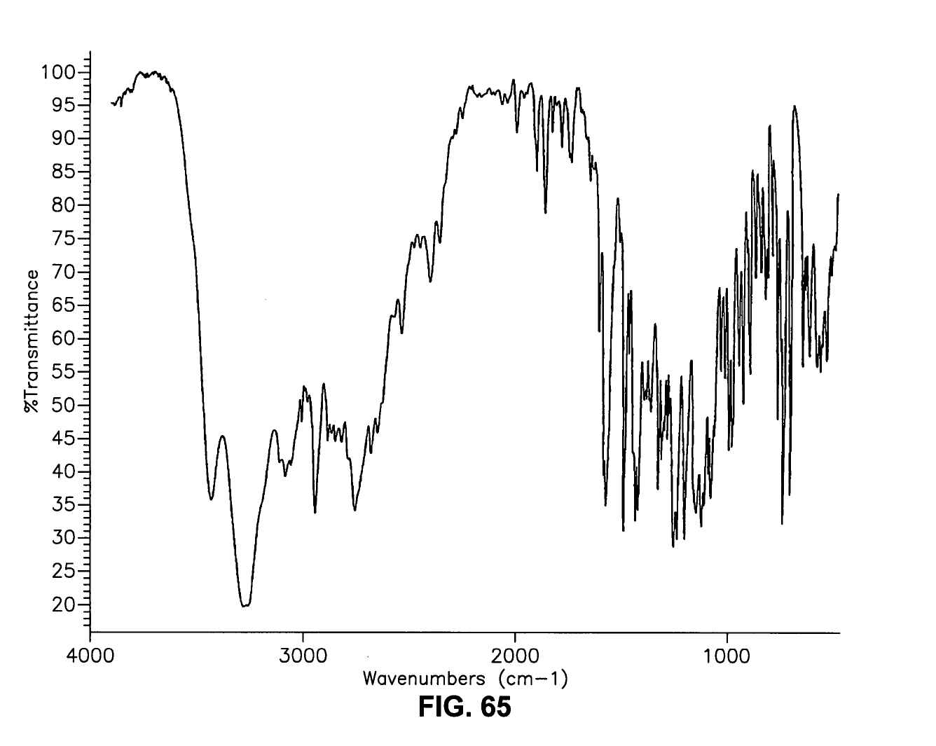

FIG. 66 is an FT-IR spectrum for carvedilol hydrobromide trifluoroethanol solvate in the 4000-2000 cm⁻¹ region of the spectrum.

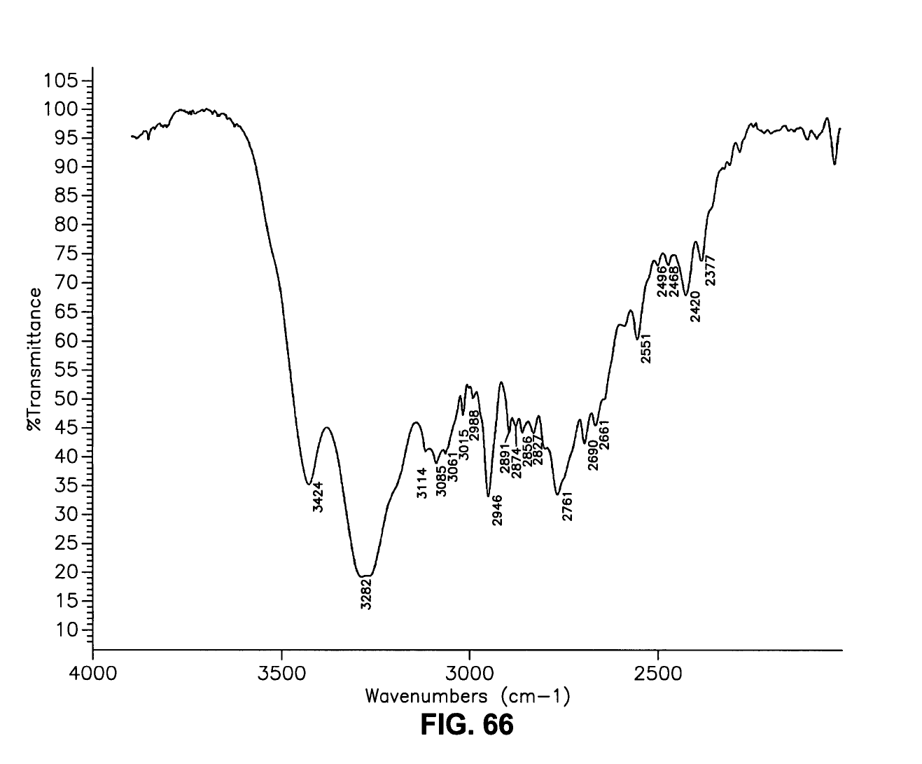

FIG. 67 is an FT-IR spectrum for carvedilol hydrobromide trifluoroethanol solvate in the 2000-500 cm⁻¹ region of the spectrum.

FIG. 68 is a differential scanning calorimetry thermogram for carvedilol hydrobromide 2-propanol solvate.

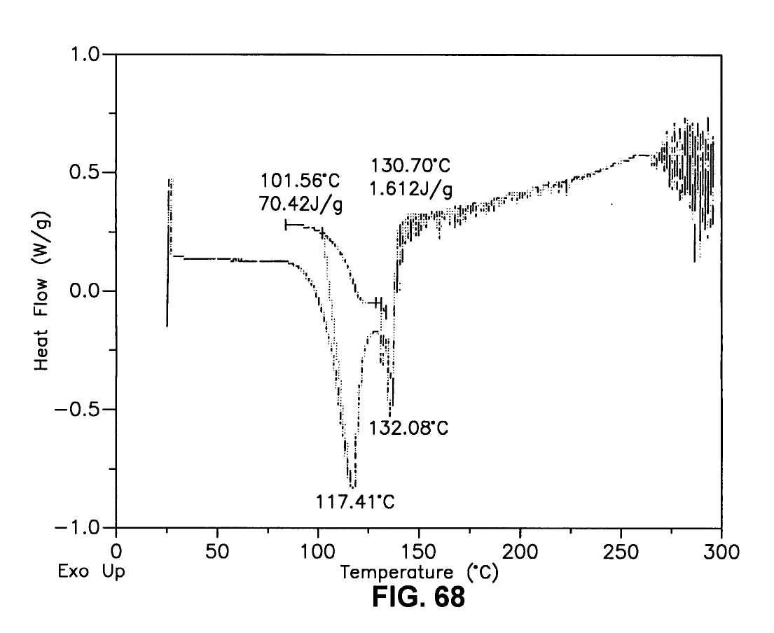

FIG. 69 is an FT-Raman spectrum for carvedilol hydrobromide 2-propanol solvate.

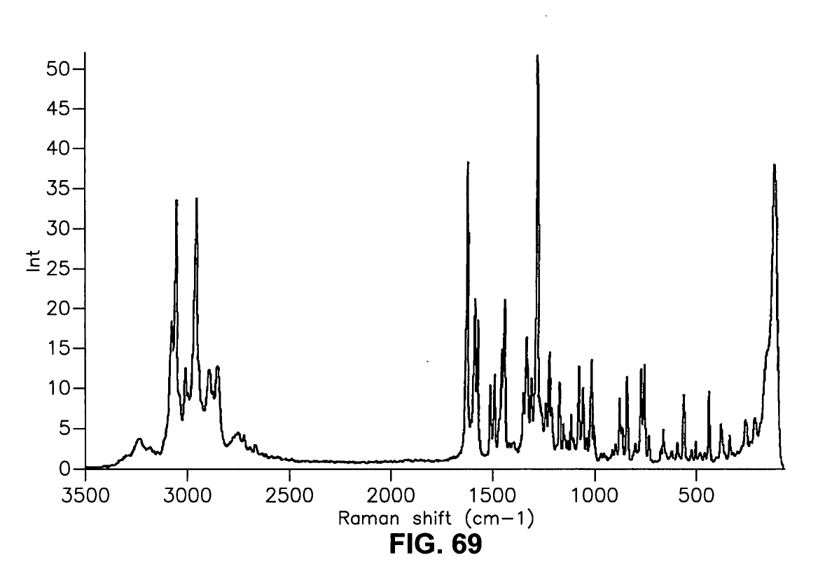

FIG. 70 is an FT-Raman spectrum for carvedilol hydrobromide 2-propanol solvate in the 4000-2000 cm⁻¹ region of the spectrum.

FIG. 71 is an FT-Raman spectrum for carvedilol hydrobromide 2-propanol solvate in the 2000-400 cm⁻¹ region of the spectrum.

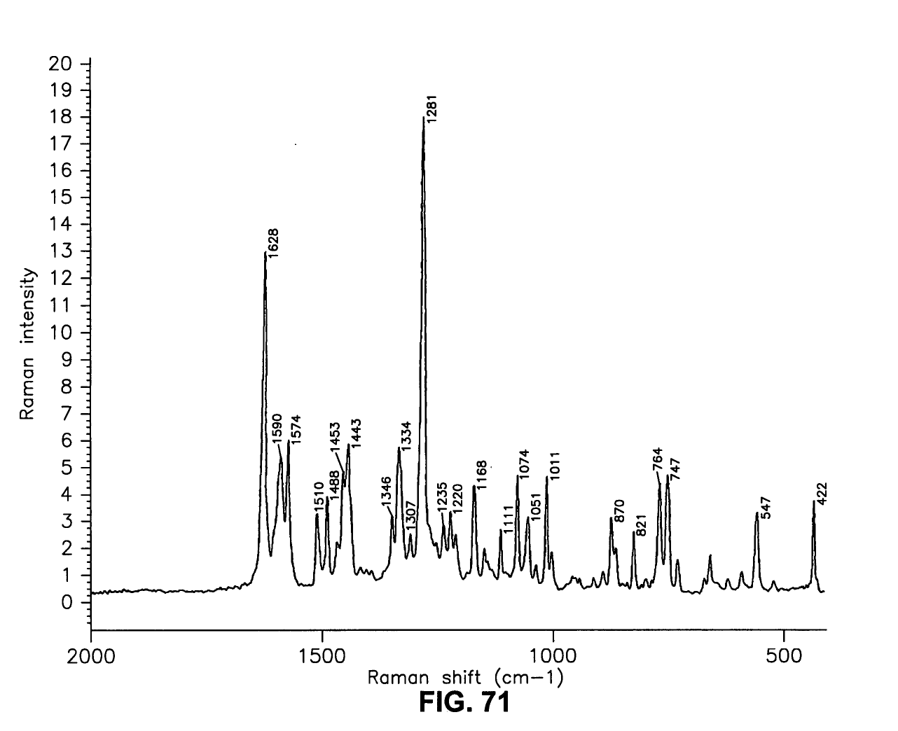

FIG. 72 is an FT-IR spectrum for carvedilol hydrobromide 2-propanol solvate.

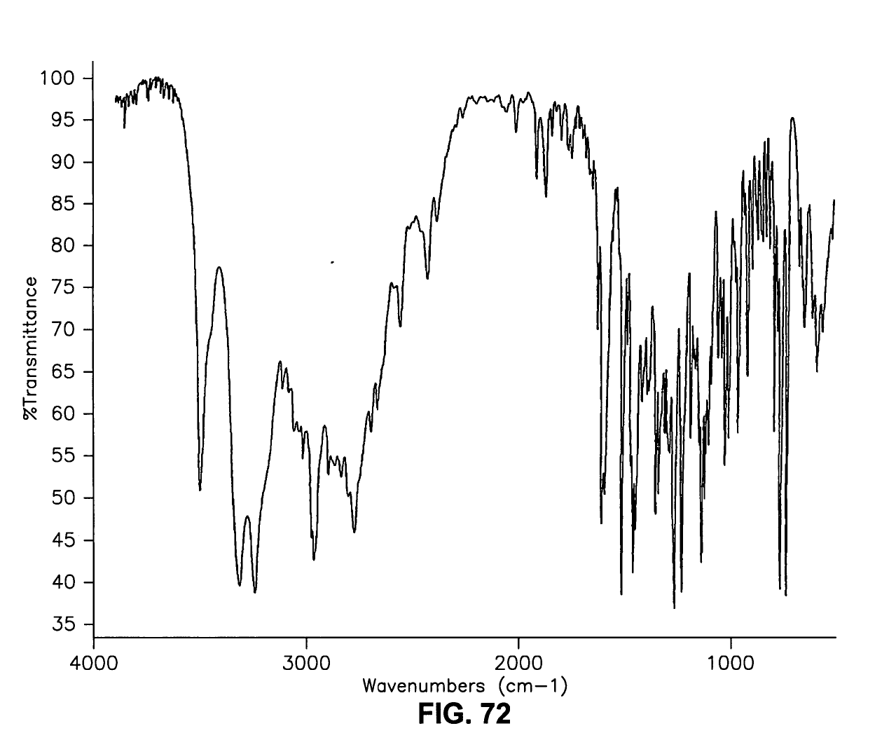

FIG. 73 is an FT-IR spectrum for carvedilol hydrobromide 2-propanol solvate in the 4000-2000 cm⁻¹ region of the spectrum.

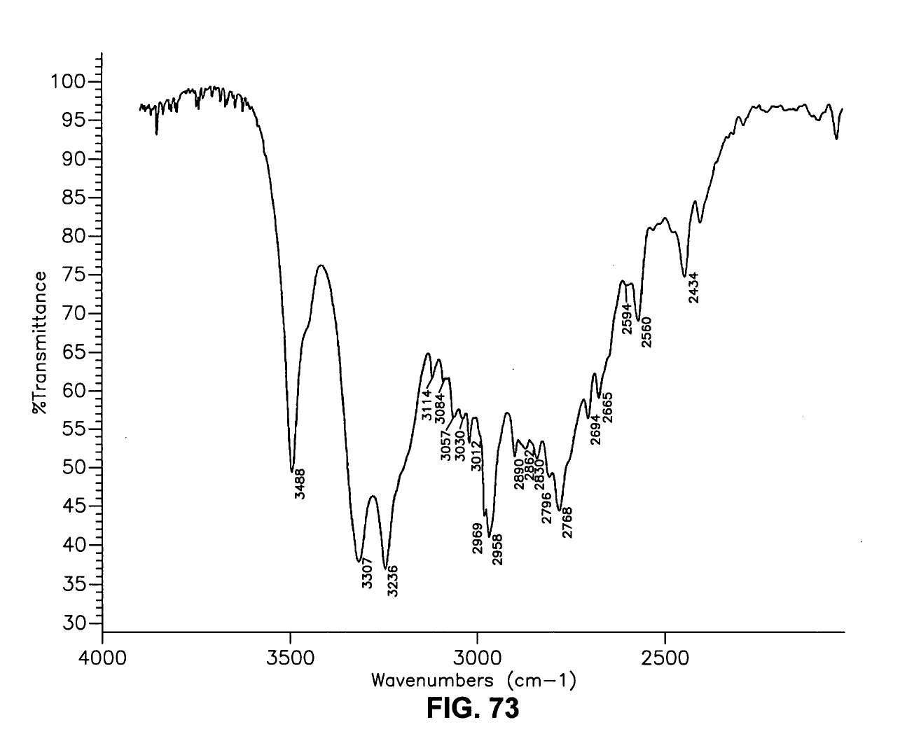

FIG. 74 is an FT-IR spectrum for carvedilol hydrobromide 2-propanol solvate in the 2000-500 cm⁻¹ region of the spectrum.

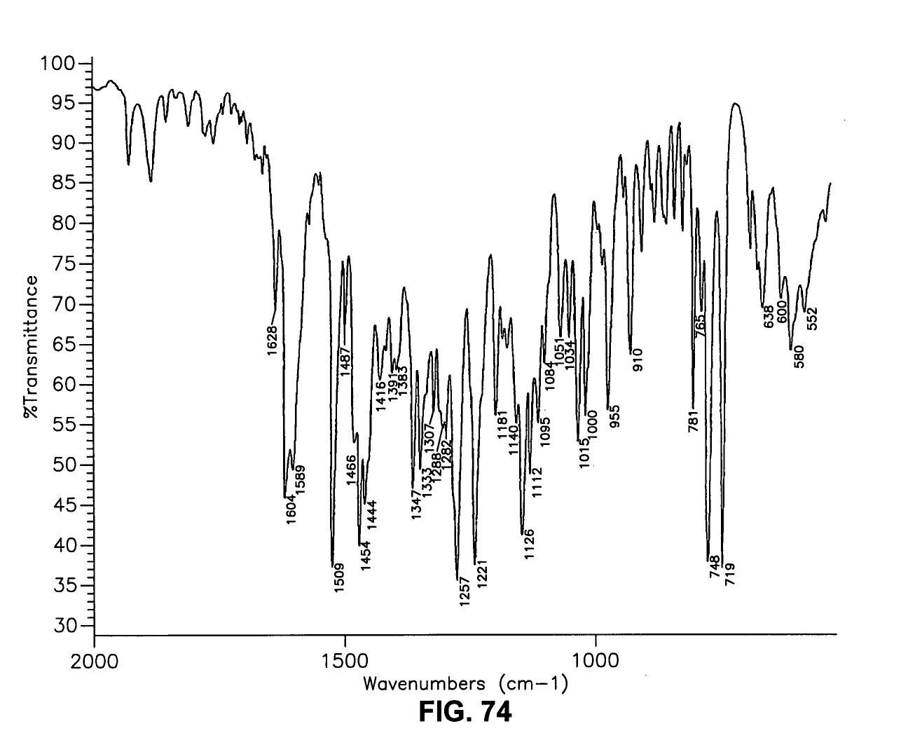

FIG. 75 is an x-ray powder diffractogram for carvedilol hydrobromide n-propanol solvate #1.

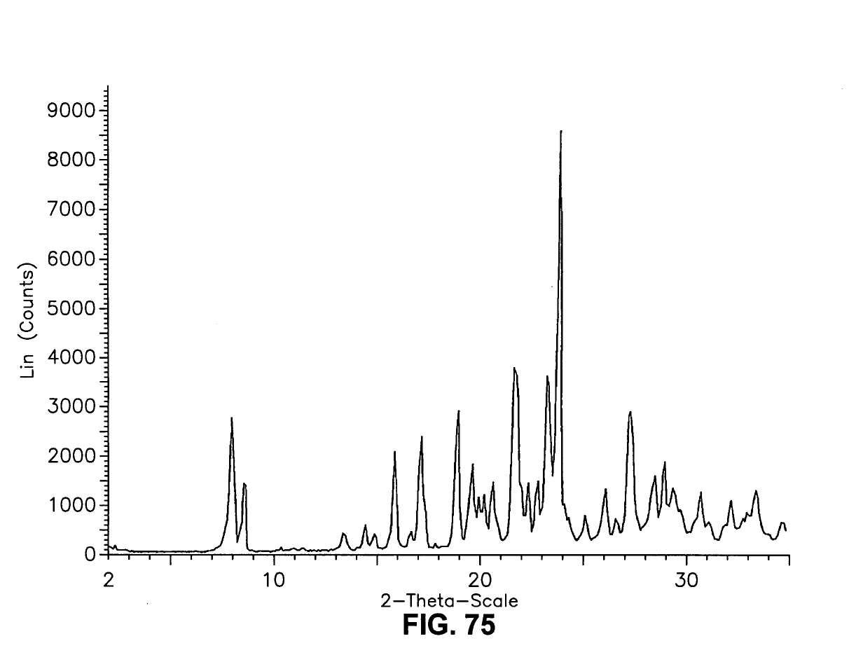

FIG. 76 shows the thermal analysis results for carvedilol hydrobromide n-propanol solvate #1.

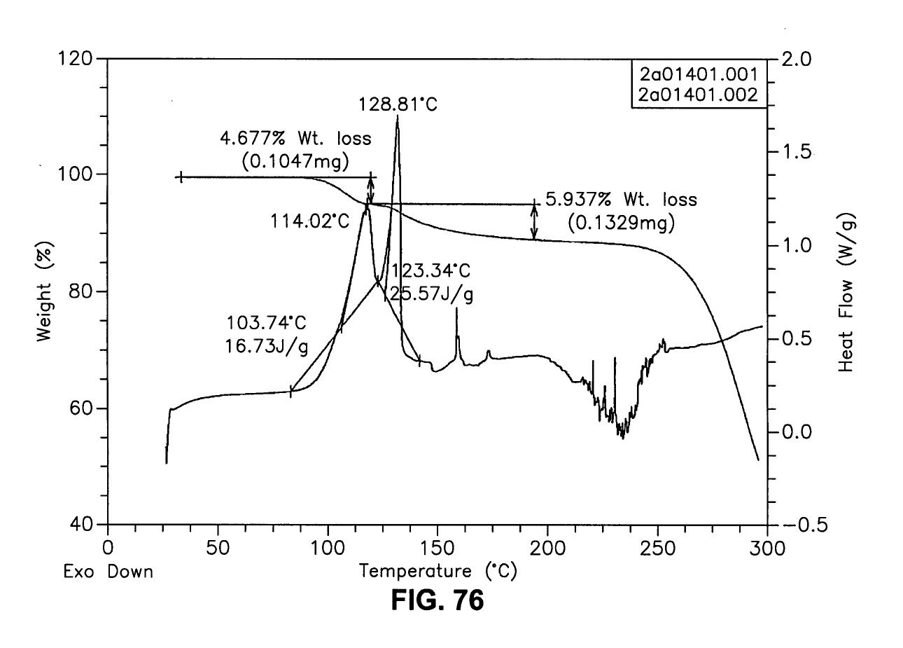

FIG. 77 is an FT-Raman spectrum for carvedilol hydrobromide n-propanol solvate #1.

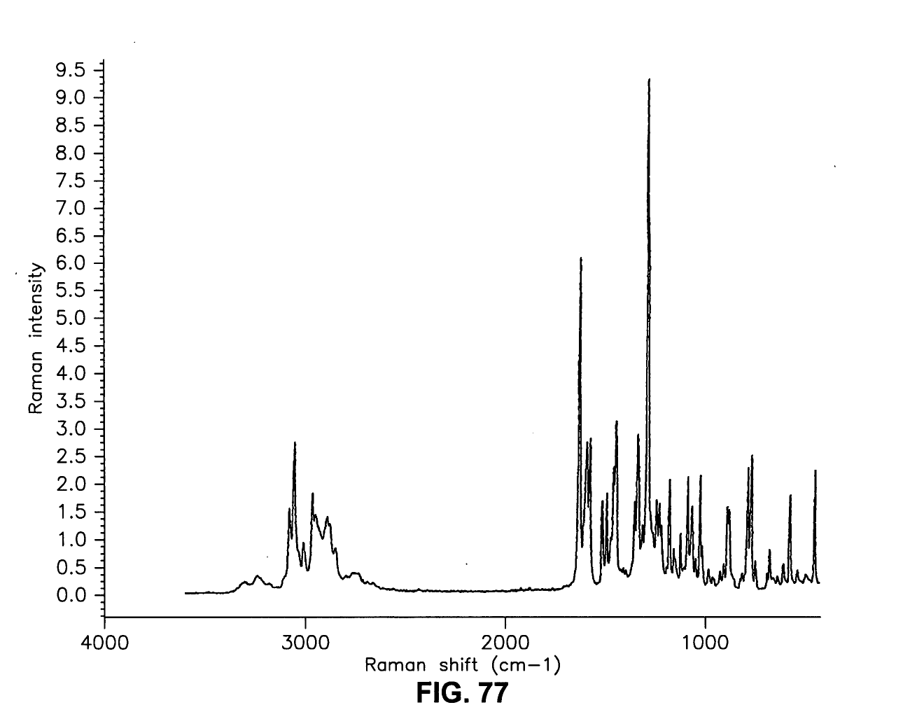

FIG. 78 is an FT-Raman spectrum for carvedilol hydrobromide n-propanol solvate #1 in the 4000-2000 cm⁻¹ region of the spectrum.

FIG. 79 is an FT-Raman spectrum for carvedilol hydrobromide n-propanol solvate #1 in the 2000-400 cm⁻¹ region of the spectrum.

FIG. 80 is an FT-IR spectrum for carvedilol hydrobromide n-propanol solvate #1.

FIG. 81 is an FT-IR spectrum for carvedilol hydrobromide n-propanol solvate #1 in the 4000-2000 cm⁻¹ region of the spectrum.

FIG. 82 is an FT-IR spectrum for carvedilol hydrobromide n-propanol solvate #1 in the 2000-500 cm⁻¹ region of the spectrum.

FIG. 83 is an x-ray powder diffractogram for carvedilol hydrobromide n-propanol solvate #2.

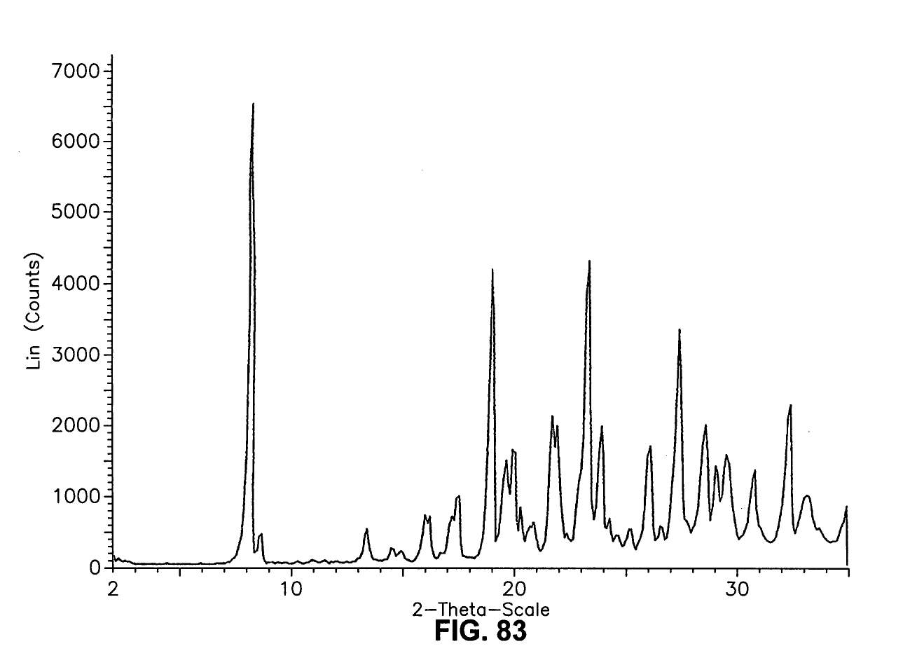

FIG. 84 shows the thermal analysis results for carvedilol hydrobromide n-propanol solvate #2.

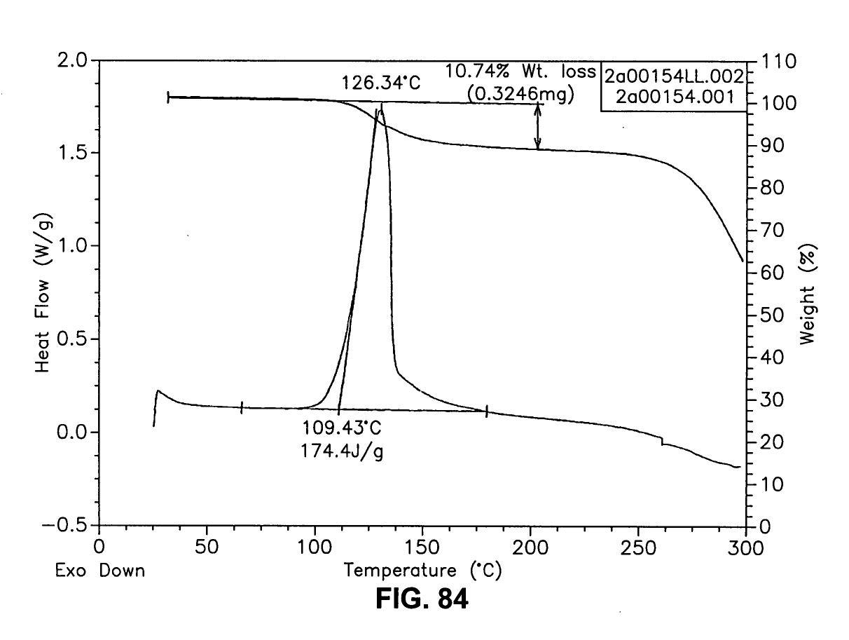

FIG. 85 is an FT-Raman spectrum for carvedilol hydrobromide n-propanol solvate #2.

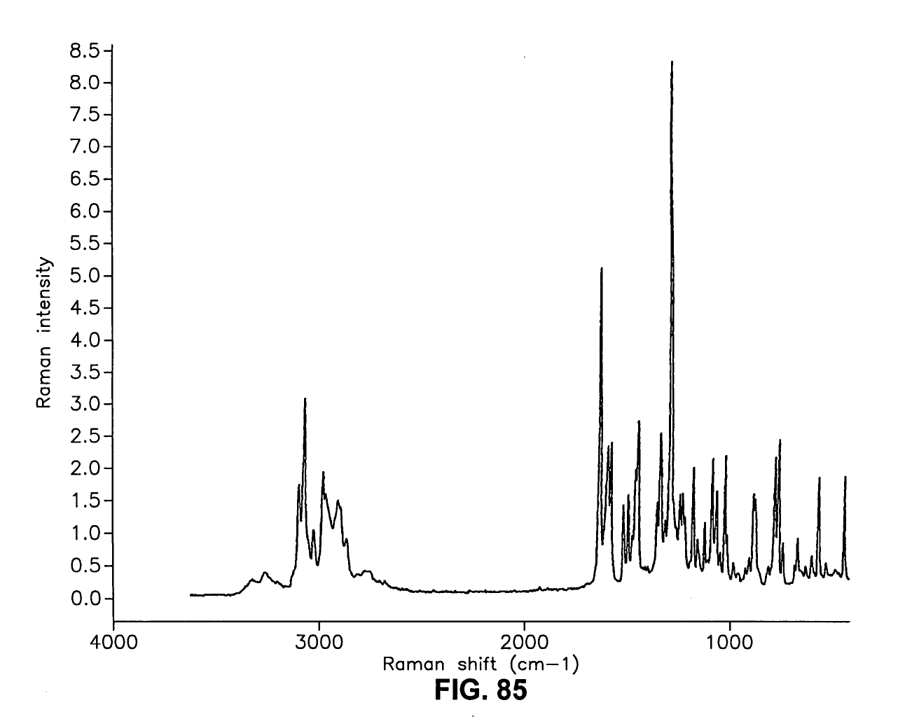

FIG. 86 is an FT-Raman spectrum for carvedilol hydrobromide n-propanol solvate #2 in the 4000-2000 cm⁻¹ region of the spectrum.

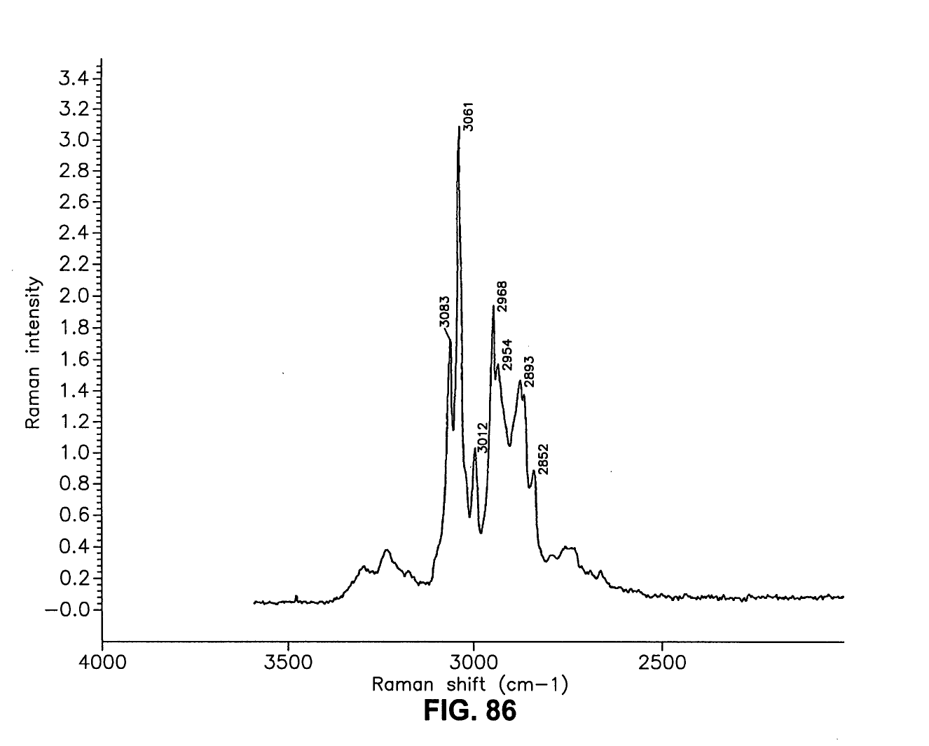

FIG. 87 is an FT-Raman spectrum for carvedilol hydrobromide n-propanol solvate #2 in the 2000-400 cm⁻¹ region of the spectrum.

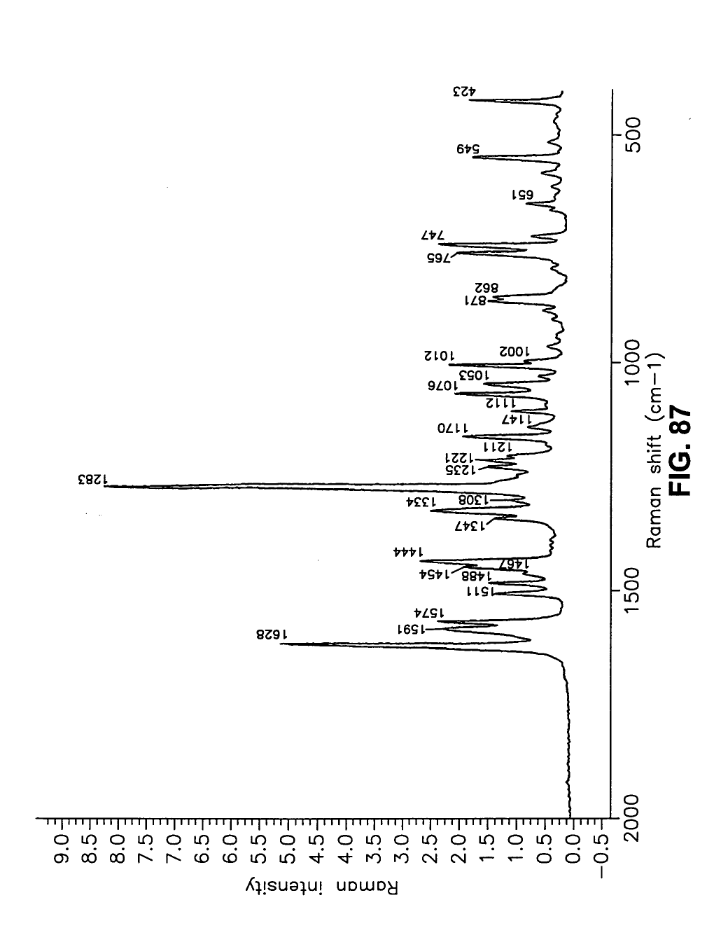

FIG. 88 is an FT-IR spectrum for carvedilol hydrobromide n-propanol solvate #2.

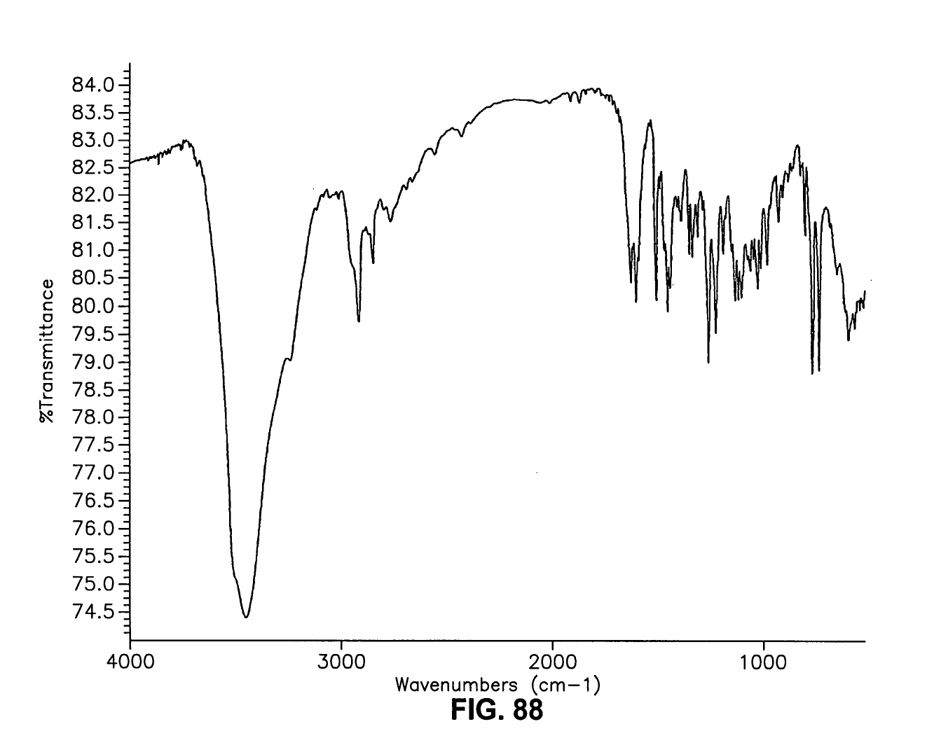

FIG. 89 is an FT-IR spectrum for carvedilol hydrobromide n-propanol solvate #2 in the 4000-2000 cm⁻¹ region of the spectrum.

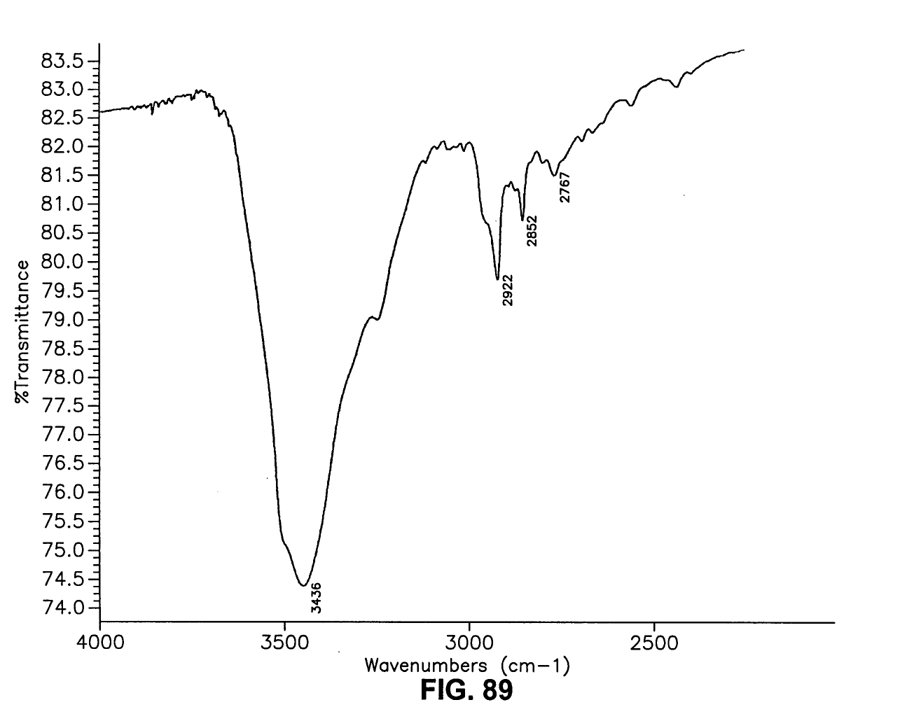

FIG. 90 is an FT-IR spectrum for carvedilol hydrobromide n-propanol solvate #2 in the 2000-500 cm⁻¹ region of the spectrum.

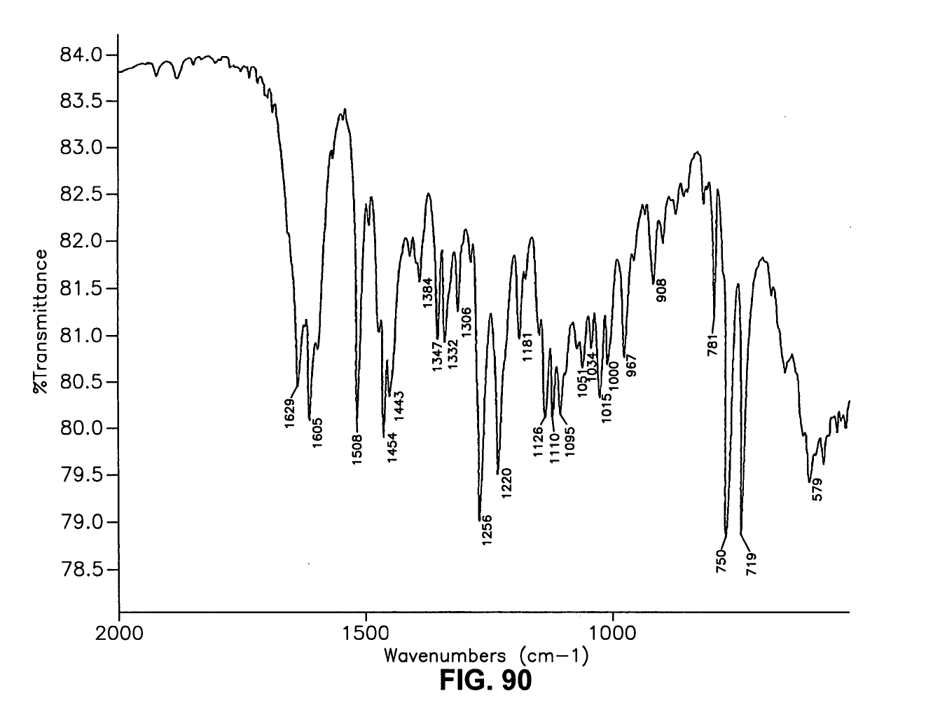

FIG. 91 is an x-ray powder diffractogram for carvedilol hydrobromide anhydrous forms.

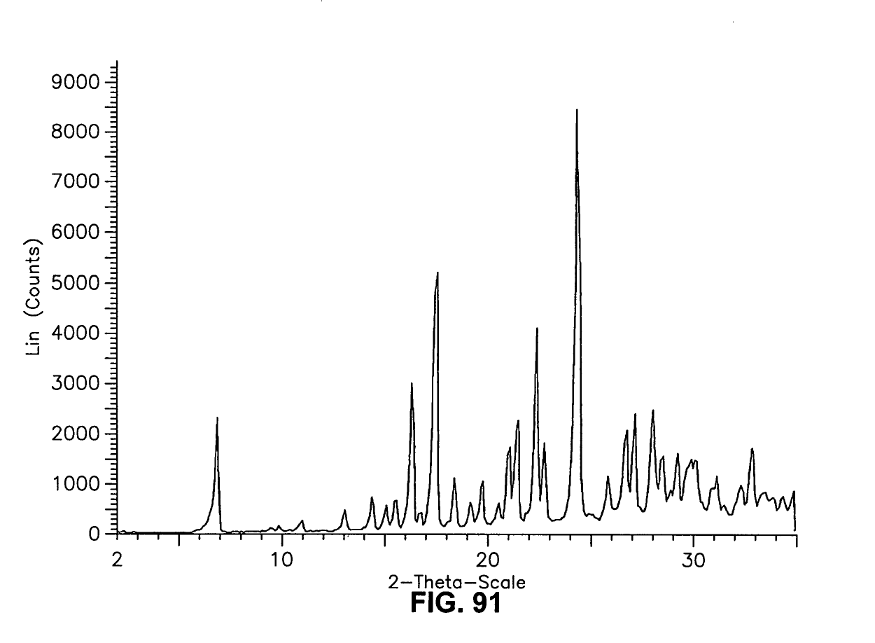

FIG. 92 shows the thermal analysis results for carvedilol hydrobromide anhydrous forms.

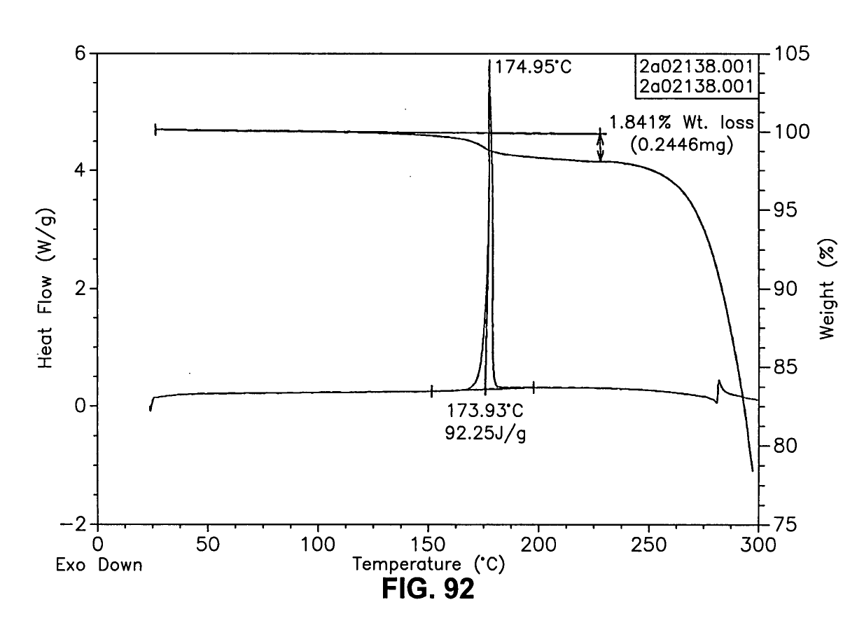

FIG. 93 is an FT-Raman spectrum for carvedilol hydrobromide anhydrous forms.

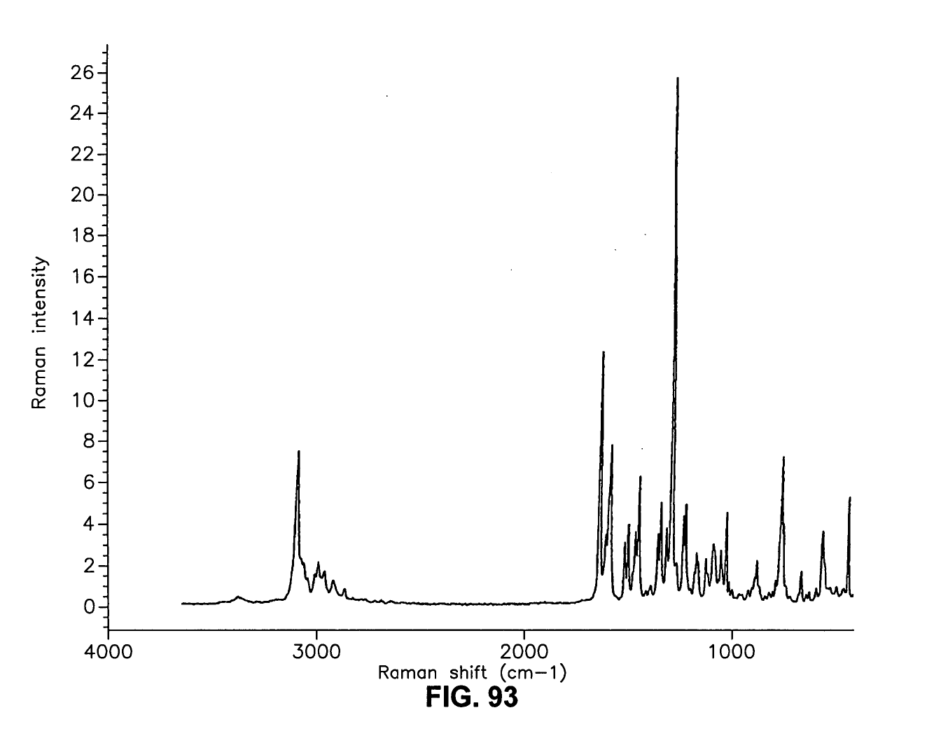

FIG. 94 is an FT-Raman spectrum for carvedilol hydrobromide anhydrous forms in the 4000-2000 cm⁻¹ region of the spectrum.

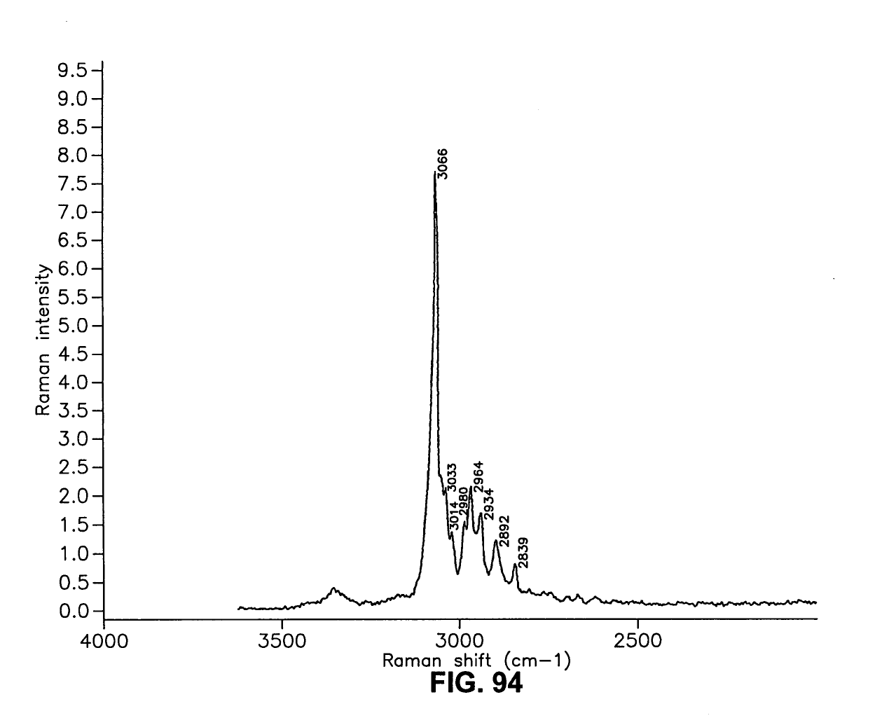

FIG. 95 is an FT-Raman spectrum for carvedilol hydrobromide anhydrous forms in the 2000-400 cm⁻¹ region of the spectrum.

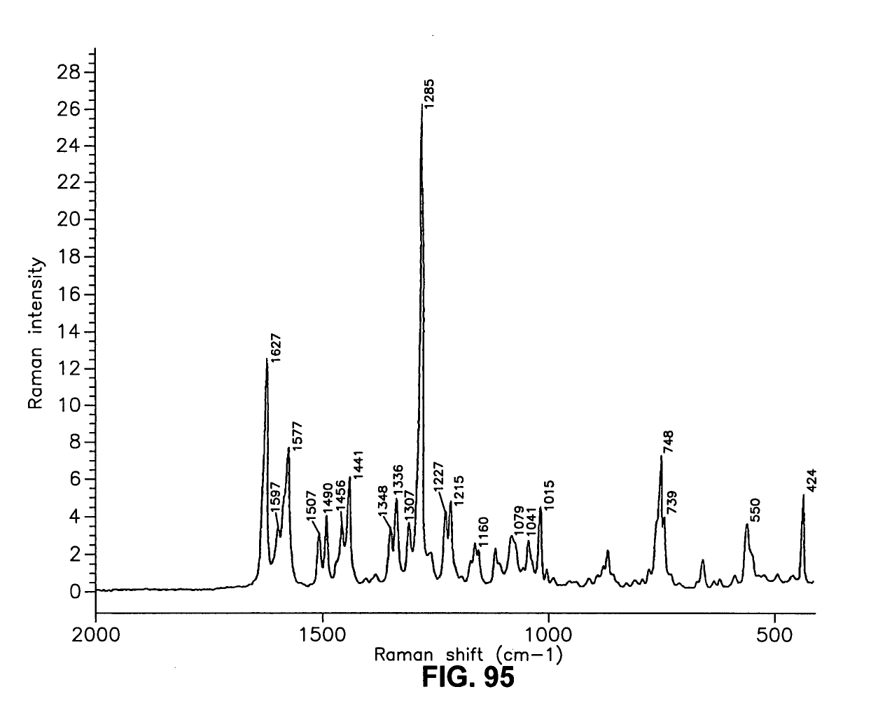

FIG. 96 is an FT-IR spectrum for carvedilol hydrobromide anhydrous forms.

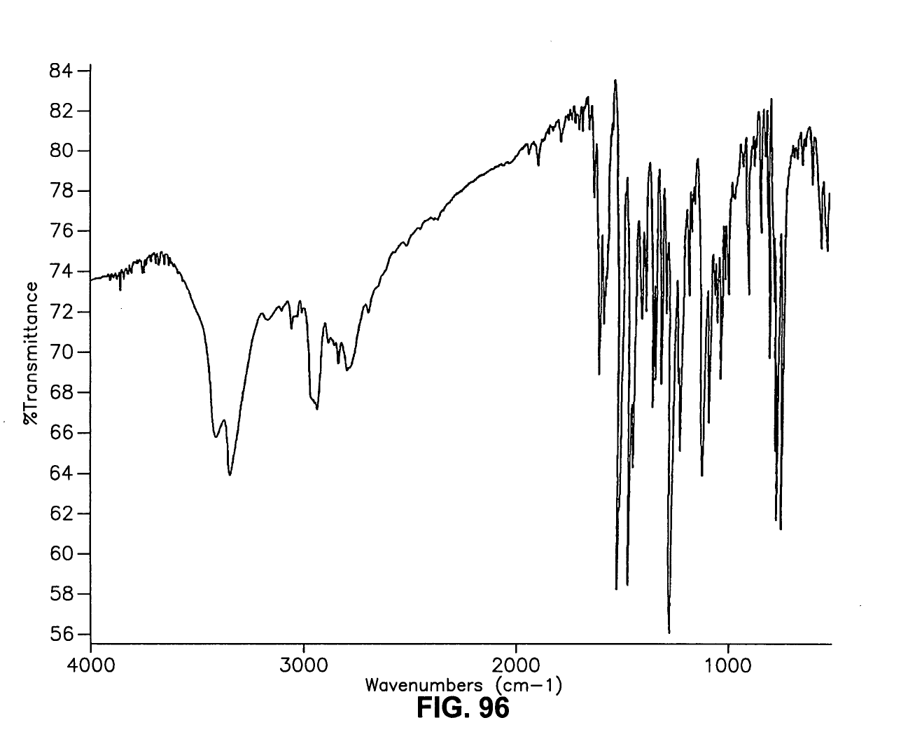

FIG. 97 is an FT-IR spectrum for carvedilol hydrobromide anhydrous forms in the 4000-2000 cm⁻¹ region of the spectrum.

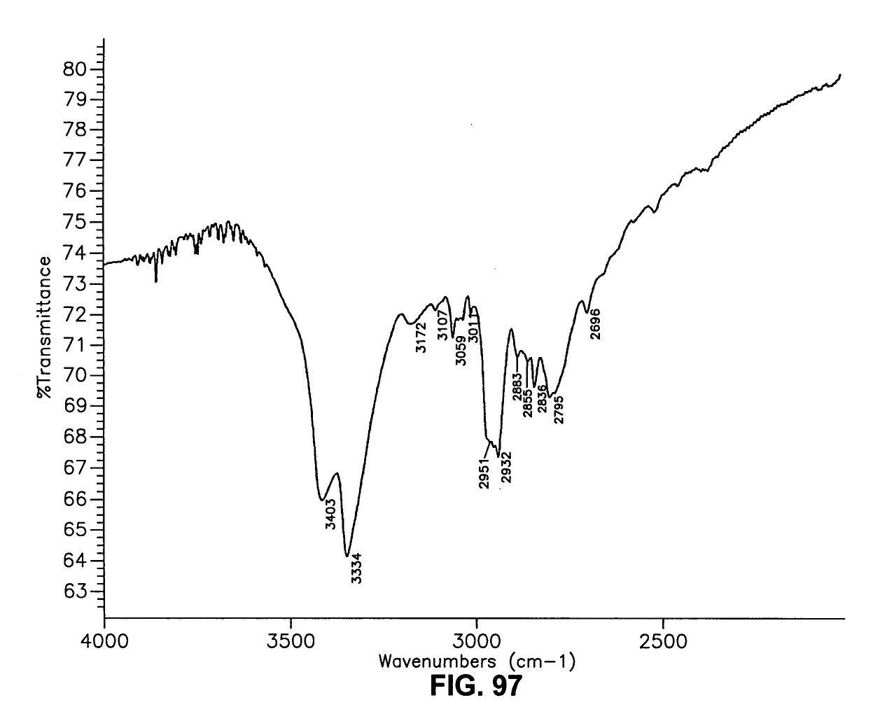

FIG. 98 is an FT-IR spectrum for carvedilol hydrobromide anhydrous forms in the 2000-500 cm⁻¹ region of the spectrum.

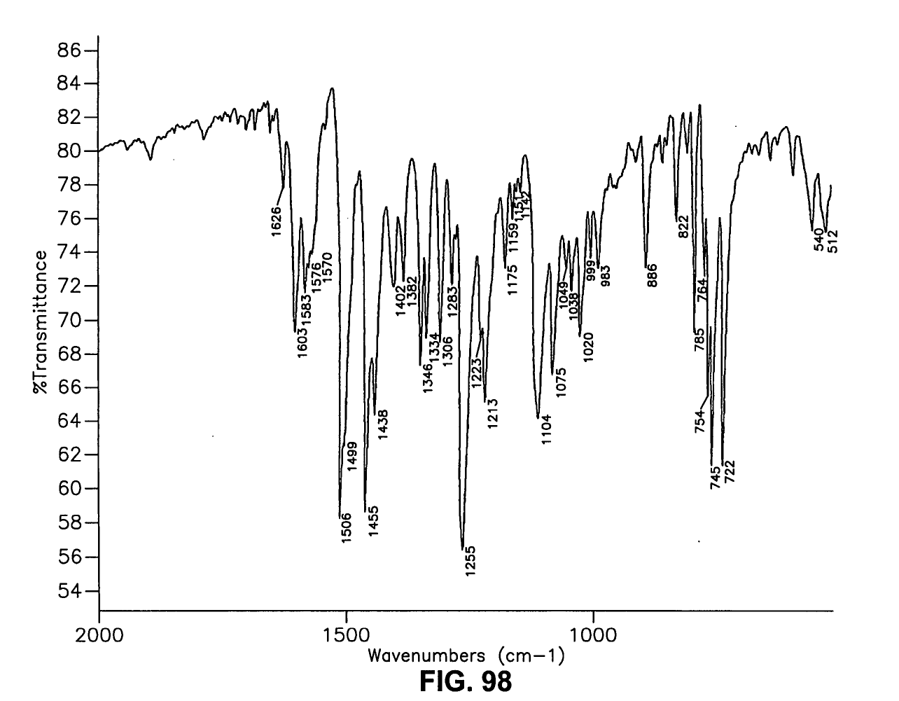

FIG. 99 is an x-ray powder diffractogram for carvedilol hydrobromide ethanol solvate.

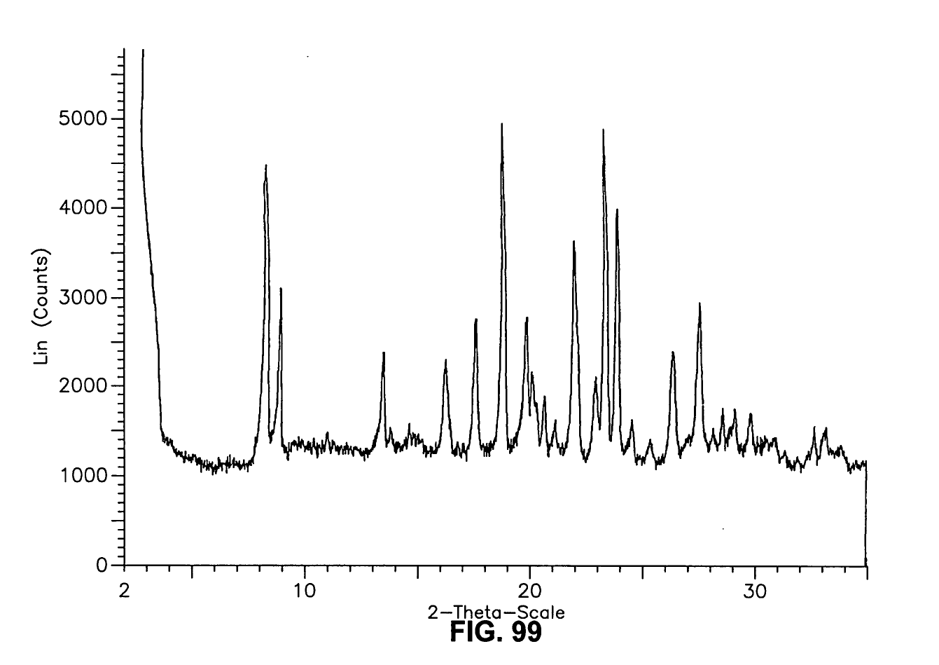

FIG. 100 shows the thermal analysis results for carvedilol hydrobromide ethanol solvate.

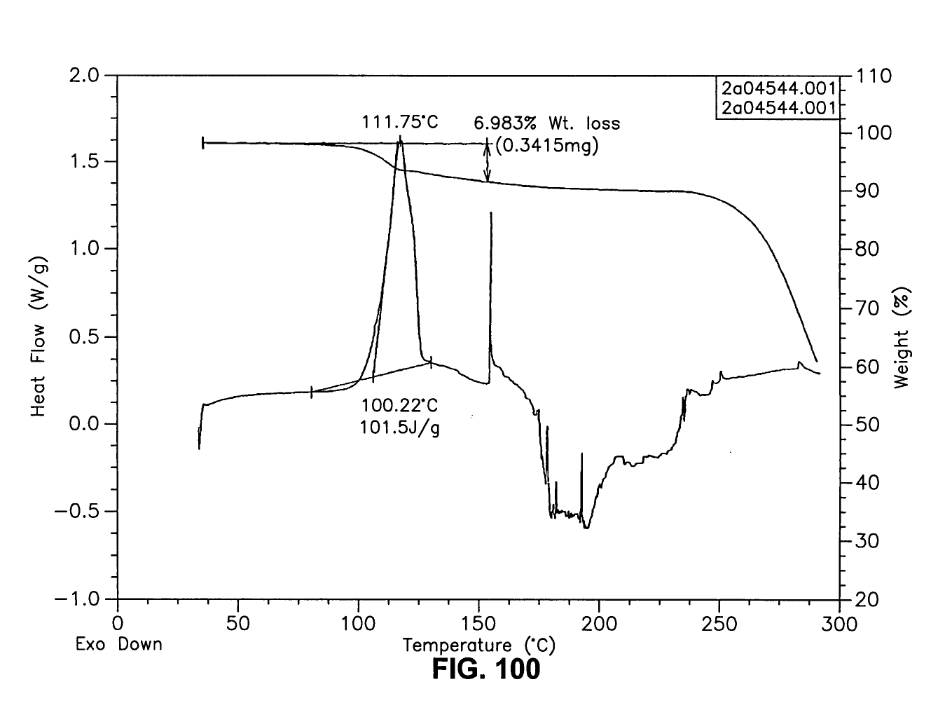

FIG. 101 is an FT-Raman spectrum for carvedilol hydrobromide ethanol solvate.

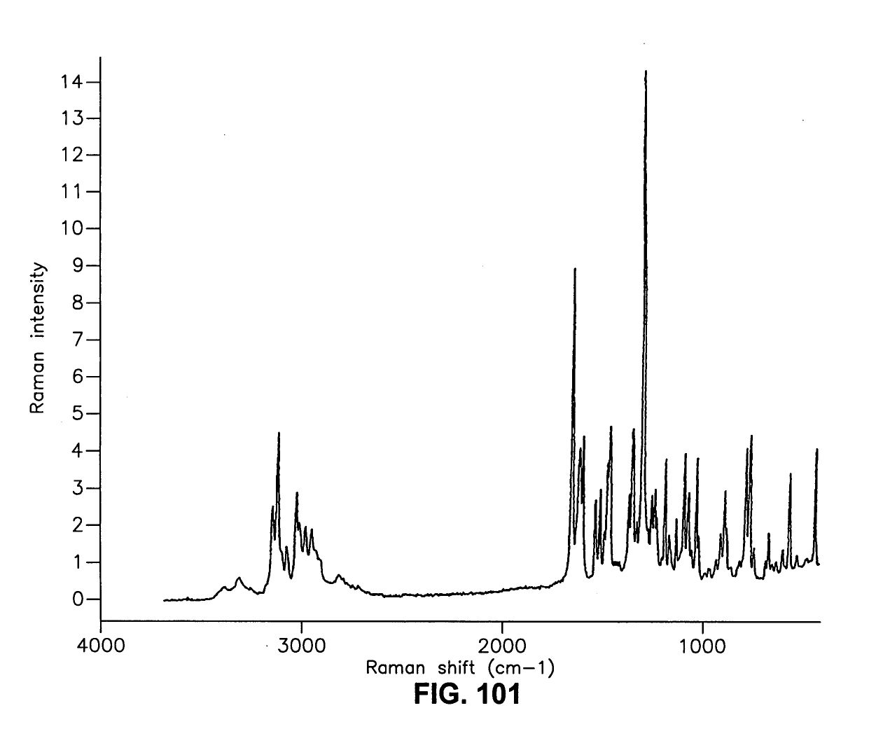

FIG. 102 is an FT-Raman spectrum for carvedilol hydrobromide ethanol solvate in the 4000-2000 cm⁻¹ region of the spectrum.

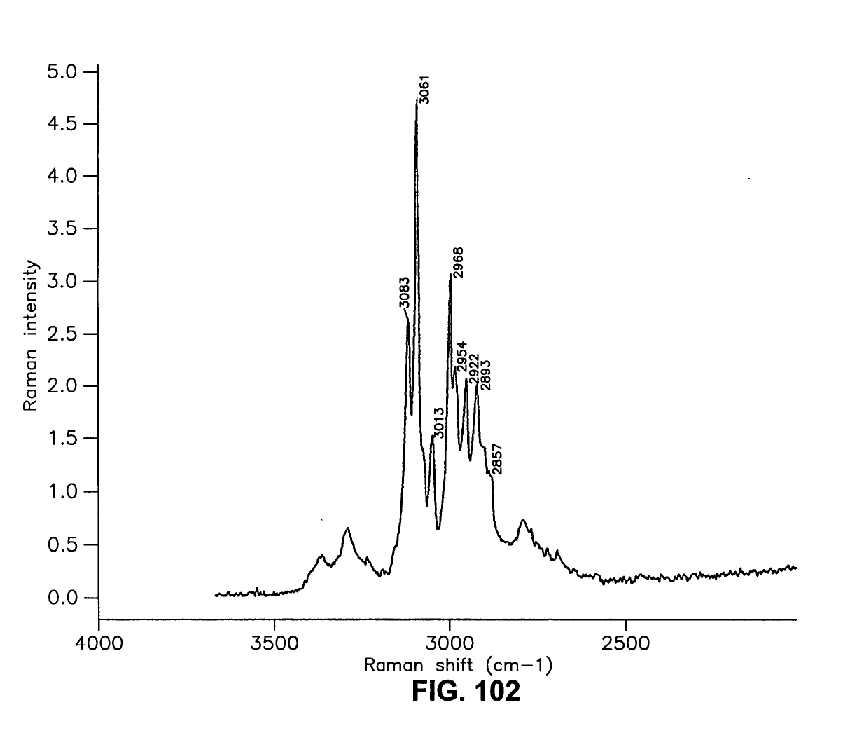

FIG. 103 is an FT-Raman spectrum for carvedilol hydrobromide ethanol solvate in the 2000-400 cm⁻¹ region of the spectrum.

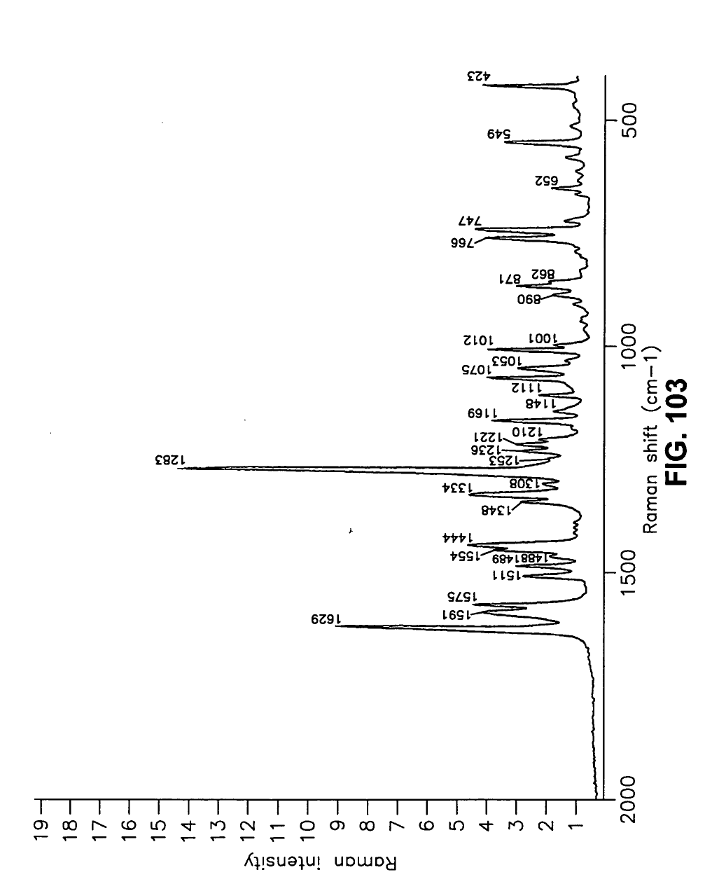

FIG. 104 is an FT-IR spectrum for carvedilol hydrobromide ethanol solvate.

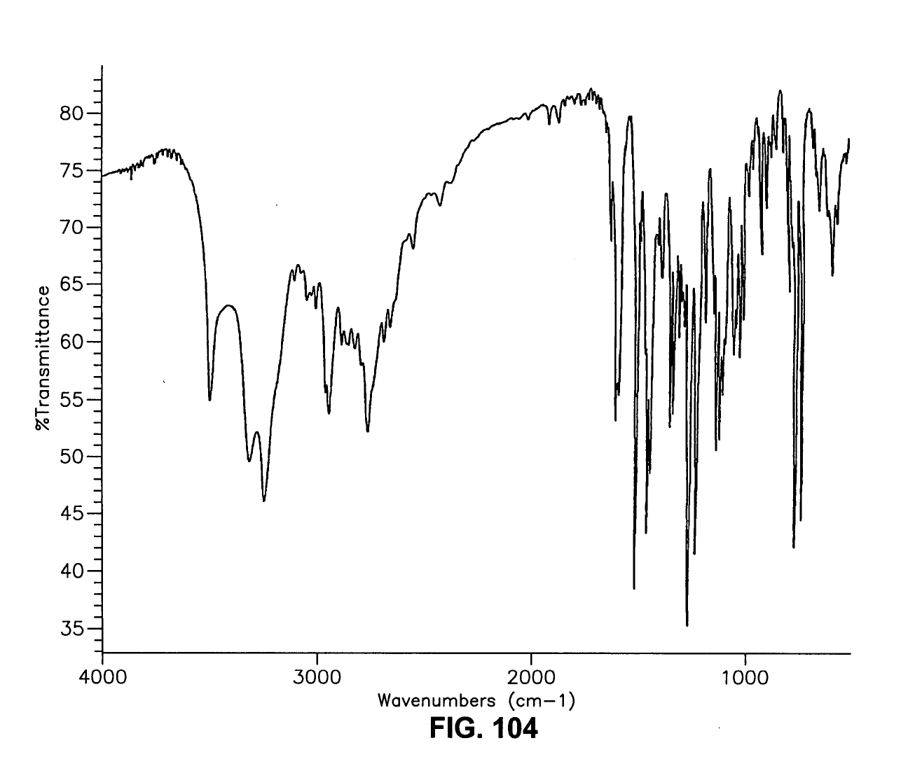

FIG. 105 is an FT-IR spectrum for carvedilol hydrobromide ethanol solvate in the 4000-2000 cm⁻¹ region of the spectrum.

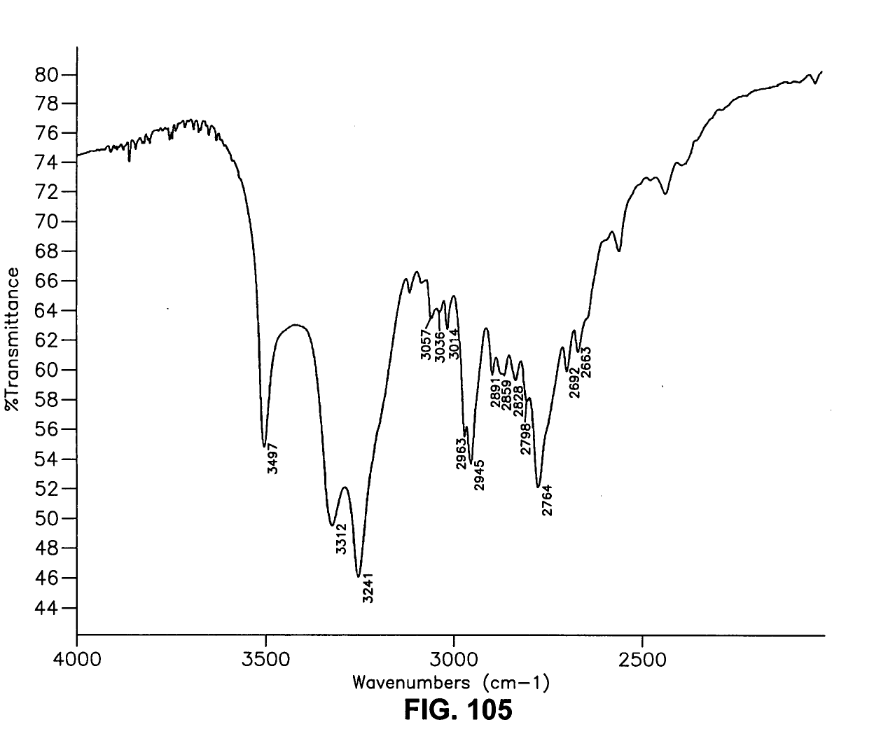

FIG. 106 is an FT-IR spectrum for carvedilol hydrobromide ethanol solvate in the 2000-500 cm⁻¹ region of the spectrum.

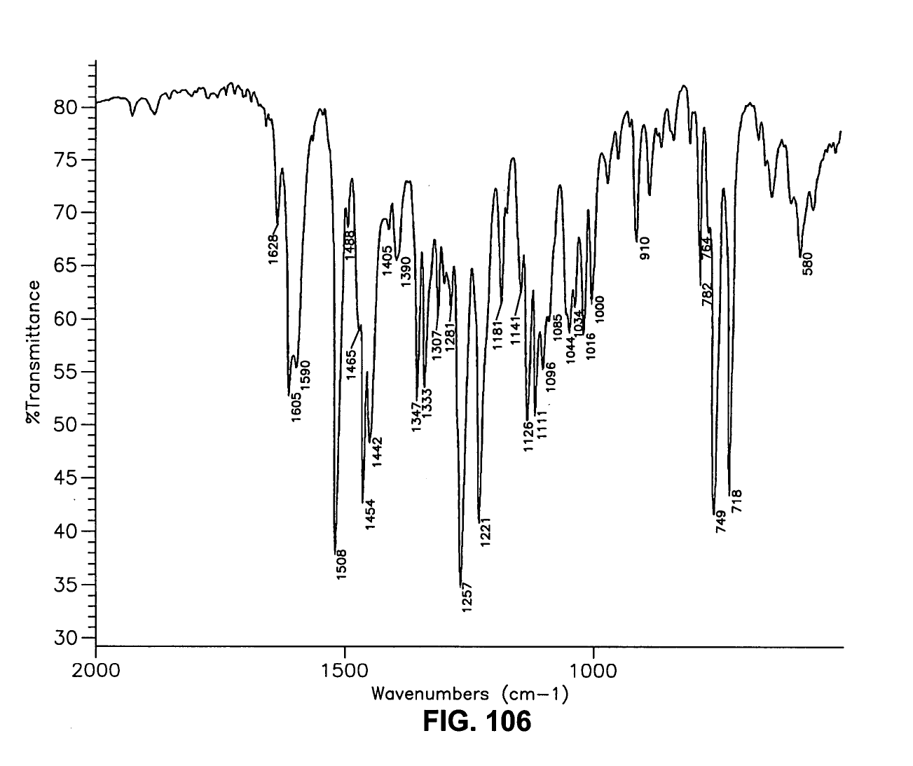

FIG. 107 is an x-ray powder diffractogram for carvedilol hydrobromide dioxane solvate.

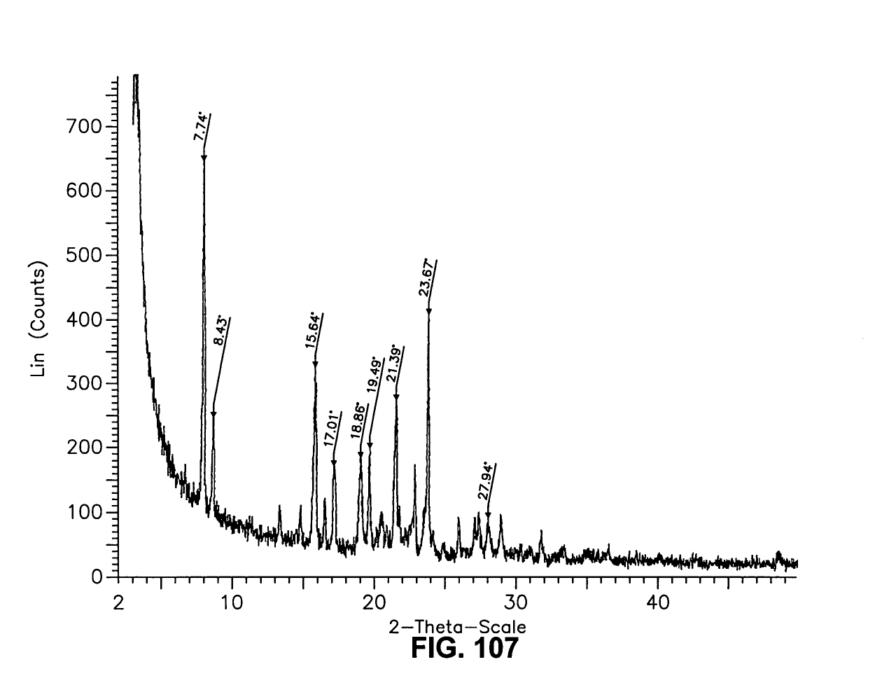

FIG. 108 is an x-ray powder diffractogram for carvedilol hydrobromide 1-pentanol solvate.

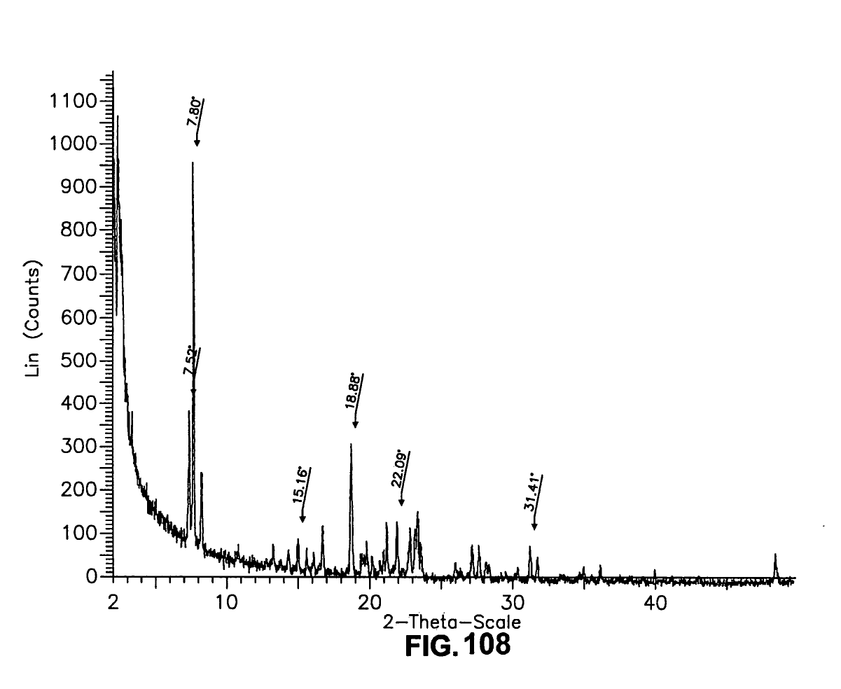

FIG. 109 is an x-ray powder diffractogram for carvedilol hydrobromide 2-methyl-1-propanol solvate.

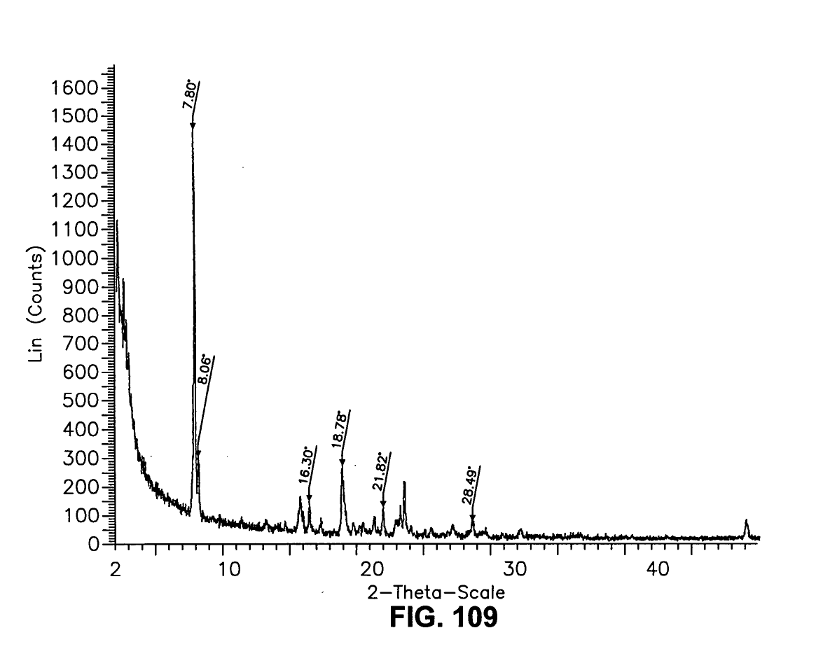

FIG. 110 is an x-ray powder diffractogram for carvedilol hydrobromide trifluoroethanol solvate.

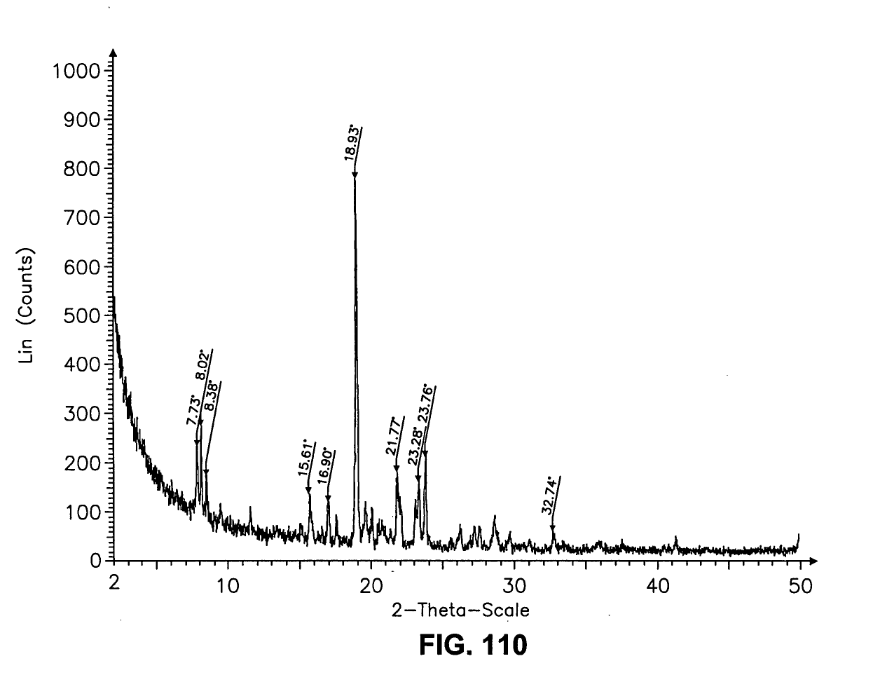

FIG. 111 is an x-ray powder diffractogram for carvedilol hydrobromide 2-propanol solvate.

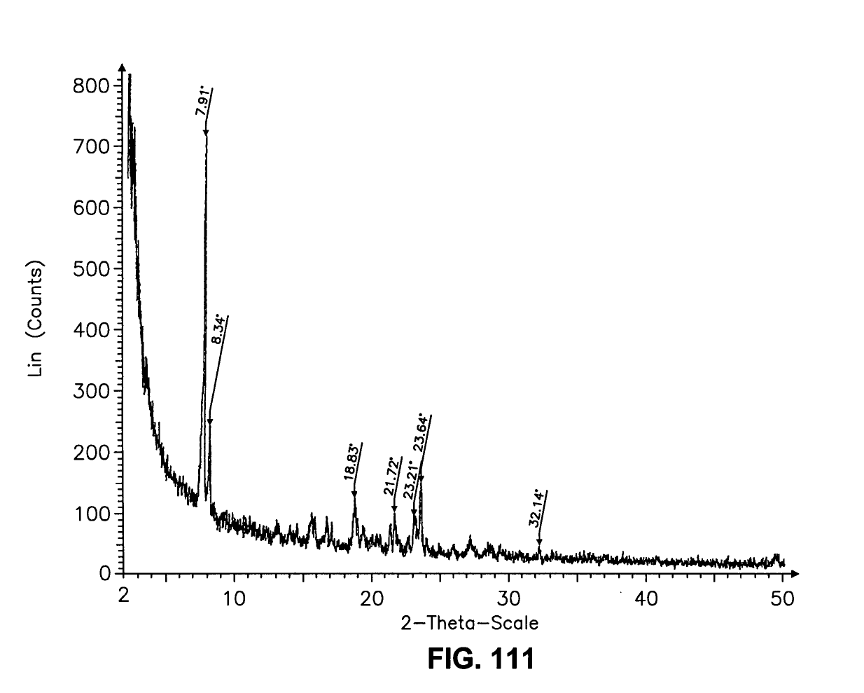

Carvedilol Citrate Salts

FIG. 112 is a FT-IR spectrum of carvedilol monocitrate salt.

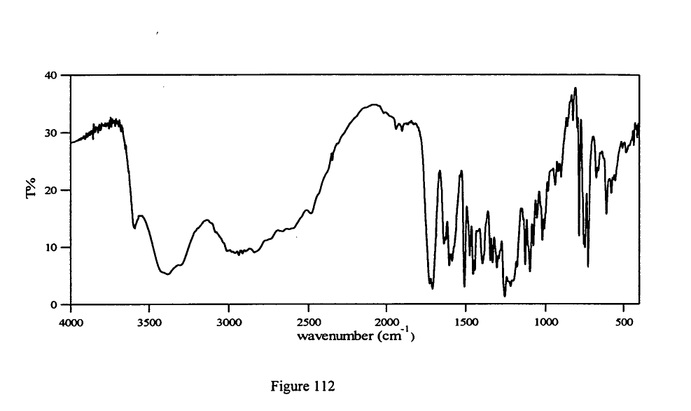

FIG. 113 depicts XRPD patterns of two different batches of Carvedilol monocitrate salt.

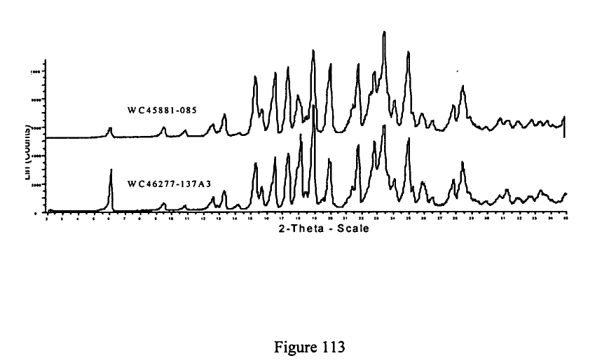

Carvedilol Mandelate Salts

FIG. 114 is a FT-IR spectrum of carvedilol mandelate salt.

FIG. 115 is a FT-Raman spectrum of carvedilol mandelate salt.

Carvedilol Lactate Salts

FIG. 116 is a FT-IR spectrum of carvedilol lactate salt.

FIG. 117 is a FT-Raman spectrum of carvedilol lacatate salt.

Carvedilol Maleate Salts

FIG. 118 is a FT-IR spectrum of carvedilol maleate salt.

FIG. 119 is a FT-Raman spectrum of carvedilol maleate salt.

Carvedilol Sulfate Salts

FIG. 120 is a FT-IR spectrum of carvedilol sulfate salt.

FIG. 121 is a FT-Raman spectrum of carvedilol sulfate salt.

Carvedilol Glutarate Salts

FIG. 122 is a FT-IR spectrum of carvedilol glutarate salt.

FIG. 123 is a FT-Raman spectrum of carvedilol glutarate salt.

Carvedilol Benzoate Salts

FIG. 124 is a FT-IR spectrum of carvedilol benzoate salt.

FIG. 125 is a FT-Raman spectrum of carvedilol benzoate salt.

Drug Solubility Enhancement in GI tract

FIG. 126 depicts a pH-solubility profile for carvedilol.

FIG. 127 depicts mean plasma profiles in beagle dogs following intra-colonic administration of a carvedilol solution containing captisol or carvedilol in aqueous suspension.

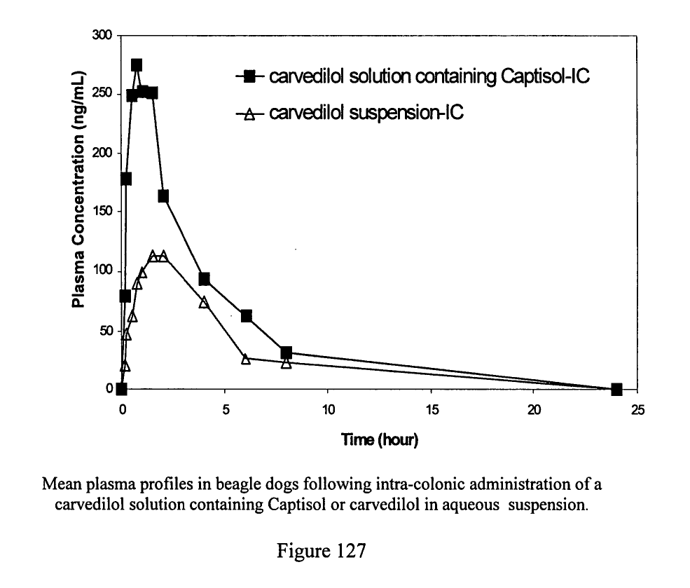

FIG. 128 depicts dissolution/solubility profile of carvedilol phosphate in pH=7.1 tris buffer.

FIG. 129 depicts mean plasma profiles in beagle dogs following oral administration of the formulations listed in Table 15.

FIG. 130 depicts mean plasma profiles following oral administration of companion capsules filled with four formulations at 10 mg strength to beagle dogs and also as described in Table 16.

Pharmacodynamic Profiles

FIG. 131 depicts a plasma profile from tablets formulated according to Example 29 (A).

FIG. 132 depicts a plasma profile of subjects for formulation described in Example 33.

### DETAILED DESCRIPTION OF THE INVENTION

In general, the present invention relates to carvedilol free base or carvedilol salts, anhydrous forms, or solvates thereof, corresponding pharmaceutical compositions or controlled release dosage forms or formulations, and delivery methods of carvedilol forms to the gastrointestingal tract or methods to treat cardiovascular diseases, which may include, but are not limited to hypertension, congestive heart failure, atherosclerosis, and angina.

The present invention relates to control release formulations, which comprise various cavedilol forms, which may include, but are not limited to carvedilol free base or corresponding carvedilol salts, anhydrous forms or solvates thereof.

In particular the present invention relates to a controlled release formulation or delivery device, which comprises:

a core containing a carvedilol free base or a carvedilol salt, solvate or anhydrous form thereof; a release modifying agent; and an outer coating covering the core;
where thickness of the outer coating is adapted: for substantial impermeability to entry of fluid present in an environment of use and for substantial impermeability toward release of the carvedilol free base or the carvedilol salt, solvate or anhydrous form thereof during a predetermined dosing interval; and for a controlled release dispensing exit of the carvedilol free base or the carvedilol salt, solvate or anhydrous form thereof after the predetermined dosing interval;
where the outer coating includes at least one orifice in at least one face area of the controlled delivery device extending substantially through the outer coating but not penetrating the core that communicates from the environment of use to the core allowing for release of the carvedilol free base or the carvedilol salt, solvate or anhydrous form thereof into the environment of use;
   where the at least one orifice in the at least one face area of the controlled release delivery device has a substantially dependent rate limiting release factor dependent upon exit of the carvedilol free base or the carvedilol salt, solvate or anhydrous form thereof from the at least one orifice via dissolution, diffusion or erosion; and
   where the release modifying agent enhances or hinders release of the carvedilol free base or the carvedilol salt, solvate or anhydrous form thereof depending upon solubility or effective solubility of the carvedilol free base or the carvedilol salt, solvate or anhydrous form thereof in the environment of use.

The present invention generally also relates to a controlled release formulation, which comprises at least one of these components:

[a] carvedilol free base, and [b] a solubility enhanced carvedilol salt, solvate or anhydrous forms; or

[a] carvedilol free base; or [b] a solubility enhanced carvedilol salt, solvate or anhydrous forms;

where the aforementioned controlled release formulation following oral dosage exhibits a substantially biphasic plasma profile which exhibits a first plasma concentration peak level and a first Tₘₐₓ pulse within 1-4 hours of ingestion and a second a plasma concentration peak level and second Tₘₐₓ pulse within 5-8 hours after ingestion.

Carvedilol Salts, Anhydrous Forms, or Solvates Thereof

In general, the present invention relates to carvedilol salts, anhydrous forms or solvates thereof.

In particular, the present invention relates to carvedilol free base or a novel carvedilol salt, anhydrous, or solvate forms thereof, which may include, but are not limited to crystalline or other solid forms, such as a salt form of 1-(carbazol-4-yloxy-3-[[2-(o-methoxyphenoxy)ethyl] amino]-2-propanol).

Carvedilol free base or all carvedilol salt, anhydrous or solvate compound forms suitable for use in the present invention, which include starting materials (i.e., such as carvedilol or carvedilol free base), intermediates or products, etc., are prepared as described herein, or by the application or adaptation of known methods, which may be methods used heretofore or described in the literature.

Carvedilol is disclosed and claimed in U.S. Pat. No. 4,503,067 to Wiedemann et al. (“U.S. '067 Patent”). Reference should be made to U.S. '067 Patent for its full disclosure, which include methods of preparing or using the carvedilol compound. The entire disclosure of the U.S. '067 Patent is incorporated herein by reference in its entirety.

U.S. Pat. No. 6,515,010 to Franchini et al., which is hereby incorporated by reference in its entirety, discloses a novel salt form of carvedilol, namely carvedilol methanesulfonate salt form, pharmaceutical compositions containing carvedilol methanesulfonate and the use of the aforementioned compound in the treatment of hypertension, congestive heart failure and angina.

The present invention relates to a carvedilol compound, which is a free base or a novel salt, solvate or anhydrous form of carvedilol, which may include, but is not limited to a crystalline salt or other solid form.

In accordance with the present invention, it has been unexpectedly found that the aforementioned carvedilol compound forms may be isolated readily, but not limited to novel crystalline or other solid forms, which display much higher solubility when compared to the free base form of carvedilol. The present invention is related to pharmaceutically acceptable acid addition salts of carvedilol free base or corresponding forms.

Such pharmaceutically acceptable acid addition salts of carvedilol free base or corresponding forms thereof are formed by reaction with appropriate organic acids or mineral acids, which may include, but are not limited to formation by such methods described herein or conventionally known in the chemical arts.

For example, such acid addition salts may be formed via the following conventional chemical reactions or methods:

reaction of carvedilol free base with a suitable organic acid or mineral acid in an aqueous miscible organic solvent with isolation of the formed acid addition salt by removing the organic solvent by conventional art known techniques; or

reaction of carvedilol free base with a suitable organic acid or mineral acid in an aqueous immiscible organic solvent where the formed acid addition salt is separated directly or isolated by removing the solvent by conventional art known techniques, such as by filtration.

For example, an acid addition salt of carvedilol free base or carvedilol salt, solvate or anhydrous form thereof is an acid addition salt formed from mineral acids or organic acids.

Representative examples of such suitable organic or mineral acids may include, but are not limited to maleic acid, fumaric acid, benzoic acid, ascorbic acid, pamoic acid, succinic acid, bismethylenesalicyclic acid, methane sulphonic or sulfonic acid, acetic acid, propionic acid, tartaric acid, salicyclic acid, citric acid, gluconic acid, aspartic acid, stearic acid, palmitic acid, itaconic acid, glycolic acid, p-aminobenzoic acid, glutamic acid, benzene sulfonic acid or sulphonic acid, hydrochloric acid, hydrobromic acid, sulfuric acid or sulphuric acid, cyclohexylsulfamic acid, phosphoric acid, nitric acid and the like.

In accordance with the present invention, mineral acids may be selected from, but are not limited to hydrobromic acid, hydrochloric acid, phosphoric acid, sulfuric acid or sulphuric acid, and the like; and organic acids may be selected from, but not limited to methansulphuric acid, tartaric acid,- maleic acid, acetic acid, citric acid, benzoic acid and the like.

As indicated above, the present invention further relates to carvedilol salt forms, which may include, but are not limited to novel crystalline salt or other solid forms of carvedilol mandelate, carvedilol lactate, carvedilol maleate, carvedilol sulfate, carvedilol glutarate, carvedilol mesylate, carvedilol phosphate, carvedilol citrate, carvedilol hydrogen bromide, carvedilol oxalate, carvedilol hydrogen chloride, carvedilol hydrogen bromide, carvedilol benzoate, or corresponding solvates thereof.

More particularly, the present invention relates to carvedilol salt forms, which may include, but are not limited to carvedilol hydrogen phosphate, carvedilol dihydrogen phosphate, carvedilol dihydrogen phosphate hemihydrate, carvedilol dihydrogen phosphate dihydrate, carvedilol dihydrogen phosphate methanol solvate, carvedilol hydrobromide monohydrate, carvedilol hydrobromide dioxane solvate, carvedilol hydrobromide 1-pentanol solvate, carvedilol hydrobromide 2-methyl-1-propanol solvate, carvedilol hydrobromide trifluoroethanol solvate, carvedilol hydrobromide 2-propanol solvate, carvedilol hydrobromide n-propanol solvate #1, carvedilol hydrobromide n-propanol solvate #2, carvedilol hydrobromide anhydrous forms or anhydrous forms, carvedilol hydrobromide ethanol solvate, carvedilol hydrobromide dioxane solvate, carvedilol monocitrate monohydrate, carvedilol mandelate, carvedilol lactate, carvedilol hydrochloride, carvedilol maleate, carvedilol sulfate, carvedilol glutarate, or corresponding anhydrous forms, solvates thereof.

In accordance with the present invention, other salts or solvates of carvedilol of the present invention may be isolated, but not limited to different solid or crystalline forms. Moreover, a specific identified species of such carvedilol salts (or a specific identified corresponding solvate species) also may be isolated as, but not limited to various different crystalline or solid forms, which may include anhydrous forms or solvate forms. For example, suitable solvates of carvedilol phosphate as defined in the present invention, include, but are not limited to carvedilol dihydrogen phosphate hemihydrate, carvedilol dihydrogen phosphate dihydrate (i.e., which include Forms II and IV, respectively), carvedilol dihydrogen phosphate methanol solvate, and carvedilol hydrogen phosphate.

In light of this, carvedilol salt forms of the present invention (i.e., which may include different polymorphs, ahydrous forms, solvates, or hydrates thereof) may exhibit characteristic polymorphism. As conventionally understood in the art, polymorphism is defined as an ability of a compound to crystallize as more than one distinct crystalline or “polymorphic” species. A polymorph is defined as a solid crystalline phase of a compound with at least two different arrangements or polymorphic forms of that compound molecule in the solid state.

Polymorphic forms of any given compound, including those of the present invention, are defined by the same chemical formula or composition and are as distinct in chemical structure as crystalline structures of two different chemical compounds. Such compounds may differ in packing, geometrical arrangement of respective crystalline lattices, etc.

In light of the foregoing, chemical and/or physical properties or characteristics vary with each distinct polymorphic form, which may include variations in solubility, melting point, density, hardness, crystal shape, optical and electrical properties, vapor pressure, stability, etc.

Solvates or hydrates of carvedilol salt forms of the present invention also may be formed when solvent molecules are incorporated into the crystalline lattice structure of the compound molecule during the crystallization process. For example, solvate forms of the present invention may incorporate nonaqueous solvents such as methanol and the like as described herein below. Hydrate forms are solvate forms, which incorporate water as a solvent into a crystalline lattice.

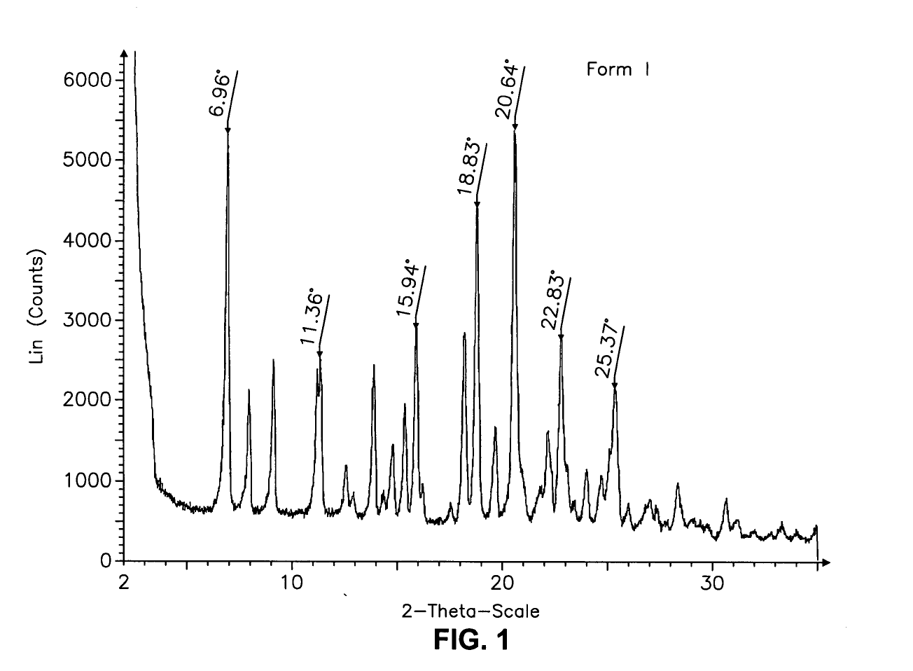

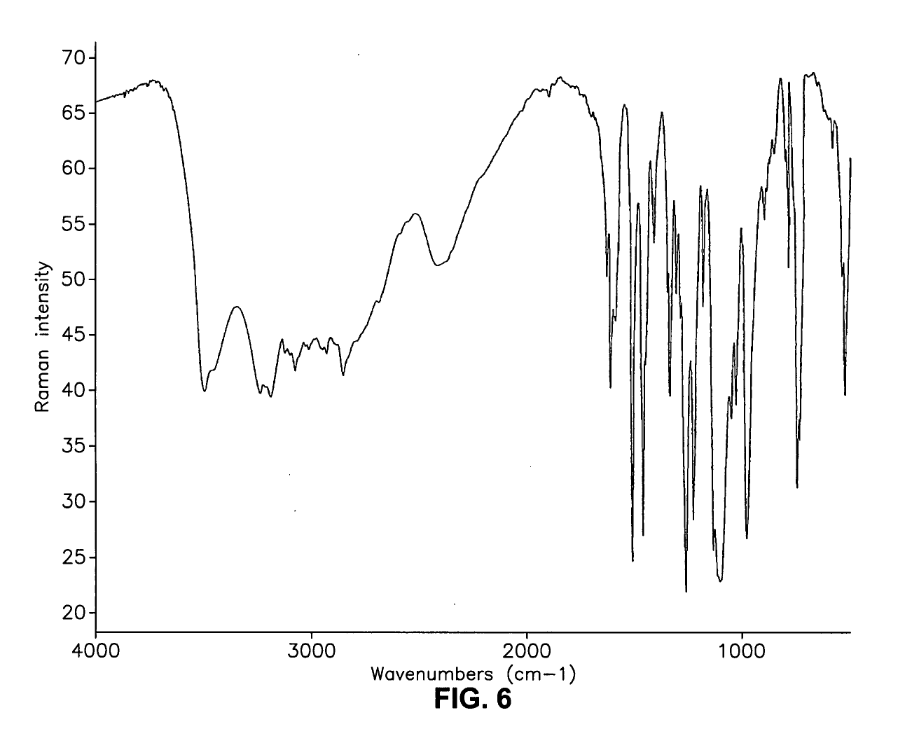

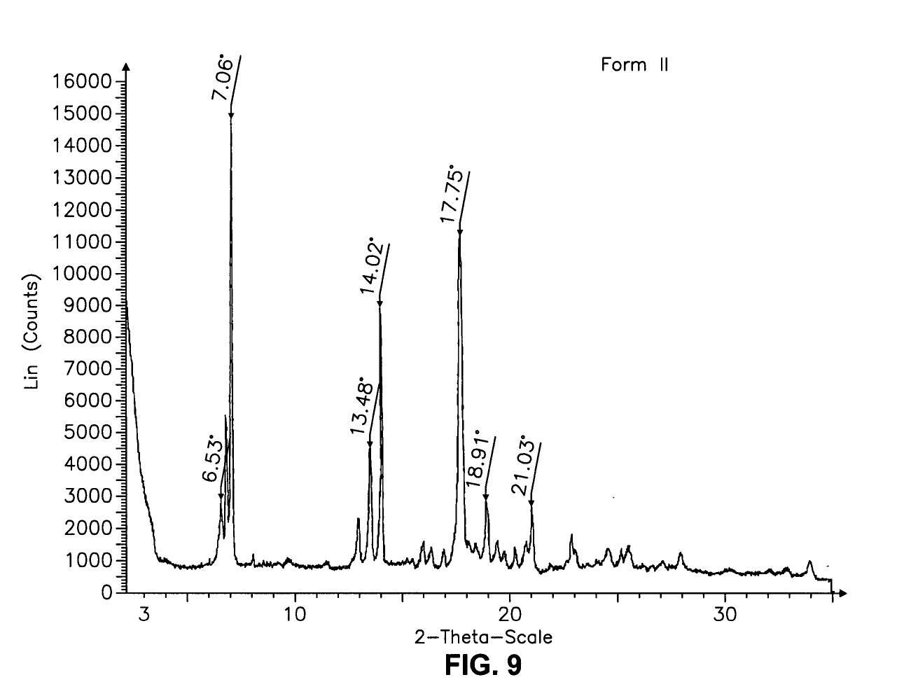

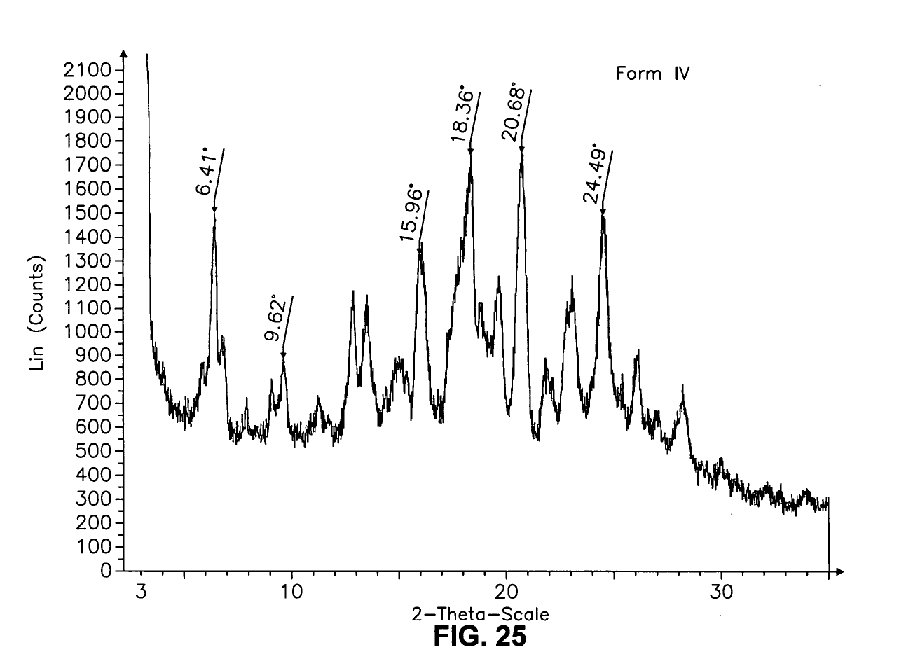

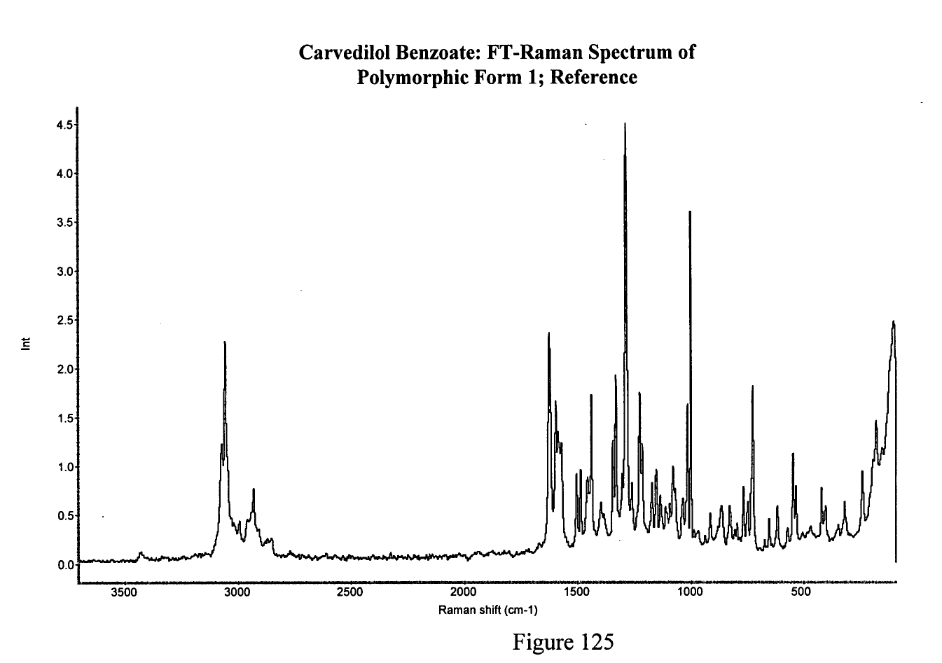

In general, FIGS. 1-125 depict spectroscopic and other characterizing data for different, specific, and distinct carvedilol salt, anhydrous forms, or solvate forms thereof, which may be include, but are not limited to crystalline or other solid forms. For example, carvedilol dihydrogen phosphate, may be isolated as two different and distinct crystalline forms, Forms II and IV, respectively represented and substantially shown FIGS. 9 to 6 (for Form II) and FIG. 25 (for Form IV), which represent spectroscopic and/or other characterizing data.

It is recognized that the compounds of the present invention may exist in forms as stereoisomers, regioisomers, or diastereiomers. These compounds may contain one or more asymmetric carbon atoms and may exist in racemic and optically active forms. For example, carvedilol may exist as racemic mixture of R(+) and S(−) enantiomers, or in separate respectively optical forms, i.e., existing separately as either the R(+) enantiomer form or in the S(+) enantiomer form. All of these individual compounds, isomers, and mixtures thereof are included within the scope of the present invention.

Carvedilol salts of the present invention may be prepared by various techniques, such as those exemplified below.

For example, crystalline carvedilol dihydrogen phosphate hemihydrate of the instant invention can be prepared by crystallization from an acetone-water solvent system containing carvedilol and H₃PO₄. Also suitable solvates of carvedilol phosphate salts of present invention may be prepared by preparing a slurrying a carvedilol phosphate salt, such as a carvedilol dihydrogen salt, in a solvent, such as methanol.

In another example, crystalline carvedilol hydrobromide monohydrate of the present invention can be prepared by crystallization from an acetone-water solvent system containing carvedilol and hydrobromic acid.

Also, suitable solvates of carvedilol hydrobromide salts may be made by preparing a slurry of the carvedilol hydrobromide salt in a solvent (i.e., such as dioxane, 1-pentanol, 2-methyl-1-propanol, trifluoroethanol, 2-propanol and n-propanol. In particular, solvates of carvedilol hydrobromide as defined in the present invention, include, but are not limited to carvedilol hydrobromide 1-pentanol solvate, carvedilol hydrobromide 2-methyl-1-pentanol solvate, carvedilol hydrobromide trifluoroethanol solvate, carvedilol hydrobromide 2-propanol solvate, carvedilol hydrobromide n-propanol solvate #1, carvedilol hydrobromide n-propanol solvate #2, carvedilol hydrobromide ethanol solvate, carvedilol hydrobromide anhydrous forms), and/or dissolving the carvedilol hydrobromide salt in the aforementioned solvents and allowing the salt to crystallize out.

Carvedilol hydrobromide anhydrous forms can be prepared by dissolving carvedilol in a solvent, such as dichloromethane, acetonitrile or isopropyl acetate, followed by the addition of anhydrous HBr (HBr in acetic acid or gaseous HBr).

In yet another example, the crystalline carvedilol citrate salt of the instant invention can be prepared by making an aqueous citric acid solution saturated with carvedilol, either by lowering the temperature of the solution, or slowly evaporating water from the solution. In addition, it can be prepared by crystallization from an acetone-water solvent system containing carvedilol and citric acid.

A particularly useful and surprising characteristic of the crystalline form of carvedilol citrate salt stems from the fact that citric acid is a prochiral molecule. Consequently, a 1 to 1 ratio of racemic diasteromers are present in the crystalline carvedilol citrate salt lattice. This avoids generation of yet more optically active forms that could potentially complicate stability, dissolution rates, in vivo absorption metabolism and possibly pharmacologic effects.

According to the instant invention, the various salt forms of carvedilol or corresponding solvates thereof are distinguished from each other using different characterization or identification techniques. Such techniques, include solid state ¹³C Nuclear Magnetic Resonance (NMR), ³¹P Nuclear Magnetic Resonance (NMR), Infrared (IR), Raman, X-ray powder diffraction, etc. and/or other techniques, such as Differential Scanning Calorimetry (DSC) (i.e., which measures the amount of energy (heat) absorbed or released by a sample as it is heated, cooled or held at constant temperature).

In general, the aforementioned solid state NMR techniques are non-destructive techniques to yield spectra, which depict an NMR peak for each magnetically non-equivalent carbon site the solid-state

For example, in identification of compounds of the present invention, ¹³C NMR spectrum of a powdered microcrystalline organic molecules reflect that the number of peaks observed for a given sample will depend on the number of chemically unique carbons per molecule and the number of non-equivalent molecules per unit cell. Peak positions (chemical shifts) of carbon atoms reflect the chemical environment of the carbon in much the same manner as in solution-state ¹³C NMR. Although peaks can overlap, each peak is in principle assignable to a single type of carbon. Therefore, an approximate count of the number of carbon sites observed yields useful information about the crystalline phase of a small organic molecule.

Based upon the foregoing, the same principles apply to phosphorus, which has additional advantages due to high sensitivity of the ³¹P nucleus.

Polymorphism also can be studied by comparison of ¹³C and ³¹P spectra. In the case of amorphous material, broadened peak shapes are usually observed, reflecting the range of environments experienced by the ¹³C or ³¹P sites in amorphous material types.

Specifically, carvedilol salts, anhydrous forms or solvates thereof, which may include, but are not limited to novel crystalline or other solid forms, which are characterized substantially by spectroscopic data as described below and depicted in FIGS. 1-125.

Examples of spectroscopic data associated with specific carvedilol salt, anhydrous forms or solvate forms are described below.

For example, crystalline carvedilol dihydrogen phosphate hemihydrate (see, Example 1: Form I) is identified by an x-ray diffraction pattern as shown substantially in FIG. 1, which depicts characteristic peaks in degrees two-theta (2θ): i.e., 7.0±0.2 (2θ), 11.4±0.2 (2θ), 15.9±0.2 (2θ), 18.8±0.2 (2θ), 20.6±0.2 (2θ), 22.8±0.2 (2θ), and 25.4±0.2 (2θ).

Crystalline carvedilol dihydrogen phosphate dihydrate (see, Example 2: Form II) is identified by an x-ray diffraction pattern as shown substantially in FIG. 9, which depicts characteristic peaks in degrees two-theta (2θ): i.e., 6.5±0.2 (2θ), 7.1±0.2 (2θ), 13.5±0.2 (2θ), 14.0±0.2 (2θ), 17.8±0.2 (2θ), 18.9±0.2 (2θ), and 21.0±0.2 (2θ).

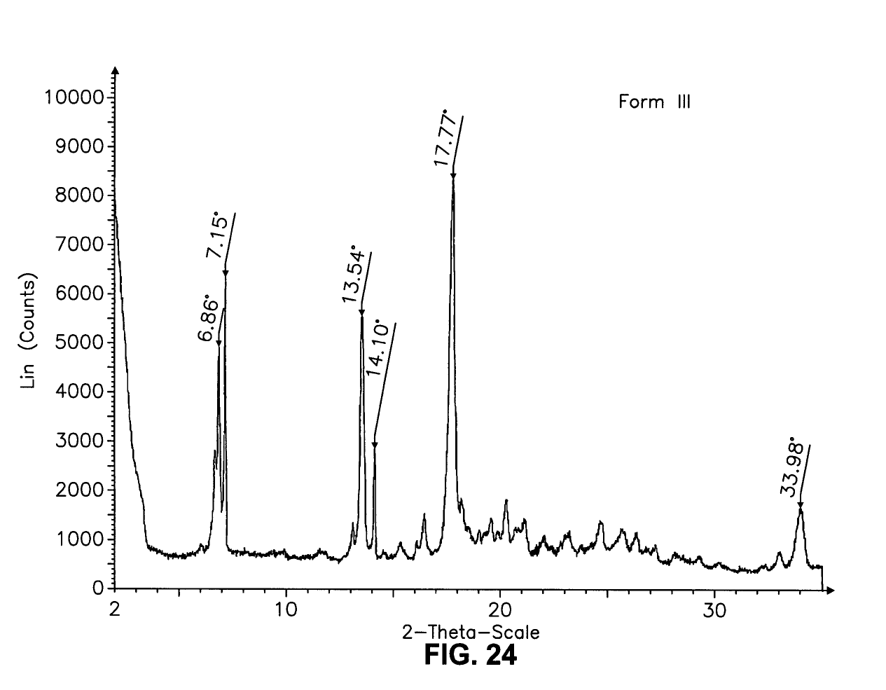

Crystalline carvedilol dihydrogen phosphate methanol solvate (see, Example 3: Form III) is identified by an x-ray diffraction pattern as shown substantially in FIG. 24, which depicts characteristic peaks in degrees two-theta (2θ): i.e., 6.9±0.2 (2θ), 7.2±0.2 (2θ), 13.5±0.2 (2θ), 14.1±0.2 (2θ), 17.8±0.2 (2θ), and 34.0±0.2 (2θ).

Crystalline carvedilol dihydrogen phosphate dihydrate (see, Example 4: Form IV) is identified by an x-ray diffraction pattern as shown substantially in FIG. 24, which depicts characteristic peaks in degrees two-theta (2θ): i.e., 6.4±0.2 (2θ), 9.6±0.2 (2θ), 16.0±0.2 (2θ), 18.4±0.2 (2θ), 20.7±0.2 (2θ), and 24.5±0.2 (2θ).

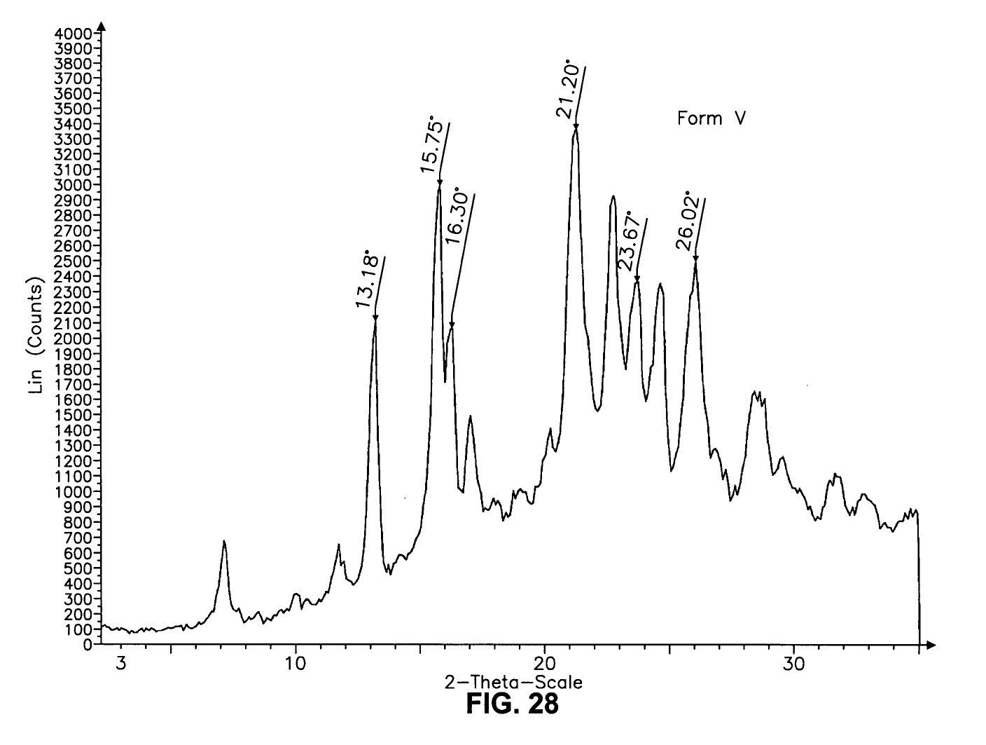

Crystalline carvedilol dihydrogen phosphate preparation (see, Example 5: Form V) is identified by an x-ray diffraction pattern as shown substantially in FIG. 28, which depicts characteristic peaks in degrees two-theta (2θ): i.e., 13.2±0.2 (2θ), 15.8±0.2 (2θ), 16.3±0.2 (2θ), 21.2±0.2 (2θ), 23.7±0.2 (2θ), and 26.0±0.2 (2θ).

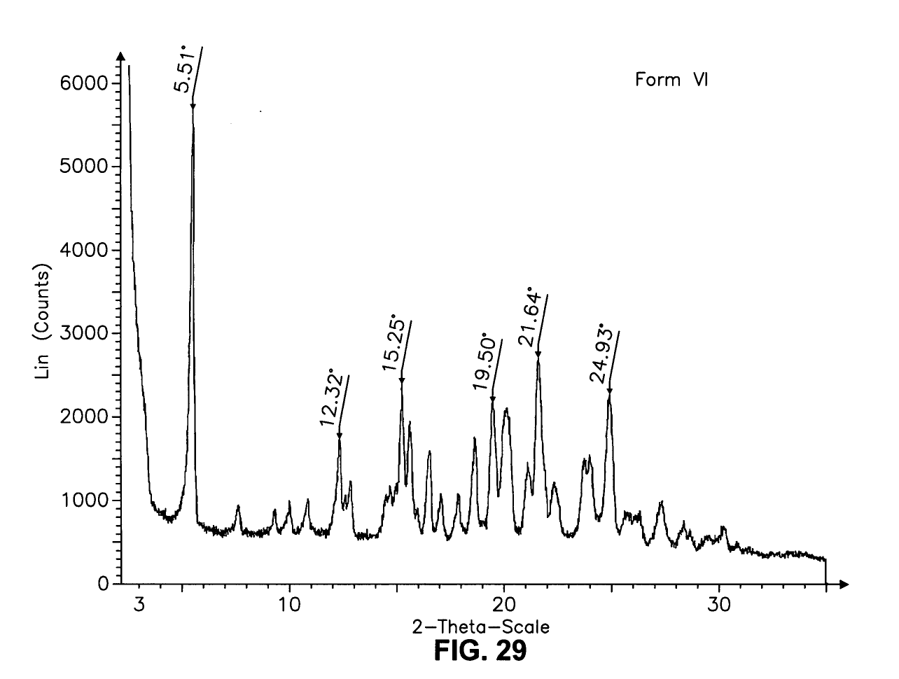

Crystalline carvedilol hydrogen phosphate preparation (see, Example 6: Form VI) is identified by an x-ray diffraction pattern as shown substantially in FIG. 29, which depicts characteristic peaks in degrees two-theta (2θ): i.e., 5.5±0.2 (2θ), 12.3±0.2 (2θ), 15.3±0.2 (2θ), 19.5±0.2 (2θ), 21.6±0.2 (2θ), and 24.9±0.2 (2θ).

Crystalline carvedilol hydrobromide monohydrate (see, Example 8: Form 1) is identified by an x-ray diffraction pattern as shown substantially in FIG. 1, which depicts characteristic peaks in degrees two-theta (2θ): i.e., 6.5±0.2 (2θ), 10.3±0.2 (2θ), 15.7±0.2 (2θ), 16.3±0.2 (2θ), 19.8±0.2 (2θ), 20.1±0.2 (2θ), 21.9±0.2 (2θ), 25.2±0.2 (2θ), and 30.6±0.2 (2θ).

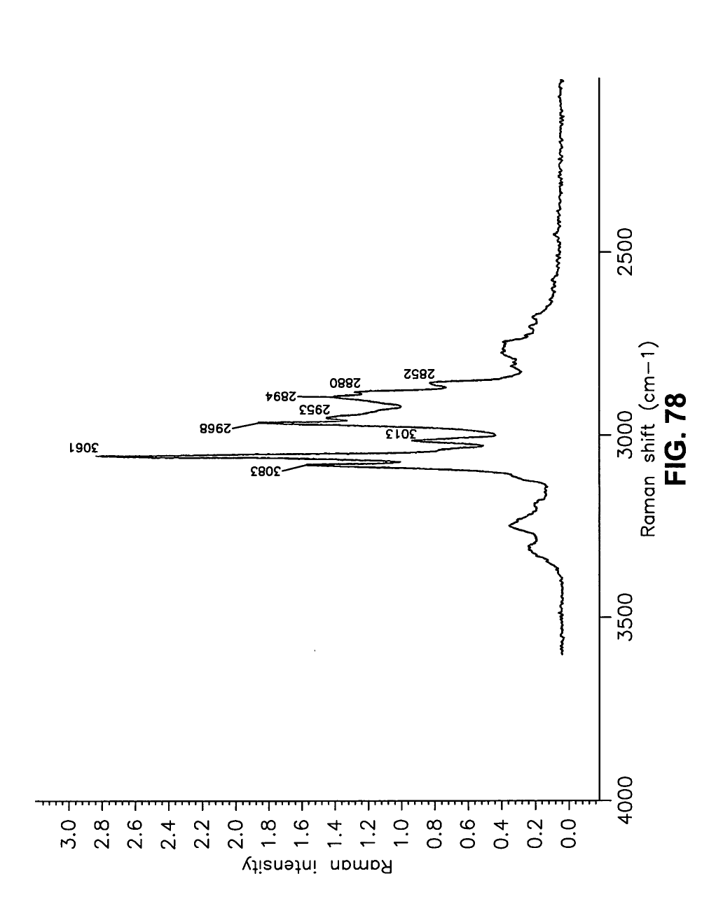

Crystalline carvedilol hydrobromide dioxane solvate (see, Example 9: Form 2) also is identified by an x-ray diffraction pattern as shown substantially in FIG. 78, which depicts characteristic peaks in degrees two-theta (2θ): i.e., 7.7±0.2 (2θ), 8.4±0.2 (2θ), 15.6±0.2 (2θ), 17.0±0.2 (2θ), 18.7±0.2 (2θ), 19.5±0.2 (2θ), 21.4±0.2 (2θ), 23.7±0.2 (2θ), and 27.9±0.2 (2θ).

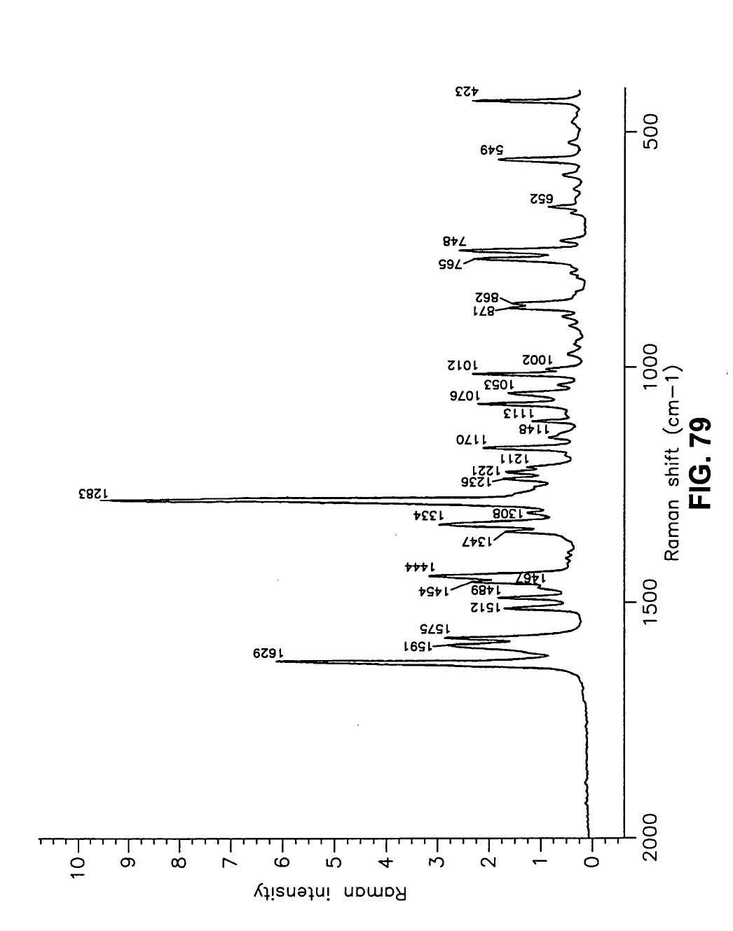

Crystalline carvedilol hydrobromide 1-pentanol solvate (see, Example 10: Form 3) also is identified by an x-ray diffraction pattern as shown substantially in FIG. 79, which depicts characteristic peaks in degrees two-theta (2θ): i.e., 77.5±0.2 (2θ), 7.8±0.2 (2θ), 15.2±0.2 (2θ), 18.9±0.2 (2θ), 22.1±0.2 (2θ), and 31.4±0.2 (2θ).

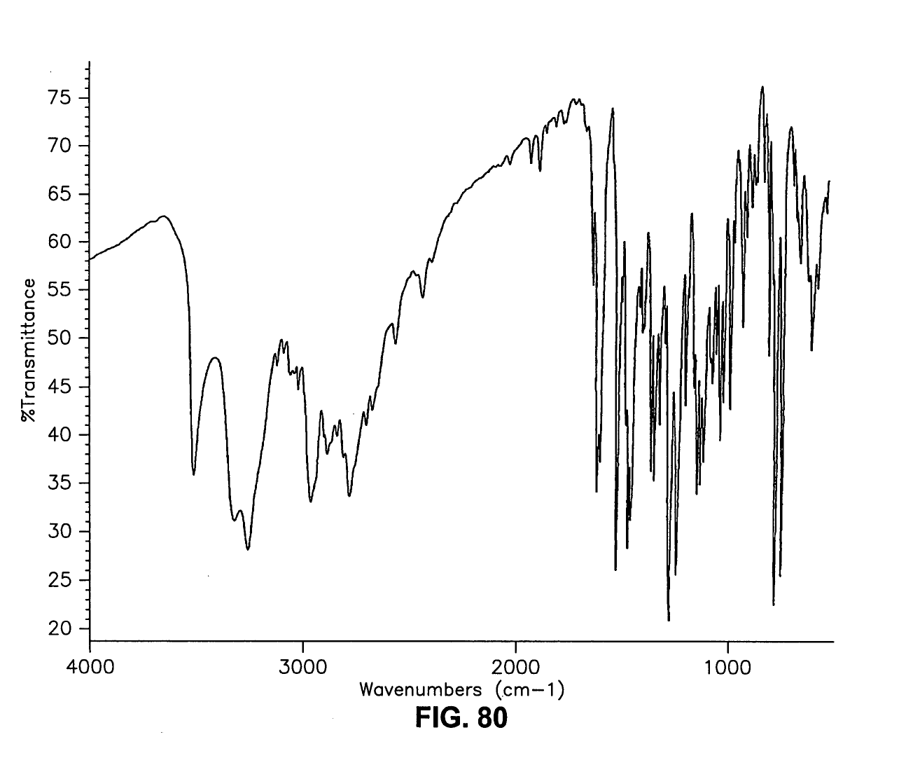

Crystalline carvedilol hydrobromide 2-methyl-1-propanol solvate (see, Example 11: Form 4) also is identified by an x-ray diffraction pattern as shown substantially in FIG. 80, which depicts characteristic peaks in degrees two-theta (2θ): i.e., 7.8±0.2 (2θ), 8.1±0.2 (2θ), 16.3±0.2 (2θ), 18.8±0.2 (2θ), 21.8±0.2 (2θ), and 28.5±0.2 (2θ).

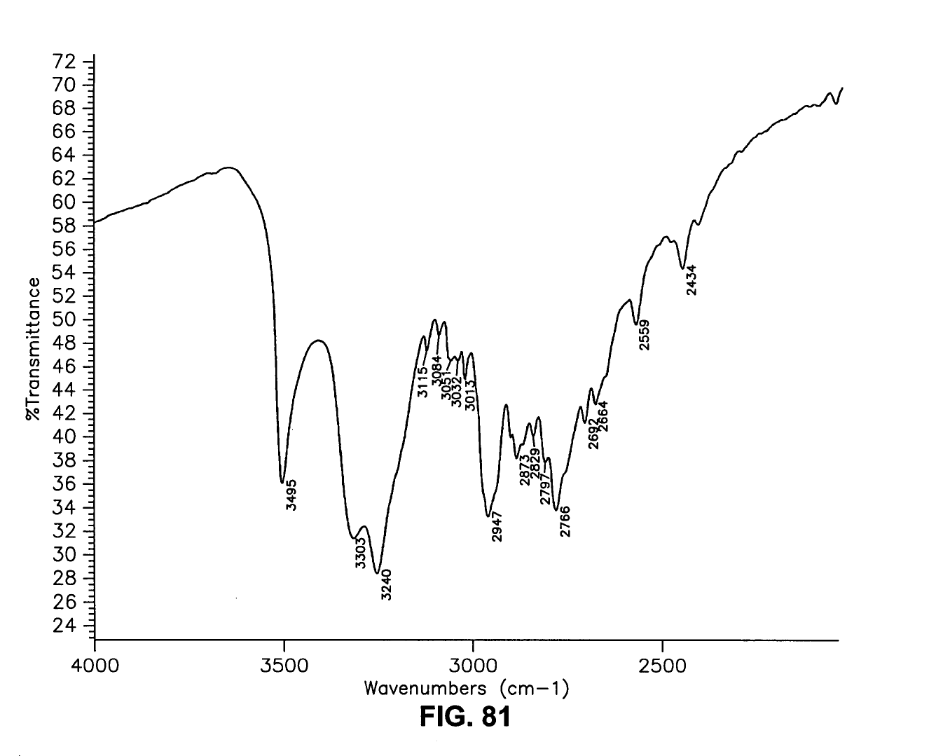

Crystalline carvedilol hydrobromide trifluoroethanol solvate (see, Example 12: Form 5) also is identified by an x-ray diffraction pattern as shown substantially in FIG. 81, which depicts characteristic peaks in degrees two-theta (2θ): i.e.,. 7.7±0.2 (2θ), 8.4±0.2 (2θ), 15.6±0.2 (2θ), 16.9±0.2 (2θ), 18.9±0.2 (2θ), 21.8 i 0.2 (2θ), 23.8±0.2 (2θ), 23.7±0.2 (2θ), and 32.7±0.2 (2θ).

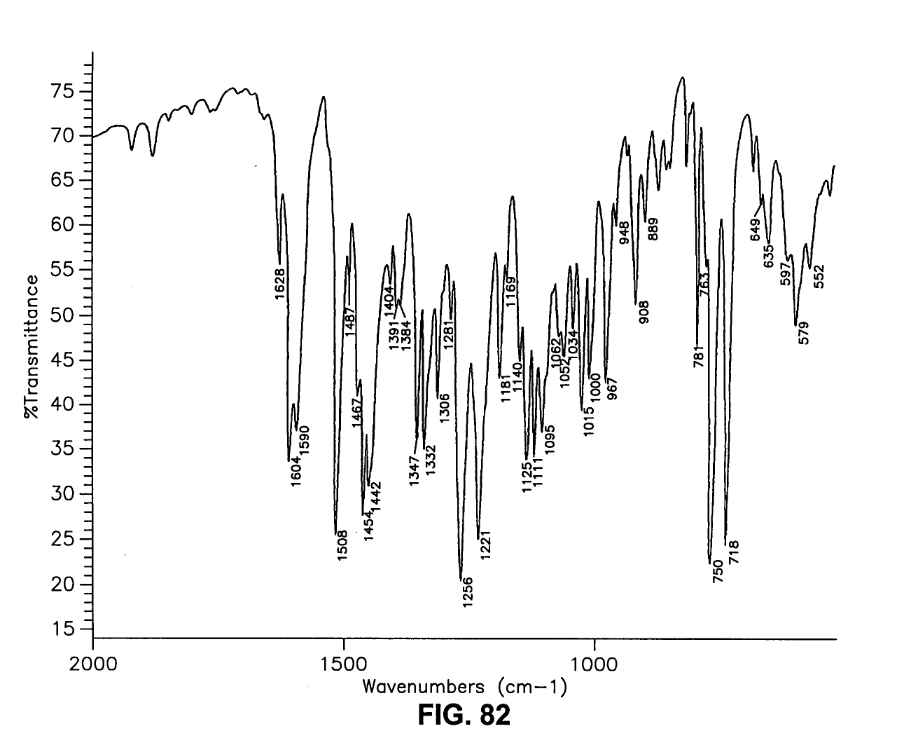

Crystalline carvedilol hydrobromide 2-propanol solvate (see, Example 13: Form 6) also is identified by an x-ray diffraction pattern as shown substantially in FIG. 82, which depicts characteristic peaks in degrees two-theta (2θ): i.e.,. 7.9±0.2 (2θ), 8.3±0.2 (2θ), 18.8±0.2 (2θ), 21.7±0.2 (2θ), 23.2±0.2 (2θ), 23.6±0.2 (2θ), and 32.1±0.2 (2θ).

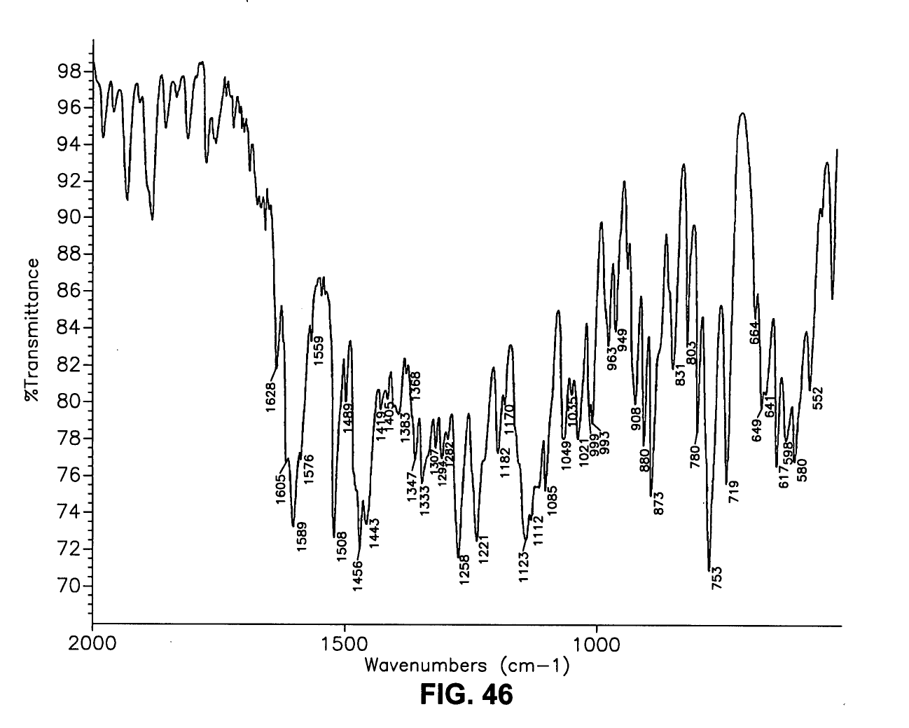

Crystalline carvedilol hydrobromide n-propanol solvate #1 (see, Example 14: Form 7) also is identified by an x-ray diffraction pattern as shown substantially in FIG. 46, which depicts characteristic peaks in degrees two-theta (2θ): i.e., 7.9±0.2 (2θ), 8.5±0.2 (2θ), 17.0±0.2 (2θ), 18.8±0.2 (2θ), 21.6±0.2 (2θ), 23.1±0.2 (2θ), 23.6±0.2 (2θ), and 21.2±0.2 (2θ).

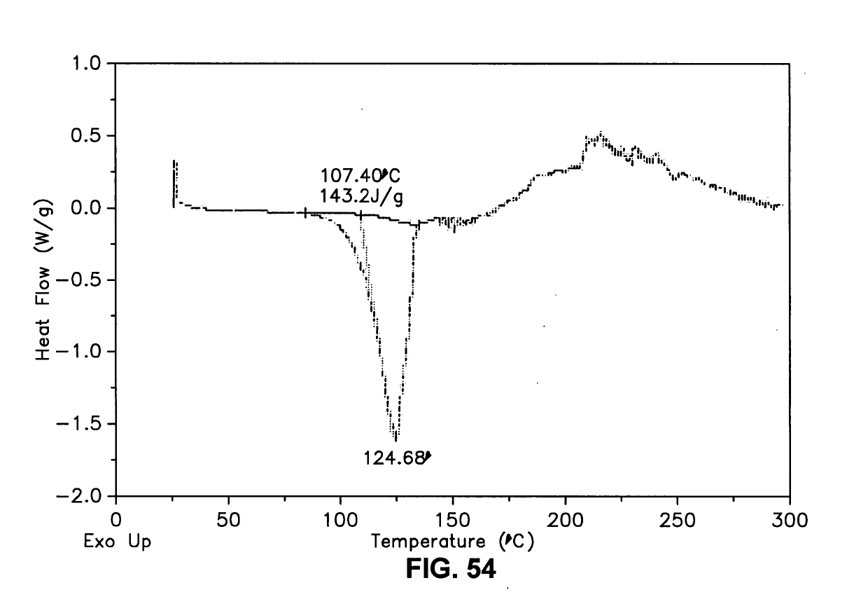

Crystalline carvedilol hydrobromide n-propanol solvate #2 (see, Example 15: Form 8) also is identified by an x-ray diffraction pattern as shown substantially in FIG. 54, which depicts characteristic peaks in degrees two-theta (2θ): i.e., 8.0±0.2 (2θ), 18.8±0.2 (2θ), 21.6±0.2 (2θ), 23.1±0.2 (2θ), 25.9±0.2 (2θ), 27.2±0.2 (2θ), 30.6±0.2 (2θ), and 32.2±0.2 (2θ).

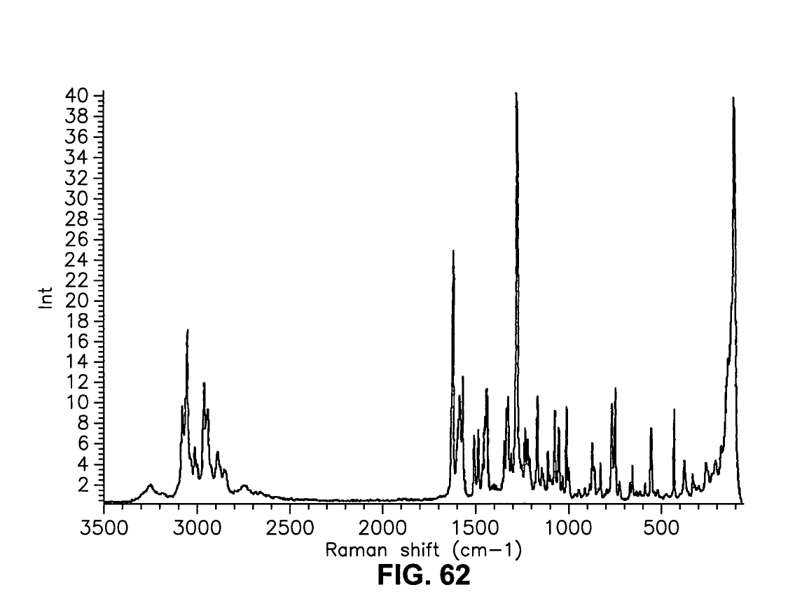

Crystalline carvedilol hydrobromide anhydrous forms (see, Example 16: Form 9) also is identified by an x-ray diffraction pattern as shown substantially in FIG. 62, which depicts characteristic peaks in degrees two-theta (2θ): i.e.,. 6.6±0.2 (2θ), 16.1±0.2 (2θ), 17.3±0.2 (2θ), 21.2±0.2 (2θ), 21.1±0.2 (2θ), 24.1±0.2 (2θ), and 27.9±0.2 (2θ).

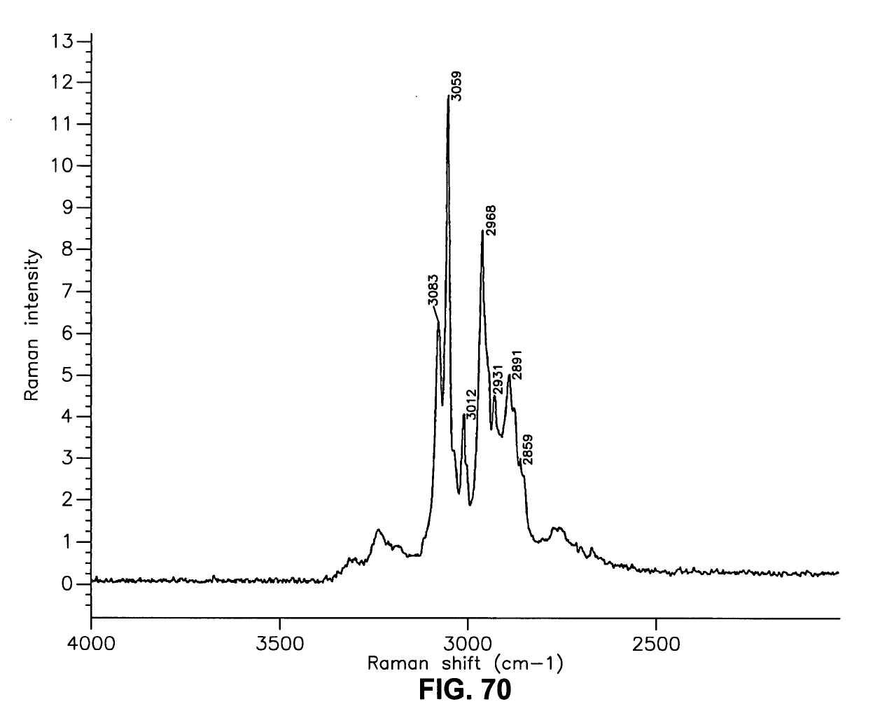

Crystalline carvedilol hydrobromide ethanol solvate (see, Example 17: Form 10) also is identified by an x-ray diffraction pattern as shown substantially in FIG. 70, which depicts characteristic peaks in degrees two-theta (2θ): i.e., 8.1±0.2 (2θ), 8.6±0.2 (2θ), 13.2±0.2 (2θ), 17.4±0.2 (2θ), 18.6±0.2 (2θ), 21.8±0.2 (2θ), 23.2±0.2 (2θ), 23.7±0.2 (2θ), and 27.4±0.2 (2θ).

Crystalline carvedilol hydrobromide monohydrate further is identified by an infrared spectrum as shown substantially in FIG. 6.

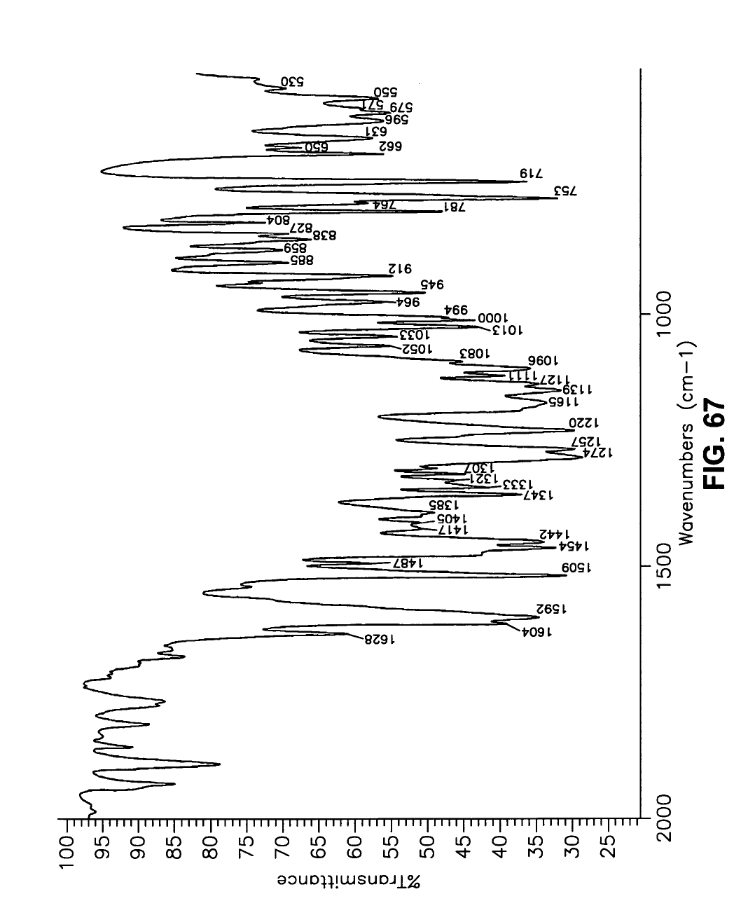

Carvedilol hydrobromide anhydrous forms also an infrared spectrum, which comprises characteristic absorption, bands expressed in wave numbers as shown substantially in FIG. 67.

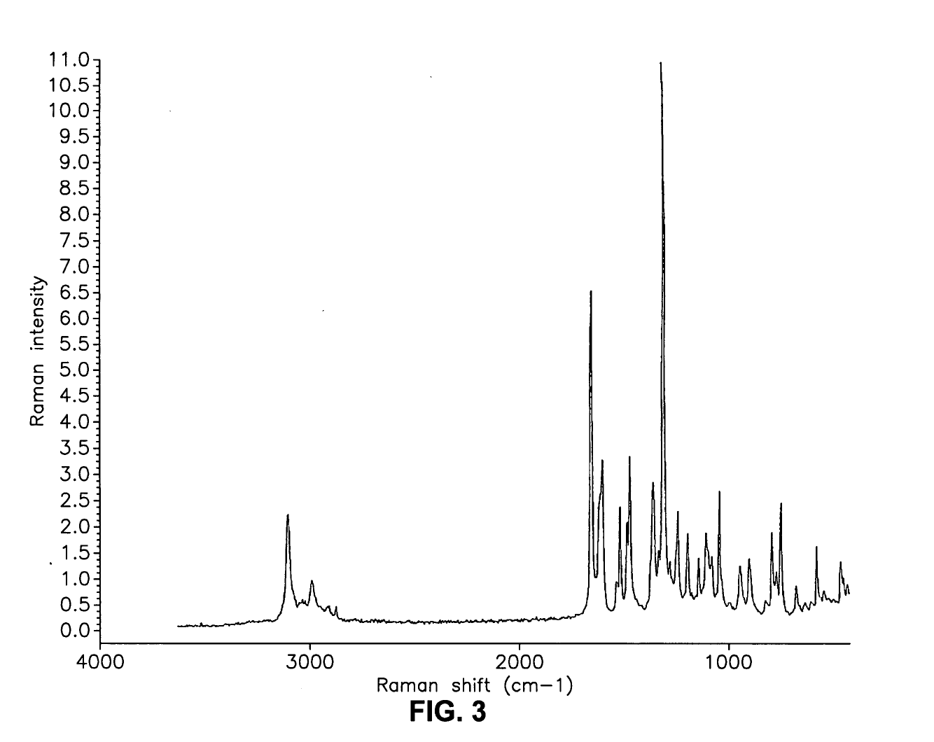

Crystalline carvedilol hydrobromide monohydrate is identified also by a Raman spectrum as shown substantially in FIG. 3.

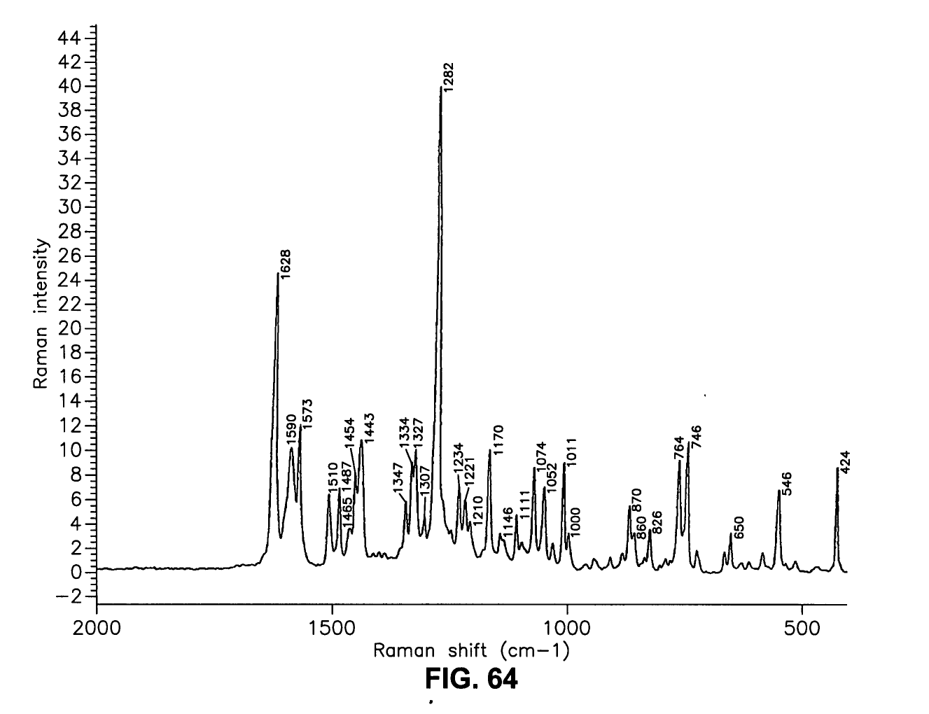

Carvedilol hydrobromide anhydrous forms also a Raman spectrum which comprises characteristic peaks as shown substantially in FIG. 64.

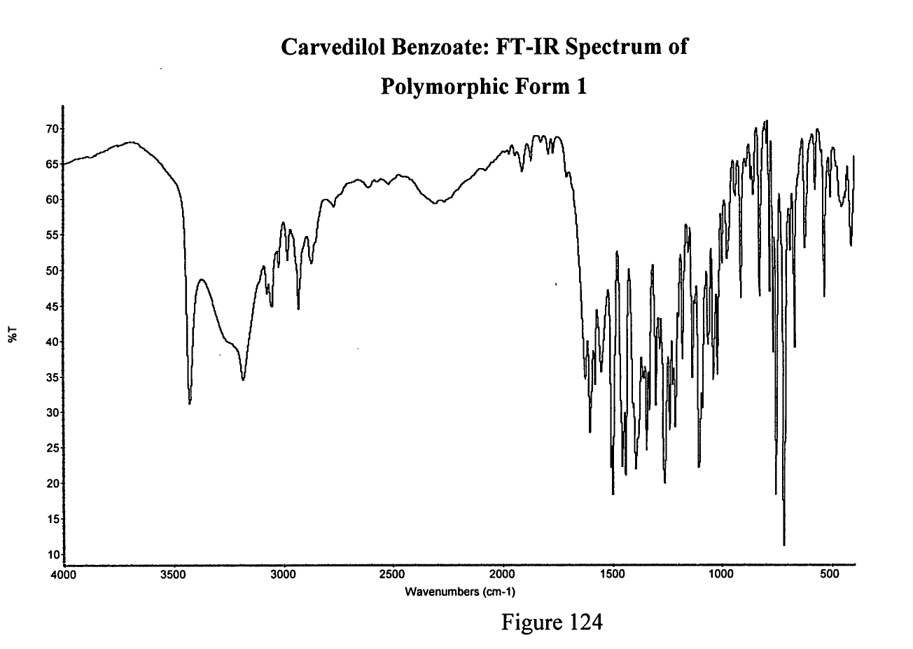

Crystalline carvedilol benzoate (see, Example 22) is identified by an FT-IR spectrum pattern as shown substantially in FIG. 124, which depicts characteristic peaks in wavenumbers (cm⁻¹): i.e., 672 cm⁻¹, 718 cm⁻¹, 754 cm⁻¹, 767 cm⁻¹, 1022 cm⁻¹, 1041 cm⁻¹, 1106 cm⁻¹, 1260 cm⁻¹, 1498 cm⁻¹, 1582 cm⁻¹, 1604 cm⁻¹, 1626 cm⁻¹, 2932 cm⁻¹, 3184 cm⁻¹, and 3428 cm¹. Also, crystalline carvedilol benzoate (see, Example 22) is identified by an FT-Raman spectrum pattern as shown substantially in FIG. 125, which depicts characteristic peaks in wavenumbers (cm⁻¹): i.e., 108 cm⁻¹, 244 cm⁻¹, 424 cm⁻¹, 538 cm⁻¹, 549 cm⁻¹, 728 cm⁻¹, 1001 cm⁻¹, 1015 cm⁻¹, 1128 cm⁻¹, 1286 cm⁻¹, 1598 cm⁻¹, 1626 cm⁻¹, 2934 cm⁻¹, 3058 cm⁻¹, and 3072 cm⁻¹.

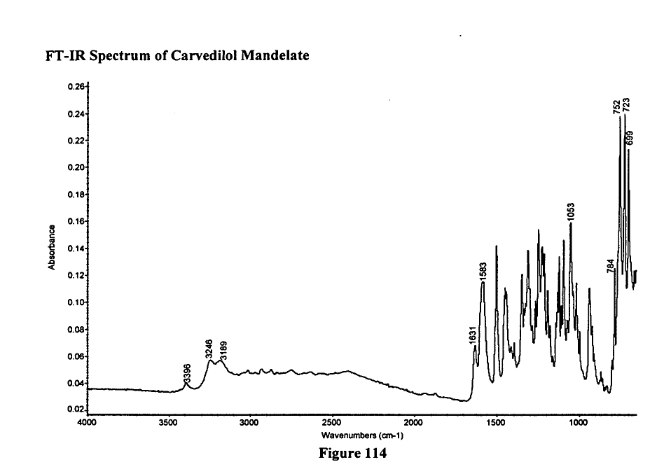

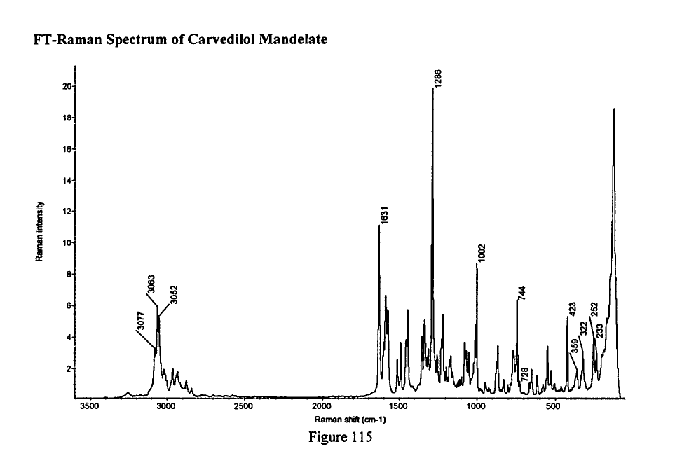

Crystalline carvedilol mandelate (see, Example 23) is identified by an FT-IR spectrum pattern as shown substantially in FIG. 114, which depicts characteristic peaks in wavenumbers (cm⁻¹): i.e., 699 cm⁻¹, 723 cm⁻¹, 752 cm⁻¹, 784 cm⁻¹, 1053 cm⁻¹, 1583 cm⁻¹, 1631 cm⁻¹, 3189 cm⁻¹, 3246 cm⁻¹, and 3396 cm⁻¹. Also crystalline carvedilol mandelate (see, Example 23) is identified by an FT-Raman spectrum pattern as shown substantially in FIG. 115, which depicts characteristic peaks in wavenumbers (cm⁻¹): i.e., 233 cm⁻¹, 252 cm⁻¹, 322 cm⁻¹, 359 cm⁻¹, 423 cm⁻¹, 744 cm⁻¹, 1002 cm⁻¹, 1286 cm⁻¹, 1631 cm⁻¹, 3052 cm⁻¹, 3063 cm⁻¹, and 3077 cm⁻¹.

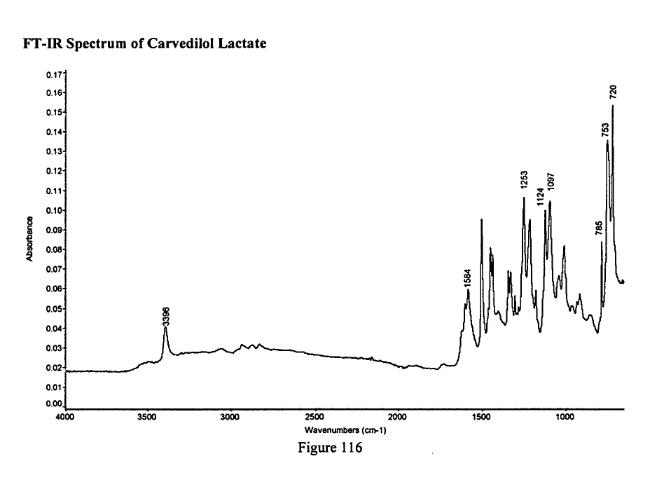

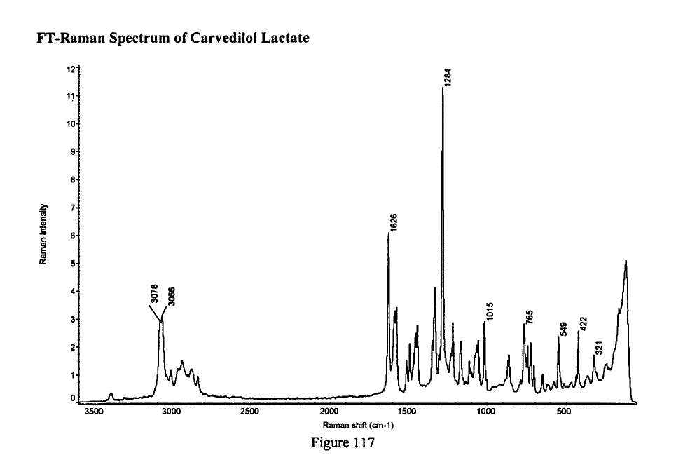

Crystalline carvedilol lactate (see, Example 24) is identified by an FT-IR spectrum pattern as shown substantially in FIG. 116, which depicts characteristic peaks in wavenumbers (cm⁻¹): i.e., 720 cm⁻¹, 753 cm⁻¹, 785 cm⁻¹, 1097 cm⁻¹, 1124 cm⁻¹, 1253 cm⁻¹, 1584 cm⁻¹, and 3396 cm⁻¹. Also, crystalline carvedilol lactate (see, Example 24) is identified by an FT-Raman spectrum pattern as shown substantially in FIG. 117, which depicts characteristic peaks in wavenumbers (cm⁻¹): i.e., 321 cm⁻¹, 422 cm⁻¹, 549 cm⁻¹, 765 cm⁻¹, 1015 cm⁻¹, 1284 cm⁻¹, 1626 cm⁻¹, 3066 cm⁻¹, and 3078 cm⁻¹.

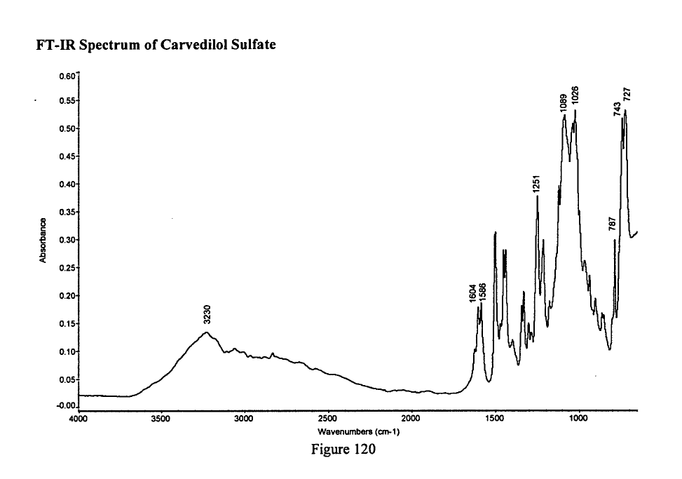

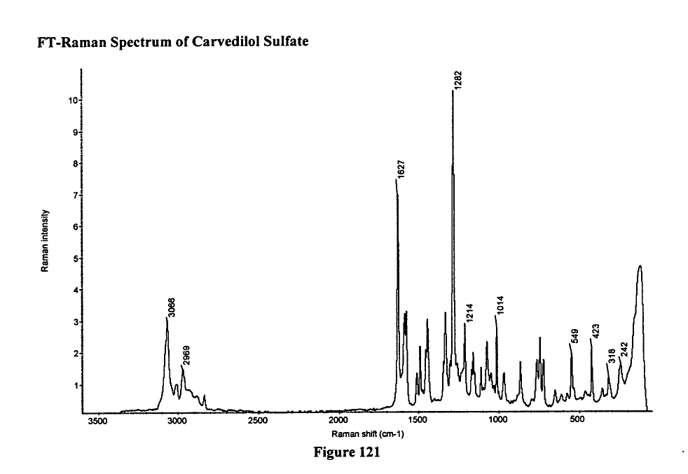

Crystalline carvedilol sulfate (see, Example 25) is identified by an FT-IR spectrum pattern as shown substantially in FIG. 120, which depicts characteristic peaks in wavenumbers (cm⁻¹): i.e., 727 cm⁻¹, 743 cm⁻¹, 787 cm⁻¹, 1026 cm⁻¹, 1089 cm⁻¹, 1251 cm⁻¹, 1215 cm⁻¹, 1586 cm⁻¹, 1604 cm⁻¹, and 3230 cm⁻¹. Also, crystalline carvedilol sulfate (see, Example 25) also is identified by an FT-Raman spectrum pattern as shown substantially in FIG. 121, which depicts characteristic peaks in wavenumbers (cm⁻¹): i.e., 242 cm⁻¹, 318 cm⁻¹, 423 cm⁻¹, 549 cm⁻¹, 1014 cm⁻¹, 1214 cm⁻¹, 1282 cm⁻¹, 1627 cm⁻¹, 2969 cm⁻¹, and 3066 cm⁻¹.

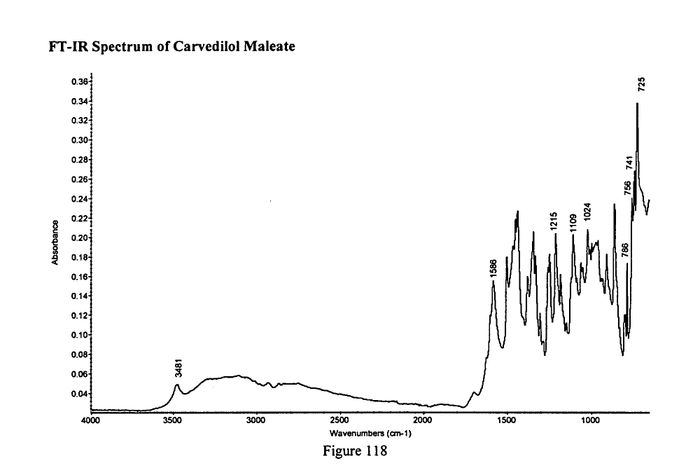

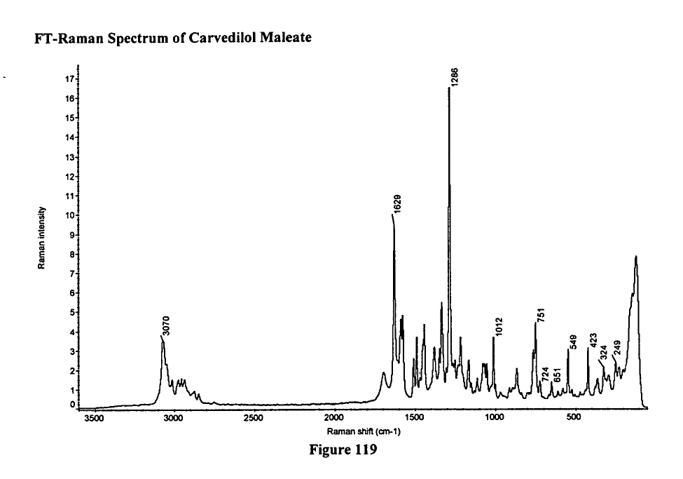

Crystalline carvedilol maleate (see, Example 26) is identified by an FT-IR spectrum pattern as shown substantially in FIG. 118, which depicts characteristic peaks in wavenumbers (cm⁻¹): i.e., 725 cm⁻¹, 741 cm⁻¹, 756 cm⁻¹, 786 cm⁻¹, 1024 cm⁻¹, 1109 cm⁻¹, 1215 cm⁻¹, 1586 cm⁻¹, and 3481 cm⁻¹. Also crystalline carvedilol maleate (see, Example 26) also is identified by an FT-Raman spectrum pattern as shown substantially in FIG. 119, which depicts characteristic peaks in wavenumbers (cm⁻¹): i.e., 249 cm⁻¹, 324 cm⁻¹, 423 cm⁻¹, 549 cm⁻¹, 751 cm⁻¹, 1012 cm⁻¹, 1216 cm⁻¹, 1286 cm⁻¹, 1629 cm⁻¹, and 3070 cm⁻¹.

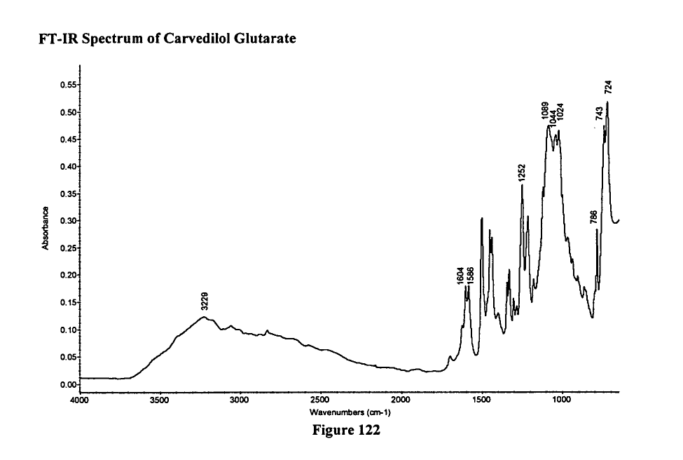

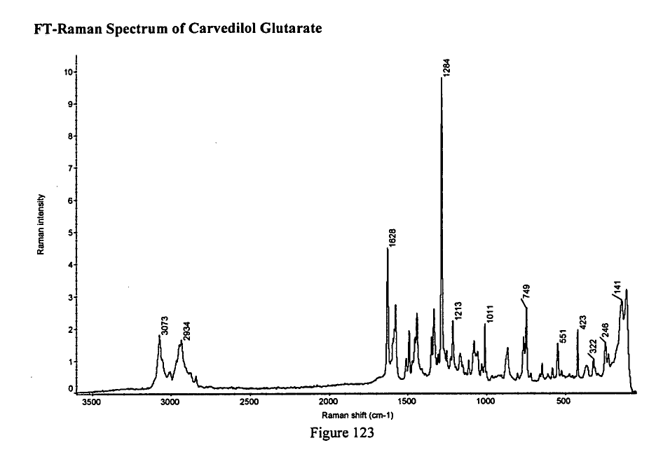

Crystalline carvedilol glutarate (see, Example 27) is identified by an FT-IR spectrum pattern as shown substantially in FIG. 122, which depicts characteristic peaks in wavenumbers (cm⁻¹): i.e., 724 cm⁻¹, 743 cm⁻¹, 786 cm⁻¹, 1024 cm⁻¹, 1044 cm⁻¹, 1089 cm⁻¹, 1251 cm⁻¹, 1586 cm⁻¹, 1604 cm⁻¹, and 3229 cm⁻¹. Also, crystalline carvedilol glutarate (see, Example 27) is identified by an FT-Raman spectrum pattern as shown substantially in FIG. 123, which depicts characteristic peaks in wavenumbers (cm⁻¹): i.e., 141 cm⁻¹, 246 cm⁻¹, 322 cm⁻¹, 423 cm⁻¹, 551 cm⁻¹, 749 cm⁻¹, 1011 cm⁻¹, 1213 cm⁻¹, 1284 cm⁻¹, 1628 cm⁻¹, 2934 cm⁻¹, and 3073 cm⁻¹.

Pharmaceutical Compositions, Controlled-Release Formulations, Dosage Regimens and Dosage Forms

In general, the present invention also relates to different dosage forms, pharmaceutical compositions or controlled-release formulations, which may contain carvedilol free base or a carvedilol salt, solvate, or anhydrous forms thereof as described herein.

For medications to be optimally effective it is important that administration complies with the stipulated dosage regimen. Poor compliance can compromise safety and efficacy. Compliance is usually a problem with medications for chronic asymptomatic illnesses and where patients are elderly and/or infirm.

As previously discussed, carvedilol is known as an effective medication for treating hypertension, congestive heart failure, and other cardiovascular conditions. Its unique mode of action is a consequence of it being a mixture of R and S isomers with complimentary pharmacological effects. Vasodilation and reduced peripheral resistance are a consequence of the alpha blockade associated with the R isomer. Blood pressure reduction is ascribed to the beta blockade contributed by both R and S isomers.

Currently, carvedilol is administered to treat cardiovascular diseases to a subject in need thereof and is usually administered twice daily.

Cardiovascular diseases treatable by methods of the present invention, include, but are not limited to hypertension, congestive heart failure, atherosclerosis, angina, etc.

However, for chronic diseases such as cardiovascular diseases, a once-daily dosage regimen is desirable, to enhance patient compliance and reduce “pill burden”. Medication that is dosed once daily facilitates greater compliance with the dosage regimen. This applies especially to chronic asymptomatic illnesses. It follows that medication for a condition like hypertension, atherosclerosis, or some other cardiac conditions is most effective, from a safety and efficacy perspective if dosed once daily.

In many cases the pharmacokinetics or pharmacodynamics of a drug are such that once a day dosage, using conventional dosage forms provides adequate therapy.

With some drugs it may be necessary to formulate a dosage form that releases the drug over an extended period, to provide sustained plasma levels that evince the desired duration of action. Such modified release dosage forms are invariably designed to provide plasma levels that do not fluctuate significantly over time.

It is well established that there is a strong relationship between frequency of dosage and compliance. Medication that is dosed once daily is considered best from a convenience and compliance perspective than when more frequent doses are necessary. Thus, medication for a chronic and “silent” condition like hypertension, and other cardiac conditions is most effective, from a safety and efficacy perspective if dosed once daily.

The pharmacokinetics or pharmacodynamics of a drug may be such that once-a-day dosage, using conventional dosage forms provides adequate therapy. However, with some drugs it may be necessary to formulate so that the dosage form releases the drug over an extended period, in order to sustain plasma levels to provide the desired duration of action. Such modified release dosage forms are traditionally designed to provide plasma levels of drug that do not fluctuate significantly over time.

However, a medication providing constant plasma levels may not always be optimal for treating hypertension, atherosclerosis or related conditions. Blood pressure is influenced by cirdadian rhythm. It rises in the morning on awakening (so-called “morning surge”), is maximum during daytime activities and falls at night, particularly between around midnight to 3 am (see, Anar. Y. A, White. W. B; Drugs (1998) 55 (5) 631-643; Chronotherapeutics for Cardiovascular Disease). “Morning-surge may be a factor in the higher incidence of cardiovascular incidents like stroke, acute myocardial infarction and angina pectoris that occur in the early morning.

Blood pressure also can remain elevated at night in some hypertensives, particularly the elderly. These have been termed “non-dippers” and such a condition is associated with increased cardiovascular morbidity (see, Kario. K, Matsuo. T, Kobayashi. H, Imiya. M, Matsuo. M, Shimida. K; Hypertension (1996) 27 (1) 130-135. Nocturnal Fall of Blood Pressure and Silent Cerebrovascular Damage in Elderly Hypertensive Patients).

However, the dose response and time course of carvedilol in the body is such that a conventional dosage form, releasing all the drug immediately on ingestion does not provide once-a-day therapy. Release from the dosage form needs to be slowed down so that absorption and subsequent systemic residence is prolonged. This however requires that release and dissolution occurs along the GI tract, not just in the stomach.

Drug absorption following oral dosage requires that drug first dissolves in the gastro-intestinal milieu. In most cases such dissolution is primarily a function of drug solubility. If solubility is affected by pH it is likely that absorption will vary in different regions of the gastro intestinal tract, because pH varies from acidic in the stomach to more neutral values in the intestine.

Such pH-dependent solubility can complicate dosage form design when drug absorption needs to be prolonged, delayed or otherwise controlled, to evince a sustained or delayed action effect. Variations in solubility can lead to variable dissolution, absorption and consequent therapeutic effect.

A case can therefore be made that plasma levels ought be optimal at times of high risk, provided that there is an association between plasma level and pharmacodynamic effect. In general, cardiovascular medication has been designed with such requirements in mind (see, White. W. H, Andes. R. J, MacIntyre. J. M, Black. H. R, Sica. D. A; The American Journal of Cardiology (1995) 76, 375-380. Nocturnal Dosing of a Novel Delivery System of Verapamil for Systemic Hypertension). For example, the beta blockade-associated effect on blood pressure is proportional to dose (see, De May. C. D, Breithaupt. K, Schloos. J, Neugebauer. G, Palm. D, Beiz. G. G; Clinical Pharmacology & Therapeutics ((1994) 55, (3) 329-337. Dose-Effect and Pharmacokinetic and Pharmacodynamic Relationships of Beta-Adrenergic Receptor Blocking Properties of Various Doses of Carvedilol in Healthy Humans).

It would be beneficial therefore, in cases where there is good association between plasma level and clinical response, that optimal levels of drug be present before and during times of high risk so that the cardiovascular system is stabilized and not vulnerable to dramatic change, or that levels of the pharmacological agent are not sub therapeutic. Some recent cardiovascular medications have been designed to provide for “early morning cover” (White. W. H, Andes. R. J, MacIntyre. J. M, Black. H. R, Sica. D. A; The American Journal of Cardiology (1995) 76 375-380. Nocturnal Dosing of a Novel Delivery System of Verapamil for Systemic Hypertension.) but none appear to be available that provide cover for the “non-dipping” period as well as protecting against morning surge.

The beta blockade-associated effect on blood pressure is proportional to dose (De May. C. D, Breithaupt. K, Schloos. J, Neugebauer. G, Palm. D, Belz. G. G; Clinical Pharmacology & Therapeutics ((1994) 55 (3)329-337. Dose-Effect and Pharmacokinetic and Pharmacodynamic Relationships of Beta-Adrenergic Receptor Blocking Properties of Various Doses of Carvedilol in Healthy Humans.

Hence, therapy is likely to be more effective if adequate plasma levels are provided before and during times of greatest risk. Thus, a dosage form taken at night (bedtime), that delivers drug in two phases viz during the midnight-3 am period and prior to and during “morning surge” activities ought provide optimum pharmacological-based therapy. At the same time it is important that adequate levels are maintained throughout the full dosage period, to provide reliable and stable control.

A further advantage of an optimally designed dosage form concerns rate of release of drug from the unit immediately after ingestion. Alpha blockade evinces a vasodilation effect and associated reduction of peripheral resistance. If drug plasma levels rise too rapidly this can lead to postural hypotension and risk of falling over. More gradual rise in plasma levels would, conceivably make for a safer medication.

With the above considerations, it will be evident that an optimally designed, once daily dosage form of carvedilol, taken at night should have the following features:

release drug at a slower rate following ingestion so that plasma buildup is gradual, thereby avoiding rapid fall in blood pressure and minimizing risk of orthostatic hypotension-related adverse events;

provide adequate plasma levels of drug about 1-3 hours after dosing, with subsequent falloff as time progresses;

provide a “later or second peak”, about 5-10 hours after dosing with gradual reduction of plasma levels thereafter; and/or

provide that plasma levels that do not fall below the minimum level for effectiveness such that plasma levels after 24 hours should be comparable to those obtained when dosing twice daily dosage (as current commercial COREG® medication in the United States).

Moreover, a profile associated with such a once daily dosage of carvedilol, would exhibit a first peak at about 1 hours to about 3 hours, which should be lower than the later or second peak as physiological activity is at a minimum during sleep so control requires less drug.

In contrast, plasma levels in the morning ought to reflect the greater activity and associated cardiovascular stress at this time.

Such a profile is consistent with current thinking that medications for cardiovascular conditions, that provide near constant drug concentrations over time may not be optimally designed. Because of circadian variations in blood pressure it may be more appropriate to provide high concentrations of drug at times of greatest need. Furthermore, blood pressure lowering should not be excessive during night time, so as to reduce potential for night-time hypotension and ischaemic stroke (Smith David. G. H: American Journal of Hypertension: (2001) 14 296S-301S. Pharmacology of Cardiovascular Therapeutic Agents).

The physico chemical properties, and pharmacokinetics of carvedilol make it difficult to design the kind of delivery system described above, for the following reasons:

both R and S isomer carvedilol forms are cleared relatively rapidly from the systemic circulation (alpha elimination phase is about 1.5 hours); and/or

plasma levels are depleted rapidly and substantially following attainment of peak plasma concentrations.

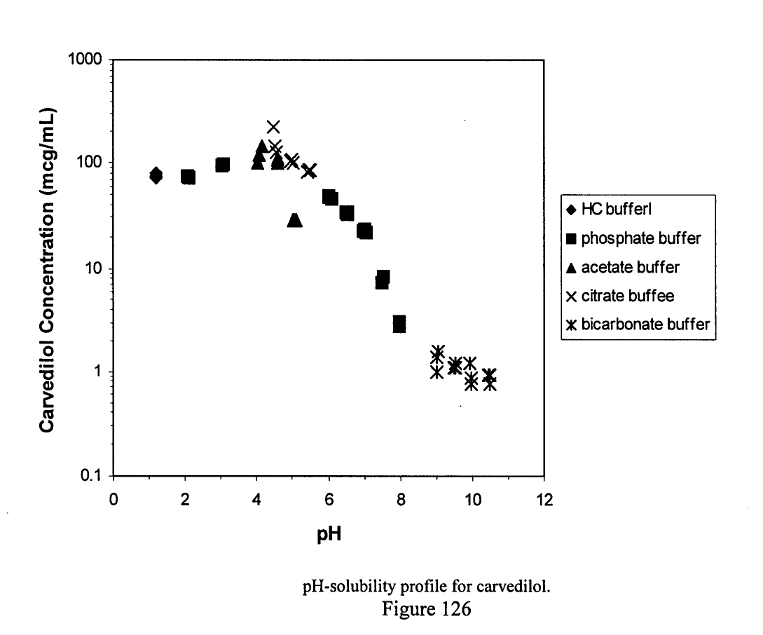

The pH-aqueous solubility of the free base form of carvedilol is such (FIG. 126) that absorption is likely to be low, or even non-existent from the neutral regions of the gastro intestinal tract. A drug needs to be in solution if it is to pass from the intestine to systemic circulation and it is generally accepted that, where aqueous solubility is less than about 5 mg/ml, absorption following oral dosage can be problematical (Ritschel W. A. Arzneim Forsch (1975), 25, p. 853)). At the pH values encountered in the distal small intestine and colon, solubility of carvedilol free base does not exceed 0.1 mg (100 mcg) per ml).

Such a solubility profile makes it difficult to design a dosage form to sustain absorption for long periods by providing slow release of drug from the dosage form as it transits the gastro intestinal tract. At pH values in the middle and lower parts of the small intestine solubility is likely to be insufficient to enable sufficient drug to dissolve to provide adequate absorption flux. This constraint could theoretically be surmounted if it were possible to design a unit that remained in the stomach or upper small intestine, such that drug was released to an environment more conducive to dissolution and absorption. However, the maximum period that a dosage form is retained in the fed stomach is about three hours. This time period possibly might be prolonged if a high fat content meal were consumed at the time of dosage. However, this is probably impractical for “before bedtime” dosage, especially where in any case such a diet is inadvisable for patients with cardiovascular disease.

Thus, it will be obvious to a person skilled in the art that the rapid systemic clearance combined with poor solubility at neutral pH of carvedilol constrain possibilities for designing a unit to provide prolonged absorption and sustained plasma levels. In effect there are formidable, if not insurmountable challenges in the design of a unit incorporating delayed and time-specific release features as well as providing adequate plasma levels over a once-a day dosing period. The absence of any commercially available modified release dosage form of carvedilol, designed for optimal chronotherapeutic effect supports this view.

However, the dose response and time course of carvedilol in the body is such that a conventional dosage form, releasing all the drug immediately on ingestion does not provide once-a-day therapy.

Release from the dosage form needs to be slowed down so that absorption and subsequent systemic residence is prolonged. This however requires that release and dissolution occurs along the GI tract, not just in the stomach.

Hence, it would be expected that therapy would be more effective if peak plasma levels were provided times of greatest risk.

In such a context a carvedilol based dosage form that is taken at night (at bedtime), that delivers drug in two phases to cover the midnight-3 am period, and the early morning surge ought provide optimum therapy, while maintaining a once-daily dosage regimen.

However, the properties of carvedilol drug substance, as well as its pharmacokinetics make it difficult to design such a delivery system, for the following reasons:
[1] absorption from the lower gastro intestinal tract is less efficient than from the stomach. This is probably related to the very low solubility of carvedilol at neutral pH, making it difficult to design a dosage form to sustain absorption for long periods. The maximum period that a dosage form is retained in the fed stomach is around three hours; and/or
[2] the relatively rapid clearance (alpha elimination phase) means that plasma levels are reduced rapidly and substantially following attainment of the peak plasma concentration.

These considerations, taken together teach that the provision of a dosage form, delivering carvedilol at times of maximum patient risk, and potential optimum benefit is difficult if not impossible.

Nevertheless, it has now, surprisingly been shown that, when a carvedilol salt, solvate or anhydrous forms thereof is utilized, and, when such a carvedilol salt, anhydrous or solvate thereof is formulated using appropriate modified release technology, plasma profiles are obtained in human volunteers that are aligned with what knowledge of the chronobiology suggests may be optimally beneficial in hypertension and congestive heart failure.

Therefore, solubility of carvedilol free base or various carvedilol salts, anhydrous or solvate forms thereof as those described herein may facilitate provision or development of a dosage form, such as a controlled-release formulation, from which the drug substance becomes available for bioabsorption throughout the gastrointestinal tract (i.e., in particular the lower small intestine and colon). See Example 28 herein and corresponding discussion at pages 94-98 of the instant specification.

Parts of the gastrointestinal tract are defined to include generally the stomach (i.e. which includes the antrum and pylorus bowel), small intestine (i.e., which has three parts: the duodenum, jejunum, illeum), large intestine (i.e., which has three parts: the cecum, colon, rectum), liver, gall bladder and pancreas.

Treatment regimen for the administration of compounds, pharmaceutical compositions, or controlled-release formulations or dosage forms of the present invention may also be determined readily by those with ordinary skill in art. The quantity of the compound, pharmaceutical composition, or controlled-release formulation or dosage form of the present invention administered may vary over a wide range to provide in a unit dosage in an effective amount based upon the body weight of the patient per day to achieve the desired effect and as based upon the mode of administration.

In light of the foregoing, the present invention relates to an embodiment where a compound, pharmaceutical composition, or controlled-release formulation or dosage form is presented as a unit dose taken preferably from 1 to 2 times daily, most especially taken once daily to achieve the desired effect.

Importantly, the chemical and/or physical properties of carvedilol forms described herein, which include, but are not limited to the above-identified carvedilol free base or carvedilol salts, anhydrous forms or solvates thereof indicate that those forms may be particularly suitable for inclusion in medicinal agents, pharmaceutical compositions, etc.

The scope of the present invention includes all compounds, pharmaceutical compositions, or controlled-release formulations or dosage forms, which is contained in an amount effective to achieve its intended purpose. While individual needs vary, determination of optimal ranges of effective amounts of each component is within the skill of the art.

In accordance with a pharmaceutical composition, dosage form or controlled release formulation of the present invention as described herein (i.e., which include any of the specific embodiment described for various delivery systems or technologies applicable with the present invention), a specific embodiment may include a carvedilol free base or which may be, but is not limited to, be in a combination with a solubility enhanced carvedilol salt, solvate or anhydrous forms form or forms.

In accordance with a pharmaceutical composition, dosage form or controlled release formulation of the present invention as described herein (i.e., which include any of the specific embodiment described for various delivery systems or technologies applicable with the present invention), a specific embodiment may include pharmaceutically acceptable acid addition salts of carvedilol free base or corresponding forms.

General definitions suitable to define aspects of the present invention are set forth below.

Such pharmaceutically acceptable salts of carvedilol free base or corresponding forms are formed with appropriate organic acids or mineral acids, which may include, but are not limited to formation by methods described herein or conventionally known in the art.

Representative examples of such suitable organic or mineral acids may include, but are not limited to maleic acid, fumaric acid, benzoic acid, ascorbic acid, pamoic acid, succinic acid, bismethylenesalicyclic acid, methane sulphonic or sulfonic acid, acetic acid, propionic acid, tartaric acid, salicyclic acid, citric acid, gluconic acid, aspartic acid, stearic acid, palmitic acid, itaconic acid, glycolic acid, p-aminobenzoic acid, glutamic acid, benzene sulfonic acid or sulphonic acid, hydrochloric acid, hydrobromic acid, sulfuric acid or sulphuric acid, cyclohexylsulfamic acid, phosphoric acid, nitric acid and the like.

In accordance with the present invention, mineral acids may be selected from, but are not limited to hydrobromic acid, hydrochloric acid, phosphoric acid, sulfuric acid or sulphuric acid, and the like; and organic acids may be selected from, but not limited to methansulphuric acid, tartaric acid, maleic acid, acetic acid, citric acid, benzoic acid and the like.

Also in accordance with a pharmaceutical composition, dosage form or controlled release formulation of the present invention as described herein (i.e., which include any of the specific embodiment described for various delivery systems or technologies applicable with the present invention), a specific embodiment may include a solubility enhanced carvedilol salt, solvate or anhydrous forms form or forms, which may include, but are not limited to novel crystalline or other solid forms, selected from the group consisting of carvedilol mandelate, carvedilol lactate, carvedilol maleate, carvedilol sulfate, carvedilol glutarate, carvedilol mesylate, carvedilol phosphate, carvedilol citrate, carvedilol hydrogen bromide, carvedilol oxalate, carvedilol hydrogen chloride, carvedilol hydrogen bromide, carvedilol benzoate, or corresponding solvates thereof.

Further in accordance with a pharmaceutical composition, dosage form or controlled release formulation of the present invention as described herein (i.e., which include any of the specific embodiment described for various delivery systems or technologies applicable with the present invention), a specific embodiment may include, but are not limited to novel crystalline salt or other solid forms of carvedilol hydrogen phosphate, carvedilol dihydrogen phosphate, carvedilol dihydrogen phosphate hemihydrate, carvedilol dihydrogen phosphate dihydrate, carvedilol dihydrogen phosphate methanol solvate, carvedilol hydrobromide monohydrate, carvedilol hydrobromide dioxane solvate, carvedilol hydrobromide 1-pentanol solvate, carvedilol hydrobromide 2-methyl-1-propanol solvate, carvedilol hydrobromide trifluoroethanol solvate, carvedilol hydrobromide 2-propanol solvate, carvedilol hydrobromide n-propanol solvate #1, carvedilol hydrobromide n-propanol solvate #2, carvedilol hydrobromide anhydrous forms or anhydrous forms, carvedilol hydrobromide ethanol solvate, carvedilol hydrobromide dioxane solvate, carvedilol monocitrate monohydrate, carvedilol mandelate, carvedilol lactate, carvedilol hydrochloride, carvedilol maleate, carvedilol sulfate, carvedilol glutarate, or corresponding anhydrous forms, solvates thereof.

Also suitable for use in any of the pharmaceutical compositions, dosage forms or controlled release formulations of the present invention are solubility enhanced carvedilol salt, solvate or anhydrous forms, which may include, but are not limited to novel crystalline salt or other solid forms, selected from the group consisting of carvedilol hydrogen phosphate, carvedilol dihydrogen phosphate, carvedilol dihydrogen phosphate hemihydrate, carvedilol dihydrogen phosphate dihydrate, carvedilol dihydrogen phosphate methanol solvate.

In particular, in accordance with a pharmaceutical composition, dosage form or controlled release formulation of the present invention as described herein (i.e., which include any of the specific embodiment described for various delivery systems or technologies applicable with the present invention), a specific embodiment may include a carvedilol salt, solvate, or anhydrous forms thereof, such as a carvedilol phosphate salt, which may include, but is not limited to or selected from the group consisting of a carvedilol dihydrogen phosphate hemihydrate (Form I), carvedilol dihydrogen phosphate dihydrate (Form II), carvedilol dihydrogen phosphate methanol solvate (Form III), carvedilol dihydrogen phosphate dihydrate (Form IV), carvedilol dihydrogen phosphate (Form V) and carvedilol hydrogen phosphate (Form VI), and the like.

Also, suitable for use in any of the pharmaceutical compositions, dosage forms or controlled release formulations of the present invention is carvedilol dihydrogen phosphate hemihydrate.

Thus, this invention also relates to a pharmaceutical composition comprising an effective amount of carvedilol dihydrogen phosphate salts or solvates thereof, with any of the characteristics noted herein, in association with one or more non-toxic pharmaceutically acceptable carriers or diluents thereof, and if desired, other active ingredients.

Also, suitable for use in any of the pharmaceutical compositions, dosage forms or controlled release formulations of the present invention is carvedilol dihydrogen phosphate hemihydrate or carvedilol phosphate anhydrous.

Depending upon the treatment being effected, the compounds, or compositions of the present invention can be administered orally, intraperitoneally, or topically, etc. Preferably, the composition is adapted for oral administration.

In general, pharmaceutical compositions of the present invention are prepared using conventional materials and techniques, such as mixing, blending and the like.

In accordance with the present invention, compounds or pharmaceutical composition can also include, but are not limited to, suitable adjuvants, carriers, excipients, or stabilizers, etc. and can be in solid or liquid form such as, tablets, capsules, powders, solutions, suspensions, or emulsions, etc.

Typically, the composition will contain a compound of the present invention, such as carvedilol free base or a carvedilol salt, anhydrous form or solvate thereof or active compound(s), together with the adjuvants, carriers or excipients. In particular, a pharmaceutical composition of the present invention comprises an effective amount of a salt of carvedilol (i.e., such as carvedilol dihydrogen phosphate salts), anhydrous or corresponding solvates (i.e., as identified herein) forms thereof, with any of the characteristics noted herein, in association with one or more non-toxic pharmaceutically acceptable carriers or diluents thereof, and if desired, other active ingredients.

These active compounds may also be administered parenterally. Solutions or suspensions of these active compounds for use in such parental administrations can be prepared in water suitably mixed with a surfactant such as hydroxypropylcellulose.

Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil, etc. In general, water, saline, aqueous dextrose and related sugar solution, and glycols such as, propylene glycol or polyethylene glycol, etc., are preferred liquid carriers, particularly for injectable solutions. Under ordinary conditions of storage and use, these preparations contain a preservative to prevent the growth of microorganisms.

In accordance with the present invention, solid unit dosage forms can be conventional types known in the art. The solid form can be a capsule and the like, such as an ordinary gelatin type containing the compounds of the present invention and a carrier, for example, lubricants and inert fillers such as, lactose, sucrose, or cornstarch, etc. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate, etc.

The tablets, capsules, and the like can also contain a binder, such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin, etc. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both, etc. A syrup can contain, in addition to active ingredient, sucrose as a sweetening agent, methyl and propylparabens as preservatives, a dye, and flavoring such as cherry or orange flavor, etc.

For oral therapeutic administration, these active compounds can be incorporated with excipients and used in the form of tablets, capsules, elixirs, suspensions, syrups, and the like.

The percentage of a carvedilol free base or carvedilol salt, solvate or anhydrous form thereof compound in compositions can, of course, be varied as the amount of active compound in such therapeutically useful compositions is such that a suitable dosage will be obtained.

Typically in accordance with the present invention, the oral maintenance dose is between about 25 mg and about 70 mg, preferably given once daily. In accordance with the present invention, the preferred unit dosage forms include tablets or capsules.

It will be appreciated that the actual preferred dosages of the compounds being used in the compositions of this invention will vary according to the particular composition formulated, the mode of administration, the particular site of administration and the host being treated.

In particular, dosing in humans for treatment of diseases according to the present invention should not ordinarily or normally exceed a dosage range of from about 5 mg to about 75 mg of carvedilol free base or an equivalent amount of a carvedilol salt, solvate or anhydrous form thereof. As one of ordinary skill in the art will readily comprehend, the patient should be started on a low dosage regimen of a compound of the present invention and monitored for well-known symptoms of intolerance, e.g., fainting, to such compound. Once the patient is found to tolerate such compound amount, the patient should be brought slowly and incrementally up to the maintenance dose. The preferred course of treatment is to start the patient on a dosage regimen of either approximately or about 8 mg to about 16 mg, given once daily, for approximately two weeks. The choice of initial dosage most appropriate for the particular patient is determined by the practitioner using well-known medical principles, including, but not limited to, body weight. In the event that the patient exhibits medically acceptable tolerance of a compound, corresponding composition or formulation of the present invention for two weeks, the dosage is doubled at the end of the two weeks and the patient is maintained at the new, higher dosage for two more weeks, and observed for signs of intolerance. This course is continued until the patient is brought to a maintenance dose. The preferred maintenance dose for carvedilol free base or an equivalent amount of a carvedilol salt, solvate or anhydrous form thereof is about 32.5 mg to about 65 mg given once daily for patients having a body weight of up to 85 kg. For patients having a body weight of over 85 kg, the maintenance dose is about 65 mg if given once daily.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet, etc.

In addition, compounds or pharmaceutical compositions of the present invention may incorporated into controlled or modified release forms, which may incorporate the use of or modification of various controlled release development processes, which may include, but are not limited to technologies such as those conventionally known in the art.

Specific examples of technologies related or used in the formation of pharmaceutical compositions, controlled-release formulations or dosage forms of the present invention are described below.

General Formulation Technologies

Delivery systems suitable for use in accordance with the present invention, may include, but are not limited to materials as described generally in this section.

The term “active agent” is defined for purposes of the present invention as any chemical substance or composition of the present invention, such as carvedilol free base, or a carvedilol salt, anhydrous forms, or solvate thereof, which can be delivered from the device into an environment of use to obtain a desired result. When the active agent is a biologically active drug, such as carvedilol free base, or a carvedilol salt, anhydrous form, or solvate thereof, or corresponding pharmaceutical composition of the present invention, which is taken orally and the external fluid is gastric fluid, it is preferred that the drug exhibits a between the solubility defined in the United States Pharmacopeia (USP) XXI, page 7 as “freely soluble” (i.e., 1-10 parts solvent per 1 part solute) and “sparingly soluble” (i.e., 30-1000 parts solvent per 1 part solute).

The dosage form, which includes a device or delivery system associated with the present invention can be used in conjunction with a wide range of drugs (i.e., which includes carvedilol free base, or a carvedilol salt, anhydrous forms, or solvate thereof) and is especially well-suited for drugs having a wide therapeutic window, since precise dosing is not very critical for the same. The therapeutic window is commonly defined as the difference between the minimum effective blood concentration and the maximum effective blood concentration and the toxic concentration of the drug.

Depending upon the solubility and the amount of active agent to be included in the core, any generally accepted soluble or insoluble inert pharmaceutical filler (diluent) material may be used to bulk up the core or to solubilize the active agent.

Suitable materials for use in the present invention, include, but are not limited to sucrose, dextrose, lactose, fructose, xylitol, mannitol, sorbitol, dicalcium phosphate, calcium sulfate, calcium carbonate, starches, cellulose, polyethylene glycols, polyvinylpyrollidones, which may include, but are not limited to non-cross-linked polyvinylpyrollidones or cross-linked polyvinylpyrollidones, polyvinyl alcohols, sodium or potassium carboxmethylcelluloses, gelatins, mixtures of any of the above, and the like.

In addition, it is possible to directly compress an active agent with a small amount of lubricant when the active agent is soluble in the external fluid and is included in such an amount to provide a suitably sized core.

Lubricant may be mixed with the active agent and excipients prior to compression into a solid core. Any generally accepted pharmaceutical lubricant may be used, which may include, but are not limited to calcium or magnesium soaps and the like.

Active agents can be formulated with a small amount of a binder material such as, for example, gelatin or polyvinylpyrollidone (i.e. 94%-99.75% of the core comprises the active agent). In such cases, the components of the core may be subjected to wet granulation. For example, highly soluble pharmaceutically active compounds such as potassium chloride may be directly compressed into an acceptable core with the inclusion of 0.25 percent magnesium stearate without being in admixture with an excipient.

The particular excipient chosen is dependent in part upon the solubility of the active agent in the environmental fluid. The ratio of active agent to excipient is based in part upon relative solubility of the active agent in the external fluid and the desired rate of release. If the active agent is relatively soluble, it may be desirable to slow down the eroding of the core by using a relatively insoluble excipient such as dicalcium phosphate.

Representative materials suitable for use in the present invention as a coating include those materials commonly considered to be insoluble in the art, which may include, but are not limited to materials, such as ethyl cellulose, acrylate polymers, polyamides (nylons), polymethacrylates, polyalkenes (polyethylene, polypropylene), bio-degradable polymers (including homo- or hetero-polymers of polyhydroxy butyric or valeric acids and homo or hetero-polymers of polylactic, polyglycolic, polybutyric, polyvaleric, and polycaprolactic acids), waxes, natural oils, other hydrophobic insoluble materials such as polydimethylsiloxane, hydrophilic materials such as cross-linked sodium carboxymethyl cellulose and cross-linked sodium or uncross-linked carboxy-methyl starch and the like. Many other polymers considered to be relatively insoluble as conventionally used in the art also would be useful in the present invention.

It is also possible to use relatively thick coatings of materials in the present invention, which are considered in the art to be relatively soluble in, environmental fluid, which may include, but are not limited to materials, such as polyvinylpyrrolidone, cross-linked polyvinylpyrrolidone, cellulose ethers including hydroxypropylmethylcellulose, methylcellulose, hydroxyethylcellulose, hydroxypropylcellulose, sodium carboxymethyl cellulose, sodium carboxymethyl starch, enteric materials (such as cellulose acetate phthallate, polyvinylalcohol phthallate, shellac, zein, hydroxypropylmethyl cellulose phthallate, cellulose acetate trimaleate, etc) and the like.

In certain embodiments of the present invention, it may be advantageous to include one or more release modifying agents which aids in the release of the active agent from a suitable device of the present invention in the environment of use.

For example, the inclusion of a surfactant or an effervescent base may be helpful in certain cases to overcome surface tension effects, etc. Other releasing modifying agents may include osmagents (i.e., which osmotically deliver the active agent from the device by providing an osmotic pressure gradient against the external fluid and are particularly useful when the active agent has limited solubility in the environment of use), swelling agents (i.e., provided in an amount sufficient to facilitate the entry of the environmental fluid without causing the disruption of the impermeable coating) or other pharmaceutically acceptable adjuvants, excipients or carriers. Alternatively, release modifying agents, such as hydrophobic materials and insoluble polymers, may be used to slow the release of active agent from the device or release modifying agents may be used in conjunction with the present invention to include ion exchange resins.

Surfactants useful as release modifying agents in the present invention can be anionic, cationic, nonionic, or amphoteric. Examples of such surfactants or release modifying agents, may include, but are not limited to sodium lauryl sulfate, sodium dodecyl sulfate, sorbitan esters, polysorbates, pluronics, potassium laurate, and the like.

Effervescent bases useful as release modifying agents in the present invention, may include, but are not limited to, sodium glycine carbonate, sodium carbonate, potassium carbonate, sodium bicarbonate, potassium bicarbonate, calcium bicarbonate, and the like.

Osmagents useful as release modifying agents in the present invention may include, but are not limited to sodium chloride, calcium chloride, calcium lactate, sodium sulfate, lactose, glucose, sucrose, mannitol, urea, and many other organic and inorganic compounds known in the art and the like.

Examples of suitable swelling agents for use in the present invention, include synthetic gums, which further may include, but are not limited to hydroxypropylmethylcelluloses (HPMC) hydroxypropyl cellulose, carboxymethyl cellulose, and natural gums such as xanthan gum, locust bean gum, acacia, tragacanth, guar gum, carrageenan, and propylene glycol alginate, and the like.

Examples of suitable hydrophobic materials useful as release modifying agents in the present invention, include vegetable oils, which may include, but are not limited to hydrogenated cottonseed oil, hydrogenated castor oil, and the like. An example of insoluble polymers includes ethyl cellulose, etc.

A device may be designed such that the rate of release of the active agent varies with time which may be used to achieve a chronotherapeutic effect not normally possible with some conventional art-known sustained release devices.

Matrix Core or Tablet-Type Technology Section An example of a delivery system for use in accordance with the present invention, may include, but is not limited to a tablet formulation, comprising a core, formulated as a matrix core base with hydroxypropyl cellulose, hydroxyethyl cellulose or other such release-modifying polymer and the like, ensuring that drug is gradually made available at a pre-determined rate.

The tablet is coated with a pH-sensitive polymer that is insoluble at gastric pH but soluble at neutral pH. Each tablet is perforated by laser beam, or mechanically to provide an aperture of pre-determined size that allows release of drug in a controlled way. Such controlled release occurs while the unit is in an acidic environment. When the tablet passes to the more neutral region of the gastrointestinal tract the polymeric coat is dissolved and release of drug is controlled by the polymer in the tablet core matrix.

Such a delivery system as described above is exemplified by U.S. Pat. No. 5,004,614 to Staniforth, which is hereby incorporated by reference in its entirety.

In particular, U.S. Pat. No. 5,004,614 to Staniforth discloses controlled release devices having a core, which includes an active agent and an outer coating, which is substantially impermeable to the entrance of an environmental fluid and substantially impermeable to the release of the active agent during a dispensing period to allow controlled release of the active agent through an orifice in the outer coating.

In light of the foregoing, the present invention relates to a controlled delivery device for an active agent, which comprises a core comprising an active agent and an outer coating covering said core which includes an orifice communicating from the environment of use to the core for allowing the release of the active agent into the environment of use. The thickness of the coating is adapted such that it is substantially impermeable to the release of the active agent during a predetermined dispensing period.

The outer coating may be comprised of any acceptable material which can be adapted to provide the above-mentioned properties. Thus, a material may be suitable for use as the outer coating even if it is somewhat soluble in or somewhat permeable to the surrounding external fluid, as long as a sufficiently thick coating is applied such that the external fluid does not contact the core except through the orifice for a period sufficient to allow substantially all of the active agent to be released through the orifice.

The outer coating may be chosen so as to eventually dissolve in the external fluid, or be degraded thereby after substantially all of the active agent has been released from the device.

The active agent may comprise a wide variety of chemical compounds or compositions, and may have a wide range of solubilities in the external fluid. The active agent may be combined with one or more excipients to form the core in order to solubilize the core when it is exposed to the external fluid, in order to provide bulk to the core, etc. Conventional tableting excipients can be used to form the core of a tablet in accordance with the present invention. Even freely soluble excipients such as sugars which would not normally be expected to have a role in a sustained release system may be employed.

In a specific embodiment, the active agent is soluble in the external fluid, or the composition is errodable and therefore capable of being carried out of the device as a suspension. Preferably, the components of the core are solid when dry.

In one embodiment of the present invention, the device is a hemispherical or near-hemispherical tablet with a hole located centrally in the flat or shallow convex side. In another embodiment, the device is a biconvex tablet with at least one concentric hole.

The core of the device of the present invention may be prepared using conventional tablet excipients and formulation methods. Depending upon the solubility and the amount of active agent to be included in the core, any generally accepted soluble or insoluble inert pharmaceutical filler (diluent) material may be used to bulk up the core or to solubilize the active agent.

Suitable materials for use in the present invention, include, but are not limited to sucrose, dextrose, lactose, fructose, xylitol, mannitol, sorbitol, dicalcium phosphate, calcium sulfate, calcium carbonate, starches, cellulose, polyethylene glycols, polyvinylpyrollidones, polyvinyl alcohols, sodium or potassium carboxmethylcelluloses, gelatins, mixtures of any of the above, and the like.

In addition, it is possible to directly compress an active agent with a small amount of lubricant when the active agent is soluble in the external fluid and is included in such an amount to provide a suitably sized core.

Lubricant may be mixed with the active agent and excipients prior to compression into a solid core. Any generally accepted pharmaceutical lubricant may be used, which may include, but are not limited to calcium or magnesium soaps and the like.

Active agents can be formulated with a small amount of a binder material such as, for example, gelatin or polyvinylpyrollidone (i.e. 94%-99.75% of the core comprises the active agent). In such cases, the components of the core may be subjected to wet granulation. For example, highly soluble pharmaceutically active compounds such as potassium chloride may be directly compressed into an acceptable core with the inclusion of 0.25 percent magnesium stearate without being in admixture with an excipient.

The particular excipient chosen is dependent in part upon the solubility of the active agent in the environmental fluid. The ratio of active agent to excipient is based in part upon relative solubility of the active agent in the external fluid and the desired rate of release. If the active agent is relatively soluble, it may be desirable to slow down the eroding of the core by using a relatively insoluble excipient such as dicalcium phosphate.

The complete mixture of active agent, lubricant, excipient, etc., in an amount sufficient to make a uniform batch of cores, is subjected to compression in a conventional production scale tableting machine at normal compression pressures, i.e. such as about 2000-16000 lbs/sq. in.

The term “active agent” is defined for purposes of the present invention as any chemical substance or composition of the present invention, such as carvedilol free base, or a carvedilol salt, anhydrous forms, or solvate thereof, which may include, but are not limited to novel crystalline or other solid forms, which can be delivered from the device into an environment of use to obtain a desired result.

The active agent can be soluble in the external fluid which enters the device through the orifice, or it can have limited solubility in the external fluid. Preferably, an excipient which is readily soluble in the external fluid is induced when the active agent has limited solubility in the external fluid. When the active agent is relatively soluble in the external fluid, the choice of excipient is less critical to obtaining a desired controlled release pattern.

When the active agent is a biologically active drug, such as carvedilol free base, or a carvedilol salt, anhydrous forms, or solvate thereof, or corresponding pharmaceutical composition of the present invention, which is taken orally and the external fluid is gastric fluid, it is preferred that the drug exhibits a between the solubility defined in the United States Pharmacopeia (USP) XXI, page 7 as “freely soluble” (i.e., 1-10 parts solvent per 1 part solute) and “sparingly soluble” (i.e., 30-1000 parts solvent per 1 part solute).

The dosage form, which includes a device or delivery system associated with the present invention can be used in conjunction with a wide range of drugs (i.e., which includes carvedilol free base, or a carvedilol salt, anhydrous forms, or solvate thereof) and is especially well-suited for drugs having a wide therapeutic window, since precise dosing is not very critical for the same. The therapeutic window is commonly defined as the difference between the minimum effective blood concentration and the maximum effective blood concentration and the toxic concentration of the drug.

The compacted masses which comprise the cores are then coated with a suitable amount of a material such that the coating is substantially impermeable to the environmental fluid during the desired release time.

Representative materials suitable for use in the present invention as the coating include those materials commonly considered to be insoluble in the art, which may include, but are not limited to materials, such as ethyl cellulose, acrylate polymers, polyamides (nylons), polymethacrylates, polyalkenes (polyethylene, polypropylene), bio-degradable polymers (including homo- or hetero-polymers of polyhydroxy butyric or valeric acids and homo or hetero-polymers of polylactic, polyglycolic, polybutyric, polyvaleric, and polycaprolactic acids), waxes, natural oils, other hydrophobic insoluble materials such as polydimethylsiloxane, hydrophilic materials such as cross-linked sodium carboxymethyl cellulose and cross-linked sodium or uncross-linked carboxy-methyl starch and the like. Many other polymers considered to be relatively insoluble as conventionally used in the art also would be useful in the present invention.

While some of the above materials do exhibit a certain degree of permeability to environmental fluids such as water, the coating is applied at such a thickness that they do not expose the core to the environmental fluid and are not removed by dissolution or otherwise disrupted before the desired duration of the controlled release of the active agent has passed.

For example, while ethylcellulose has in the past been used as a coating for devices such as pharmaceutical controlled release tablets, the thickness of the ethyl cellulose coating has generally been in the neighborhood of 4 percent by weight of the tablet core and possibly containing a proportion of a soluble polymer, e.g. hydroxypropylmethylcellulose or a plasticizer, e.g. glycerol. In contrast, the ethyl cellulose coat usable with compounds, compositions, or formulations of the present invention in such circumstances would generally be 2-3 times thicker (i.e. 10 percent to 12 percent or more by weight of the tablet core).

It is also possible to use relatively thick coatings of materials in the present invention, which are considered in the art to be relatively soluble in, environmental fluid, which may include, but are not limited to materials, such as polyvinylpyrrolidone, cellulose ethers including hydroxypropylmethylcellulose, methylcellulose, hydroxyethylcellulose, hydroxypropylcellulose, sodium carboxymethyl cellulose, sodium carboxymethyl starch, enteric materials (such as cellulose acetate phthallate, polyvinylalcohol phthallate, shellac, zein, hydroxypropylmethyl cellulose phthallate, cellulose acetate trimaleate, etc) and the like.

It is also possible to use coatings in the present invention, which comprise combinations of relatively insoluble and relatively soluble materials. In accordance with the present invention, thickness of the coating necessary to provide results simply may be determined by one of ordinary skilled in the art via the preparation of devices with differing coating thicknesses, performing dissolution tests in the devices without the inclusion of an orifice in the device, and choosing the coating thickness which does not allow the release of the active agent from the device during the desired duration of controlled release.

For example, in one embodiment, the impermeable coating comprises ethyl cellulose. In another embodiment, the impermeable coating comprises from about 90 to about 96.5 percent hydrogenated vegetable oil, from about 3 to about 5 percent polyvinylpyrollidone, and from about 0.5 to about 5 percent magnesium stearate or other similar lubricant.

The impermeable coating may be formed by film formation from a polymer in solution, or suspension using pouring or spraying onto a pre-formed tablet core. Preferably, this process is carried out by spraying the coating onto the tablet core in a rotating pan coater or in a fluidized bed coater until the desired coating thickness is achieved. Alternatively, a tablet core may be dip coated or melt coated. This is especially useful with waxes and oils. In another embodiment, the core may be compression coated. In other words, a suitable impermeable coating material may be pressed onto a preformed tablet core.

In another embodiment, an adhesive coat such as shellac or polyvinyl acetate phthallate (PVAP) is applied to the core prior to applying the impermeable coating in order to improve adhesion of the impermeable coating to the core.

Next, an orifice is made in the coated device. For purposes of the present invention, the term “orifice” is synonymous with hole, passageway, outlet, aperture, etc. The orifice may be formed using any technique known in the art. For instance, the orifice may be made using a needle or other form of boring instrument such as a mechanical drill or a laser to remove a section of the impermeable layer of the tablet core.

Alternatively, the impermeable layer may be prevented from covering a patch of a pre-formed core to thereby provide an orifice. This may be achieved using chemical protection or a modified coating method. If compression coating is employed, an eccentric or assymetrical core may be employed so that the core automatically reveals a portion of its surface, as the impermeable layer is compressed thereon. Alternatively, a specially designed punch tip may be incorporated into the compressing equipment, in order to pierce through the impermeable layer at the point of compaction.

It is preferred that the orifice extend through the entire impermeable layer such that there is immediate exposure of the core to the environmental fluid when the device is placed in the desired environment of use.

The orifice is made in the sealed device so that the active agent is released from the device at the desired rate. The desired rate of release is achieved by providing the proper diameter of the orifice relative to the diameter of the device and taking into account parameters such as the properties of the active agent and the excipients used (if any). Such properties include solubility, matrix formation, etc. Preferably, the orifice is dimensioned to allow the entrance of environmental fluid (e.g., gastric fluid) such that the active agent is released from the device at a predetermined controlled rate.

The device of the present invention may be of any preselected shape, such as biconvex, hemispherical or near-hemispherical, oval, oblong, round, cylindrical, triangular, etc. However, it is presently preferred that the device is biconvex, hemispherical, or near-hemispherical. By “near-hemispherical”, it is meant that one face of the device is substantially flat, shallow convex or shallow concave, and the opposite face is deeply convex (i.e., the deeply convex face has a greater radius of curvature than the shallow convex, shallow concave, or substantially flat face). It is most preferred presently that the device is biconvex due to complexities involved with the coating of hemispherical or near-hemispherical devices.

The orifice can have any shape, including round, triangular, square, elliptical, irregular, and the like. However, for purposes of reproducibility, it is preferred that the orifice be round. Similarly, the orifice may be located at any point on the coated surface of the device, but reproducibility has been found to be substantially improved when the orifice is centrally located. For example, reproducibility has been found to be improved when a biconvex tablet according to the present invention includes a concentrically located orifice rather than an orifice that is eccentric or in the side wall of the tablet.

In other embodiments of the present invention, more than one orifice may be provided in the device for the release of active agent. The orifices may be located on the same face of the tablet, or on each or different faces.

The orifice has a diameter which normally corresponds to from about 10 to about 60 percent of the diameter of the device. Preferably, the orifice has a diameter which is about 30 percent of the diameter of the device. On the other hand, the device may be provided with a number of orifices, the sum of whose diameters comprise about the same diameter as a single orifice which has been determined to provide an acceptable release rate. Of course, the diameter of the orifice is dependent in part upon the active agent and the desired release rate. In cases where the orifice is non-circular, the orifice will correspond to from 1 to about 40 percent of the corresponding surface of the device, and preferably about 10 percent.

The device of the present invention is preferably an oral tablet, although it may be adapted for buccal, cervical, rectal, intrauterine, nasal, artificial gland, implant use and the like. When the device is an implant, it is preferable that the impermeable coating is either physiologically inert or biodegradable. The device also can be sized, shaped structured and adapted for delivering an active agent in streams, aquariums, fields, factories, reservoirs, laboratory facilities, hot houses, transportation means, naval means, for veterinary use, chemical reactions and other environments of use.

The amount of agent present in the device, whether soluble in the environmental fluid or a derivitized soluble form thereof, is generally non-limited and it is an amount larger than or equal to the amount of agent that is necessary to be effective for bringing about the desired effect upon its release in the environment of use. Since the invention contemplates a variety of uses, there is no critical upper limit on the amount of agent incorporated in the device. The lower limit will depend on the span of the release of the product and the activity of the product.

In the case of an orally taken biconvex tablet, once the tablet is exposed to the gastric fluid within the stomach, the drug and any excipient is dissolved via gastric fluid which passes through the orifice and contacts the exposed portion of the tablet core. The rate of release of drug through the orifice remains constant as the drug and excipient is continually eroded, in part because the exposed surface of the drug and excipient moves away from the orifice and simultaneously increases the surface area of exposed core.

In certain embodiments of the present invention, it may be advantageous to include one or more release modifying agents in the tablet core which aids in the release of the active agent from the device in the environment of use.

For example, the inclusion of a surfactant or an effervescent base may be helpful in certain cases to overcome surface tension effects, etc. Other releasing modifying agents known as osmagents osmotically deliver the active agent from the device by providing an osmotic pressure gradient against the external fluid. Such agents are particularly useful when the active agent has limited solubility in the environment of use. Still other release modifying agents are swelling agents provided in an amount sufficient to facilitate the entry of the environmental fluid without causing the disruption of the impermeable coating. Alternatively, release modifying agents may be used to slow the release of active agent from the device. Examples of such agents include hydrophobic materials and insoluble polymers. Other release modifying agents which may be used in conjunction with the present invention include ion exchange resins.

Surfactants useful as release modifying agents in the present invention can be anionic, cationic, nonionic, or amphoteric. Examples of such surfactants or release modifying agents, may include, but are not limited to sodium lauryl sulfate, sodium dodecyl sulfate, sorbitan esters, polysorbates, pluronics, potassium laurate, and the like.

Effervescent bases useful as release modifying agents in the present invention, may include, but are not limited to sodium glycine carbonate, sodium carbonate, potassium carbonate, sodium bicarbonate, potassium bicarbonate, calcium bicarbonate, and the like.

Osmagents useful as release modifying agents in the present invention may include, but are not limited to sodium chloride, calcium chloride, calcium lactate, sodium sulfate, lactose, glucose, sucrose, mannitol, urea, and many other organic and inorganic compounds known in the art and the like.

Examples of suitable swelling agents for use in the present invention, include synthetic gums, which further may include, but are not limited to hydroxypropylmethylcelluloses (HPMC) hydroxypropyl cellulose, carboxymethyl cellulose, and natural gums such as xanthan gum, locust bean gum, acacia, tragacanth, guar gum, carrageenan, and propylene glycol alginate, and the like.

Examples of suitable hydrophobic materials useful as release modifying agents in the present invention, include vegetable oils, which may include, but are not limited to hydrogenated cottonseed oil, hydrogenated castor oil, and the like. An example of insoluble polymers includes ethyl cellulose, etc.

Other release modifying agents which may be useful in the present invention provide a soluble or insoluble polymer backbone to the core. Such agents may decrease unequal density areas of the core formed during the compression molding of the same.

Suitable soluble polymers for use in the present invention, which may be incorporated into the core include those which melt upon compression and fuse upon cooling to provide nearly uniform cross-sectional density, such as polyethylene glycols having a molecular weight of from about 6 to about 20,000 and the like. Other water soluble polymers are sufficiently viscous upon contacting the front of environmental fluid which enters through the orifice to provide the same effect, such as high molecular weight polyvinylpyrollidone (i.e., K90 grade commercially available from GAF Corporation and having a molecular weight of about 360,000).

In another embodiment of the present invention, the device may be multi-layered and preferably bi- or tri-layered. This may be desirable, for example in order to provide a loading dose of an active agent, or for releasing two or more different agents.

By means of the present invention, it is possible to obtain a zero-order release of a pharmaceutical composition, or other active agent, i.e., a constant amount of drug is released per unit time in vitro by erosion of the tablet core.

On the other hand, the device may be designed such that the rate of release of the active agent varies with time which may be used to achieve a chronotherapeutic effect not normally possible with sustained release devices. This is in addition to the other parameters of the present invention that govern the rate of release, such as the size and location of the orifice.

In light of the foregoing technologies, it may be possible to develop stable pharmaceutical compositions or controlled release or modified dosage forms, containing such carvedilol free base or carvedilol salts, solvates, or anhydrous forms thereof of the present invention, for once-per-day dosage, delayed release or pulsatile release to optimize therapy by matching pharmacokinetic performance (i.e., which relates to the time-dependent changes of plasma drug concentration and the time dependent changes of the total amount of drug in a body following various routes of administration) with pharmacodynamic requirements (i.e., which relates to the biochemical and physiologic effects of drugs and their mechanisms of action).

As previously indicated herein, it would be expected that therapy would be more effective if peak plasma levels were provided times of greatest risk. In such a context a carvedilol based dosage form that is taken at night (at bedtime), that delivers drug in two phases to cover the midnight-3 am period, and the early morning surge ought provide optimum therapy, while maintaining a once-daily dosage regimen.

Therefore, in a specific embodiment of the present invention, such units, dosage forms, pharmaceutical compositions or controlled-release formulations of the present invention are formulated or prepared so that drug is released in “pulses”, separated in time such that the first “peak” or Tₘₐₓ occurs within 1-4 hours of dosage, preferably the first “peak” or Tₘₐₓ occurs 1-2 hours of dosage or preferably the first “peak” or Tₘₐₓ occurs within 2-4 hours of dosage, with the second “peak” or Tₘₐₓ occurring 5-10 hours later or preferably the second “peak” or Tₘₐₓ occurring 5-8 hours later.

In particular, such a pharmaceutical composition or controlled-release formulation of the present invention following oral dosage would be depicted by a unique biphasic pharmacokinetic/pharmacodynamic plasma profile, which exhibits a first Tₘₐₓ pulse and a plasma concentration peak level within 1-4 hours of ingestion and a second Tₘₐₓ pulse and a plasma concentration peak level within, 5-10 hours after ingestion. In another specific embodiment, such a pharmaceutical composition or controlled-release formulation of the present invention following oral dosage would be depicted by a unique biphasic pharmacokinetic/ pharmacodynamic plasma profile, which exhibits a first Tₘₐₓ pulse and a plasma concentration peak level within 2-4 hours of ingestion and a second Tₘₐₓ pulse and a plasma concentration peak level within, 5-8 hours after ingestion.

In a specific embodiment, the aforementioned oral dosage or administration associated with a pharmaceutical composition or controlled-release formulation of the present invention, preferably occurs at night.

It is important that release from the first “pulse” or Tₘₐₓ occurs gradually, so that subsequent absorption is gradual, thereby avoiding a rapid fall in blood pressure. This would minimize the risk of orthostatic hypotension-related adverse events.

Such a profile can be obtained by formulating drug as tablets with differential release, capitalizing on a combination of approaches to operate sequentially. It may be, for instance that a tablet is formulated as separate layers, each layer affording release characteristics that are influenced by factors such as gastrointestinal pH, or time, to provide differentiated absorption profiles. The same effect could be evinced by formulating in pellets that are coated with different release-modifying components, such pellets being contained in capsule dosage forms.

In light of the foregoing discussion, the present invention relates to and is exemplified by, but not limited to the following embodiments present below, which include corresponding pharmaceutical compositions, different controlled release formulations, respectively comprised or formed from the following components, such as carvedilol free base, carvedilol salts, anhydrous forms or solvates thereof, and which also may include, but are not limited to the various components (i.e., such as conventionally known, adjuvants, carriers, diluents, excipients, agents, plasticizers, polymers, etc. as described herein) which may be in or formed into different dosage forms (i.e., which may include, but are not limited to, tablets, capsules and the like) as described herein.

Embodiments

In light of the foregoing, a first general embodiment of the present invention, may include, but is not limited to a controlled release formulation or delivery device, which comprises:

a core containing a carvedilol free base, salt, solvate or anhydrous form thereof;

a release modifying agent; and

an outer coating covering the core;
where outer coating or thickness of the outer coating is adapted:
   for substantial impermeability to entry of fluid present in an environment of use and for substantial impermeability toward release of the carvedilol free base, salt, solvate or anhydrous form thereof during a predetermined dosing interval; and
   for a controlled release dispensing exit of the carvedilol free base, salt, solvate or anhydrous form thereof after the predetermined dosing interval;
where the outer coating or thickness of the outer coating includes at least one orifice in at least one face area of the controlled delivery device extending substantially through the outer coating or thickness of the outer coating but not penetrating the core that communicates from the environment of use to the core allowing for release of the carvedilol free base, salt, solvate or anhydrous form thereof into the environment of use;
   where the at least one orifice in the at least one face area of the controlled release delivery device has a substantially dependent rate limiting release factor dependent upon exit of the carvedilol free base, salt, solvate or anhydrous form thereof from the at least one orifice via dissolution, diffusion or erosion; and
   where the release modifying agent enhances or hinders release of the carvedilol free base, salt, solvate or anhydrous form thereof depending upon solubility or effective solubility of the carvedilol free base, salt, solvate or anhydrous form thereof in the environment of use.

In yet another or second general embodiment of the present invention relates to a controlled release delivery formulation or device, which comprises:

a core containing a carvedilol free base, salt, solvate or anhydrous form thereof;

a release modifying agent, and
an outer coating layer covering the core;
where the outer coating layer:
   is substantially impermeable to the entrance of gastrointestinal fluid and substantially impermeable to release of the carvedilol free base, salt, solvate or anhydrous form thereof agent during a predetermined dosing interval; and
   is adapted for a controlled release dispensing exit of the carvedilol free base, salt, solvate or anhydrous form thereof after the predetermined dosing interval;
where the outer coating layer includes at least one orifice for release of the carvedilol free base or corresponding carvedilol salt, anhydrous form or solvate thereof during the dosing interval;
   where the orifice extends substantially completely through the coating but not penetrating the core,
   where a release rate limiting step is dependent substantially on exit of the carvedilol free base or corresponding carvedilol salt, anhydrous form or solvate thereof through the at least one orifice via dissolution, diffusion or erosion of the carvedilol free base or corresponding carvedilol salt, anhydrous form or solvate thereof in solution or suspension, and
   where the release modifying agent enhances or hinders release of the carvedilol free base or corresponding carvedilol salt, anhydrous form or solvate thereof depending upon solubility
   or effective solubility in gastrointestinal fluid.

In yet another or third embodiment, the present invention relates to a controlled release formulation, which comprises:

a solubility enhanced carvedilol salt, solvate or anhydrous form thereof;

where the controlled release formulation following oral dosage exhibits a biphasic plasma profile with a first plasma concentration peak level and a first Tₘₐₓ pulse within 1-4 hours of ingestion and a second plasma concentration peak level and a second Tₘₐₓ pulse within 5-10 hours after ingestion. In an embodiment of the present invention a first Tₘₐₓ pulse may occur within 2-4 hours of ingestion and the second Tₘₐₓ pulse may occur within 5-8 hours after ingestion.

The present invention and aforementioned general or different specific embodiments relate to a formulation in an oral dosage form. An oral dosage form of the present invention may be, but is not limited to a oral tablet dosage form. In particular, such an oral tablet dosage form may be in a mono-layer or single conventional core tablet form or a bilayer tablet dosage form.

Also, in accordance with the present invention and corresponding embodiments as defined herein, a substantially biphasic profile is shown by a tablet as described herein with at least one drug release rate controlling aperture in at least one face of the tablet, where such a tablet may include, but is not limited to the following examples:
[1] a tablet core containing active drug agent in a controlled or delayed release form, which may be overcoated with, but not limited to a time or pH dependent film coat or other pharmaceutically acceptable excipients; or
[2] a tablet core, which may comprise a bilayer tablet which may incorporate:
   [a] a rapidly releasing or an immediate-release layer(s) which exhibit(s) a rapid release rate of active drug component(s), i.e., carvedilol salt, solvate, or anhydrous form(s), which may be comprised of, but is not limited to, compressible granular forms to form such an immediate-release core layer, to provide a first peak plasma concentration between 1 to 3 hours after dosing the composition or formulation; and
   [b] modified release or delayed-controlled release layer(s) which exhibit(s) a controlled or delayed release rate of active drug component(s), i.e., carvedilol free base or a carvedilol salt, solvate, or anhydrous form(s) thereof, which may be comprised of, but is not limited to, compressible granular forms to form such a modified-release layer, which may include, but is/are not limited to containing polymer materials or components that aid or determine release rate of the aforementioned active drug component in the modified-release layer(s) that exhibit(s) a release rate of the carvedilol free base or carvedilol salt, solvate, or anhydrous form to provide a second peak plasma concentration between 5 to 10 hours after dosing the composition or formulation;

where the first peak plasma concentration level and the second Plasma peak concentration level are in a mean ratio of about at least 1:1 to about at least 1:4.

In yet another or fourth embodiment, the present invention also relates to a controlled release formulation, comprising at least one of the following components:

[a] carvedilol free base; and [b] a solubility enhanced carvedilol salt, solvate or anhydrous forms; or

[a] carvedilol free base; or [b] a solubility enhanced carvedilol salt, solvate or anhydrous forms;

where the controlled release formulation following oral dosage exhibits a biphasic plasma profile which exhibits a first plasma concentration peak level and a first Tₘₐₓ pulse within 1-4 hours of ingestion and a second plasma concentration peak level and a second Tₘₐₓ pulse within 5-8 hours after ingestion.

In yet another or fifth embodiment, the present invention also relates to a controlled release formulation, comprising at least one of these components:

[a] carvedilol free base form; and [b] solubility enhanced carvedilol salt, solvate or anhydrous forms;

[a] carvedilol free base form; or [b] solubility enhanced carvedilol salt, solvate or anhydrous forms;

where the controlled release formulation is in a oral tablet dosage form, wherein each face of the oral tablet dosage form includes no aperatures or a number of aperatures of varying diameters to control rate of the carvedilol form release; and

where the controlled release formulation following oral dosage exhibits a biphasic plasma peak concentration profile which exhibits a first pharmacokinetic plasma concentration peak level and first Tₘₐₓ pulse within 1-4 hours of ingestion and a second plasma concentration peak level and a second Tₘₐₓ pulse within 5-8 hours after ingestion.

In accordance with the present invention and the aforementioned first to fifth embodiments, an oral tablet dosage form may be comprised of a coated surface layer and a matrix core base layer. The formulation exhibits upon dissolution a plasma peak concentration release profile based upon a first controlled release of the carvedilol carvedilol free base form or a solubility enhanced carvedilol salt, solvate or anhydrous forms form as controlled by the aperature size in the coated surface layer face in combination with a second controlled release of the carvedilol free base form or a solubility enhanced carvedilol salt, solvate or anhydrous forms form from a matrix-based tablet.

In yet another or sixth embodiment, the present invention also relates to a controlled release formulation, which comprises:

a solubility enhanced carvedilol free base or carvedilol salt, solvate or anhydrous forms thereof;

where the controlled release controlled release formulation is in a oral tablet dosage form comprised of a coated surface layer and a matrix core base layer, wherein each face of the oral tablet dosage form includes no aperatures or a number of aperatures of varying diameters to control rate of the carvedilol form release;

where the controlled release formulation exhibits upon dissolution a plasma peak concentration release profile based upon a first controlled release of the carvedilol form as controlled by the aperature size in the coated surface layer face in combination with a second controlled release of the carvedilol form from a matrix-based tablet; and

where the controlled release formulation following oral dosage exhibits a biphasic plasma peak concentration profile which exhibits a first pharmacokinetic plasma concentration peak level and first Tₘₐₓ pulse within 1-4 hours of ingestion and a second plasma concentration peak level and a second Tₘₐₓ pulse within 5-8 hours after ingestion.

In accordance with the present invention and the aforementioned first to sixth embodiments, an oral tablet dosage form of the present invention may be an over-encapsulated tablet. The oral tablet dosage form also may be overcoated with pH sensitive or drug release rate controlling polymer(s). Such polymers also may be included, but not limited to modified release layer coatings or overcoating materials. The coated surface layer may be coated with an enteric coat. The number of aperatures in the oral tablet dosage form preferrably is, but is not limited to two, one in each face of each oral dosage form. An aperature may be defined with aperature or orifice diameter size range from at least about 0.0 mm to at least about 7.0 mm. For example, coated tablets of the present invention may have an aperature or orifice of 6 mm in diameter. Such an oral tablet dosage form may also be formulated as separate sequential layers with a tablet matrix core base layer, where the matrix core base layer is a hydrophilic matric core. The coated surface layer also may be coated with a film coat. The oral tablet dosage form may be formulated as separate sequential layers, where each layer has different release-modifying components or characteristics based upon gastrointestinal environment, pH or time.

In yet another or seventh embodiment, the present invention also relates to a controlled release formulation, comprising at least one of these components:

[a] carvedilol free base; and [b] a solubility enhanced carvedilol salt, solvate or anhydrous forms thereof; or

[a] carvedilol free base; or [b] a solubility enhanced carvedilol salt, solvate or anhydrous forms thereof;

where the controlled release formulation is in an oral tablet dosage form which exhibits upon dissolution a plasma peak concentration release profile based upon a controlled release of the carvedilol form as controlled by a number of aperatures and/or aperature depth or aperature size drilled into a tablet dosage form formed from a coated surface layer face in combination with a matrix base layer; and

where the controlled release formulation following oral dosage exhibits a biphasic plasma peak concentration profile which exhibits a first pharmacokinetic plasma concentration peak level and a first Tₘₐₓ pulse within 1-4 hours of ingestion and a second plasma concentration peak level and a second Tₘₐₓ pulse within 5-8 hours after ingestion.

In accordance with the present invention and the aforementioned first to seventh embodiments, the number of aperatures in the oral tablet dosage form is preferrably two, one in each face of each oral dosage form. Moreover, such aperatures have an aperature size with an aperature diameter size range from about 0.0 mm to about 7.0 mm. Moreover, an oral tablet dosage form of the present invention is formulated as separate sequential layers with a tablet matrix core base, where the matrix core base may be, but is not limited to being a hydrophilic matric core. An oral tablet dosage form of the present invention may have a surface layer is coated with an enteric coat, wherein enteric coat may be, but is not limited to being a film coat. An oral tablet dosage form of the present invention may also be overcoated with a pH sensitive polymer. An oral tablet dosage form may be formulated as separate sequential layers, where each layer has different release-modifying component properties based upon gastrointestinal environment, pH or time. An oral tablet dosage form of the present invention may also be overcoated with a pH sensitive polymer.

In yet another or eighth embodiment, the present invention also relates to a controlled release formulation, which comprises at least one of these components:

[a] carvedilol free base; and [b] a solubility enhanced carvedilol salt, solvate or anhydrous forms; or

[a] carvedilol free base; or [b] a solubility enhanced carvedilol salt, solvate or anhydrous forms;

where the controlled release formulation is a bilayer tablet dosage form;

where the controlled release formulation following oral dosage administration that exhibits a biphasic pharmacokinetic plasma peak concentration release effected by a first controlled release of the carvedilol form from one layer with a first Tₘₐₓ pulse within 1-3 hours and a second controlled release of the carvedilol form from a second layer with a second Tₘₐₓ pulse 3-5 hours after the first Tₘₐₓ pulse.

In accordance with the present invention and the aforementioned first to eighth embodiments, a bilayer tablet dosage form of the present invention may be formulated as two separate sequential layers with one layer defined as a tablet core matrix. The two separate sequential layers may be characterized by different release-modifying components or characteristics based upon gastrointestinal environment, pH or time. A specific embodiment of the present invention, defines that the two separate sequential layers may be comprised of a immediate release layer and a modified release layer. The immediate release layer may be formed from carvedilol free base form, where the carvedilol free base form is formed from immediate release granules formed from carvedilol free base. The modified release layer is formed from a carvedilol salt, solvate or anhydrous forms thereof. Moreover, such a bilayer tablet dosage form may be overcoated with a pH sensitive polymer.

In yet another or ninth embodiment, the present invention also relates to a controlled release formulation, comprising at least one of these components:

[a] carvedilol free base; and [b] a solubility enhanced carvedilol salt, solvate or anhydrous forms; or

[a] carvedilol free base; or [b] a solubility enhanced carvedilol salt, solvate or anhydrous forms;

where the controlled release formulation is a bilayer tablet dosage form comprised of an immediate release layer and a modified release layer; and

where the controlled release formulation following oral dosage administration that exhibits a biphasic pharmacokinetic plasma peak concentration release effected by a first controlled release of the carvedilol form from one layer with a first Tₘₐₓ pulse within 1-3 hours and a second controlled release of the carvedilol form from a second layer with a second Tₘₐₓ pulse 3-5 hours after the first Tₘₐₓ pulse.

In accordance with the present invention and the aforementioned embodiments, an oral tablet dosage form of the present invention may include, but is not limited to a an alternative unit, where active drug release is constrained or delayed by a time or pH-dependent coat, with or without an aperture, through which such drug is released at a controlled rate. The coat composition in such an oral tablet dosage form may be, but not limited to being varied such that the aforementioned coat composition is eroded or dissolved at a desired pH, or after a defined time following ingestion such that drug is released “later” to provide the required “early morning” plasma levels or to sustain levels to cover the full dosage interval. For example, the present invention may include, but is not limited to, an over-encapsulated tablet which may be, but not limited to an overcoating material that contains a pH sensitive polymer as described herein.

Methods of Treatment

The compounds or pharmaceutical compositions prepared according to the present invention can be used to treat warm-blooded animals, such as mammals, which include humans.

The present invention relates to methods of treating hypertension, congestive heart failure or angina which comprises administering to a subject in need thereof an effective amount of carvedilol free base or a carvedilol salt, anhydrous forms, or solvate thereof, a pharmaceutical composition, or controlled release formulation as described herein.

For example, the present invention further relates to a method of treating hypertension, congestive heart failure and angina, which comprises administering to a subject in need thereof an effective amount of a carvedilol phosphate salt (which may include, but are not limited to novel crystalline or other solid forms), anhydrous forms, or solvates thereof, a pharmaceutical composition or controlled release formulation (i.e., which contains such salts or solvates of carvedilol phosphate), etc. In a specific embodiment, the present invention relates to a method of treating hypertension, which comprises administering to a subject in need thereof an effective amount of a carvedilol phosphate salt (which may include, but are not limited to novel crystalline or other solid forms), anhydrous forms, or solvates thereof, a pharmaceutical composition or controlled release formulation (i.e., which contains such salts or solvates of carvedilol phosphate), etc.

The present invention also relates to a method of delivering carvedilol to gastrointestinal tract of a subject in need thereof, which comprises administering an effective amount of carvedilol free base or a carvedilol salt, anhydrous forms, or solvate thereof, which may be in, but not limited to being in combination with carvedilol free base, corresponding pharmaceutical compositions or control-release formulations or dosage forms as described herein.

In a specific embodiment, the present invention relates to a method of delivering carvedilol to the lower intestinal tract, which comprises administering an effective amount of a carvedilol salt, anhydrous forms, or solvate thereof, which may be in, but not limited to being in combination with carvedilol free base, corresponding pharmaceutical compositions or control-release formulations or dosage forms as described herein.

Conventional administration methods as described in examples and throughout this application above may be suitable for such use in methods of treatment or delivery of the present invention.

Methods of Treatment and Combination Therapies

The compounds or pharmaceutical compositions prepared according to the present invention can be used to treat warm-blooded animals, such as mammals, which include humans.

The present invention relates to methods of treating cardiovascular diseases, which may include, but is not limited to hypertension, congestive heart failure, atherosclerosis, or angina, which comprises administering to a subject in need thereof an effective amount of carvedilol free base or a carvedilol salt, anhydrous forms, or solvate thereof as defined herein, a pharmaceutical composition, or controlled release formulation as described herein.

For example, the present invention further relates to a method of treating hypertension, congestive heart failure, atherosclerosis and angina, which comprises administering to a subject in need thereof an effective amount of a carvedilol phosphate salt (which may include, but are not limited to novel crystalline or other solid forms), anhydrous forms, or solvates thereof, a pharmaceutical composition or controlled release formulation (i.e., which contains such salts or solvates of carvedilol phosphate), etc.

In a specific embodiment, the present invention relates to a method of treating hypertension, which comprises administering to a subject in need thereof an effective amount of a carvedilol phosphate salt (which may include novel crystalline or other solid forms), anhydrous forms, or solvates thereof, a pharmaceutical composition or controlled release formulation (i.e., which contains such salts or solvates of carvedilol phosphate), etc.

In another specific embodiment, the present invention relates to a method of treating atherosclerosis, which comprises administering to a subject in need thereof an effective amount of a carvedilol phosphate salt (which may include novel crystalline or other solid forms), anhydrous forms, or solvates thereof, a pharmaceutical composition or controlled release formulation (i.e., which contains such salts or solvates of carvedilol phosphate), etc.

The present invention also relates to a method of delivering carvedilol to gastrointestinal tract of a subject in need thereof, which comprises administering an effective amount of a carvedilol salt, anhydrous forms, or solvate thereof, which may be in, but not limited to being in combination with carvedilol free base, corresponding pharmaceutical compositions or control-release formulations or dosage forms as described herein.

In a specific embodiment, the present invention relates to a method of delivering carvedilol to the gastrointestinal tract, which comprises administering an effective amount of a carvedilol salt, anhydrous forms, or solvate thereof, which may be in, but not limited to being in combination with carvedilol free base, corresponding pharmaceutical compositions or control-release formulations or dosage forms as described herein.

In another embodiment, the present invention relates to a method of orally dosing a modified release composition, dosage form or formulation as described herein, which comprises progressive release of a drug amount of carvedilol free base or a carvedilol salt, solvate or anhydrous form thereof from each microcapsule of the modified release composition, dosage form or formulation, which are absorped as the microparticles transit the GI tract to provide sustained and controlled release levels of the drug amount for maintenance of prolonged plasma levels.

The present invention also relates to a method of dosing a carvedilol dosage unit to a patient in need thereof, which comprises administering to a subject in need thereof, which comprises administering to a subject in need thereof an effective amount of a controlled release composition, dosage form or formulation of the present invention, an effective amount of a controlled release composition, dosage form or formulation of the present invention, where release of the carvedilol dosage unit transits through a lower gastrointestinal tract.

In accordance with any of the methods of administration of the present invention, the term a “therapeutically effective amount”, as used herein, generally includes within its meaning a non-toxic but sufficient amount of the particular drug to which it is referring to provide the desired therapeutic effect. The exact amount required will vary from subject to subject depending on factors such as the patient's general health, the patient's age, etc.

Also, the present invention relates to combination therapy methods for treatment of cardiovascular disorders to a subject in need thereof, which comprises a compound or controlled release composition, dosage form or formulation as described herein in a synergistic combination with other drug agents, which may, but not limited to a group selected from the group consisting of calcium channel blockers, beta blockers, diuretics, ACE inhibitors, Angiotensin II receptor antagonists, statin agents and or the like, or pharmaceutically acceptable adjuvant(s), carrier(s), diluent(s), and/or excipient(s).

In particular, compounds or controlled release composition, dosage forms or formulations of the present invention may be employed alone or in combination with each other or other suitable therapeutic agents useful in treatment of the aforementioned cardiovascular disorders, which may include, but are not limited to hypertension, congestive heart failure, atherosclerosis, angina and the like.

Examples of suitable calcium channel blocker agents (both L-type and T-type) for use in combination with compounds or a controlled release composition, dosage form or formulation of the present invention, may include, but are not limited to diltiazem, verapamil, nifedipine, amlodipine, mybefradil or any other calcium channel blocker and the like.

Suitable beta-blockers for use in combination with compounds or a controlled release composition, dosage form or formulation of the present invention, may include, but are not limited to atenolol, metoprolol, and the like .

Suitable statin agents, such as HMG-CoA reductase inhibitors, for use in combination with compounds or a controlled release composition, dosage form or formulation of the present invention, may include, but are not limited to lovastatin, simvastatin, pravastatin, fluvastatin, cerivastatin, atorvastatin or any other suitable statin agent and the like.

Suitable adrenoreceptor agents for use in combination with compounds or a controlled release composition, dosage form or formulation of the present invention, may include, but are not limited to may include metoprolol (toprol-XL), metoprol succinate, metoprol tartrate or any other suitable adrenoreceptor agents and the like, Suitable ACE inhibitors for use in combination with compounds or a controlled release composition, dosage form or formulation of the present invention, may include, but are not limited to alacepril, benazepril, captopril, ceronapril, cilazepril, cilazopril, delapril, enalapril, enalaprilat, fosinopril, imidapril, libenzapril, lisinopril, moexipril, monopril, moveltipril, pentopril, perindopril, quinapril, ramipril, spirapril, temocapril, teprotide, trandolapril, zofenopril or any other suitable ACE inhibitor and the like.

Suitable diuretics for use in combination with compounds or a controlled release formulation of the present invention, may include, but are not limited to acetazolamide, flumethiazide, hydroflumethiazide, bendroflumethiazide, brinzolamide, dichlorphenamide, dorzolamide, methazolamide, azosemide, bumetanide, ethacrynic acid, etozolin, frusemide, piretanide, torasemide, isosorbide, mannitol, amiloride, canrenoate potassium, canrenone, spironolactone, triamterene, althiazide, bemetizide, bendrofluazide, benzthiazide, buthiazide, chlorothiazide, chlorthalidone, clopamide, cyclopenthiazide, cyclothiazide, epithiazide, hydrochlorothiazide, hydroflumethiazide, indapamide, mebutizide, mefruside, methylcothiazide, meticrane, metolazone, polythiazide, quinethazone, teclothiazide, trichlormethiazide, tripamide, xipamide, furosemide, musolimine, triamtrenene, amiloride, and spironolactone or other suitable diuretics and the like.

Suitable angiotensin II receptor antagonists for use in combination with compounds or a controlled release formulation of the present invention, may include, but are not limited to losartan, irbesartan, valsartan or any other angiotensin II receptor antagonist and the like.

Active drug or therapeutic agents or compounds, such as those described above may be prepared according to processes or methods taught by either the present disclosure and/or processes or methods known to those of skill in the art.

Active drug or therapeutic agents, when employed in combination with the compounds, controlled release compositions, dosage forms or formulations of the present invention, may be used or administered, for example, in dosage amounts indicated in the Physicians' Desk Reference (PDR) or as otherwise determined by one of ordinary skill in the art.

In the context of this specification, the term “simultaneously” when referring to simultaneous administration of the relevant drugs means at exactly the same time, as would be the case, for example in embodiments where the drugs are combined in a single preparation. In other embodiments, “simultaneously” can mean one drug taken a short duration after another, wherein “a short duration” means a duration which allows the drugs to have their intended synergistic effect.

In light of the foregoing, the present invention also relates to a combination therapy, which may be a comprised of a simultaneous or co-administration, or serial administration of a combination of compounds, controlled release compositions, dosage forms or formulations of the present invention with other active drug or therapeutic agents, such as described above, and where such administration also is determined by one of ordinary skill in the art. As previously indicated active drug compounds, controlled release compositions, dosage forms or formulations of the present invention, may include, but are not limited to a carvedilol free base or a carvedilol salt, solvate or anhydrous form thereof.

In addition, the present invention also relates to a combination therapy for the treatment or prevention of cardiovascular diseases as described herein, which is comprised of a composition, dosage form or formulation formed from a synergistic combination or mixture of compounds, controlled release compositions, dosage forms or formulations of the present invention and another active drug or therapeutic agent or agents as those described above and optionally which comprises pharmaceutically acceptable carrier, diluent or adjuvent. In such an aforementioned combination composition, dosage form or formulation of the present invention, each of the active drug components are contained in therapeutically effective and synergistic dosage amounts.

In yet another embodiment, the present invention further relates to a combination therapy for the treatment of cardiovascular diseases, such as diseases described herein, which comprises administering a synergistic combination of:
[1] a therapeutically effective amount of a carvedilol free base or a carvedilol salt, solvate or anhydrous form thereof; or
   a corresponding controlled release composition, dosage form or formulation thereof, which comprises a therapeutically effective amount of a carvedilol free base or a carvedilol salt, solvate or anhydrous form thereof; and
[2] a therapeutically effective amount of another active drug or therapeutic agent selected from the group consisting of at least one calcium channel blockers, beta blockers, diuretics, ACE inhibitors, Angiotensin II receptor antagonists, statin agents and or the like, any other drugs suitable for the treatment of cardiovascular diseases, or combinations thereof; and
   further comprising a pharmaceutically acceptable carrier, diluent or adjuvant.

Conventional administration methods as described in examples and throughout this application above may be suitable for such use in methods of treatment or delivery of various forms of the present invention, including any combination therapy methods.

The Examples set forth below are illustrative of the present invention and are not intended to limit, in any way, the scope of the present invention.

### EXAMPLES

Carvedilol Salt, Solvate, or Anhydrous forms Examples

Carvedilol Phosphate Examples

### Example 1

Form I Carvedilol Dihydrogen Phosphate Hemihydrate Preparation

A suitable reactor is charged with acetone. The acetone solution is sequentially charged with carvedilol and water. Upon addition of the water, the slurry dissolves quickly. To the solution is added aqueous H₃PO₄. The reaction mixture is stirred at room temperature and carvedilol dihydrogen phosphate seeds are added in one portion. The solid precipitate formed is stirred, then filtered and the collected cake is washed with aqueous acetone. The cake is dried under vacuum to a constant weight. The cake is weighed and stored in a polyethylene container.

### Example 2

Form II Carvedilol Dihydrogen Phosphate Dihydrate Preparation

Form I is slurried in acetone/water mixture between 10 and 30° C. for several days.

### Example 3

Form III Carvedilol Dihydrogen phosphate Methanol Solvate Preparation

Form I is slurried in methanol between 10 and 30° C. for several days.

### Example 4

Form IV—Carvedilol Dihydrogen Phosphate Dihydrate Preparation

Carvedilol dihydrogen dihydrogen phosphate is dissolved in an acetone/water mixture. The acetone is removed by distillation. A solid crystallizes during acetone removal and is filtered and dried.

### Example 5

Form V—Carvedilol Dihydrogen Phosphate Preparation

Carvedilol dihydrogen phosphate hemihydrate (Form I) was suspended in water, and the suspension was placed on a mechanical shaker at room temperature. After 48 hours of shaking, the solid was isolated from suspension by filtration, then dried in a desiccator under vacuum for a few days.

### Example 6

Form VI—Carvedilol Hydrogen Phosphate Preparation

A suitable reactor is charged with acetone. The acetone solution is sequentially charged with SK&F 105517 and water. Upon addition of the water, the slurry dissolves quickly. To the solution is added aqueous H₃PO₄ (at ½ the molar quantity of Carvedilol). The reaction mixture is stirred and allowed to crystallize. The solid precipitate formed is stirred and cooled, then filtered and the collected cake is washed with aqueous acetone.

### Example 7

¹³C and ³¹P Solid State NMR Data Analysis of Carvedilol Dihydrogen Phosphate

A sample of carvedilol dihydrogen phosphate was analyzed by solid-state ¹³C NMR and ³¹P NMR (i.e., to probe solid compound form structure).

Carvedilol dihydrogen phosphate (Parent MW=406.5; Salt MW=504.5) has the following structure and numbering scheme: 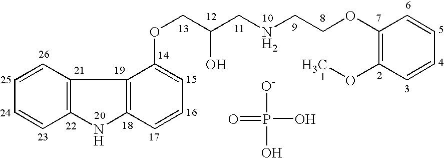
Experimental Details and ¹³C and ³¹P Analysis

The solid state ¹³C NMR methods used to analyze compounds of the present invention produce a qualitative picture of the types of carbon sites within the solid material. Because of variable polarization transfer rates and the need for sideband suppression, the peak intensities are not quantitative (much like the case in solution-state ¹³C NMR).

However, the ³¹P spectra are inherently quantitative.

For the ¹³C analysis, approximately 100 mg of sample was packed into a 7-mm O.D. magic-angle spinning rotor and spun at 5 kHz. The ¹³C spectrum of the sample was recorded using a CP-TOSS pulse sequence (cross-polarization with total suppression of sidebands). An edited spectrum containing only quaternary and methyl carbons was then obtained using an CP-TOSS sequence with NQS (non-quaternary suppression). The ¹³C spectra are referenced externally to tetramethylsilane via a sample of solid hexamethylbenzene.

For ³¹P Solid State NMR, approximately 40 mg of sample was packed into a 4-mm O.D. rotor and spun at 10 kHz. Both CP-MAS and single-pulse MAS ³¹P pulse sequences were used with ¹H decoupling. The ³¹P data are externally referenced to 85% phosphoric acid by a secondary solid-state reference (triphenylphosphine oxide). The Bruker AMX2-360 spectrometer used for this work operates at ¹³C, ³¹P and ¹H frequencies of 90.556, 145.782 and 360.097 MHz, respectively. All spectra were obtained at 298 K.

Results and Discussion

The highly sensitive ¹³C and ³¹P Solid State NMR identification methods were used for the analysis and characterization of a polymorphic form of Carvedilol phosphate, which confirms its chemical structure in the solid-state.

The form of Carvedilol dihydrogen phosphate is defined by these spectra, where both ¹³C and ³¹P spectra show clear and distinct differences.

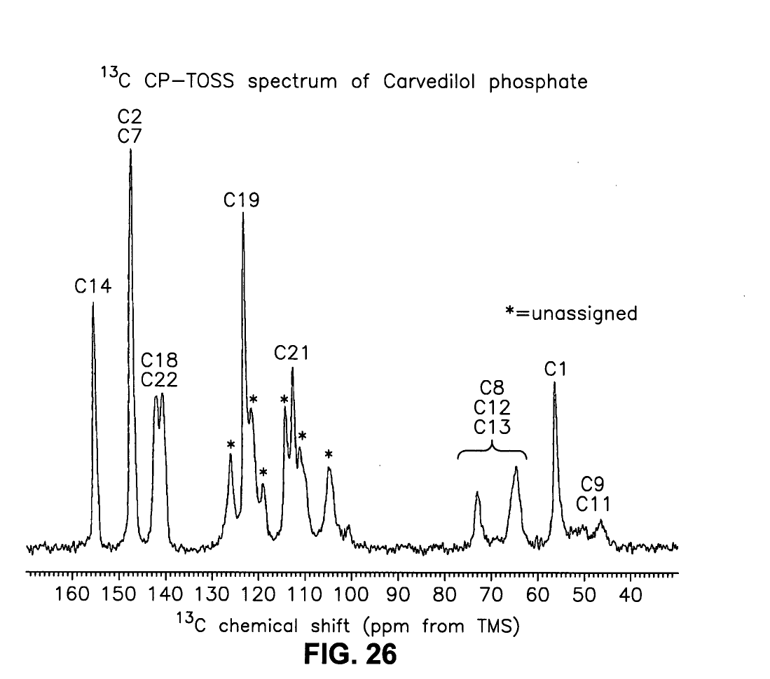

In particular, FIG. 26 shows the ¹³C CP-TOSS spectrum of carvedilol dihydrogen phosphate. An assignment of the numerous ¹³C resonances in FIG. 1 can be made by chemical shift assignment, the NQS spectrum and comparisons with solution-state ¹³C assignments. At least two non-equivalent molecules per unit cell are observed in this form of Carvedilol phosphate.

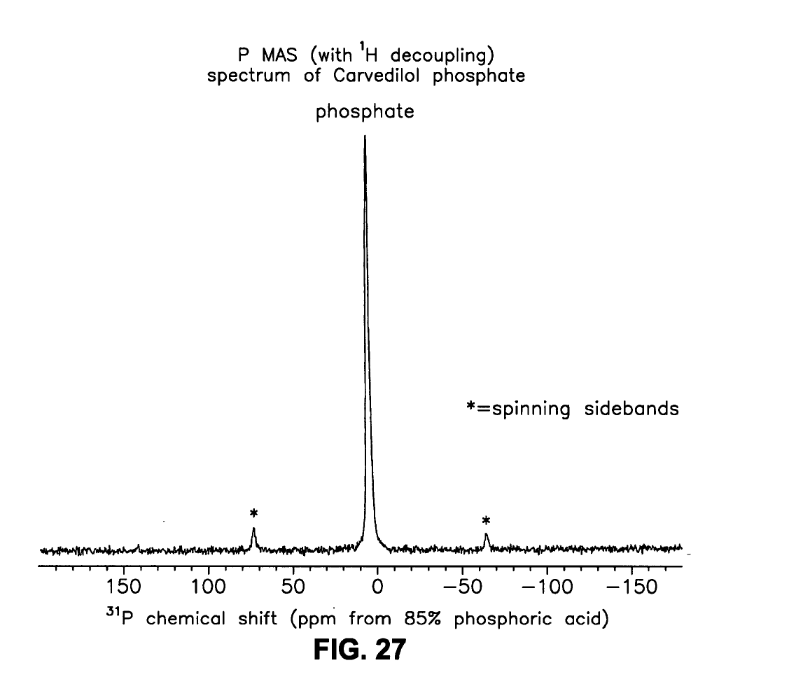

FIG. 27 shows the ³¹P MAS spectrum of carvedilol dihydrogen phosphate. A single phosphorus signal is observed at 4.7 ppm, which is characteristic of phosphate salts.

Carvedilol Hydrogen Bromide Examples

### Example 8

Form 1. Carvedilol HBr Monohydrate.

A suitable reactor is charged with acetone. The acetone solution is sequentially charged with carvedilol, water and 48% aqueous HBr. On addition of the water, the acetone slurry becomes a solution. The reaction mixture is stirred at room temperature. A solid precipitates during the course of the stir. The precipitate is filtered and the collected cake is washed with acetone. The cake is dried under vacuum to a constant weight. The cake is weighed and stored in a polyethylene container.

The single crystal x-ray data for carvedilol hydrobromide monohydrate is provided below.

TABLE 1
Sample and Crystal Data for Carvedilol
Hydrobromide Monohydrate.

| Crystallization solvents | Acetone, water | |
|---|---|---|
| Crystallization method | Slow cooling | |
| Empirical formula | C₂₄H₂₉BrN₂O₅ | |
| Formula weight | 505.40 | |
| Temperature | 150(2) K | |
| Wavelength | 0.71073 Å | |
| Crystal size | 0.18 × 0.14 × 0.08 mm | |
| Crystal habit | Clear colorless prism | |
| Crystal system | Monoclinic | |
| Space group | C2/c | |
| Unit cell dimensions | a = 18.0356(3) Å | α = 90° |
| | b = 20.8385(5) Å | β = 103.5680(10)° |
| | c = 12.9342(3) Å | γ = 90° |
| Volume | 4725.46(18) Å³ | |
| Z | 8 | |
| Density (calculated) | 1.421 Mg/m³ | |
| Absorption coefficient | 1.777 mm⁻¹ | |
| F(000) | 2096 | |

TABLE 2
Data collection and structure refinement
for Carvedilol Hydrobromide Monohydrate.

| | |
|---|---|
| Diffractometer | KappaCCD |
| Radiation source | Fine-focus sealed tube, MoK_{α} |
| Data collection method | CCD; rotation images; thick slices |
| Theta range for data collection | 3.42 to 23.27° |
| Index ranges | 0 ≦ h ≦ 20, 0 ≦ k ≦ |
| | 23, −14 ≦ I ≦ 13 |
| Reflections collected | 30823 |
| Independent reflections | 3404 [R(int) = 0.042] |
| Coverage of independent | 99.7% |
| reflections | |
| Variation in check reflections | N/A |
| Absorption correction | Symmetry-related measurements |
| Max. and min. transmission | 0.8709 and 0.7404 |
| Structure solution technique | Direct methods |
| Structure solution program | SHELXTL V5.10 UNIX |
| | (Bruker, 1997) |
| Refinement technique | Full-matrix least-squares on F² |
| Refinement program | SHELXTL V5.10 UNIX |
| | (Bruker, 1997) |
| Function minimized | Σ w(Fₒ² − F_{c}²)² |
| Data/restraints/parameters | 3404/11/336 |
| Goodness-of-fit on F² | 1.020 |
| Δ/σₘₐₓ | 0.000 |
| Final R indices | |
| 3071 data; I > 2σ(I) | R1 = 0.0353, wR2 = 0.0797 |
| all data | R1 = 0.0405, wR2 = 0.0829 |
| Weighting scheme | w = 1/[σ²(Fₒ²) + |
| | [ (0.0304P)² + 14.1564P ] |
| | where P = [MAX(Fₒ², 0) + |
| | 2F_{c}²]/3 |
| Largest diff. peak and hole | 0.786 and −0.914 e.Å⁻³ |

Refinement Summary:

| | |
|---|---|
| Ordered Non-H atoms, XYZ | Freely refined |
| Ordered Non-H atoms, U | Anisotropic |
| H atoms (on carbon), XYZ | Idealized positions |
| | riding on attached atom |
| H atoms (on carbon), U | Appropriate constant |
| | times Ueq of attached atom |
| H atoms (on heteroatoms), XYZ | Freely refined |
| H atoms (on heteroatoms), U | Refined Isotropically |
| Disordered atoms, OCC | See Table 10 |
| Disordered atoms, XYZ | Refined with distance |
| | restraints |
| Disordered atoms, U | Anisotropic |

TABLE 3
Atomic Coordinates and Equivalent Isotropic Atomic Displacement
Parameters (Å²) for Carvedilol Hydrobromide Monohydrate.
U(eq) is defined as one third of the trace of the orthogonalized Uᵢⱼ tensor.

| | x/a | y/b | z/c | U(eq) |
|---|---|---|---|---|
| Br1 | 0.5000 | 0.22079(2) | −0.2500 | 0.04329(15) |
| Br2 | 0.0000 | 0.40821(2) | −0.2500 | 0.04510(16) |
| O1 | 0.19543(10) | 0.37037(10) | −0.00168(15) | 0.0328(5) |
| O2 | 0.08660(19) | 0.48508(15) | 0.1085(2) | 0.0312(7) |
| O2′ | 0.0825(3) | 0.4816(3) | −0.0328(4) | 0.0311(13) |
| O3 | −0.19428(10) | 0.39492(10) | −0.01310(15) | 0.0347(5) |
| O4 | −0.24723(12) | 0.46974(11) | 0.11008(16) | 0.0404(5) |
| O99A | −0.0880(5) | 0.4236(3) | 0.1967(7) | 0.0430(19) |
| O99B | −0.0833(5) | 0.4514(4) | 0.1784(7) | 0.0431(19) |
| N1 | 0.34092(16) | 0.25072(13) | −0.1793(2) | 0.0390(7) |
| N2 | −0.03151(14) | 0.39706(13) | −0.0026(2) | 0.0314(6) |
| C1 | 0.26859(15) | 0.35551(14) | −0.0070(2) | 0.0301(7) |
| C2 | 0.33380(16) | 0.38188(15) | 0.0568(2) | 0.0339(7) |
| C3 | 0.40553(17) | 0.36537(16) | 0.0409(3) | 0.0402(8) |
| C4 | 0.41433(17) | 0.32249(16) | −0.0364(3) | 0.0401(8) |
| C5 | 0.34850(16) | 0.29538(15) | −0.0986(2) | 0.0343(7) |
| C6 | 0.26499(17) | 0.23737(14) | −0.2202(2) | 0.0343(7) |
| C7 | 0.23145(19) | 0.19604(15) | −0.3022(2) | 0.0401(8) |
| C8 | 0.15313(19) | 0.19096(15) | −0.3275(2) | 0.0412(8) |
| C9 | 0.10866(18) | 0.22594(14) | −0.2721(2) | 0.0364(7) |
| C10 | 0.14185(17) | 0.26731(14) | −0.1910(2) | 0.0323(7) |
| C11 | 0.22085(16) | 0.27356(13) | −0.1639(2) | 0.0300(7) |
| C12 | 0.27490(16) | 0.31103(13) | −0.0855(2) | 0.0294(6) |
| C13 | 0.18523(16) | 0.41746(14) | 0.0740(2) | 0.0301(7) |
| C14 | 0.10181(16) | 0.43671(13) | 0.0452(2) | 0.0305(7) |
| C15 | 0.05016(15) | 0.37919(14) | 0.0363(2) | 0.0289(6) |
| C16 | −0.08143(16) | 0.33991(14) | −0.0272(2) | 0.0361(7) |
| C17 | −0.16200(16) | 0.35626(16) | −0.0833(2) | 0.0380(7) |
| C18 | −0.27156(15) | 0.40680(14) | −0.0445(2) | 0.0300(6) |
| C19 | −0.30049(16) | 0.44705(14) | 0.0236(2) | 0.0316(7) |
| C20 | −0.37754(18) | 0.46060(16) | 0.0007(3) | 0.0409(8) |
| C21 | −0.42545(18) | 0.43467(17) | −0.0895(3) | 0.0499(9) |
| C22 | −0.39733(18) | 0.39593(17) | −0.1567(3) | 0.0504(9) |
| C23 | −0.31949(17) | 0.38199(15) | −0.1342(3) | 0.0388(7) |
| C24 | −0.2743(2) | 0.50999(17) | 0.1833(3) | 0.0482(9) |

TABLE 4
Selected Bond Lengths (Å) for Carvedilol
Hydrobromide Monohydrate.

| | | | |
|---|---|---|---|
| O1-C1 | 1.373(3) | O1-C13 | 1.428(3) |
| O2-C14 | 1.366(4) | O2′-C14 | 1.360(6) |
| O3-C18 | 1.380(3) | O3-C17 | 1.435(3) |
| O4-C19 | 1.376(4) | O4-C24 | 1.433(4) |
| N1-C6 | 1.376(4) | N1-C5 | 1.381(4) |
| N2-C16 | 1.482(4) | N2-C15 | 1.488(4) |
| C1-C2 | 1.382(4) | C1-C12 | 1.399(4) |
| C2-C3 | 1.399(4) | C3-C4 | 1.378(5) |
| C4-C5 | 1.388(4) | C5-C12 | 1.415(4) |
| C6-C7 | 1.389(4) | C6-C11 | 1.416(4) |
| C7-C8 | 1.377(5) | C8-C9 | 1.399(4) |
| C9-C10 | 1.381(4) | C10-C11 | 1.391(4) |
| C11-C12 | 1.458(4) | C13-C14 | 1.517(4) |
| C14-C15 | 1.506(4) | C16-C17 | 1.503(4) |
| C18-C23 | 1.374(4) | C18-C19 | 1.403(4) |
| C19-C20 | 1.380(4) | C20-C21 | 1.388(5) |
| C21-C22 | 1.368(5) | C22-C23 | 1.396(4) |

TABLE 5
Selected bond angles (°) for Carvedilol
Hydrobromide Monohydrate.

| | | | |
|---|---|---|---|
| C1-O1-C13 | 118.0(2) | C18-O3-C17 | 116.5(2) |
| C19-O4-C24 | 117.2(2) | C6-N1-C5 | 109.9(3) |
| C16-N2-C15 | 112.0(2) | O1-C1-C2 | 125.0(3) |
| O1-C1-C12 | 115.4(2) | C2-C1-C12 | 119.6(3) |
| C1-C2-C3 | 120.1(3) | C4-C3-C2 | 122.3(3) |
| C3-C4-C5 | 117.1(3) | N1-C5-C4 | 129.2(3) |
| N1-C5-C12 | 108.5(3) | C4-C5-C12 | 122.4(3) |
| N1-C6-C7 | 129.4(3) | N1-C6-C11 | 108.9(3) |
| C7-C6-C11 | 121.7(3) | C8-C7-C6 | 117.9(3) |
| C7-C8-C9 | 121.1(3) | C10-C9-C8 | 121.0(3) |
| C9-C10-C11 | 119.1(3) | C10-C11-C6 | 119.1(3) |
| C10-C11-C12 | 134.7(3) | C6-C11-C12 | 106.2(3) |
| C1-C12-C5 | 118.6(3) | C1-C12-C11 | 134.8(3) |
| C5-C12-C11 | 106.6(3) | O1-C13-C14 | 107.0(2) |
| O2′-C14-O2 | 83.4(3) | O2′-C14-C15 | 116.4(3) |
| O2-C14-C15 | 115.2(3) | O2′-C14-C13 | 115.6(3) |
| O2-C14-C13 | 112.0(3) | C15-C14-C13 | 111.6(2) |
| N2-C15-C14 | 111.8(2) | N2-C16-C17 | 113.0(3) |
| O3-C17-C16 | 108.1(2) | C23-C18-O3 | 125.0(3) |
| C23-C18-C19 | 120.1(3) | O3-C18-C19 | 114.9(2) |
| O4-C19-C20 | 125.4(3) | O4-C19-C18 | 115.1(2) |
| C20-C19-C18 | 119.4(3) | C19-C20-C21 | 119.8(3) |
| C22-C21-C20 | 120.9(3) | C21-C22-C23 | 119.7(3) |
| C18-C23-C22 | 120.0(3) | | |

TABLE 6
Hydrogen Bonds and Short C—H . . . X Contacts for Carvedilol
Hydrobromide Monohydrate (Å and °).

| D-H . . . A | d(D-H) | d(H . . . A) | d(D . . . A) | <(DHA) |
|---|---|---|---|---|
| N1-H1N . . . Br1 | 0.76(3) | 2.53(4) | 3.269(3) | 166(3) |
| N2-H2NA . . . O99A | 0.83(4) | 2.29(4) | 3.037(10) | 149(3) |
| N2-H2NA . . . O99B | 0.83(4) | 2.13(4) | 2.943(10) | 165(4) |
| N2-H2NB . . . O2#1 | 0.89(5) | 2.17(4) | 2.873(4) | 135(4) |
| O2′-H2O′ . . . Br2 | 0.67(5) | 2.65(7) | 3.237(6) | 149(12) |
| O99A-H99A . . . Br1#2 | 0.94(3) | 2.49(4) | 3.395(8) | 163(6) |
| O99B-H99B . . . Br2#1 | 0.94(3) | 2.38(3) | 3.320(8) | 173(6) |
| C15-H15A . . . O1 | 0.99 | 2.38 | 2.783(3) | 103.2 |
| C15-H15B . . . Br1#2 | 0.99 | 2.85 | 3.738(3) | 149.3 |
| C16-H16A . . . Br1#2 | 0.99 | 2.88 | 3.760(3) | 148.2 |

Symmetry transformations used to generate equivalent atoms:
#1−x, −y + 1, −z
#2−x + ½, −y + ½, −z

TABLE 7
Selected torsion angles (°) for Carvedilol
Hydrobromide Monohydrate.

| | | | |
|---|---|---|---|
| C13-O1-C1-C2 | 1.2(4) | C13-O1-C1-C12 | −177.5(2) |
| O1-C1-C2-C3 | −177.0(3) | C12-C1-C2-C3 | 1.7(4) |
| C1-C2-C3-C4 | −0.8(5) | C2-C3-C4-C5 | −0.5(5) |
| C6-N1-C5-C4 | −179.7(3) | C6-N1-C5-C12 | 0.8(3) |
| C3-C4-C5-N1 | −178.6(3) | C3-C4-C5-C12 | 0.8(4) |
| C5-N1-C6-C7 | 179.4(3) | C5-N1-C6-C11 | −0.9(3) |
| N1-C6-C7-C8 | 179.5(3) | C11-C6-C7-C8 | −0.1(4) |
| C6-C7-C8-C9 | −0.4(5) | C7-C8-C9-C10 | 0.8(5) |
| C8-C9-C10-C11 | −0.6(4) | C9-C10-C11-C6 | 0.0(4) |
| C9-C10-C11-C12 | −179.9(3) | N1-C6-C11-C10 | −179.4(3) |
| C7-C6-C11-C10 | 0.3(4) | N1-C6-C11-C12 | 0.6(3) |
| C7-C6-C11-C12 | −179.7(3) | O1-C1-C12-C5 | 177.4(2) |
| C2-C1-C12-C5 | −1.4(4) | O1-C1-C12-C11 | −2.4(5) |
| C2-C1-C12-C11 | 178.8(3) | N1-C5-C12-C1 | 179.6(2) |
| C4-C5-C12-C1 | 0.1(4) | N1-C5-C12-C11 | −0.5(3) |
| C4-C5-C12-C11 | 180.0(3) | C10-C11-C12-C1 | −0.3(6) |
| C6-C11-C12-C1 | 179.8(3) | C10-C11-C12-C5 | 179.9(3) |
| C6-C11-C12-C5 | −0.1(3) | C1-O1-C13-C14 | 166.1(2) |
| O1-C13-C14-O2′ | −82.6(4) | O1-C13-C14-O2 | −175.8(2) |
| O1-C13-C14-C15 | 53.4(3) | C16-N2-C15-C14 | 171.3(2) |
| O2′-C14-C15-N2 | −38.6(4) | O2-C14-C15-N2 | 56.6(3) |
| C13-C14-C15-N2 | −174.2(2) | C15-N2-C16-C17 | −170.5(2) |
| C18-O3-C17-C16 | −170.7(2) | N2-C16-C17-O3 | −63.3(3) |
| C17-O3-C18-C23 | 3.3(4) | C17-O3-C18-C19 | −177.3(3) |
| C24-O4-C19-C20 | 1.0(4) | C24-O4-C19-C18 | −178.7(3) |
| C23-C18-C19-O4 | −179.2(3) | O3-C18-C19-O4 | 1.4(4) |
| C23-C18-C19-C20 | 1.0(4) | O3-C18-C19-C20 | −178.3(3) |
| O4-C19-C20-C21 | 179.9(3) | C18-C19-C20-C21 | −0.4(5) |
| C19-C20-C21-C22 | −0.3(5) | C20-C21-C22-C23 | 0.3(6) |
| O3-C18-C23-C22 | 178.2(3) | C19-C18-C23-C22 | −1.1(5) |
| C21-C22-C23-C18 | 0.4(5) | | |

TABLE 8
Anisotropic Atomic Displacement Parameters (Å²) for Carvedilol Hydrobromide Monohydrate.
The anisotropic atomic displacement factor exponent takes the form: −2π² [h²a*²U₁₁ + . . . + 2hka*
b* U₁₂ ]

| | U₁₁ | U₂₂ | U₃₃ | U₂₃ | U₁₃ | U₁₂ |
|---|---|---|---|---|---|---|
| Br1 | 0.0484(3) | 0.0447(3) | 0.0464(3) | 0.000 | 0.0306(2) | 0.000 |
| Br2 | 0.0707(3) | 0.0413(3) | 0.0234(2) | 0.000 | 0.0111(2) | 0.000 |
| O1 | 0.0272(11) | 0.0408(12) | 0.0323(11) | 0.0067(9) | 0.0108(9) | −0.0009(9) |
| O2 | 0.0416(18) | 0.0306(18) | 0.0215(17) | −0.0006(14) | 0.0077(15) | 0.0059(14) |
| O2′ | 0.038(3) | 0.028(3) | 0.031(3) | 0.001(3) | 0.014(3) | 0.000(3) |
| O3 | 0.0254(11) | 0.0473(13) | 0.0308(11) | −0.0091(9) | 0.0058(9) | −0.0001(9) |
| O4 | 0.0400(12) | 0.0500(14) | 0.0323(11) | −0.0076(10) | 0.0108(10) | 0.0019(10) |
| O99A | 0.042(3) | 0.044(5) | 0.040(4) | −0.004(4) | 0.004(3) | 0.002(4) |
| O99B | 0.033(3) | 0.061(6) | 0.035(4) | −0.004(4) | 0.007(2) | −0.010(4) |
| N1 | 0.0384(17) | 0.0449(17) | 0.0393(16) | 0.0053(13) | 0.0203(14) | 0.0112(13) |
| N2 | 0.0270(13) | 0.0341(15) | 0.0332(15) | 0.0015(13) | 0.0075(12) | 0.0033(11) |
| C1 | 0.0283(16) | 0.0324(16) | 0.0321(16) | 0.0078(13) | 0.0124(13) | 0.0005(12) |
| C2 | 0.0321(17) | 0.0381(17) | 0.0327(16) | 0.0056(13) | 0.0100(13) | −0.0014(13) |
| C3 | 0.0301(17) | 0.048(2) | 0.0412(18) | 0.0104(16) | 0.0051(14) | −0.0044(14) |
| C4 | 0.0290(17) | 0.0471(19) | 0.0470(19) | 0.0133(16) | 0.0148(15) | 0.0064(14) |
| C5 | 0.0324(17) | 0.0390(17) | 0.0343(16) | 0.0113(14) | 0.0132(14) | 0.0065(14) |
| C6 | 0.0391(18) | 0.0334(17) | 0.0339(17) | 0.0099(14) | 0.0161(14) | 0.0088(14) |
| C7 | 0.056(2) | 0.0324(17) | 0.0362(18) | 0.0011(14) | 0.0204(16) | 0.0098(15) |
| C8 | 0.055(2) | 0.0337(18) | 0.0357(18) | −0.0020(14) | 0.0119(16) | 0.0003(15) |
| C9 | 0.0411(18) | 0.0344(17) | 0.0348(17) | 0.0030(14) | 0.0111(14) | −0.0009(14) |
| C10 | 0.0362(17) | 0.0321(16) | 0.0323(16) | 0.0038(13) | 0.0155(14) | 0.0022(13) |
| C11 | 0.0377(17) | 0.0275(15) | 0.0277(15) | 0.0079(12) | 0.0136(13) | 0.0040(13) |
| C12 | 0.0305(16) | 0.0309(16) | 0.0295(15) | 0.0085(13) | 0.0122(13) | 0.0017(12) |
| C13 | 0.0311(16) | 0.0331(16) | 0.0265(15) | −0.0019(12) | 0.0078(12) | −0.0021(12) |
| C14 | 0.0325(16) | 0.0307(16) | 0.0290(16) | 0.0010(13) | 0.0083(13) | 0.0015(13) |
| C15 | 0.0263(15) | 0.0327(16) | 0.0289(15) | 0.0031(12) | 0.0090(12) | 0.0043(12) |
| C16 | 0.0322(16) | 0.0347(17) | 0.0390(18) | −0.0078(14) | 0.0036(14) | 0.0016(13) |
| C17 | 0.0298(16) | 0.0477(19) | 0.0342(17) | −0.0106(15) | 0.0031(13) | 0.0023(14) |
| C18 | 0.0246(15) | 0.0317(16) | 0.0337(16) | 0.0031(13) | 0.0069(13) | −0.0014(12) |
| C19 | 0.0299(16) | 0.0352(17) | 0.0313(16) | 0.0063(13) | 0.0103(13) | −0.0031(13) |
| C20 | 0.0379(18) | 0.0382(18) | 0.051(2) | 0.0048(15) | 0.0194(16) | 0.0033(15) |
| C21 | 0.0245(17) | 0.050(2) | 0.073(3) | 0.0038(19) | 0.0059(17) | 0.0012(15) |
| C22 | 0.0326(18) | 0.053(2) | 0.057(2) | −0.0075(18) | −0.0052(16) | −0.0012(16) |
| C23 | 0.0317(17) | 0.0407(18) | 0.0407(18) | −0.0045(14) | 0.0021(14) | −0.0004(14) |
| C24 | 0.065(2) | 0.050(2) | 0.0325(18) | −0.0027(15) | 0.0176(17) | 0.0098(17) |

TABLE 9
Hydrogen Atom Coordinates and Isotropic Atomic Displacement
Parameters (Å²) for Carvedilol Hydrobromide Monohydrate.

| | x/a | y/b | z/c | U |
|---|---|---|---|---|
| H2O | 0.086(3) | 0.471(3) | 0.155(4) | 0.047 |
| H2O′ | 0.082(6) | 0.465(5) | −0.077(6) | 0.047 |
| H99A | −0.073(4) | 0.3802(19) | 0.201(6) | 0.064 |
| H99B | −0.060(4) | 0.490(2) | 0.205(6) | 0.065 |
| H99 | −0.1344(19) | 0.4409(13) | 0.157(3) | 0.065 |
| H1N | 0.373(2) | 0.2411(16) | −0.205(3) | 0.039(10) |
| H2NA | −0.043(2) | 0.4188(18) | 0.045(3) | 0.058(12) |
| H2NB | −0.036(2) | 0.422(2) | −0.060(4) | 0.077(14) |
| H2A | 0.3299 | 0.4112 | 0.1114 | 0.041 |
| H3A | 0.4497 | 0.3844 | 0.0850 | 0.048 |
| H4A | 0.4633 | 0.3119 | −0.0468 | 0.048 |
| H7A | 0.2616 | 0.1720 | −0.3395 | 0.048 |
| H8A | 0.1289 | 0.1632 | −0.3836 | 0.049 |
| H9A | 0.0548 | 0.2212 | −0.2906 | 0.044 |
| H10A | 0.1112 | 0.2912 | −0.1543 | 0.039 |
| H13A | 0.2180 | 0.4552 | 0.0713 | 0.036 |
| H13B | 0.1990 | 0.3994 | 0.1468 | 0.036 |
| H14 | 0.0925 | 0.4552 | −0.0281 | 0.037 |
| H14′ | 0.0943 | 0.4596 | 0.1099 | 0.037 |
| H15A | 0.0642 | 0.3477 | −0.0132 | 0.035 |
| H15B | 0.0576 | 0.3585 | 0.1069 | 0.035 |
| H16A | −0.0819 | 0.3172 | 0.0400 | 0.043 |
| H16B | −0.0599 | 0.3103 | −0.0723 | 0.043 |
| H17A | −0.1625 | 0.3802 | −0.1496 | 0.046 |
| H17B | −0.1922 | 0.3165 | −0.1021 | 0.046 |
| H20A | −0.3977 | 0.4876 | 0.0466 | 0.049 |
| H21A | −0.4785 | 0.4439 | −0.1048 | 0.060 |
| H22A | −0.4306 | 0.3786 | −0.2183 | 0.060 |
| H23A | −0.2996 | 0.3553 | −0.1809 | 0.047 |
| H24A | −0.2310 | 0.5242 | 0.2397 | 0.072 |
| H24B | −0.3101 | 0.4858 | 0.2148 | 0.072 |
| H24C | −0.3002 | 0.5475 | 0.1455 | 0.072 |

TABLE 10
Site Occupation Factors that Deviate from Unity
for Carvedilol Hydrobromide Monohydrate.

| Atom | sof | Atom | sof | Atom | sof |
|---|---|---|---|---|---|
| Br1 | 1 | Br2 | 1 | O1 | 1 |
| O2 | 0.65 | H2O | 0.65 | O2′ | 0.35 |
| H2O′ | 0.35 | O99A | 0.50 | H99A | 0.50 |
| O99B | 0.50 | H99B | 0.50 | H99 | 1 |
| H14 | 0.65 | H14^{′} | 0.35 | | |

### Example 9

Form 2. Carvedilol HBr (dioxane solvate)

Form 1 is slurried in dioxane between 0 and 40° C. for 2 days. The product is filtered and mildly dried.

### Example 10

Form 3. Carvedilol HBr (1-pentanol solvate)

Form 1 is slurried in 1-pentanol between 0° C. and 40° C. for 2 days. The product is filtered and mildly dried.

### Example 11

Form 4. Carvedilol HBr (2-Methyl-1-Propanol solvate)

Form 1 is slurried in 2-Methyl-1-Propanol between 0° C. and 40° C. for 2 days. The product is filtered and mildly dried.

### Example 12

Form 5. Carvedilol HBr (trifluoroethanol solvate)

Form 1 is slurried in trifluoroethanol between 0° C. and 40° C. for 2 days. The product is filtered and mildly dried.

### Example 13

Form 6. Carvedilol HBr (2-propanol solvate)

Form 1 is slurried in 2-propanol between 0° C. and 40° C. for 2 days. The product is filtered and mildly dried.

### Example 14

Form 7. Carvedilol HBr (n-propanol solvate #1)

Carvedilol free base is dissolved in n-propanol/water (95:5), and stoichiometric hydrobromic acid is added. The solution is cooled, and crystallization ensues. The product is filtered, washed with process solvent, and dried.

### Example 15

Form 8. Carvedilol HBr (n-propanol solvate #2)

Carvedilol HBr monohydrate (Form 1) is dissolved in n-propanol at ambient temperature. The n-propanol is slowly evaporated off, giving a white solid.

### Example 16

Form 9. Carvedilol HBr (anhydrous forms and solvent free)

Carvedilol free base is dissolved in a solvent (dichloromethane, isopropyl acetate, and acetonitrile have been used) and anhydrous forms HBr is added (HBr in acetic acid or gaseous HBr). The solution is cooled, and crystallization ensues. The product is filtered, washed with process solvent, and dried.

### Example 17

Form 10. Carvedilol HBr (ethanol solvate)

Carvedilol free base is dissolved in ethanol, and anhydrous forms HBr is added (HBr in acetic acid). The solution is cooled, and crystallization ensues. The product is filtered, washed with process solvent, and dried.

### Example 18

Carvedilol Monocitrate Monohydrate Preparation

In a 150 mL glass beaker, 100 gram of 20% w/w citric acid solution was prepared and 2.2 gram of carvedilol was added. The solution became slightly brownish after 15 minutes stirring, with only a little solid sticking on the bottom of the beaker. The beaker was then placed in a fume hood for evaporation. After staying in the hood overnight, large single crystals appeared in the beaker. The solid crystals were isolated and dried in a desiccator under vacuum. Similarly single crystals of citrate salt could be obtained by slow evaporation of carvedilol/citric acid solutions (containing citric acid 5%, 10% or 20% w/w) in Petri dishes (150 mm diameter) placed in a desiccator connected to a house vacuum.

### Example 19

Carvedilol Monocitrate Monohydrate Preparation

A 250 mL three-necked flask equipped with stirrer bar, thermometer, and an addition funnel is charged with acetone (20 mL, 2.5 volumes). The solution is sequentially charged with carvedilol (8 g, 19.7 mmol), and 2 M citric acid solution (40 mL, 5 volumes). Upon addition of the citric acid solution, the slurry dissolves quickly. The solution is filtered through a Buchner funnel fitted with Whatman filter paper and the solution is returned to a 250 mL flask fitted with a stirrer. To the light brown solution is added water (20 mL, 2.5 volumes). No exotherm is noted. The reaction mixture becomes cloudy but disappears upon stirring (heating up to 40° C. maybe needed to remove cloudiness). The mixture is stirred at room temperature and when judged clear is charged with carvedilol monocitrate monohydrate seeds (80 mgs) in one portion. An immediate cloudiness is observed (solid starts to precipitate out over 12-24 hours). The precipitate formed is stirred for 24-48 hours and is filtered through a Buchner funnel fitted with Whatman filter paper and the collected cake is washed with water (2×16 mL). The cake is dried in the oven under house vacuum at 50° C. to a constant weight. The cake (7.95 g, 67%) is weighed and stored in a polyethylene container.

### Example 20

Carvedilol Monocitrate Monohydrate Preparation

A suitable reactor is charged with acetone. The solution is sequentially charged with carvedilol, and aqueous citric acid solution. Upon addition of the citric acid solution, the slurry dissolves quickly. To the solution is added water. The mixture is stirred at room temperature and is charged with carvedilol seeds in one portion. The precipitate formed is stirred for a period of time, filtered and the collected cake is washed with water. The cake is dried under vacuum to a constant weight and stored in a polyethylene container.

### Example 21

Characterization of Carvedilol Monocitrate Monohydrate Preparation

The HPLC assay and ¹H-NMR revealed that the molar ratio of carvedilol and citric acid in carvedilol citrate salt prepared was approximately 1:1. The characterization by several other techniques are listed below:

Scanning Electron Microscopy (SEM)

The SEM used for the study was a Hitachi S-3500N. SEM was performed using an acceleration voltage of 5 kV. The samples were gold sputtered.

The carvedilol monocitrate salt consists of crystals with plate-shape, and various sizes depending on the preparation method. Crystals as large as 1 mm width and length were observed.

Differential Scanninci Calorimetry (DSC)

DSC measurements were performed with a MDSC 2920 (TA Instruments, Inc.). Approximately 5 mg of the sample was placed in an open aluminum pan. The sample was scanned at 10° C./min. An endothermic event was observed with an onset temperature near 82-83° C. The heat of fusion was calculated as 63 kJ/mol.

Fourier Transform Infrared Spectroscopy (FT-IR)

Approximately 2 mg of sample was diluted with 300 mg of dried potassium bromide (KBr). The mixture was ground with a mortar and pestle, then transferred to a die that is placed under high pressure for 3 minutes. The instrument was a PerkinElmer Spectrum GX FTIR instrument. Forty scans were collected at 4 cm⁻¹resolution. The typical FT-IR spectrum of carvedilol monocitrate salt is shown in FIG. 1. The characteristic peaks in the 1800 to 600 cm⁻¹region are found at about 1727, 1709, 1636, 1625, 1604, 1586, 1508, 1475, 1454, 1443, 1396, 1346, 1332, 1305, 1256, 1221, 1129, 1096, 1077, 1054, 1021, 1008, 984, 939, 919, 902, 826, 787, 755, 749, 729, 676, 664, 611 cm⁻¹.

X-Ray Powder Diffraction (XRPD)

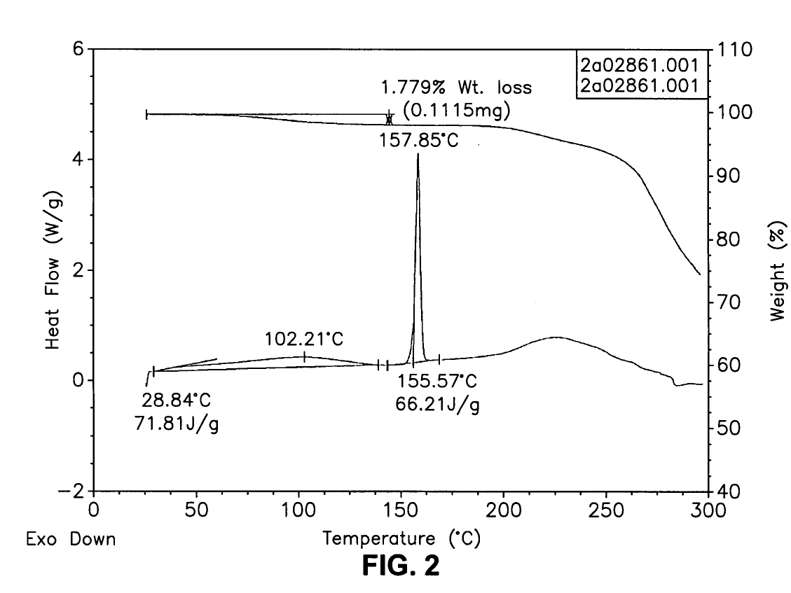

XRPD patterns were collected using a Philips X'Pert Pro Diffractometer. Approximately 30 mg of sample was gently flattened on a silicon sample holder and scanned from 2-35 degrees two-theta, at 0.02 degrees two-theta per step and a step time of 2.5 seconds. The sample was rotated at 25 rpm. The XRPD patterns of two different batches of Carvedilol monocitrate salt are shown in FIG. 2.

Solubility in Water Glass vials containing water and excess amount of carvedilol salts were shaken by a mechanical shaker at ambient conditions. Aliquots were taken out at various time-point, filtered through 0.45 μm Acrodisc GHP filter. The pH of the filtered solutions was measured and suitable dilution was performed prior to UV-Vis analysis of carvedilol concentration.

The solubility of carvedilol monocitrate salt in water at room temperature was determined. The drug concentrations and pH values at different time-points are presented in Table 11. This crystalline form of carvedilol monocitrate salt exhibited high solubility in water (1.63 mg/mL at 1 hour and 1.02 mg/mL at 48 hour).

TABLE 11
Aqueous Solubility (expressed as mg of carvedilol
free base/mL of solution) over time at 25° C. for
Carvedilol Free Base and Its Monocitrate Salt.

| | Carvedilol | Carvedilol |
|---|---|---|
| Time, hr | Free Base | Mono-Citrate Salt |
| 1 | 0.0098 | 1.63 (pH = 3.5) |
| 4 | | 1.47 (pH = 3.4) |
| 24 | 0.0116 | 1.07 (pH = 3.0) |
| 48 | | 1.02 (pH = 3.0) |

Carvedilol monocitrate salt has two free carboxylic acid groups in one unit salt, which contributes the low pH value (near pH 3) observed for monocitrate salt when dissolved in water. This may potentially lead to improved formulations by providing a low pH microenvironment within the formulation as it traverses the GI tract. This may provide an environment at a molecular level that is more conductive to dissolution, particularly in the lower GI tract, where the pH of the environment is near neutral pH and the intrinsic solubility of the drug substance is limited. Such a microenvironmental pH should lead to greater dissolution rate because of higher solubility in the solid/liquid interface, leading to improved absorption of drug in the lower GI tract thereby sustaining overall absorption and, in consequence providing prolonged blood levels and allowing less frequent dosing. Therefore, a once-per-day carvedilol formulation may be possible by incorporating carvedilol monocitrate salt. Such a unit is more convenient for patients and result in higher patient compliance with the dosage regimen and hence a better therapeutic effect.
Crystalline Structure of Carvedilol Monocitrate Salt

The crystalline structure of carvedilol citrate salt was determined by Single Crystal X-Ray Diffraction analysis on the large crystals formed by evaporation. The result indicated that the salt form was a carvedilol monocitrate, where the molar ratio of carvedilol and citric acid was 1:1. Surprisingly, the hydroxyl of carvedilol is disordered in the crystalline packing. In other words, the monocitrate salt has both R(+) and S(−) carvedilol enantiomers at 1:1 molar ratio, and the two enantiomers are randomly distributed, without any specific order.

This crystalline packing habit is very unusual for a salt formed between a chiral compound and a chiral counter-ion (monocitrate). Typically, chiral counter-ion tends to differentiate the two stereoisomers of the compound when forming crystals. However, in the case of the monocitrate salt, there seems to be enough space in the crystal packing to allow the carbonyl group of the terminal carboxylic acid group of citrate to form equivalent hydrogen bond with the hydroxyl from either the R(+) or the S(−) carvedilol stereoisomer.

This avoids generation of yet more optically active forms that could potentially complicate stability, dissolution rates and possibly in vivo absorption and pharmacologic effects.

The above data demonstrates that a novel crystalline form of carvedilol monocitrate monohydrate can be prepared with a unique crystalline packing habit, which exhibits high aqueous solubility and can provide a low pH microenvironment for enhanced dissolution.

### Example 22

Crystalline Carvedilol Benzoate Preparation

A suitable reactor is charged with acetone. The solution is sequentially charged with carvedilol (4.1 grams, 0.1 moles), and benzoic acid solution. Upon addition of the benzoic acid (1.4 grams, 0.011 moles) solution, all material dissolves into the solution. To the stirred solution is added tert-butyl methyl ether (60 ml). The precipitate formed is stirred for a period of time, filtered and the collected cake is washed with water. The cake is dried under vacuum to a constant weight and stored in a polyethylene container.

### Example 23

Crystalline Carvedilol Mandelate Preparation

A suitable reactor is charged with acetone (38 mL). The acetone solution is sequentially charged with carvedilol (11.08 grams) and water (8 mL) Upon addition of the water, the slurry dissolves completely with heating. To the solution, 1 N Mandelic acid in methanol (1 Equiv. 27.3 mL.) is added. The resulting mixture is stirred at the range between 17° C. and 35° C., and the solid precipitate is formed over 10 hours to 24 hours. Later, the mixture filtered and the cake is washed with a mixture of acetone and water (10 to 1) at 3 volumes or 33 mL. The cake is then dried under vacuum to a constant weight. The final weight is 8.34 g, 54,5% yield.

### Example 24

Crystalline Carvedilol Lactate Preparation A suitable reactor is charged with acetone (50 mL). The acetone solution is sequentially charged with carvedilol (15.0 grams) and water (7 mL). Upon addition of the water, the slurry dissolves completely with heating. To the solution is added 1N aqueous D, L-Lactic acid (1 equiv., 36.9 mL). The reaction mixture is stirred at between 17° C. and 35° C. and seeded in one portion. The solid precipitate is formed over 10 hours to 24 hours. Later, the mixture is filtered and the cake is washed with a mixture of acetone and water (10 to 1) at 2 volume or 30 mL. The cake is dried under vacuum to a constant weight. The final weight is 9.16 grams.

### Example 25

Crystalline Carvedilol Sulfate Preparation

A suitable reactor is charged with acetone (38 mL). The acetone solution is sequentially charged with carvedilol (10.25 grams) and water (6 mL). Upon addition of the water, the slurry dissolves completely with heating. To the solution, 1 N aqueous sulfuric acid (1 equiv., 25.2 mL) is added. The reaction mixture is stirred at between 17° C. and 35° C. and the solid precipitate is formed over 10 hours to 24 hours. Later, the mixture is filtered and the cake is washed with a mixture of acetone and water at 2 volumes or 20.5 mL. The cake is then added a mixture of acetone and water (10 to 1) for ripening between 20° C.-35° C. over 24 hours to 48 hours. The slurry is filtered and the cake is dried under vacuum to a constant weight. The final weight is 5.48 grams.

### Example 26

Crystalline Carvedilol Maleate Preparation

A suitable reactor is charged with acetone (56 mL). The acetone solution is sequentially charged with carvedilol (15.0 grams) and water (8 mL). Upon addition of the water, the slurry dissolves completely with heating. To the solution is added 1 M of aqueous Maleic acid (1 Equiv. 36.9 mL.) The reaction mixture is stirred at between 17° C. and 35° C. The solid precipitate is formed over 10 hours to 24 hours. Later, the mixture is filtered and the cake is washed with a mixture of acetone and water (10 to 1) at 3 volume or 45.0 mL. The cake is dried under vacuum to a constant weight. The final weight is 14.08 grams.

### Example 27

Crystalline Carvedilol Glutarate Preparation

A suitable reactor is charged with 2 grams of carvedilol and a mixture of acetone and water (in a 7 to 1 ratio) at 8 mL. The contents were warmed to 35° C. to 40° C to a clear solution. 1N D,L-Glutaric acid in water (1 equivalent. 4.9 mL.) is added to the solution. The resulting mixture is stirred at the temperature between 17° C. and 35° C. until the solid precipitate is formed over 10 hours to 24 hours. Subsequently, the mixture filtered and the cake is washed with a mixture of acetone and water (in a 10 to 1) at about 5 mL. The cake is then dried under vacuum to a constant weight. The final weight is 1.35 grams.

### Example 28

Solubility Enhancement in the GI Tract

Background:

Drug absorption following oral dosage requires that drug first dissolves in the gastrointestinal milieu. In most cases such dissolution is primarily a function of drug solubility. If solubility is affected by pH it is likely that absorption will vary in different regions of the gastro intestinal tract, because pH varies from acidic in the stomach to more neutral values in the intestine.

Such pH-dependent solubility can complicate dosage form design when drug absorption needs to be prolonged, delayed or otherwise controlled, to evince a sustained or delayed action effect. Variations in solubility can lead to variable dissolution, absorption and subsequent therapeutic effect.

Carvedilol is a drug used to treat hypertension and congestive heart failure, being usually administered twice daily. For chronic diseases such as these a once-daily dosage regimen is desirable, to enhance patient compliance and reduce “pill burden”. However, the dose response and time course of carvedilol in the body is such that a conventional dosage form, releasing all the drug immediately on ingestion does not provide once-a-day therapy. Release from the dosage form needs to be slowed down so that absorption and subsequent systemic residence is prolonged. This however requires that release and dissolution occurs along the GI tract, not just in the stomach.

The pH-dependent solubility of the currently used form of carvedilol (free base) is such that, while gastric solubility is adequate, solubility is much poorer at pH values encountered in the small intestine and beyond (see, FIG. 126), which depicts a pH-solubility profile for carvedilol.

Consequently, while drug dissolution rate and extent from an immediate release dosage form is likely to be acceptable (such dissolution occurring in the stomach) it could be inadequate in regions beyond the stomach, with absorption compromised as a consequence.

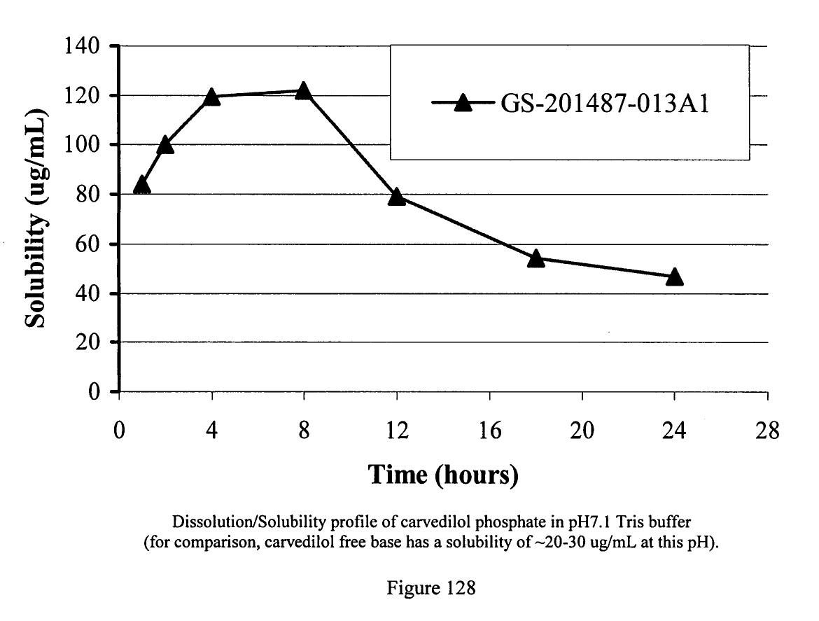

However, when drug is administered as a solution (in cyclodextrin in this example), directly to the colon it can be seen that absorption is significantly improved (FIG. 128, which depicts mean plasma profiles in beagle dogs following intra-colonic administration of a carvedilol solution containing Captisol or carvedilol in aqueous suspension.). All this information suggests that absorption throughout the GI tract could be significant, provided that drug can be solubilized.

Moreover, solubilization may mean that drug stability is compromised. The secondary amino group of carvedilol is prone to chemically react with excipients normally included in a dosage form to aid manufacture, maintain quality or enhance dissolution rate. For example, this type of amine groups can react with aldehydes or ester functional groups through nucleophilic reactions. Many excipients have ester functional groups. Furthermore, aldehydes and other such residues are common residues in excipients. This often results in marginal or unacceptable chemical stability of conventionally formulated carvedilol dosage forms, where drug is simply blended with excipients before being compressed to tablets. As drug-excipient interactions are likely to be even faster in the solvated state it follows that solubilization does not provide facile resolution of dissolution-limited absorption challenges. This is illustrated in Table 12. Solutions of carvedilol in oleic acid degraded rapidly. Other approaches to solubilization evince the same effect. Thus solubilization might enhance absorption but is not a practical approach because of the destabilizing effect.

TABLE 12
Drug content (mg/g) in carvedilol/Oleic
acid solution during storage.

| | Initial | 1 month at 25° C. | 3 months at 25° C. |
|---|---|---|---|
| 7.788% w/w carvedilol | 76.6 | 71.3 | 64.3 |
| solution in Oleic acid | | | |

It has now been unexpectedly shown that salts of carvedilol afford significant improvement in absorption from the lower GI tract in dogs over that seen when carvedilol base is used. There is no reason to believe that this surprising effect does not also apply to humans and it may be feasible as a consequence to design dosage forms that enable drug to be absorbed as the unit traverses the gastrointestinal tract. This ought provide more gradual absorption and prolonged plasma profiles that facilitate once-a-day dosage.

The better absorption may be partially due to the better solubilities of salts of carvedilol. It can be seen from the data in Table 13 that citrate, hydrobromide and phosphate salts have much better aqueous solubility than the free base.

TABLE 13
Aqueous Solubility (expressed as mg of Carvedilol free base/mL of
solution) at 25° C. for Carvedilol free base and three salts.

| Time | | Free Base | Citrate salt | Phosphate salt | HBr salt |
|---|---|---|---|---|---|
| 1 | hr | — | 1.64 (pH = 3.3) | 2.35 (pH = 3.0) | 0.62 (pH = 6.1) |
| 4 | hr | — | 1.74 (pH = 3.2) | 2.25 (pH = 3.0) | 0.61 (pH = 6.3) |
| 24 | hr | 0.024 | 1.46 (pH = 3.2) | 2.21 (pH = 3.0) | 0.61 (pH = 6.2) |
| | | (pH = 7.0) | | | |

Ostensibly, it can be claimed that these acidic salts simply generate low pH when dissolved in water (Table 13), leading to solubility enhancement (because of the pH/solubility relationship shown in FIG. 126). However, it is also possible that any pH-lowering effect contributed by the modest amounts of drug (that would be included in a dosage form to provide a therapeutic effect) would be readily swamped in the in vivo situation, with pH soon reverting to that of the general intestinal milieu. Consequently, any short term solubilization would be quickly negatived. However, it has been surprisingly shown that when pH is adjusted to neutral, the solubilities of salts remain higher than free base for a significant period, rather than equilibrating rapidly. Such prolonged solubility could be crucial in vivo, allowing dissolution and absorption to occur more readily at neutral pH than for free base (FIG. 128, which depicts dissolution/solubility profile of carvedilol phosphate in pH=7.1 Tris buffer (for comparison, carvedilol free base has a solubility of ˜20-30 ug/mL at this pH).

Furthermore, it has been shown that, if carvedilol salts are dissolved in solubilizing agents, stability is much better than when free base is used in the same system (Table 14). Thus, if solubilizing agents were to be required in the formulation, to provide even greater solubility enhancement, salts would be preferred to the base because of such better stability.

TABLE 14
Chemical stability data of carvedilol/Vitamin E TPGS granulation
containing carvedilol free base or carvedilol HBr salt.

Assay/Impurity after 1 month's
storage at 40° C./75% RH (open vials)

| | % of | Total Impurities |
|---|---|---|
| Formulation | initial level* | (% peak area) |
| Carvedilol free base | 81.5* | 7.77 |
| granulation containing | | |
| Vitamin E TPGS (Lot | | |
| 200412-144) | | |
| Carvedilol HBr salt | 89.9* | 0.15 |
| granulation containing | | |
| Vitamin E TPGS (Lot | | |
| 200746-102) | | |

*Lower % of nominal due to additional moisture in the system.

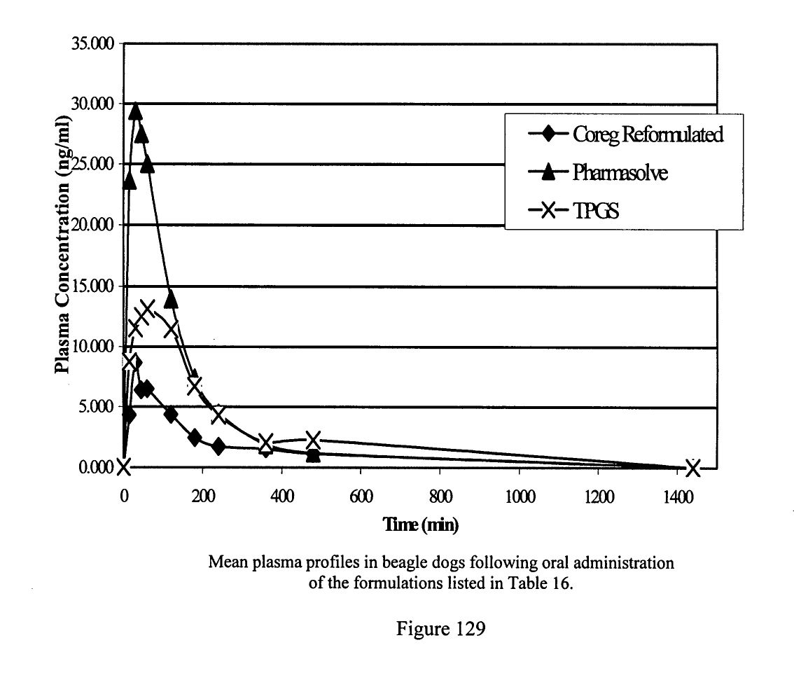

The foregoing facts and considerations suggest but do not provide conclusive proof that forms of carvedilol with superior solubility, whether effected by using a solvent to dissolve carvedilol base, or by using a carvedilol salt have better potential than conventionally formulated base for prolonged absorption along the GI tract. To provide stronger evidence that solubilization enhances absorption, formulations containing carvedilol base, formulated in a conventional manner, and also fully solvated by dissolving in n-methyl pyrrolidone were dosed to beagle dogs in units that were activated to make drug available after the dosage unit had passed the pyloric sphincter separating the stomach from the duodenum. Intestinal absorption efficiency was determined by monitoring plasma levels of carvedilol following such dosage. Results are provided in Table 5 and FIG. 129 (which depicts mean plasma profiles in beagle dogs following oral administration of the formulations listed in Table 15).

TABLE 15
Pharmacokinetic values following dosage of 10 mg
carvedilol (base) to three fasted beagle dogs.

| | Solubility in pH 6.8 | | | |
|---|---|---|---|---|
| | Phosphate Buffer Over | Cₘₐₓ | Tₘₐₓ | AUC (0-t) |
| Formulation | 4-hour Period (ug/mL) | (ng/mL) | (min) | (ug · min/mL) |
| Carvedilol Pharmasolve ® | 86-120 | 31.32 ± 3.43 | 15^{b.}, | 4.03 ± 1.34 |
| Granulation | | (n = 3) | 30, | (n = 3) |
| | | | 45^{a.} | |
| | | | (n = 3) | |
| Carvedilol Vitamin | 108-94 | 16.26 ± 1.20 | 30, | 2.75 ± 0.55 |
| ETPGS Granulation | | (n = 3) | 120, | (n = 3) |
| | | | 45^{a.} | |
| | | | (n = 3) | |
| Carvedilol in | 29-36 | 13.08, | 45, | 2.14, |
| conventional granules | | 12.74, | 30, | 1.19, |
| | | 2.89^{a.} | 120^{a.} | 0.60^{a.} |
| | | (n = 3) | (n = 3) | (n = 3) |

^{a.}= values listed individually due to large variability; animals always listed in the same order. AUC(0-t) refers to the area from time 0 to the last quantifiable concentration.
^{b.}= Pharmasolve ® capsule was leaking slightly before firing in-vivo.

It can be seen that, when drug was fully dissolved absorption was rapid and high, contrasting with lower concentrations in dogs that were dosed intraduodenally with base in a conventional solid dosage unit. These findings indicated that bioavailability from carvedilol base in the small intestine is constrained by its low solubility at neutral pH. When units are introduced to the stomach the low gastric pH can be expected to facilitate dissolution and absorption but this will not be the case in the more neutral small intestine or beyond.

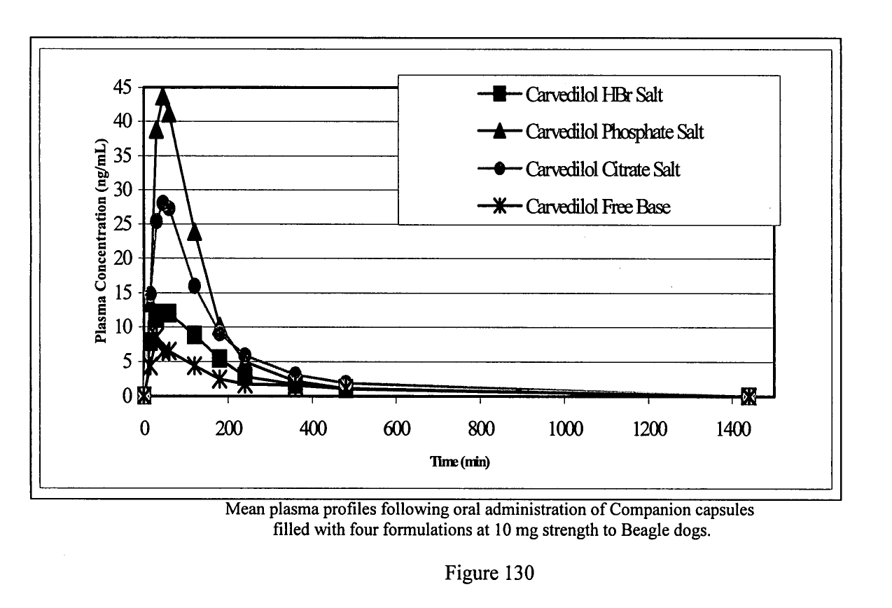

A further dog study utilized salts of carvedilol, formulated using conventional (non-solubilizing) excipients. The mode of dosage was the same as for the first dog study, the formulations being delivered such that drug did not become available until units were beyond the gastric milieu. Results are provided in Table 16 and FIG. 130 (which depicts mean plasma profiles 20 following oral administration of Companion capsules filled with four formulations at 10 mg strength to Beagle dogs).

TABLE 16
Pharmacokinetic analysis of 10 mg dose formulations
in three fasted beagle dogs from study.

| | Cₘₐₓ | Tₘₐₓ | AUC (0-t)^{a} | AUC (0-inf) |
|---|---|---|---|---|
| Formulation | (ng/mL) | (min) | (ug · min/mL) | (ug · min/mL) |
| Carvedilol HBr Salt granules | 12.9 ± 7.11 | 45 ± 15 | 2.22 ± 1.37 | 2.35 ± 1.46 |
| Carvedilol Phosphate Salt | 61.8, | 45, | 6.69, | 6.75, |
| Granules^{b} | 28.4 | 60 | 4.56 | 4.90 |
| Carvedilol Citrate Salt | 30.4 ± 16.9 | 45 ± 15 | 4.41 ± 2.43 | 4.66 ± 2.54 |
| Granules | | | | |
| Carvedilol Base Granulesx^{c} | 13.08, | 45, | 2.14, | — |
| | 12.74, | 30, | 1.19, | |
| | 2.89 | 120 | 0.60 | |

^{a}AUC(0-t) refers to the area from time 0 to the last quantifiable concentration
^{b}n = 2 only, due to malfunction of one InteliSite ® Companion capsule; animals always listed in the same order
^{c}data from dog study DI01251; values listed individually due to large variability; animals always listed in the same order.

The findings from the second dog study, illustrated graphically in FIG. 130 conclusively showed that drug, administered in salt form was rapidly and more completely absorbed than the free base form.

### Example 29

The present invention relates to dosage forms of carvedilol to match drug delivery with pharmacodynamic requirements

Accordingly the present invention provides a unit dose composition that comprises:
Example [A]. A delayed/controlled release component delivering plasma levels 20 that increase gradually following ingestion. This component would most probably deliver a lower dose than the later-releasing component. Ideally this component provides a peak plasma level about 1-3 hours after dosage.

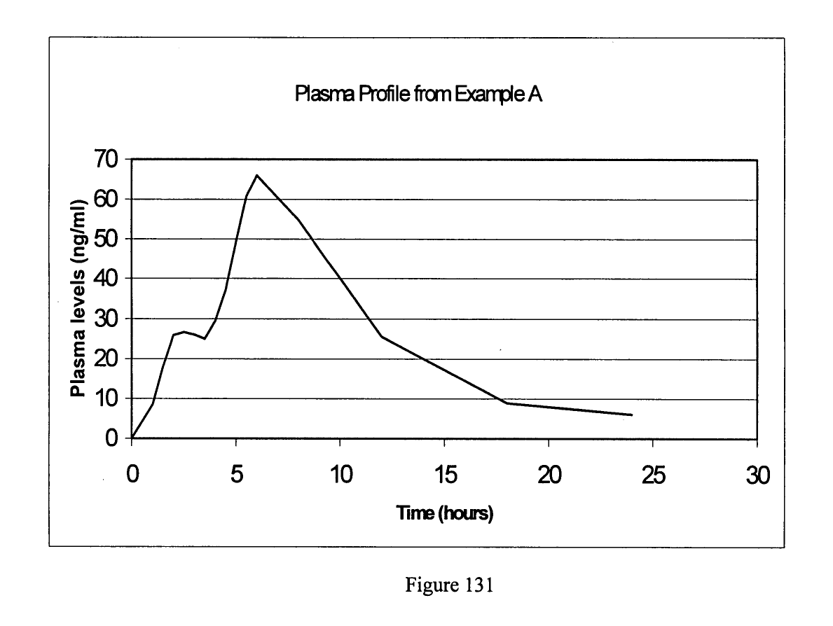

Plasma profiles obtained following dosage to the volunteers of tablets, formulated according to the premises outlined in Example [A] are shown in FIG. 131.

Units, formulated as described in the example described above has been evaluated for a corresponding biopharmaceutical profiles in human subjects and provide the requisite biphasic pulsed profiles.

It can be seen that the above-identified dosage form type provides distinctive substantially biphasic profiles, and time courses aligned with those defined in earlier discussions.

### Example 30

Dosage Forms Utilized in PK Studies

Dosage forms were tablets, that comprised conventional (immediate release) cores, film coated, to restrict release in an acidic environment. Apertures of varying diameters were drilled on both faces of the units to control the rate of drug release from the tablet. One batch did not have apertures. Unit composition is detailed in Table 17 below.

TABLE 17
Tablet composition
Component

Carvedilol (free base)
Lactose monohydrate
Sucrose
Povidone (poly vinyl pyrrolidone)
Cross-linked Povidone
Colloidal Silicon Dioxide
Magnesium Stearate
Clear Opadry YS-1-19025-A
Methacrylic Acid Copolymer
(Eudragit ® L30 D-55)
Triethyl Citrate
Glyceryl Stearate
Polysorbate 80

Tablet Manufacture

The active ingredient was blended with lactose, PVP, sucrose and colloidal silicon dioxide. Water was added to provide a wet mass that was converted to granules by screening and drying. The granules were then blended with cross linked povidone, colloidal silicon dioxide and magnesium stearate and compressed to tablets on a rotary tablet machine.

The tablets were coated with a clear coat comprising a proprietary coating composition (Opadry). A further coat was then applied from a suspension comprising methacrylic acid copolymer, triethyl citrate and glyceryl stearate. Apertures were then drilled on each face of the tablets according to the dimensions given in Table 18. One set of tablets did not have apertures.

TABLE 18

| Formula | Aperture (mm) |
|---|---|
| B | no aperture |
| C | 2 mm |
| D | 3 mm |
| E | 4 mm |

A Phase 1 volunteer study was performed to determine the impact of the presence of an aperture, and aperture size on in vivo performance.

### Example 31

Dosage Forms Utilized in PK Studies

Dosage forms comprised tablets, with drug embedded in a hydrophilic matrix core to retard release. Tablets were then film coated, to restrict release. Two apertures were drilled on each face of the tablet, thereby restricting drug release at the tablet surface to the apertures. Rate of release from the tablet was controlled by aperture diameter and by the level of HPMC in the core tablets as detailed in Table 19 below.

TABLE 19
Tablet composition
Component

Carvedilol Phosphate
Mannitol
Hydroxypropyl methylcellulose (HPMC)
Microcrystalline Cellulose
Povidone (poly vinyl pyrrolidone)
Colloidal Silicon Dioxide
Magnesium Stearate
Clear Opadry YS-1-19025-A
Methacrylic Acid Copolymer
(Eudragit ® L30 D-55) (Formulations D, E, F, G)
Triethyl Citrate
Glyceryl Stearate
Polysorbate 80
Ethylcellulose (Formulations B, C)

Tablet Manufacture The active ingredient was blended with the HPMC, mannitol and PVP. Water was added, providing a wet mass that was converted to granules by screening and drying. The granules were then blended with microcrystalline cellulose, colloidal silicon dioxide and magnesium stearate and compressed to tablets on a rotary tablet machine.

The tablets were coated with a clear coat comprising a proprietary coating composition (Opadry). A further coat was then applied, comprising either methacrylic acid copolymer or ethylcellulose (see Table 20) to confine release of drug to the apertures on each face. Apertures were then drilled on each face of the tablets according t the dimensions given in Table 20.

TABLE 20

| | level of HPMC in matrix | aperture diameter |
|---|---|---|
| Formulation | (%) | (mm) |
| B | 5 | 7 |
| C | 5 | 5 |
| D | 15 | 6 |
| E | 20 | 4 |
| F | 20 | 7 |
| G | 25 | 6 |

A Phase 1 volunteer study was performed to determine the impact of the level of release modifier in the tablet matrix and the aperture size on in vivo performance.

### Example 32

Dosage Forms Utilized in PK Studies Dosage forms comprised bilayer tablets consisting of a conventional (immediate release) layer and a modified release layer that delivered drug in a controlled manner. Tablets were film coated and had apertures of diameter 6mm on both faces of the units to control the rate of drug release from the tablet. Two formulations, delivering drug slowly and more quickly were prepared, the rate of release being determined by the polymers included in the modified release layer. . Unit composition is detailed in Table 22 below.

TABLE 22

Carvedilol (Free Base)
Mannitol
Sucrose
Povidone (polyvinyl pyrrolidone)
Amorphous Colloidal Silicon
Dioxide
Microcrystalline Cellulose
Hypromellose (HPMC)
Premium Grade (K100LV)
Hypromellose (HPMC) K4M *
Carboxymethylcellulose
Sodium (Na CMC) *
Crospovidone (cross-linked PVP)
Magnesium Stearate
Methacrylic acid co[polymer
(Eudragit L30D-55
Triethyl citrate
Polysorbate 80
Glyceryl monostearate
* present only in the formulation that delivered drug more slowly from the modified release layer.

Tablet Manufacture:

The active ingredient was dispersed in a aqueous suspension, along with sucrose, mannitol, colloidal silicon dioxide and PVP. Granules were then prepared by spray granulating this dispersion with a blend of solids comprising microcrystalline cellulose, mannitol, crospovidone and PVP to provide “immediate release” granules. These were blended with additional excipients prior to compression.

Modified release granules were prepared by dispersing the active ingredient in aqueous sucrose, PVP, mannitol and colloidal silicon dioxide and spray granulating with a blend of solids comprising microcrystalline cellulose, mannitol, PVP and HPMC. NaCMC was also included in the slower releasing granules. The granules prepared in this way were then blended with additional excipients prior to compression.

The immediate and modified release granules were then compressed to bilayer tablets using a bilayer rotary press. Tablets were then film coated with a low pH-resistant coat comprising methacrylic acid copolymer as the film former and triethyl citrate, Polysorbate 80 and glyceryl monostearate as other coat components. Finally, apertures. 6 mm in diameter were drilled in both faces of the tablets.

A Phase 1 volunteer study was performed to determine the impact of the release modifier in the tablet matrix on in vivo performance.

### Example 33

An alternative example concerns a unit, where drug release is constrained or delayed by a time or pH-dependent coat, with or without an aperture through which drug is released at a controlled rate. The coat composition may be varied such that it is eroded or dissolved at a desired pH, or after a defined time following ingestion such that drug is released “later” to provide the required “early morning” plasma levels or to sustain levels to cover the full dosage interval. Such an approach is summarized below.

A tablet containing ingredients listed below* is made using conventional manufacturing techniques (moist granulation, granulation and compression).

| Ingredient | Quantity (mg) |
|---|---|
| Carvedilol Phosphate hemihydrate | 41.4 |
| Mannitol | 261.6 |
| Hypromellose | 120.4 |
| Microcrystalline cellulose | 120.6 |
| Povidone | 47.0 |
| Colloidal Silicone dioxide | 6.0 |
| Magnesium stearate | 6.0 |

The tablet is coated by spraying an aqueous suspension of the following ingredients (approximate mg per tablet)

| Ingredient | Quantity (mg) |
|---|---|
| Opadry II Color | 12.1 |
| Methacrylic acid copolymer Type C | 39.2 |
| (Eudragit L30-55) | |
| Triethyl Citrate | 4.0 |
| Glyceryl Monostearate | 1.3 |
| Polysorbate 80 | 4.0 |

*level of carvedilol is expressed as carvedilol phosphate anhydrous equivalent: Quantities of the inactive ingredients are approximate.

An aperture is drilled mechanically in each of the coated tablets to provide an orifice of 6 mm diameter.

Biopharmaceutical Performance of the Dosage Form Described in Example 33

Tablets were formulated according to descriptions as described herein to contain a total dose of 32.5 mg carvedilol phosphate (anhydrous equivalent), i.e. amount of carvedilol in the test (modified release) unit and were was assessed or evaluated by dosage to human volunteer study to determine human plasma profiles.

Volunteers were administered one tablet after food. Plasma samples were withdrawn at regular intervals over regular hour periods for determination of drug content, thereby enabling profiles to be constructed. One conventional, immediate release dosage form (commercial Coreg Tablet) containing 25 mg of drug, was dosed twice, at an interval of 12 hours (giving a total dose of 50 mg) to provide comparative data.

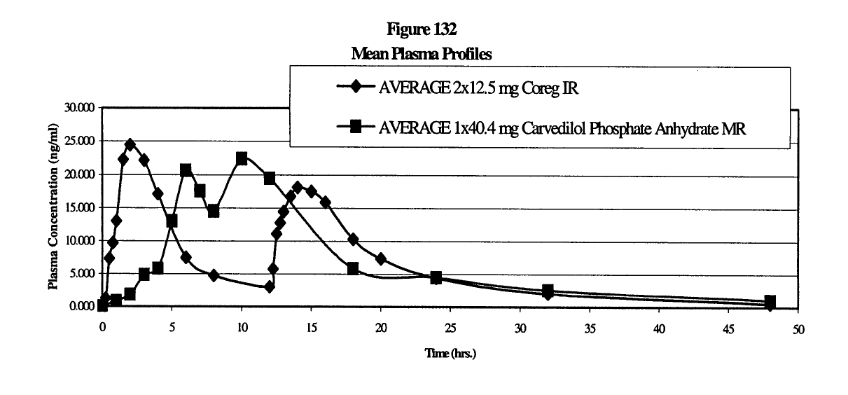

Mean plasma profiles are shown in FIG. 132 illustrating the unique plasma-time profile that meets the requirements stated herein.

It can be seen that above described dosage form type shows a distinctive biphasic-delivery, and plasma-time profiles aligned with those defined in earlier discussions.

It is also noteworthy that these volunteer studies indicate that the ratio of the two isomers (R and S) of carvedilol in plasma were not altered by formulation in modified release mode. Thus, it can be concluded that the metabolic or pharmacologic profile is not altered by the said formulation and that there would be no consequences for efficacy and safety.

In summary the mean and individual profiles indicate that a single dose of the test formulation delivered a plasma profile incorporating the following characteristics:
more gradual release of drug at the early stages than the conventional product;
a “first peak” 1-3 hours after dosage and a second peak after about 5 hours; and
levels at 24 hours that were comparable to those obtained after twice daily dosage of the current commercial product.
   Thus, data from dosage to humans show that the required plasma level profile is attainable with this dosage form.

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

1. A controlled release delivery formulation or device, comprising:
a core containing a carvedilol free base, salt, solvate or anhydrous form thereof;
a release modifying agent; and
an outer coating covering the core;
wherein thickness of the outer coating is adapted:
for substantial impermeability to entry of fluid present in an environment of use and for substantial impermeability toward release of the carvedilol free base, salt, solvate or anhydrous form thereof during a predetermined dosing interval; and
for a controlled release dispensing exit of the carvedilol free base, salt, solvate or anhydrous form thereof after the predetermined dosing interval;
wherein the outer coating includes at least one orifice in at least one face area of the controlled delivery device extending substantially through the outer coating but not penetrating the core that communicates from the environment of use to the core allowing for release of the carvedilol free base, salt, solvate or anhydrous form thereof into the environment of use;
wherein the at least one orifice in the at least one face area of the controlled release delivery device has a substantially dependent rate limiting release factor dependent upon exit of the carvedilol free base, salt, solvate or anhydrous form thereof from the at least one orifice via dissolution, diffusion or erosion; and
wherein the release modifying agent enhances or hinders release of the carvedilol free base, salt, solvate or anhydrous form thereof depending upon solubility or effective solubility of the carvedilol free base, salt, solvate or anhydrous form thereof in the environment of use.

2. A controlled release delivery formulation or device, comprising:
a core containing a carvedilol free base, salt, solvate or anhydrous form thereof;
a release modifying agent, and
an outer coating layer covering the core;
wherein the outer coating layer:
is substantially impermeable to the entrance of gastrointestinal fluid and substantially impermeable to release of the carvedilol free base, salt, solvate or anhydrous form thereof agent during a predetermined dosing interval; and
is adapted for a controlled release dispensing exit of the carvedilol free base, salt, solvate or anhydrous form thereof after the predetermined dosing interval;
wherein the outer coating layer includes at least one orifice for release of the carvedilol free base, salt, anhydrous form or solvate thereof during the dosing interval;
wherein the orifice extends substantially completely through the coating but not penetrating the core,
wherein a release rate limiting step is dependent substantially on exit of the carvedilol free base, salt, anhydrous form or solvate thereof through the at least one orifice via dissolution, diffusion or erosion of the carvedilol free base, salt, anhydrous form or solvate thereof in solution or suspension, and
wherein the release modifying agent enhances or hinders release of the carvedilol free base, salt, anhydrous form or solvate thereof depending upon solubility or effective solubility in gastrointestinal fluid.

3. The controlled release formulation according to claim 1, wherein the solubility enhanced carvedilol free base, salt, solvate or anhydrous form thereof include an acid addition salt of carvedilol free base or carvedilol salt, solvate and/or anhydrous forms thereof.

4. The controlled release formulation or device according to claim 1, wherein the acid addition salt of carvedilol free base, salt, solvate or anhydrous form thereof is an acid addition salt formed from mineral acids or organic acids.

5. The controlled release formulation according to claim 3, wherein the mineral acid is selected from hydrobromic acid, hydrochloric acid, phosphoric or sulphuric acid, and the organic acid is selected from methansulphuric acid, tartaric acid, maleic acid, acetic acid, citric acid, benzoic acid and the like.

6. The controlled release formulation or device according to claim 1, wherein the carvedilol salt, solvate or anhydrous form thereof is selected from the group consisting of carvedilol mandelate, carvedilol lactate, carvedilol maleate, carvedilol sulfate, carvedilol glutarate, carvedilol mesylate, carvedilol phosphate, carvedilol citrate, carvedilol hydrogen bromide, carvedilol oxalate, carvedilol hydrogen chloride, carvedilol hydrogen bromide, carvedilol benzoate, or corresponding solvates thereof.

7. The controlled release formulation or device according to claim 3, wherein the carvedilol salt, solvate or anhydrous form is selected from the group consisting of carvedilol hydrogen phosphate, carvedilol dihydrogen phosphate, carvedilol dihydrogen phosphate hemihydrate, carvedilol dihydrogen phosphate dihydrate, carvedilol dihydrogen phosphate methanol solvate, carvedilol hydrobromide monohydrate, carvedilol hydrobromide dioxane solvate, carvedilol hydrobromide 1-pentanol solvate, carvedilol hydrobromide 2-methyl-1-propanol solvate, carvedilol hydrobromide trifluoroethanol solvate, carvedilol hydrobromide 2-propanol solvate, carvedilol hydrobromide n-propanol solvate #1, carvedilol hydrobromide n-propanol solvate #2, carvedilol hydrobromide anhydrous forms or anhydrous forms, carvedilol hydrobromide ethanol solvate, carvedilol hydrobromide dioxane solvate, carvedilol monocitrate monohydrate, carvedilol mandelate, carvedilol lactate, carvedilol hydrochloride, carvedilol maleate, carvedilol sulfate, carvedilol glutarate, or corresponding anhydrous forms, solvates thereof.

8. The controlled release formulation or device according to claim 6, wherein the carvedilol salt, solvate or anhydrous form is selected from the group consisting of carvedilol hydrogen phosphate, carvedilol dihydrogen phosphate, carvedilol dihydrogen phosphate hemihydrate, carvedilol dihydrogen phosphate dihydrate, carvedilol dihydrogen phosphate methanol solvate.

9. The controlled release formulation or device according to claim 6, wherein the carvedilol salt, solvate or anhydrous form is carvedilol dihydrogen phosphate hemihydrate.

10. The controlled release formulation or device according to claim 1, wherein the outer coating further is coated with materials selected from the group consisting of a film coat and a pH sensitive polymer.

11. The controlled release formulation or device according to claim 1, wherein the controlled release delivery device has two face areas.

12. The controlled release formulation or device according to claim 10, wherein at least one of the two face areas contains an aperature or office.

13. The controlled release formulation or device according to claim 1, wherein the at least one orifice has an area from at least about 10 percent to at least about 60 percent in the face area(s) of the controlled release delivery device.

14. The controlled release formulation or device according to claim 12, wherein the at least one orifice has a diameter which is about 30 percent of the diameter of the controlled release delivery device.

15. The controlled release formulation or device according to claim 1, wherein the at least one orifice is an aperature, hole, passage way or outlet.

16. The controlled release formulation or device according to claim 14, wherein the orifice has an aperature diameter size range or orifice diameter size range from at least about 0.0 mm to at least about 7.0 mm.

17. The controlled release formulation or device according to claim 15, wherein the orifice has an aperature diameter size or orifice diameter size of at least about 6.0 mm.

18. The controlled release delivery formulation or device according to claim 1, wherein the delivery device is in an oral dosage form.

19. The controlled release formulation or device according to claim 17, wherein the oral dosage form is a tablet dosage form.

20. The controlled release formulation or device according to claim 18, wherein the tablet dosage form is selected from a single core tablet matrix dosage form or a bilayer tablet dosage form.

21. The controlled release formulation or device according to claim 19, wherein the single core tablet matrix dosage form has an immediate release core.

22. The controlled release formulation or device according to claim 19, wherein the single core tablet matrix dosage form has orifices or aperatures on at least two faces with a diameter range from 0.0 mm to 4 mm.

23. The controlled release formulation or device according to claim 19, wherein the oral tablet dosage form is a bilayer tablet dosage form.

24. The controlled release formulation or device according to claim 22, wherein the bilayer tablet dosage form has two separate sequential layers.

25. The controlled release formulation or device according to claim 23, wherein one of the two separate sequential layers is defined as a tablet core matrix.

26. The controlled release formulation or device according to claim 24, wherein the two separate sequential layers are comprised of an immediate release layer and a modified release layer.

27. A method of treating cardiovascular diseases, which comprises administering to a subject in need thereof an effective amount of the controlled release formulation or device according to claim 1.

28. The method of treating cardiovascular diseases of claim 26, wherein cardiovascular diseases are selected from the group consisting of hypertension, atherosclerosis, congestive heart failure and angina.

29. A method of treating hypertension, congestive heart failure, atherosclerosis, or angina which comprises administering to a subject in need thereof an effective amount of the controlled release formulation or device according to claim 1.

30. A method of treating hypertension, congestive heart failure, atherosclerosis, or angina which comprises administering to a subject in need thereof an effective amount of the controlled release formulation or device according to claim 28.

31. A method of delivering carvedilol to lower gastrointestinal tract of a subject in need thereof, which comprises administering a controlled release device or formulation or device according to claim 1.

32. A method of delivering carvedilol to lower gastrointestinal tract of a subject in need thereof, which comprises administering a controlled release device or formulation or device according to claim 2.
